SEQUENCE LISTING (353 sequences)

SEQ ID NO: 1 - DNA, 762 bp, Brassica napus
cttgtacagagcggctggtaccggtccgtcattcccgggatatcgtcgacccacgcgtccgtgatcgatgcgctgatcctgacaaagaacacagaaattcgcttgctttgctattgggaacgctgcttaccatagtgacaagctgtacgaagagggtggaggatctataacgcagctagcaaacgttttgacctcagccgaagaagacaagacaaaagcgaacgctgctggtgctttgagcaaccttgtccgaaactccaacaagctttgtgaagatatagtctccaaaggagctttacagacgttactgaagttggttgcggattgctcggctactgccctgaaccccagcaagaaagagagtggagagtgagtcaccgctcaagatagcactcttctcgctggctaaaatgtgttcgaaccaccagatttgcagacagttcgtcaagtcatcggagttgttccctgtcatcgcgaggcttaaacactcgcctgagactaacattgctcactatgcttcagttattgtcgccaaagttggtggtgattcttaagaaaatgagatgtacgtccctgtaaattcattatgttacaacactgtttcattacttcctagtattgttatttacgtttgagaaagagaaaaaaaaaaaaaaaaaaatttaaaaaaaagaaaaaaaaaaagaaaaaaaaaaaaaaaaaaggggggggcggccgctctagagtatccctcgaggggcccaagcttacgcgtacccagtt

SEQ ID NO: 2 - DNA, 605 bp, Brassica napus
gtgtgaaagacagagaaatgttcggtttgagggacatccatgagactcatcagagagaatcatccactcgcacgagtgaattagaagcccagctggaatcctcagagaagcgggtctcagatttgaatacgagtcttcagtctgcagaggaagaaatcaaagccatctcctcgaaaaatgtgaaaactctagacaagctagaacaggcgcagaagacgatacatgaactcatggctgagttgggagagttgaaaaaccgacatgaagagaaagagaatgagctttctagtttgttggaggtacatgaggcacatcagagagattcaacaagtcgagtgaaagaattagtagcagtggtggattcagcagagcaacaggttgcagatttgaaacagagtttggacagtgcagaggaagagaaaaagctgttatctcagaaaatatcagaaatctccaaacagattcaagaggcgcagaacaccatacaagaactcacttctgagtctgaacagttgaaagagagccacagtgtgaaagaaagagaattttctggtttgagggacatccacgagactcatcagaaagaatcatcccctcgcacgagt

SEQ ID NO: 3 - DNA, 651 bp, Brassica napus
gggcctgtgtctgggcctgttgttactgttaaggctggtgttggggatgatggtgaagagcaggaggtagaaaaagaggcaaccgcagaacttcaaactgatggagaagagcgagagtcggtgaaagtgccaaatgaagccaagtctgatggagaagatctaaagactgcaaacaagtcaactgcagaatctgatattgaaggagaagagcaggaggtagaaaaagaggcaactgcagaacctctaactgatggaggagagcgagagtcagtgctagagcaaaatgaagaaccggaaactgaggtacaagtgcgagattcagcaaaagagccagctgcagacacaaatttgattaaggaggatcgttctgaggagaaggagactggtaaagtcgaaaatgaagctgaagaagaggtacagaaaatagttgaggaactggaagaagacactgataaagcagaaggcgggactattcccgtttcaggttgatagatgaacaaaagcattttgttaattcagagaacgatgtcgttattgtggtgtacctattaactttcttgtttactgaagctgcttgtttagagacgtagtgttattgttagtaaagacttctggacatatatgtcttatcaaggaactagtttgatgcgt

SEQ ID NO: 4 - DNA, 910 bp, Brassica napus
cagcatgaagaaaagagagggattcaaaaaaggctggctccttagaagggatttgagtgctttagactaagaagtgagattagataggcttgtcttaggatttgatgcacattcaagggcaacttctgcttatgggtaggcacctagtgctacaatggctgtaacagattcagagattacatttgcccttggatacacaccacacattgattggatttgaaaggtacgaagggtccataacggacaggaactacaactattgatactcggtctagactagggggggggaaggcactgcaggaaacgggactgctgtactgtaaaggggttaccgtcgaaggtaaggacagagactgattaaatgggggaggacagatataggcagacacttccaaaaggcggaagcattggcttttaggacgcactcaaacttccattaaaactccaggtggcaaaacacaactccatccaataagaaagaggaatgtgctaacgccaataagcgtataagaaaaggactagaactggctgaaagcacttaccactagaaataaattggcctgacagaaaaagtcaaacccctcctctccctctcgcaataaatgctcgagttcccgaggaacgcctctcctttcagtcgagtcatgacttcgccctcaggtgggaggagaaactgtacaggacatagctttccgtctctttctttcacaaaaaaatgaaccaccaaaaactgtagatttcttttttgaaaaggccacaaaactcttttcaaaaaagaaagaagtgctactcaaaatggcggaactagatccttacggatcgtggattcggagtggcgctcgctttattaaaaccggaaagactttatcgtagaagcgggacttagaaaagtcctcaaatctctttctga

SEQ ID NO: 5 - DNA, 720 bp, Brassica napus
ggtggtttatcgatcctgaaccctaatggatcatcatcaagaggacagcgagaaacaagacgacgagaaggaagctcttgctcgtcttgaggagattaagaagtcaatcgaagctaagatggctcttcgtcaaagcaatctcaatcctgaacgacctgattcggcgtatcttaggactctggattctagtatcaagcgcaatacagcagttatcaagaagttgaagcagatcaacgaggagcagcgtgaagggctcatggatgatctgcgaagtgtaaaccttagcaagttcgttagtgaagcagttactgccatttgtgaggccaagctcaaaagctcagatattcaagcagctgtccagatatgctcactacttcatcagaggtataaagagttctctcctagtctaacacaagggctcttgaaagtattccttcctggaaaatccgccgacgacgtagatgcagacagaaactcaaaggccatgaaaaagcgcagtaccctaaaacttcttctggagctctactatgttggagtaatagaagatagtaacatcttcatcagtattattaaggaccttaccagtgctgaacacatgaaagatcgagatacgacccagacaaatttgacacttcttgccggttttgctaggcaaggaagagtttttcttgggcttcccatatctagacaagacgaagatttttttaagggggctcggcg

SEQ ID NO: 6 - DNA, 757 bp, Brassica napus
ggccgctgcaacctcgtctgtggaggttaaggaggacaagcaagaagaatcagtagctgagccactgcaggggaagaagaaggatgcaaagggtaaagcagctgagaagaaacttcctaaacatgttagagagatgcaagaggctcttgcacgaaggcaagaagctgaagagagaaagaagaacgaagaggaagagaaattgagaaaggaggaagaggagcgtcttaggcaggaagagcttgagaggcaagctgaggaggctaagcgcaagaagaaggaaaaggaaaaggagaaacttttgaggaagaagctagagggcaagcttctaacagcaaagcaaaagagtgaagctcttaagagggaggcttttaagaaccagttgcttgctaatggtggaggtctcccacttgcagatgagggcgagcctgccacaaagcgtcccgtttatgccaacaagaaaaagtcagctcgccaaaagggcaatgactctgcctctgttcaggtggaagaggaggtagacccaaaagagagtcatgcagatgagccagatactttaggtgaagtgggttcagcagaaactgaaaaggttgatttagtagagtcagcagacactgatgagaaatcaggtactgctgatgtagctgcagagaatggggctgaggaagatgaggaagaagacttcaaaatcgaaacagtgaacatttacttgtatctttattaaagccacttaaaatatttgattaaacgcctacttggt

SEQ ID NO: 7 - DNA, 1103 bp, Brassica napus
ggctgaaccatctacgatggggagagcgaatgcacagggaagaagcttacagaagagaatacgttctcgtatcgctttttttgtagaaagctcgaccagcgagaaaaagtgtttggccgaagccaaaaataggttgacccacttcattcgcttggcgaatgatcttcgcttcgcgggaacaactaaaaccaccatctcgctctttccattcttcggtgccacctttttctttctaagagatggggttggggtgtataataaccttgatgcccgggaacaactactcaatcaattaagggtcaaatgttggaaccttttgggtaaggataaggtaatggaattgatagagaaattcaaaaacctaggtgggatagaagaattgataaaggtaatagatatgatgatagaaatcatactgagaaagagaggaattccgtataggtacaactcttatttttacgaagtaaaaaaaatgcgatctttcttgtctaatagaacaaacactaagaccttaattgagtcagtcaaaataaaatctgtttatcaaagcgcttctctgattgctcaagacatctcttttcaactgaagaacaaaagaagatcatttcattccatttttgctaaaatagtgaaggagattccaaaaagggtggagggaatccgtatatgtttttctggtcgattaaaagacgcagcagaaaaagctcaaactaaatgctataagcatagaaaaacttcttgtaatgtatttaaccagaaaatcgatcatgcttctgtggaagtatctactcgttacggaatcttaggtgtcaaagtgtggatttcatatagtcaaaaaaaagggggacgtgctatatccgaaacgtacgaaatatagtaaatatcgtaaaggcagatgtagtaggggttgcaaaccggatggtacaaaactgggttttggaagatatggcattaaaagttgtaaagctggtcgtctttcatatcgagccattgaagcagcgcgtcgggctataatcggacacttccatcgtgctatgagcggacaattccgaagaaatggtaagatatgggtaagagttttcgcggatctccctattaccggg

SEQ ID NO: 8 - DNA, 701 bp, Brassica napus
cacctgagtttggaccaggtcgaccacgtccctcacaagaccatcatcaatctggccggtcttcttgttgatatacgccctcctcattagggcgagatcatcaaccggctcgccatcattttcttccgccttgaaaaaaaacataaaattaaagaaacattagaaattagaagaaatgcacaataaattaaaattctgaaactcaaataattgaagaaaaagcggttgaacttaccatgcgatctcccagagtggcaatagattgagcacccaagttatgcttgtagatgcccttccctttacggtcgctcctgcggttggtggagttggtggaagaagtttctttcgtctcctccttatcccaatgcgcacacaactccttccagaccgtgtcgttcatcgactttgggaccttttaataaaagaaaaaagaaaatagtttaataaattaaaaaatagtttaataaattaaaaatttgtttaataaattaaatcgaaccttattgatttcccacttcttcttccactcgtggatctgcttcccatagttgtccataactttatggacgaagtggtgatagataaaaagcgtctcatcggaattccagttgaactcttgctgaaaaaaaaacacaattagtagaaaatttatattaaagattaaaaatataaaaaaaaattagaatacttaccgcaaactg

SEQ ID NO: 9 - DNA, 713 bp, Brassica napus
ggtaactcgtttcacaatagaccacaaaactcagaagtgagaagttgcagtggagccattcgtctctctctcgacaaaatcgaaagctctctctacgaaacctctctaattgcatcattcatagcgtttatcaaaggtctcagcttcaattctctccttccgaatccatttcgaaagctcaaagtgaaatcaagatttcagattttatcggcaaaggagctctttttatcgctggatcgtggttctgatctctcctgcttgatcttcgattttcagattttgatctggcgaaggagtaagctttctggaaatcatagggattgagattgatttttagatctagggattgatggaagggaaggtggctagacctagaggtagacctaggaaacgccctagggcagaggatcagaacggtgtctctaaccgagggaagagaccggttctggagattaaaccggcagttcccagatccttactcggtagctacgtgttgaaagagtttgatgacagtagagtctctctagggaaagtagtttcctacagttctgggctgtacatagttgagtatgaggatggttgttttgaggatttgaaaacttgctatcttcggaagttgattattggggatggttactttgatgatgagttgcgttgtagaagaggtaaactcgatcatcttgtaatgaaaaaaaaaaaaaaaaaaaaaaaaa

SEQ ID NO: 10 - DNA, 729 bp, Brassica napus
gctcttggcaagtaggagacttggttgattgggatagagatggatgtttctggtgtgcaacggtcttgacagtgaagagaaacgaaccattccagattgagttgtttccaccaccactcggtgaaggagaaacttataaagctttgcgtaaggacgtgcgtccaacattggattggtctcctgaagatggctggacactgccttctacggatggacgaaaaagtcaaagtgttaggctcgtgaaacgtgagaaaggagtagatcatgtacctggaaaggagaaaacagagagagccaagaagcagaagacagagagacacacgcagcacaggactgagagagacgcacagcagaagacagagggagacatgcagcaagagaaaggcaacggagagattcgacaaaatatcgatgagtcggattcaattgaagctgcagtttatgacttggaagagcttatcgttcgaattgagtggataaaaaatatgctaagtccagatgttggagaaggttcaacgtggaaatatcaagactaccgcccatcttctaagtgaatgtgaattcatcatggtggtaggtttaggaggtgaaactgctggtgtgctactgaaagtttgttaaaaaaattttcaagttcgatggtgaaatggggaagagacgtagagttttgtgcaacaccagaaaagacatgatgatgtaggctgtgtatatgcttttccaagcgcccaa

SEQ ID NO: 11 - DNA, 592 bp, Brassica napus
gaactggcatcacacgcggtttcacaagctggaatacttgtttcaatgggtcatcctccgtcgctcaataagctggtggaggtgggtccaagtaattattggaggcaggctcaagaatctgagaaagtgcagccttgtaaagtgggtgtattagaaaaagaaactgagactactagtgatagaggttttgctagtcccagcactgcgcatactgagttggatggctccgcgagaacagctgatggtctaggtctaggtctaggtctaggtctaggtctagtttctgccactggaaagaaaacaaatggacagaaaggtcatataagtgcagacgtggctaaacacactgctggagttttcgagccagaagttgggtcaaaatcttcgattgacacccagactgaaagtgagcagattatgagaaaagcaaacgacgagtgcatcaaggaaggttctcatgtagaggttttcaaggaaggacctgaattaagaaccgcatggtactctgccaatgtattgagcttagaggatgggaaagcttatgtgctgttcagtgacctttctgtagaacaagaacagataagctgaagga

SEQ ID NO: 12 - DNA, 261 bp, Brassica napus
taatgtggacaataagtgattttccagcatatcctatgttgtctggatggacaacacatggaaggctatcatgtccatattgtcaagataacactaatgctttccaactaaaacacggaaggaaaacgtgttggtttgactgtcacaggagatttctaccaccagatcatccatatcgtaggagtaggaatttgtttacgaagaacaagagggtgtttgacagtccacctccggaaatttgtgggaaagatttgaagatac

SEQ ID NO: 13 - DNA, 717 bp, Brassica napus
agtgagcggcgtcccgatgagaactccgtctctcctcctcctcctcctcctggccttcgtatccatcattccttcgtcaagcagccatggcgaatggggtatcctcacggagcaaaacttctcttctcagatccgtctccaccctcacatcctcctcttcgtcaccgctccatggtgtggcgagtcaagatctctcaagaatgaaataactcagctggttcatactagtgacgagtatggctcgttgaagttgatggttgttataactatcgtgggaaaatccgagccttaaatatattgtcgtctcttcgtccttatttggcatctcctcctgaagaacttcctctcaagcatttgaagtctccagagagtttgtaggattttcttgaatcatctgacaaggctttgattctgttcgagttttgcggatggactagtacacttctctctgagttgaagaggaatgtcactgaagataacctttggcatggaaacttctccaaaaaagttgaaactgaccgagtgctgaagttaagaggaaaaaataaccagaaggtggcgggagaatgacagagcgaacgggggttctggacagacatgtaaccatgaacagttcaagcaattcagttcattcctctcaaaattgattgccgccgcaaaagagttttccctgcctcctggaaggcaaaaatttggtctgatcacagaaaaatcgct

SEQ ID NO: 14 - DNA, 811 bp, Brassica napus
gagccgcagaatgtattttcagttccaggagcagagccaggggagccttcacttttggcacttgaaaatgccaatggaaaatcgctggatgttgctccttttggtgttccggtaagatcagaggaaatgttcatgcctattgctcctgagatgaagttttcagagcctgttgcattatcacttcatgagacagagcaccttgaacctcacatggaagatcaaaaccaacctttgcctgaaaatcccattgttcacgagattccatctgatgttgaagctccaacggaagatttagctaaaacgagaatcaaatcttccaacaaatgaaccacaaaaagaacctgatgtagtgcctgttgctcaagagatgaagactgttatacggatcaaggtcagaccatctggtgcaaccagcagagccgaaggatcagcacgaacggtggaaagatcgcaagggattgtggtacgccgtgatatagaacgaagacaaactagttcagcttctgtagacgctccacagagaatctcagctgatgctcacatggaggaagtaaactcgtgtcatgatgttggatctcgaatgaccgcaagtatcggcagtgtaaaattacccaccgaaggtgatacttctgggaaagaactgcagtgcactgctgaatctgggaaaacttccacaagccagaaagcagataatgataaccaagaagtcgcagcacctggcttgttgccacaagacaataataacatgggaaacgaggcgcagcagaaatacggtagccttcagacactctcggttggtaaagaaagcgaa

SEQ ID NO: 15 - DNA, 2293 bp, Brassica napus
gttctagatcgcgagctacgagcaaataaaacacctttcaacagtatcaataccgtcacatgaattgtaaatgcatgaatatgatgtaccaaaaagtcggccgttcctaatggaataggtagcaaagctactttgccacccactgctactaactcaccgcccccccaagtcaaactggtgctcgctgtttcaccaggggctgttacaccaggtgctaaagcatgggtattttgtatccattgagcaaagactggttgtaattgtatagcagtatctgaaaacatatcttgtggacgccctaaagcactcatggtatcattatgaatatacaaaccaaaactgtggaagcctagaaatatacatacccagttgaggtgtgatatgattgcatcgcgatgcctcaggacacgatctaataaatcgttgtatcgattagttggatcatagtctcttaccataaaaatggctgcatgcgcagcagcaccaactatgagaaatccaccaatccacatgtgatgtgtgaacaatgatagttgtgtagcatagtcagtagctagatatggataagggggcatggaatacatatggtgagctacaacaatagttaaagagcctaacatagccaggttaagagataattgagcatgccatgatgttgttagaatttcatatagacctttatggccttggcctgtaaatggacctttatgagcctctaaaatatcttttagaccatgaccaataccccagttggtcctatacatatgacctgctattaggaaaagaattgcgatagctaaatgatgatgtgctgtatcggttaaccatagacccccggtcactggatctaatccaccacgaaaagtaagaaagtctgagtattttgaccaattcaaggtaaaaaagggggttgctccttcagcaaaacttggataaagttgagccaaaagatcccgattcaagataaattcatgaggaagtggtatttctttaggatctactccagcatttagaaattggttaatcggtaaagatacatgtacttgatgtcctgcccacgaaagggacccaagtcctagtagccctgctaaatggtgattcaacatagattctacatcttggaaccaagccaattttggagctgctttgtgataatggaaccaaccagcaaaaagcattaaggctgcgaagaccaatgcgccaattgcggtacaataaagttgtaattcactagttattccagatgctcgccaaagctgaaaaaagccagaggttatttgtattcctcggaagcctccgcccacatctccattcaggatttcttggcccactattggccaaaccacctgagcactaggtccaatgtgagtaggatcactcagccatgcttcataattggaaaaacgagcaccgtggaaatacatgccactcagccaaagaaagatgatagagagttggccaaaatgggcactaaatacttttcgagagatttcctccaaatcactggtatgactatcaaaatcgtgagcatcagcatgtaggttccagatccaagtggtagtatcaggtcccttagctattgttcttgagaaatgaccgggtttagcccattcctcgaaagaagtttttatgggatccctatctaccaaaattttgacttctggttccggcgaacgaataatcattgagtcctcctctttccggacaacacatacaaagaaacccgccaacagtcactcaaataattagtgaaccgatgatagatgcttagaattttgttctttctcttctatctcccatctattcatccattttctttagttattcactagagcaattatgatctggaagtcgatctggggcaagtgttcggatctattatgacatatccatagggtgctcaacggaccccccccttttttttttttattaaaaagcgttttcgcacctttacattagtattggtacacaaataatttttttttataacctaatctagtgtattcatatttcaattataagttccgaaatatagcctattttttatgttttaaatagaggatattatcctatttcaataaccgcttattagtcattactaagaaacattctagtattgatatttagtcattttcaaatcccttttattcgttttaatagtcgaaaagaaaaaaatagaaaaaactagatatagatattatagatattctcatattcatgtactacttatccctagagaataccagattaaatagaacgatttgagaaaaggatataatgaaatttttttctg

SEQ ID NO: 16 - DNA, 637 bp, Brassica napus
aagggggattattccgagcgggttccatggaccatggggatggaatagctgttggatggttaggacaccccgtctttagaaataaagaagggcgtgaactttttgtacgccgtatgcctactttttttgaaacatttccggttgttttggtagacggagacggaattgttagagccgacgccccgtttagaagggcagaatctaaatatagtgtcgaacaagtaggtgtaactgttgagttttatggtggtgaactcaatggagtaagttatagtgatcccgcaactgtgaaaaaatatgctagacgggctcaattgggtgagatttttgaattagatcgtgctactttgaaatccgatggtgtttttcgtagcagtccaagaggttggtttacttttgggcatgcttcgtttgctctacttttcttctttggacacatttggcatggttctagaaccctcttcagagatgtttttgctggtattgatccagatttggatgctcaggtggaatttggggcattccaaaaacttggagatccaactaccaaaagaccagccgtctgatgcaaccttgctttttttcttttagtttctgtttgcgatttttttgatttcctttaataggtagggtactg

SEQ ID NO: 17 - DNA, 555 bp, Brassica napus
cccacgcgtccgttgatgacagggcgttctaatcttcagccaaaagttgtcaacgatgaggaagagacatactgtatacacaagatcaactaacgggtattcacttagaaaatccaaggtcttaagtattggtgggtcgcatttaaaatggtccaagtccattgagagagactcgagaaaagctaatgaggaagccactttggctgtggctgcattttcaaagaaagaaaatgaaaagcattctggacaaagtagtactaggaaggcaagcagaaaccatctgacacgggagcgcattttcaggtttggttcccttcgctataaaatggacccttcaaggcgaactcttcagagaatatctgatgttgattcaccgtgctctggacctactgaaaatggaaaagccgcaaaaagaccgttcatcccaaagagattggtcataggccatgaagaatatgtacgtgttgggaatggtaaccagcttgtcagagatccaaagaaacgaacccgtgcgttggcaaatgagaaggtcagatggagcctgcataatgttcg

SEQ ID NO: 18 - DNA, 679 bp, Brassica napus
ggttttggttagtataatggaggataacaagctaggtgaagctgcagaactggctagtcagttatgggaagctgatatcaatacagagatcgtgggaaagataaggagagggaaaaggaaagagataaacatagagacagagagaaggagaagttgaaagagaagtctaaggagaaggagaaggaagcgtataaagacaaggataggtcaagagttaaggatagatcgagtaagaaaagctacgaggatgaggatgagaatgaaaaacctgcggaaacaaaagaccattatgacagtgaggataatgctgatgctgcaccaagtgggaaagagcaatctgcaacggaagtcgaggagcgcattaagaagatgaaagaagaaagaaagaagaaagctgaagaagcttctgatgctctatcatgggttgcaaggagccgtaaaatcgaggaaaaaagaaatgctgagaagcaaagagctcatcagatgtccagaatttttgaggagcaggacaatctgattcaaggtgaaaatgaagacggcgaggatggtgatcatctatctggtgttaaagttctacatggtttggagaaggtggcggaaggtggagcagttattttgacactaaaagaccagagtgtccttgccgatggagatgtcaacaatgagattgacatgctgg

SEQ ID NO: 19 - DNA, 844 bp, Brassica napus
ctcggtcaaaggaaaaaagtggaagctgaggaaaggcgatggagacagaagaaagcgcgggaagaatttgtaaagatgctagaggagtgtgaagaattatcatcatccatgaaatggagcaaagctttgagtttgtttgaaaatgatgagcgttttaaagctgttgatcgcccaagggatcgtgaagatctttttgacaattatattgtggaacttgagaggaaggaaagagagaaggcagtggcagaacataggcagaagatggcagagtatcggaagtttcttgaaacctgtgactatatcaaagcaagtacacaatggcgaaaaattcaggatagactggaggatgatgaaagatgctcatgtcttgaaaaaatagatcgtttgattggttttgaggattacattaatgacctggagaaggaggaagaggagctgaagagggtagagaaggaacatgttaggcgggctgaaagaaaaaaccgtgatgcatttcgtacactactggaagaacatgtcgctgctggcatccttacagccaagacgtactggttggaatattgcattgaggtaagagacttgcctcaataccaagctgttgcatctaatttgtctggctcaactcctaaagacttgttcgaagaaatcacggaagaattagagaagcagtatcacgaggacaagagtcgcgtaaaggatgcgatgaagtcgaggaaggtttccatggtctcctcatgggtgtttgaagattttaaatctgctctttcagaagatctcagttctcaaccaatatcagacataaatttaaagcttatatataatgagttggttgagaggatgaagg

SEQ ID NO: 20 - DNA, 699 bp, Brassica napus
ggatatcgacatgaattgcagacttctttgcaaatggttcctccaggtaagctagctagttggagatactcaaattcctggcaatgtttcacgttgtaatcttcttcttgtcacttgacttttaggagagtctccttcgtcttctgcctcggatgttgacaacgtgaaccatccagcaggaggagcagataaagttggaaactcaagggggatcagctctcctttagttgctgggaaccacgccatttctgcgcaaaaccgctttaatattttacgtctacttcaatttgtaagtcttattaatgcctcttgcaacttagatatatagagttggtgaaaatggaagatgaagtgatatgattcattactcttaaactgtttgtaggctcagaacgtgaacctgggaatggatgcctcgaggaaatcacgcgtggcattggcagcttctgttgaaaacctggaggaggcccaacaacaaggagaggggattctatccattaaaagcgccctagattacaacttccaagacttggaggggttgctacgtttagtgaagcttgcaatgaaggccaataaccggtgaaagacaagctaaaaccaagacttgcgactccactgatggtgaaaagaatcatctgtttgccttgcgactccacgtgcgttttgagcatttgttttggatcccataatcattcgtac

SEQ ID NO: 21 - DNA, 676 bp, Brassica napus
cgaagtctaagcaaagagaaagcatctcgcacctccaaagccacggtgaagatttagttgggtcgagggtcagagtctggtggcctacagataaagcgtattataaaggtgtggtcaattcatatgattctgctaagaagaagcacctggttatctatgatgatggagatcaagaaatcttgaatcttaagacacagaagtggcattttctggatgaatcagaaacagagggtgaagaagctgctgatcagacggttcacgagaaagaagcctcccgagagccccagagaaagaaagctaagactggcaagcaatcaaagatggaatcgtcagggaaaaagggtggtggagctggttccagcaagcttaaagctgctcctgcttccaagtccggtaagaagtccaaggatgagaaaacagagagcaaaccaaaggatccgaaagaagctagtagagaagaagaagaagaggatagttctgacgagttgagcgaggaagaggaaatccccaaaacagttggtaaatcaggaaccagcaagtcgaagaaagagatatcgaaatctggcacatccaaaggttcgtccaagactacaactactccaaaatccaagccaggaggaccttcaaaatcttcatcagcaaagggcaaagcagcgaaagggaaagcaaactctac

SEQ ID NO: 22 - DNA, 565 bp, Brassica napus
atcccaaatctagggttcggtttctgggcggcgtgcaatccaatggcgagtgtgtacataccggttcagaattcagaagaagaagttagggttgttcttgatcagctccctcgtgatgcctccgatatcctcgatatcctcaaagccgaacaagctcctctcgatctctggctcatcatcgcgagagagtacttcaagcaaggaaagattgatcagttccgtcaaatattggaggaaggctcaagttctgatattgatgagtactatgctgatgttaagtacgagaggatagcaatcttgaacgctctcggtgcctattactgctaccttggcaaaaccgagactaagcacaaagaaaaagaagattattttatcttggctactcaatactacaacaaagcctccaggatcaatatgtgtgaacctaccacttgggttggcaaaggtcagctcttactggctaagggtgaaatagataatgcatatcaggcatttacgattggattaggcacttctgatgacgttcctgctcttctgggtcaggcttctgtggaatttcaccgtg

SEQ ID NO: 23 - DNA, 404 bp, Brassica napus
agggacaaggtggagattgtaggaagtgtccctatatcatattacggtctctttggtgaggagaatatactgattgaaggagagggtttagacttatgttaagtgtaacccgagtcacgtgaggcacgtgagaactatatgtaaaaggctgtgatataaagccttatcttcttcgtcgttttctttagagtagagagaaactgatctgagattgtaacaaatcatgtagacgactgagagagagaggagaccaccattgtagaccgatgagagctcaagtttgagttattgtagtggattgctggctgtgcccagccccggtgatgtatagcgggttctccacattggagaagtgcggtgagcaccgggaaaaaaaaaaaaaaaaaaaaaaaaaaaaacatgtc

SEQ ID NO: 24 - DNA, 897 bp, Brassica napus
  misc_feature (710)..(710): n is a, c, g, t or u
cgagctgatacccgctcgtattaaccctactaaagggaacacaagctggagctccaccgcggtggcggccgctctagaactagtggatcccccgggctgcaggtgccaatgacaacttcacttttgtggatgctaattcactgactccaccttcaggagttactaaccagcgtgatgcagatcttgtccatttctgggataagtaccgaaaggcaccagaaggttccacaaggaaaacagaagctcagaagcaagtccttgaagccatgtctcacagacttcatgttgacaacagtgtgaaactcgtcggcaaactcttgtttggtatctcagaaggttctgaagtgctaaacaaagtaaggcctgctggacaacctctggccgatgactggacttgccttaaaaacacggtgagagcttttgagagacactgtgggtcgctgtcacagtacggtatcaagcacatgaggtcatttgcaaacatctgcaacgcagggatccaaatggagcaaatggaagaggcagcttcacaggcttgcacctcaatcccatctggtccttggagctctcttcaccgtggattcagtgcttaaaacccctaaatccctctttgcatttgtttaatactagtaccaatacccaaatattcctatttcgattcccctgtatatctcattatcatttccctcttgattatctatttgctttgnacaaaagcttgtagaaactcaagttaatgtaaacaaaggtttgcttatttatataccttcttctttgcaaaaaaaaaaaaaaaaaaaacttcgagggggggccccggtacccaaattcgcccctataaggagtcgttatttacaattcactgggccggcgtttttacaaacgtcgggacttggaaaa

SEQ ID NO: 25 - DNA, 716 bp, Brassica napus
gaacttgaatcaacaccagacaaagaagaccatggcacatgtaacggggatgcaggcaataacaacatcactgggaagaaacgcacttttactgagagcacactaactgcagagagtttgaactctgttgagtcagttggactgattcagtccaagagaactgcacattctgttcctgatgacgatgacttgttgtcttctatcttagttggaaagtcatcttttctgaagatgaggtctacacctgtgcttgaagtagcaactacaaaacggttgaagtctgctcctcgctctactaccacaaagaggaaggttctaatggatgaccctatggtcttgcatggcggccttatacgggaacagctgacaaacacggaaggtatacgccgtgtgcgtaagaaggcaccttgcaccatccctgaaatcttgatgcttcaaagacaggctttggatgaggatgggctttttaaggagccaatattcactggtatgtcagtggaattagtatctctgcacaatgagccgtatgatttaagaggaatcacagtaatcaaggaggatgacggccatgcttctgtcgaagtggtggaaggtaatgaatgctcagttagagctgtggaagaaaaagaagctgaggaaaggtctgctcctcctcaaacacacccaaatgattccgaggagcaacctgctgaggctcatactcagccacaggatca

SEQ ID NO: 26 - DNA, 937 bp, Brassica napus
ggaggggaatgatttggtacccaagaaagtcgtgtgaacgaaggcataaattattacggacatgcctctactgtcgcgagccttggagattctgatgtccattgagaagatagattatctcctcctctgagattctttcgttttaaagtgaaaagtaccaaacgcgccaacaaactttttctctctaacttttcaccgtgtgttcttaaggggtggtggctctgcggcttccggcgaccgtgccgaagcttgtagcggtcaccccctctcggatcttcgcgcaacaagacggagttaacagatctgtcgttctttggaggtgcctcttcaatctcatggcgaatcgggatggtcttggagcgtcgaccaccacgagaggctcgttgctctggtggctcgtcgtgagtacggcctcgctcccgttatgaggacgacgggagatgccggaacacgcaagttagtcatcggggctcgtacctcgtcaccgattgcgaagtagggttttggtgtcggcggttcttcatgagagatggcggttctttctggatgtttctcgccggctctggaacctcttcgtctctgccttgagtattgtagaagcacgccgttactcggacttcgaaggaggtgaagctggagcagatagcggcggctagggtttggagctagtcttgagctagcgcgttggggccgcgggacacctccctgggtggtttaacggtctctggtgacggatctggggccggatttgaacaccccggcctcgaaaagttccggatctcagatctgaagaccaccggtgttccatggaggaggaagcttgcttcccggtcttctttcccccaaccctatactctttttgcttagatctgttattagtgtttttgccttctatgtatgtaatctagccggatgtttctctgagtcgccggaaatg

SEQ ID NO: 27 - DNA, 629 bp, Brassica napus
ctctggcagcgagcaagctctggaagtagatagtatggctcttgttcaggaatgctctgatccagctgaggcttttggaaagtgtgtgacacctgattctagtgatgtgataggtcgggaagaaaacttagatcgtgatttgaagtctgtatcgatcagtgttgccgatgaagttgagagggaatcgaagtcgtgtggtggtacgcggataactggggaaattaaggagagtgttcagctgggtagatcttacgggcagaaccgtgattacgaggaattcgagtttgctgctacgaagccagatccttttgtgggaaagggaaacttgtactctcaaagtggcatacctgaagatcttcagtcaaactctttggtcagcgggatggagaaggcagaagacggagctgggtcagactctttgcagaagtcacagataaggcaaaatcatgatgtctttatattattgtttttttttatttcgtggtgcttatatagcagcagcaggttgagcttaaagtccactgtgtgaacgtcctttaggctattgacgttataaccaagactcaaagatcaacttgcggtggacagacgacagagtgcaaaaacctttggtaagttaaaattttatg

SEQ ID NO: 28 - DNA, 744 bp, Brassica napus
ggagaggcgctccaagaggcgtactgatcgcaacgcctgatcggtaatctctcttggaattattttcacaccgagacggtgtgaagtaagcctgggggaggatgctacttatgtaccatattgcttttatttcttttattttctttgtgttttaaaaaaactctctacgtatttttctcagaccctgtgatgattattagactacttccttgcgtgttgtgcattacatttcatattgaccatttttcagaactgttagcgcagacaaaccgaggaaccacttgaccaaacaacctctccttaaatattttatcaagtctcggtgaattgtaatcgactcaactatttttgaccattaatgaatctaatttcttttgaccaggaatcaacatcaggaaacggtacaccatatacacattcaggattttctttaccacattttaccactctttaataatcctgaatggccagactcatcaatagtttggtaccacacctacaccttacccttttatttcatctccatgcattcttacacactgatcatatgtgcatacatgtgcaaaaacaatggagagattagggtagacatggttcatccgactgcttaggtaggatcggaacatttaggtggttagacacggacctgtgtgtctagaggtgtaaatagaaaaattgggtgttgtaagtgtgtgattgcatcgcagtgtatatattcgatttcggtttgtatatattgtttgc

SEQ ID NO: 29 - DNA, 693 bp, Brassica napus
gtggaaggaaaagacagcatactgaggatactgctgctggtgacggacgtcgtaagagacagcagactgttgctgtcttgccgcagacccctggccaaaaacgctacaatctgaggcggaacaaagccgtagaccaagttccagcagatgttaaagataatgcagctggtggtgaagatgatgcagacattgctgcctctgcgccatcaaaggacaatgtcgaaggaactagcgaatcggtggtggaacctcttccagctagaagattagaaagttcagaagttagggttgagagagttgtaactgtggaaacaactactgctactgctaataccaaagtgggtgtttcggttgcaaatacagaagtagcacctaacatcgctatgagcccctctgttgaagatgatcagacgcagagaacagtcaatgaggataagaatgaagaatatgaggatggaaatgatgaagaagattacgaggaacaagaagatgatgacgatggtgatgatgatgatgatgatggatcaccaagacctggtgaaggatccataagaaagaagctgtggacttttctcaccacataatcatctcacgtgggatgatccaagagatgttactttcacatttattatgtagaaccattatagcttctttgtatccaaagacacccttttgatggaagatgaagttagg

SEQ ID NO: 30 - DNA, 484 bp, Brassica napus
tggaagggaattattgacggcttatacgcacttctaaacacactgaaggagaactttgttcctccggttcttatccaaaagatatttactcagacattctcattcatcaatgtccaattattcaacagccttcttttgcgacgagaatgttgtacatttagcaacggagagtatgtaaaatctggtttaacattgttagaggaatggtgcgccgaaacaaaagaagaatatgctggctcatcctgggatgaacttaaacatatcaggcaagctgttggatttttggtaatccataagaagtacagaatctcgtacgatgacattgcacttgatctatgccctattcttagtgtccagcaactttacagaatatgtacgctgtactgggatgacagctacaacacaagaagtgtatcccaagatgtgatttcaagcatgagagtgcttatgatagaggattccaacgatgctgacagcagcgcct

SEQ ID NO: 31 - DNA, 1011 bp, Brassica napus
  misc_feature (729)..(729): n is a, c, g, t or u
tggaatgagactcagtcgtattaaccatcactaaagggaacacaagctggagctccaccgcggtggcggccgctctagaactagtggatcccccgggctgcaggtgttgatggtgtcaaagagaaggttggtaatttcagagttgaaccgcctgggctcttcagaggccgtggagagcatcccaagatgggaaaattaaagaagcgtatccatccatctgatatcaccatgaatattggaaaaggttttcctgttccagaatgtccaatccctggtgaaaaatggaaagacgtaaagcatgacaacacagtcacgtggctagctttctggaatgatcccattaacccaaaagaattcaagtatgtgtttttggcagctagcagtgccttgaaagggatgagcgacaaggagaagtatgagaaagccagaaatctgacgaaccacatagacagtataagagcaacttacaccaagaatttcacatccaaggatgttgcaaagaagcaaattgccgttgctacttatctcattgataaactggcactcagggctggaaatgagaaagatgatgatgaggcagacacagttggttgttgtacattaaaggttggaaatgtggaatgtattcctccaaatcagataaagtttgactttcttggtaaagattcaatccggtatgagaacacagtcgtggttgagcctcttgtttacaaggccattggacaattncaggctggaaaatccaaagaggatgatctanttngatgagtagacacaagcaaattgaatgctcatcttaaagagcttgtacctggtctcacaggctaaggggttcggacataatatggcttcattcaccattgaatgaacaantgaantgaaaaagaagggagcctttagatgtttacttcaaaaaggtagtggttaatcagccagccaaaaccaagaagggtggccataatttggaaatccaatcaccgggcttggttcaaagtcccccaatggccacaa

SEQ ID NO: 32 - DNA, 767 bp, Brassica napus
gagccaactgcggaactcaaaattgatggagaagagaaagagccagtgaaagagattaatgaagaacctgaaactgaggcacaagagggagaatcagcaaaagagccaagtgcagacactaaattgatcgagaaggaggatatgtctgaggtacaagaggtagtagaatcagcgaaagagccaagtgcagacacaaaattgattgagaaggaggatatgtctgaggtacgcgagggagaatcagcaaaagagccaagtgaagacgccaaattgattgagaaggaggatatgtctgaggtacgagagggagaatcagcaaaagatgcaagtgaagacgccaaattgattgagaaggaggatatatctgaggaccagagtcatggagctgcaaaggagccaagtgtgactgagactggtaaagtagaaaatgaagctgaagaagatgatcagagagcacttaaggaagtgggagaagagactgataaagcagaagctggtactacccctgtttcaggttgatccattgggccatgcatggttaggtgatgataaagattgattgttgtggtttaggtaacttttatgtttagtgaagctgcttgtttaaagacttatggtgttattgttagtagagagagtcttctgacctattatgtcttattgtggaactagtttgctggtgtcacctagctaagtaacatacattttagaggacgtgtgtgtgtgcatttgctaaacacaaaacagtgataatatgtgtagtcatt

SEQ ID NO: 33 - DNA, 811 bp, Brassica napus
ctcttttgctaaactgttacactaaactgaaggatgtagaaaagctgaacacgttcattaggaaggaagatgggatcggcgagctcaagttcgatgtggaaacagccatcagggtctgtcgcgcagctaactaccacgagcatgcgatgtatgttgcgaaaaaggcaggaaaacatgagtggtatttgaaaattttgcttgaggatcttggaaactacgacgaagcgttgcaatatatttcgagtcttgaaccaagtcaagctggagtaacaataaaagagtatggtaagatccttatagagcacaagccaaaggaggcgattgatatactaatgcggctttgcactgagcaaggaacttcaaatggtgtttacctgtccatgttgccgtcacctgtagacttcatcaatgtgtttgttcagcatccacattcgctgatggattttctcgagagatatgctgaaatagttaaggattcacctgctcaagcagaaatcaacaacacgctgttggagttatacttgtccaaggacttgaactttccatcaatctctctatcagaaaatggtgtagatcagaatttcactgatcagtcagtagcagctgcgatgtccaaaactggttctggaaagaagaaaattgctgattcaaatgatacaatagaaaaggattgtgtggaaagacagcaaaaggggcttgagttgcttaaactgggctggccatcagaccaagagcaaccactttatgatgttgaccttgctataattctctgtgagatgaattcgttcaaagaaggacttctgtatctgta

SEQ ID NO: 34 - DNA, 1065 bp, Brassica napus
tgaatggctgatacccagctgtattaaccctactaaagggaacacaagctggagctccaccgcggtggcggccgctctagaactagtggatcccccgggctgcagggtgcgagcggatagtcaagagggagaagttaaaacgagagttggctatttcctcacatgagatacttgctgctagaagggaacacgctgcacgttcattaccactccgtaatccattttctcctccagaagtttcgtctgactcagctacgacgtcaataaaagggcatccggatagtaatgtatctggcagtgaagcgatacagaggtcagatgatatcaccattgacagcacagcctccgttaagcagcgccgaggcaaaggtcctattgcaatggatactgatcagaaaacagacgatagtgctacttctaaaggtcggagcaaaatattatctgggaaaacagttccacggaagcattgtatagtatcgccaagtgtttcagaggatggagatgaggagggatcaaagcccaagaaggtgagaagaatccctttgtcctaagtttactctttgttccgctcaactcagctcattcatgaaaactgataatagcagcatgtagaaacgtttgcgaaggagattgtgatgacatcggacgaagcttctttcaagaatccgcggctgccaaagggctacttttatattccggttgattgcttgcaagaagacaaggcgagcaatgagaaggcggaggaggactcgtctgacaagtgaatcaacaactacaaagctgctgcttttttcaggtgtgaactatgtaagcccagggggttcacgtcagtttttggttgacttaaccggccgaacctggaaaactttttccgggccattggaaaaccggcgggcattgggggaccggcggtttgggggattaattaatgttccccagaaaaatttggagaaaacccaaccaccttttttcttgggaaaaaaaaggcccccaattgaaaaaaccccatttgcaagaaacccgaaaaaacccattttccttttgggggtttttgggggaaaaaaa

SEQ ID NO: 35 - DNA, 460 bp, Brassica napus
cccacgcgtccggtccatggcaaccacaaaagccttttgtcaattgaccataggaaactgccctccacgccaaactaaggttgtgagcaatcgcacttctcctgaatggaaagaaagctttacttgggcatttgatgttccacccaaaggacaaaagcttcacatcatatgcaagagcaaaagcacattcgggaagacaactctgggccgtgtcactatccaaatcgacaaagtcgtgatagaaggagtttacaacggatctctaagcttgaatcatgacaacagcaaagatgcatcttccagaacactcgacattgagattgcatggtccaataggacaacggatgatactctttgagtgaagaaaacaactccatcttcacaagcacaatgcatttggtctaaaggaaggaagactttatatatctaaggctcccaatggctgaattgtggtagtaga

SEQ ID NO: 36 - DNA, 763 bp, Brassica napus
ggcgcccaaagcttcacgagggactgctggaaagccatctagatcaccaggtgttgcgccgcagcaatcaccgagtactcaatgggaaaacatacttaaattcttggattctcttatgtcccgcctgcgccaaaatcaagtaccctcgttcttcattcgcaaacttgtgactcaggttttctcatttatcaacttatcacttttcaacagtcttctccttcgacgtgaatgttgcacattttcaaatggagaatatgtgaaatctgggattgcagaattggagaagtggatagccagtgtcaaggaagagtttgcaggaacttcttggcacgagttaaattacattagacaagctgttggattcttggttatacaccagaagcgaaaaaaatcactggaagaaatcaggcaggacctatgcccggcattgacaataaggcaaatatatcgaataagtacgatgtactgggatgataaatatggaactcagagtgtcccaagtgaggtggtttctcaaatgagggtacttgttgacaaggatagccaaaaactaacatccaattccttcttgctggatgatgatatgagcattcctttctctgcagaagatatagataaggctattccagttttagacccatcagaaatagaaccaccgaagtttgtatcagaatatacttgtgcacagtcccttgtgaagaacgcctctgcagctacagcctcacagtagatcatttgatgcatatgagcttgattatgcatc

SEQ ID NO: 37 - DNA, 733 bp, Brassica napus
catttactggttcatcggcacctgaagccaggaagatagattctatgtgcgatggttcacttagactaatggcttttgagcgagaagaattatggacacgtgccaatgaacgtcttccttggcatgaaaaaatggagggagaagattgcattcagaaacaagtacacgagctgatatcagttggaaatttggaagcagctgttagttttttgcttacgtctgctccagattctccatacttctatccaaatgctctccgagccgttgcactctcttcagctgtgtctaaatctcttctcgaccttgcactgaaggttgttgcagccaacatggtaaggacggacaattcgctcactggcactcatcttctatgcgcggttggaagacatcaagaagcttgttcacagctacaagattctgggcgatggacagatgctgctaccttggctgccacacatttagaaggatcggattatgccagggtattgcagaggtgggctgatcatgttctccacgcggagcacaatgtgtggagggcacttatcttatatgtggccgctggttccctgcaagaggcgcttgcagctctcaagtaggtgcaacagccggacacagtagccatgtttgtgctggcctgtaacgagatccattcagaaatcgtaaacgagctatcaagccaagacgaagagtctactggggaattaggcacagtaatgacggatctgccaggtct

SEQ ID NO: 38 - DNA, 678 bp, Brassica napus
catcaaatatgcagctcctttctcttgagctcccgcaacaagagaattcgtttgactgtggcctctttttgctccactatttggaactttttgtggctcaagctcctgctaaattcaatccttcgcttatcacaacatcgggaaattttctaactaggaaatggtttcctgccaaggaagcttcacttaagcgtgcatacatcttagagttgctttacaatctccacaaaggtcatgatccaagtattattccagctaattccaaaagcaaaccacctcattgccgagtttccaacgagaatgatgaagaaaacgaaagcaagaatgtgactgagatctgtaaatggagaaaaccatttcatggttcctcagcaatcgtcccatacattcctcagacaaagaattgctcggcagatcaggttctcagcaaagaagttttttacacgagaggttatgacctccctgaggcttccaagcgccgaaagtcctttatgtcgcctatagtggaagaagttcaagaaagtggtgagaaagaagaaatccatttgccaatggacacagaggaatctatctgtcaagagatggaaacattgcgaaaaggggagtgcatgctctatatagaggacactgatgatgaagacgctgtagtagaatacgtttctgattcccaagattcat

SEQ ID NO: 39 - DNA, 602 bp, Brassica napus
actcacgtggagttgcttttgttgagttcaccgagcctgaccatgctcttgtggccctgagagtacttaacaacaatcctgagacgtttggccctcaacatcgtcccgttatagaatttgctgttgataacgtccagaagctgaaggtacacaaagctaaccagcagcagcaacgcaatagatacaatgaatctagggagcagcgagagaacggcgaagcgcagggagaagacaatcatcctggcaatgatctaaaacgaagaacgagggatggagataacactggttcagtggaagagaatgcaaacgggtataagaagaagaaacctatgcgcccacgtgaacagaggagagaagaatcaaagccagaggagaagagcagactctctgtgaaagaagatggagggaacaagaggcctacacgcactcaaggaaacactaaagaaccggcttcaaaccagagaggccaatggaagaagagacagcaagaggcttctgagaaaccagatgagaagatatccaaagatgtgagcgatgcgccaagaaagagaaagttttgaggaggttagaggcggagagaatgttaacgggcaaaggaagag

SEQ ID NO: 40 - DNA, 735 bp, Brassica napus
gtccgaatctctcggccgacgccggagtccttaaaggcctccggccggagctccgagaagctcagcctccccatcctccagggcaaaatcaagcgcgacccggatggatacgaaacagagctccaactaatctacaaacagttcaaagcctccgtcgatctcttccagcagcaagccgccctcagcttctcctccgtcggctccgacccttccgtcgccaaagacctcggcgaccgagccatgttcctagctcacgtcactcccttttaccctaagcagctcgcggagttcccagctcagctgactgatttgctccgcacctcgtgcctcgcgatgccctcggggctgaggaaccacgtctctcaggcgttgattcttctcatgaatcgaaagagtcttgtcatcgaggatttgctggctctgtttctggatgttcagtgtattggcgatagaaacttgaggaagcttgcattttctcacattgttcaaactattcggaagatgagtgttactgatccgaggcataagggacttcagaagattgtcatctccatgttggagcaagaagatgaaacaaaggctaagagagcacttgttactctatgtgagcttctcaagaagaaggtttggcttggtgatagacatgagagagttgctattgccatttgtgaagcttgctttcatgcttcgcctaggatcatggtatcttctcttcggtttcttcttgactacgag

SEQ ID NO: 41 - DNA, 946 bp, Brassica napus
tgagatgtcacactactatagggcgaattgggtaccgggccccccctcgaggccaagcgagaaagcggctgttgttggaggaaacgttctaacatctcagagagttacggatgttgtactaacagctttcaaggcttgtgcttgttcacaagggtgcatgaacaacctcacattcggagatgacacatttgggtactacgagaccattggaggaggatgtggagctggaccgacatgggacgggacaagcggaatccaatgccatatgacgaacacgcggatgactgatcccgagatcttcgagcagaggtacccggttctgttgcatagatttggattgagggagaagagtggaggcagtggcttgcacaagggaggagatggattggtgagagagatagagtttagaaagcctgttgtggttagtattctgtcagagagacgagttcactcgccacgtggattgaacggtggacaaaatggagctcgtggagcaaactatctaatcactaaagacaaacggagaatctatcttggaggtaagaatactgtacacgtggatgccggtgaaatcctacagattcttactccgggcggtggcggatttggctcagacgtttgagtgactcatcatcccctcgtaagtttcttatcatgttgctggtgtcttagtgtcagattattttggaagttgggcactttcagtgaaataaaagaagaagacctttggattcaagaaacaagaagctaaatttattctattcttaccccaaaaaaaatttaaaagggaatgaatccagaaatttctttcttcaaaaaaaaaattggtaaattggaaaattaaagcctcggtaaaaattgggaattttttaatggttttggggttcccccttcaaaccccattttttatggggcgaattttggataccaaaaaccggttttttct

SEQ ID NO: 42 - DNA, 785 bp, Brassica napus
gagtcttggaattcatgatctcataaactttgatttcatggaccctccacctgctgaagcacttgtgaaggctttagaacttctcttcgctctaggtgctctaaataagctcggtgaattgactaaagccggtagaaggatggcagagtttccccttgatccgatgttatcgaagatgattgttgtttcggacaagtacaagtgctcagatgagataatatcaattgctgctatgttgtcagttggagggtctatcttctaccgtcccaaggacaaacaggttcacgctgacaatgcaagaatgaatcttcacactggaaatatcggtgaccatattgctttgctaaaggtttacagctcatggaaggaaacaaactactccacacagtggtgctatgagaactacattcaggtacggagcatgaaaagagccagagatatccgtgaccagttggaaggacttctcgagagagtggaaatagaaatcagctcaaacatgaacgaattggattcagttagaaagtccattgtagctggctttcttcccgcacactgcgaagctgcagaagaatggatcatatcgaactgtgaagcatccccagacagtgcacatacatcccaattcaggcttatctcaggtgttgcaaagatggggtgtatatcatgaactagtgctcacctccaaggaatatatgcgacaggtaacggagttgaaaccagagtggttgattgagttaactcctcactactaccagcgcaaggacgttgaagatgatgcatcaaag

SEQ ID NO: 43 - DNA, 550 bp, Brassica napus
agacatatcaggaaggcagttggcttcctggttattcatcaaaagccgaaaaagacattggacgaaataacgagagaactctgtccggtgctaagtatacagcagctatacagaatcagcacaatgtactgggatgacaaatatggaactcatagcgtttcttcagacgtaattgcaaacatgagggttatgatgaccgaggactcaaacaatgctgtaagcagctcttttcttttggacgatgactcaagcattccattcacggtggaagacatatcaaagtcaatgcagcaagtggatgtaaatgacattgagcctcctcctcaactgatccgtgaaaactcaggtttcggattcttactcacacgtaaagaaggcagtgcgtcgtaagtgagctcggtacggtccaccaaatacgattgtttctgctgcatacataaatacatcgtgcgagcctgtgggcattttgtgtatattacttctcttgctttggtactcagtctctggcctttgtatttgtggaaaaggtaaagtttatttcattctttgc

SEQ ID NO: 44 - DNA, 727 bp, Brassica napus
gctccgtgctgggaacaagggtacttctgaagaagaattggagagtacccttgagaaagtcttggttttgttcagatttatccagggtaaagatgtgttcgaagcattctataaaaaggatcttgcaaagagactcctgttgggaaaaagcgcttccattgatgctgagaaatcaatgatctctaagctcaagactgagtgtggtagtcagtttacaaacaagcttgaagggatgttcaaggatattgaattgtcaaaggaaatcaacgagtcattcaaacaatcgtctcaggccagaacaaaattgccatcaggaattgagatgagtgtccacgttctaacaacagggtactggccaacatatccaccaatggatgttaaacttcctcatgagttaaatgtctatcaggatatcttcaaagagttctatttgagcaagtacagtgggaggaggttaatgtggcaaaactcattaggtcactgtgttttgaaggcagatttctccaaaggtaaaaaggagcttgctgtttccctgtttcaggctgttgtgttgatgttatttaacgatgcaatgaaacttagctttgaagacattaaagattccactggcatagaggacaaagaactgagaaggactcttcagtcgcttgcttgtgggaaagttcgtgtcctgcagaagtatcccaagggaagagatgtacaagacggggatgattttgattttaat

SEQ ID NO: 45 - DNA, 1046 bp, Brassica napus
cgatagtgatgcagcagtaccggccggaattcccgggtcgacccacgcgtccggttccttcaccaagtaataagataaattagtaattaaaaatatctagaatctatattatttataagggggcagaaataccttgcttacgtatcattaagaaatgcattaacataaatacagccgtaagaagaggtaatacaaaagtgtgtaaactataaaaacgagtcaaagtggattgtccaacactagcacttccgcgtaataattctacaagaggcgatcctattaccggaatagcgtcaggtacacctgttacaattttgactgcccaatagccaatttgatcccaaggtaaagaataacctgttacaccaaaagatgcggtcaatacacccagaaccacaccagtaacccaagttaattcgcgaggttttttaaaaccaccggtgaggtatacacgaaatacgtgcaggatcatcattaggaccatcatacttgccgaccatcgatgaactgatcggattaaccaaccaaagttagcttcagtcattatatattgaacagaagcaaaagcctcagtaacagttggacggtaataaaaagtcatagcaaatcccgtagctacttgtactaaaaaacaagtaagggtaattcctcctagacaataaaatatgttgacatgcggaggaacatatttactagttatatcgtctgcaatcgcttgaatctaaagacgttcttcggaccaatcataaaactttactcatattccgggaaatatacaatggcagaaaaaaaatttcggcggggcggaactaaccaaaagggaagaaatagggggcaaaaaattttttaaaacctctacattaaccttttaaatttttttttgggaattcggaaaaccaaaaaaagcctttggcacctttttcaggaatatccttttcttcaagggcctaaaatttcaatttgggaaaattttcccatatcccaagttggaaaaacccggaggggggaattttatttaaaaatttcttcccccaaaaaataaattt

SEQ ID NO: 46 - DNA, 1407 bp, Brassica napus
ggaaacacaattgagggaagatgttctgagggaggaatttactttaactttatttgagaaggatgaagaaattaaagagaaggcaacaaaaattgagaaggcagagcagtctctaactgttctaagatcagagttgacggcggcagagttgaaaatcaaaagctttgacgtggaaacagcatcactgaaactagagttaagggaaatgattgataggttagacagtgccaacacaaaggctctagcatatgaaaaggaggcaaacaagttggaacaggagaaaatccgcttggagcaaaagtaccgatctgactttgaaagattcgatgaagtccaagagagatgcaaaacagctgagatagaagctaaaagggctactgaactggcggacaaagctcgaactgatgccgtgacttcccaaaaagagaaaagtgagagtcagaggttggcaatggaaagacttactcagatcgagcgagctgagagacatgccgaaaacttagagaggcagaaaaacgacttggaggatgagttgcacagacttcgtgcgtctgagatggaggctgtctccaaggttacagtcttggaagcaagagtaggagagcgagagaaagagatcgagtcgttgttaaaggtaaccaacgcgcagagagcgcacaacgtgaagtcactggagaagctgttggatgaagagcggaaggctcatatagctgcaaaccgaagagccgaagctctttctctccagctgcaagccgcgcaggcaagcgttgataatcttcagcaagagttagctcaaactaggttgaaagaaactgcgcttgacaacaaaatcagagctgccagtgcttcgcgcgggaaacggaccagggtcgaagatgttgttgatatggacattggagaagcaagcgatagaatcttgaagactaataaacggtctcggagcgcaagaggagatgatgatagtggtgcttatgaggatggtgtttccgtttccagaggaggtgaagacgaggaggaagcagaagattacaagaagctcacggtgcagaatcttaggcatgaactgaccaagtatgactacggacatttgatactgaacaaagggcatcagaacaagaaagagattcttgctttgtatgaagctcatgttcttcccaagaaagaggaagagagaaagagacagagagaagctacttcttaggacaaactctgtatagagtgaggtgatctcaagtaagagttggatttagaaaggctctgactttaagtattgtttagggtttctgagtttgtctttgtgttggtattaatttgcgtggggtttgagtcggttttcgtgtcggatggaactatagatttgtaaaccatctttttcttcagaattatggtgattatggtgagtgtgag

SEQ ID NO: 47 - DNA, 602 bp, Brassica napus
gcaaagctgaggttgaaatcatctctcgtgttcatcaccgacatttggttacgcttgttggctattgcatctcggagcagcataggttgcttgtgtatgattatgtgccaaacaacactctccatttccatctccatgctccgggaagaccggttatgacgtgggaaactcgggttaaggctgctgctggtgcagctcgtggaattgcctacttacatgaagactgtcatcctcggattattcaccgtgacatcaaatcttcaaacatactcttagacaacagctttgaagccttggttgcggatttcgggctagctaaaatagcgcaggaactggatttaaacacacatgtttcgacccgtgtgatgggaacctttgggtacatggctcctgagtatgcaactagtggaaagctgtctgaaaaagcagatgtttattcgtatggtgtaattctcctggagcttatcactggtcggaaacctgtagatacgtctcagccacttggggacgaaagccttgttgaatgggcaaagcccttgttgagtcaagcaatcgaaaatgaagagttcggcgagctagttgatccgaggttagggggcaac

SEQ ID NO: 48 - DNA, 926 bp, Brassica napus
  misc_feature (907)..(907): n is a, c, g, t or u
tgaatgagcacagctactatagggcgaattggtaccgggccccccagcaagattcatgggagctaatgataaatgtccagaaaggagccattgaaggctggaaggtgcaaggagttctacttatgaatgaagctatactgccatgcttctggcgagcacctacagacaatgataaaggtggaggtgactctagttacttttcaaggtggaaagcagcacaattagacaatgttgagttcaatgttgaaagctgttcagtgaagagcaccactgataaatccgtggagatagagttcatctaccttggttcttcaccttcaaaatcagatgccttgttcaaagtcaatgtgacatatatcatctacggctctggagatatcatcaccaattggtatgtagtcccaaactctgatcttccaccgctaccacgggttggcgtagagtttcacctcgagaaaacactggaccgtgtggaatggtacggaagaggtccatttgagtgttacccggaccgaaaatcagcagcccttgtggggatatatgaacagaacgttgcggacatgcatgttccttatattgttccaggagaatgtgggggtagaactgatgttaggtgggtgacattcacaaacagggagggtgtgggaatatatgcttcaacatatggtagctcttctccaatgcagatgaatgctagttactatacaacaggcgagcttgatcgtgcaacgcatgaagaggatcttgtcaaaggacaaagcattgaggtgcatctggaccataaacacattgggattgggggagatgatagctggacccctgtgttcatgataagtatctgattccaccagaaccatactcattctttttcaggtgtgtcccattactgcagcggcttcggtctttnacatgtacaggatcaaatt

SEQ ID NO: 49 - DNA, 742 bp, Brassica napus
aacatgttacaggctgtatacagagagtgcatatctaaacgagatgttgccgagtcccgtgccagagattcagcgaacaaatctgggtaacgttgtgttgttgttgaagtcgctgaaaatagacaacttgctagagtttgatttcatggacccacctccacaagagaacatcctcaactctatgtaccagctttgggtgttgggcgctcttagcaatgtcggaggattaaccgatctcgggtggaagatggtggagttcccgttggatccacctcttgcaaagatgctcttaatgggtgaacggcttgactgcatagacgaggtcttgacgatcgtgtcaatgctttcagtaccttcagtgttcttcagaccgaaagagagagcagaagagagcgacgccgcgagggagaagtttttcgtgcctgagtcagatcacctgacgctactcaatgtgtataagcaatggaaagagcacgactacagaggagactggtgcaatgaccattacctgcaagtcaaaggtctgaggaaagctagagaagtacgatcccagcttctggatatcctcaagcaactcaagataccgctcaagtcgtgtgggcctgattgggatatcgtgagaaaagccatatgctcagcgtatttccacaactcagcgagattaaaaggtgtgggagagtacgtgaactgcagaactgggatgccttgccatttgcacccaagcagtgcactgtatggtctg

SEQ ID NO: 50 - DNA, 788 bp, Brassica napus
gtctaatcagggatctcacgagttgcagcctataatgcccagaggaacttggttgaggtttgattttgaagattcacatccggatttgccgacattatatttacaagggggatccatcatagcacttggtcctccacatctccatgttggggaatctagtctgtcagatgacttaaccctacttgtttcattagatgaaaatgggaaagctgtaggactcctatttgaagatgatggagatggatatggctacacagaaggcagatatctaattacacactatattgctgagcgacagtcttccgttgttaccgtgaagattttaaaaacggaaggagagtgggagaggccaaagcgtcgtattcatgtccagttattgcttggtggtggtgcaatgcttgatgcttggggaatggatggagagaccatccagataaagatgccttcagaaagtgaagtttcagcgttaatatctacaagcagtgagcgctttgcacttcatatggaaaatacaaaactgatacctgagaaggaagtgctacatggacaaaagggaatggaactttcaagggaaccagttgagctaaacagtggcgattggaaactgaacatagttccttggattggtggaaggatactatctatgacacatgttccatcaggagttcaatggcttcacagcaggatagagatgaatggttacgaagagtacagtggtactgagtaccggtcagctggatgtactgaggaatataatgttattgagagggatttggaacaggcaggag

SEQ ID NO: 51 - DNA, 1174 bp, Brassica napus
  misc_feature (631)..(631): n is a, c, g, t or u
ggaatgtatatacactagtattacgattcgtatagccgacccccccttcgagcaatgtaacagctttgatagacatgaagcttgaaaaatacaagaacttaaaggaggagtcccgtttttactggcaagagattcataggggcacactcaaattcaaccgtaaagaggcagaggttggtgcactgaggcttttttcataagcaagagttaatagatttcttcgaggaatacatcaagattggagctgcgaagaagaagtcattgagcataagagtgtatgggagccagcacttgaacgaaatggcaagtgacaaggatgaagtcccatcaccatctgttgaaattgaggacattgttggtttcagaaaggctcagcctcttcatgggtcgttcagaggatgcggacaacccaaactgtgaggggctgaatctcattttattatacaaagacgatcattttatatcttcttcgatcctttctcgttgtaagaataaaacaggtgaaccttatgttggcatcaatttcacaaaaaacaaatgaaatagatttaaagaaaaaaaaaaaaaaaaactcgagactagttctctctcgctctctcgtaaaagttgaagacacagagaaggaacacangaagaaaaagaaagataaggagaagaaagatgaagatgtatcacaagaagaagaagaaggtattaaaaagaagaggaaagattnggtttgattcaggtgccagatggaaaagaccnggaggaaggattatggagaagaaacccggaagggaagattcccaaggattatgaagggaaaaaggaacccttaaaaaaaagggagaaaaaggattgaagcctttggaagaaaaaaaagaaaagggggaaaaaaggaaaaggaggaaagggttgggaattcaaggttggccccccccggggaaaaaggcccccaaaaaggaaaagggacccctggggtggaagaaaaacctctggaaaaagaaaagaaaatttcccagggggtgttttttnnnaaaaaaaaaaataaaccngacgatactccccttgtgtatttactctttattgtttcctgcgtggtcccctcttaacataatcgtattttactctcttctatgnccttcctctttgtgtaaaacgaccngggtgaaactttctcctacttcgtgga

SEQ ID NO: 52 - DNA, 1886 bp, Brassica napus
catatgggtgctgtgaacgacttggctttcgctattccgaacagacagctatgtgtagttacttgcggagatgataagctaatcaaggtatgggatcttcaaggtcgaaagcattttacctttgaaggtcacgaaactcctgtttattccatttgccctcatcacaaagagaacattcagttcatattttcaacggccatagatgggaaaataaaggcgtggctttatgataatatgggttccagagttgactatgatgctcccggtaaatggtgtactacaatgctttacagcgctgatgggaccagattgttctcttgtggaacgagtaaagatggagatttttccctagttgagtggaacgaaagcgaaggatcaattaaaaggacctatcttgggtttcagaaaaagctggcaggtatggttcagtttgatacctcaaagaaccactttctggctgttggtgaagatgcgcaaatcaagttctgggatatggacaacatcaatgttctgactagcactgatgctgagggtggacttccggctcttcctcgcttgagatttaacagggaaggaaaccttctagcagttactacggcagacaacggatttaagatcctagcaaaccaagctggtttccgatctctgagagccatggaaacttcagcttttgaaaagatgaggactccagcagattcttctttaattaaagctgttcctggtgctcctgttgcatctgtcagctgtaaaattgaacgaggctctcctgttagaccctcaccaatgctggtaaattacccattaaatgttatgtttctagccatttttataaatcatattgttatgttataaagttttcactagaaaatgaaataggattacatttgtttaaaatcatggttatagcatttcagttttcttatatgtttaaggatagttgctattcttttttgttgtggcaaatcctcaagaatggagttgatccctcgaagccaagaataatagacgactcaacagacaaaccaagaccttggcaattagctgaaataatggaccctgcccagtgtcgccaggctactttacccgataccgctggttcttccacaaaggttgtccggcttctgtatactaattccggcgctggaatcctggcactaggtttgaacggtattcagaggctctggaagtgggttcgcagtgagcagaaccctagtggaaaggcaactactgctgttgttcctcagcaatggcaaccaaacagtggtcttctcatggccaacgatgtctctggtgtaaaccccgaagagtctaacccgtgcatcgctctctctaagaacgactcatatgtcatgtcggctgctggaggaaaagtctcgttgttcaacatgatgacttttaaggtgatgacaacattcatgcaacctccaccgccatcaacatttttggcgttccatcctcaggacaacaacatcattgccattggaatggaggactccaccattcacatctacaatgtccgagtggatgaggtcaaatcaaagctaaagggtcaccagaaacgcatcactggcttagcattttctacaaccctcaatatcttggtttcatctgctgctgatgcacagatatgcttttggagcattgacacatgggagaagagaaaatccgttgtaataccaatgccagcaggaagagtcgccgttggagacacgcgtgttcagtttcatgtggatcagatccgtatccttgcagtccacgagacacagctagcgatattggatgcttccaagatggaatgtatccgacagtggattcctcaagactcgttgtcttctcatataacttcagcagtgtatgcatgtaacagccagttgatctacgccact

SEQ ID NO: 53 - DNA, 803 bp, Brassica napus
cttaaactcgtttttgctttgctaaatactcagcaattctctgcgagtagatgactttgtgggctttccacctacctttgcagatttaattaatttgaagatcttgaacttgggaatgaacgacattacaaactcatgcttggtccacctcagagggttgacaaagttggagagcttgaacttggattcttgtagaattggtgacgacggactggtacatttatcaggtatgcttgggttgaaatctctggagctgtctgatactgaagtaggaagccatgggcttcgccatctctctggtctctcgaacctggagagcataaacctgtcattcactgttgtcaccgatagcggtttaaggaagttatctggtttgacatctcttcgaacgctgaacctagatgctcgtcatgtcaccgatgctggcctttccgcgctcactagtttgactggattgactcaccttgatctcttcggtgctcgtatcacggattctggaacaaatcatctacgaaacctgaagaaactgcagtcacttgagatatgtggtggtggattaactgatgctggtgtcaagaacataaaagatctttcatccctcacgctcctcaatctatcacaaaactcgaacctcacagacaaaacactggagttgatttccgggttgaccggattggtctctctaaacgtctcaaactctcgagtatcaaactcaggattgcgtcacctgaagctggatgtgctcatctcggaagtcagtttgttggggtgaaaactaactttgatgcgtttgatcggctttc

SEQ ID NO: 54 - DNA, 560 bp, Brassica napus
gcgaaaagaagaaagacaaggaaaagaagaaagagaagaagagaaaaagagaagacccggtgtatctagaaaagaaacggttaaagaaagagaagaaacaaaaggagaaagaaatgtcaaagcttttgaccagctcgtctgatccagctacaataaagatagagacaatcccggaggctaaaccagagggactaagtgataaaccaaaggctgagccatcagcagcaccagcagaagctcgtccctcgaccaagatacgaatcaagctgaaaagcaaagcgttcaacaatcactaagggttcactgattcgagccaggtctttttttttcaaatccaaatctgccactcgtttgtcactgtggtgggtctttgttatctgattaatgcatagtttcgatgtgtgtagcttgtaagtgataagctttgttgttgcaaagtgtgatatgtcaaaatgtgaactagtaacttccatgagtagtcgatgagttgtttacaagtatttttaaaaaaaaaaaaaaaaaaaaaaaaaaaaaaaaaaaaaaaaaaaaaaaaaaaaaaa

SEQ ID NO: 55 - DNA, 897 bp, Brassica napus
  misc_feature (29)..(29): n is a, c, g, t or u
agttggtacgcctgcaggtaccggtccgngattcccgggtcgacccacgcgtccgacgacacattcgatggaggaagctgatattttaagtgatcgaatagggataatggccaagggtaggctccgctgcattggaacctccatcaggttgaaatcccgcttcggcacaggcttcattgctaacatcagcttcactgaaagcaacaaccaagaaaacaacggcaatggtgaagctggtgctgcagactcacgtgagccagtgaagaaattcttcaaagatcatctcaatgtcaaaccagtagaagaaaccaaagccttcatgacttttgttataccgcacgacaaagagaatcttttgacaagatttttcgctgagctgcatgacagagaaaccgaatttgggatctcagacatccaacttggtctagcaactcttgaagaagtcttcttgaacatagcaagaaaagctgaactcgaaagcgctgctgttgatggtacaatggtcactctcgaactaacatctggcccatccgtcgagataccggtcggggcaagatttgttgggataccaggaaccgaaaatgcagagaatccacggggagtaatggtggaagtgtattggcagcaagacgagtcgggatcattgtgcatctcaggacattccacggagatgccagttcctgacaatgttcctgcgacagatccggtggcaccaggacatggcggagtaagcttgttgggacggagacaacaagttcaggggattgtgattgatcctgagtttgtcggctccgctgcttcccgtcgtgttagtagaagcggctcctttgcttccaacgttcttcactttgaatgagaatctacacacacccactgttcctagcttaccggactcgt

SEQ ID NO: 56 - DNA, 697 bp, Brassica napus
gactccaagaccacccctgctgctgtctcatatttccactcagtgtctagtgaagatgaagatgatgagaaagaactccgtagaggtggttcaggcattcaaaccaagacgtctatatccatctcaagaagaacaaaggggcaagagagaagatcatcctccctggctactgcaaaaactgagaaagactctcatgatcaggactctatcccaaagccaatacctgaggccaagccaatggctaagaaacaggagtcagctgcttcgagctctcttccaaaaacagaaaaggcttctcaagaagctcctccaaagctagcttataaggcgaaaccacaggctgatcaagaaaggccagcagcttcatcttcaagttctattccagaaaccgtgtcatcaccagaacaagtaactctagcgaaggagaaagctagccatgttcatcccaagctcccagattacgacgacatatttgccaagctcggggctctgcgtcgctgaacttttactgatctctaccgtctgttgctacattattatgttcattgttttgtcagctcttattcttttgtttgagtatttgcttaaaagattgaacttgagagtatcctgaggattattttcttgccgtgagattacttcatttgttatatgtacaaaacatatacttgactttcttataagattgttgccttgt

SEQ ID NO: 57 - DNA, 792 bp, Brassica napus
cgcgatttcaattgtgttttctagggttttcgtcgtttaggtttccgacaagatcttaggcgtaagattcgcaggcagtgagagagatatggatggtgagagaagagttcatccggactgtataaacgcctcgaatccttaccacgagtgtgtggagtactgctttaagaaaatagctgaagcaaaggccaaactagagaagcaaggattagttgaagctgtcagtgggcagtcaagggagtcacttgctgataaaagaatagaggaagcgagttccgaggaagaagaagaagatgaggaggataaccaagaaccccaagtagacgtcaccaaacttacagggaggaagaagaagttgtttgagctgaaacttaagatgaatgaagcaagaaagtccaatcagacagatgtcgggtctgaaaagaagaagatggaagcaccaactgagccaaaaggaatctcgaaacagaaatgggtggaggcgaggcagaaaaagattgggaagattctcgatgctaatggtttagatatgtcaaaggcttacatgctcgatactcaagaagctgcggaaacaaaatacaaaaaatgggagaaggaacccacaccttctggttgggatgtcttcaaccagaagacgttatacaacgcatacaagaaacggacaaagaacattcaggttgacatagaggagtacaacagaatgagagcagctgatccagagttttaccgcgaggcctctagcttgcaatacggcaaggcaccaaaaatttcaaaagataagatagataagat

SEQ ID NO: 58 - DNA, 759 bp, Brassica napus
agttgtcaagagcaggctggtaccggtccggtcttcccgggagatcgtcgacccacgcgtctcagttggtaacggggaagcaaccaacagatctagaacttggagataaagatatggtgaagtgggtgtgcactacccttgaccaaagcggtttagaaacggtgattgatcccaaacttgatcttgggttcaaagaagagattagcaaagtcattcacattggtcttctctgtacgagtcctctccctctaaaccgaccttccatgagaaaagttgtgatcatgcttcaagaagtctctggtgctgtttctattagcggtccaaatgcgtccaaacgctctaagagcagtgggaaactctcgccttactatgtggaagacgtaaacagcgtttgagcattcaaatatgggccggtatgggttgcgctgggccagaaatagttatttggtttgtattaagttaggtgaattgtaagaaatcagaaacagaaaaatggagctttctgattaggtaatagcttttaatgctatgatgtttggaaaaaaaaaaaaaaaaagggcggccgctctagagtatccctcgaggggcccaagcttacgcgtacccagctttcttgtacaaagtggtccctatagtgagtcgtattataagctaggcactggccgtcgttttacaacgtcgtgactggaaaaactgctagcttgggatctttgtgaaggaaccttacttctgtggtgtgacataattggacaaactacc

SEQ ID NO: 59 - DNA, 776 bp, Brassica napus
acaaaccaaaacagcagctggggtcaagcgactgttgtaaatccaaacatgggatgggctggtcctcctcaagctggaatgagcgtaaactgggctgcatcttcggttccaccaactggtcagggaatgcctaatcctggatggggcgggcccgcccaagtacaacaaccacaagcttattcggcaaactccgggtggggtgtaacaggccaaactcaatcccaagcccaagttcaagctccagggattagtacaagctctggatggatgcagacaggtcaagggatgcagtctgggaacagtaatcaaaactggggaactcaaaaccagtcagcaatgcaaagcggtgggtcaggagggaaccaaactggttactggggaaaccaacagaaccagaatggagattctgggtatggttggaataggcagtcaagtggaagtggtgggcataacaacttcaaagggcaaagagtatgcaagttccatcgagaaaatggaaattgcaggaaaggagcagcctgcacttacctccacaactaaaccagcttttctcaatctcctcctgtttttgggtgatgtttgctgcatcctaaaaagcagttttaggcgttttagacacactatgcacaagagtcttttgttgagagcctttcactcagaattagcttgaaatgtttctttgtttttgggggttaagagcttagtactgaaatgtcgtggtggtgaatatggtggtagagaatatccctattgcatatagtatttgctagaccaac

SEQ ID NO: 60 - DNA, 1020 bp, Brassica napus
atacgctatttctttcatcctcgttctcaacttcattgctgtcgttgttgaaaccacgcttgatattgaagaaagctcagctcagaagccctggcaggttgcagagtttgtctttggttggatatatgtgttggagatggctctgaagatctattcatatggattcgagaattactggagggatggtcaaaaccggtttgattttatagtcacatgggtcatagttattggtgaaacagctaccttcataactccagacgagaacactttcttctcaaacggagaatggatccgataccttctccttgcaagaatgttgagactgataaggcttcttatgcacgtccagagataccgagcgtttattgcgacattcatcactcttattccgagtttgatgccttatttgggtaccatcttctgcgtgctgtgtatctactgctctattggtgtacaggtctttggtgggcttgtgaacgctgggaacaaacaactcttcaaaactgagatggctgaggaagactatctactgttcaacttcaatgactatcccaatggaatggtcacactcttcaatctgctagtgatgggtaactggcaagtctggatggagagctataaagatttgaccgggacgtggtggagcattacgtatttcgtcagtttctacattatcactgttttgcttctgttgaatttgattgttgcctttgtcttggaggccttctttactgagctggatcttgaggaagaggagaaatgtgaaggacaggattctcaagaaagaagaaacaggcgtcgatctgcagggtcgaagtctaggagtcaaagagttgacacacttcttcatcacatgttgggtgatgaactcagcaaaccagagtgttctactactgctactgatacataaaaagcccgttaaaaagttttttttgttctcttttaatattttgtttttggaagcgttcttgattctatgtagagagagtcaaagagaatctagaagttaaactttgcatt

SEQ ID NO: 61 - DNA, 991 bp, Brassica napus
  misc_feature (765)..(786): n is a, c, g, t or u
ggaagctatacccgctctaatacgactactatagggaaagctgtacgcctgcaggtaccggtccggaattcccgggtcgacccacgcgtccggcttcaccagatactggaagaaaagaaaccgatcctctgttaaacaaagaggttgtcatgaccgttaatgacataggtgactggccaaggaagtcttgtataccaatacagtctgcccaaacattcaggtctcgtcagactgttctctttatcgctacaattgctgtctgttttgcggtctgtgcggttctctaccacccaaacaaggtcacgcagcttgcagtgtctatccggacgagattggcacacaaactatgaataaaaagtccaatggcagagactctcattttaaccggtgaaggtgtgttcatttaaagaagcttgaagggagctgtacaaaaaagctagtctcagctttcaacagagctcaggtacagcttctcggatagattaccttgagaaagagacaaaatgattaatgatcagatgcgtttacttgctctttctctgtgttcttcgtcctaatttcttatagacttggtggtgatactgcaactgtaaaacaggaaatgatctggtcactgacatgattatccctcatcgaccagtgttatatatgtagattagggtttgctttcgctcactgtaacatgtttttttttcttggtcttgaaaacgattcttggacaaatcctaatcgtaacttagtattatacatttttctgcttcaaannnnnnnnnnnnnnnnnnnnnnggggcggccgttctaaggatccaagcttacgtacgcgtgcattcgacgtcatagcttttctatatgtcacctaaattcattcactgccggcgtttaaaacgtgggactggaaaaccctgcgttaccaactaatcgcttgagaaaatccccttttgccagtgcgaataccaaaggccccacgacgcccttcaaagtgccaactgag

SEQ ID NO: 62 - DNA, 917 bp, Brassica napus
gaatcctgagatggtgcagatgccaaagacgatgaatttcgttttttctttttctctatttttgttcataccaccgataatgcttgataactcacaaattttcaatttaattttttgattcttggaactagtatttgtatctatctttactttaaaaaatttttttattgaaacttcgggaagtactttagaaacatatgtataaaaaaacatattttattgagtcccttcatgcttactataactagttatttcggttttctactagcagctttaactataacctcagttctatttattggtctaagcaaaatacgacttatttgaaattaattgaatgaatcttttttgatcaaaaaagatttatatggtatttcatatgttcgatagttccttaccgtgttaattacccaattttggtcattgagattcgtcggcaatacagattaagagctaggaatagatagtacctctcttttctccctttcaaaaatgaaaacaaaataaaattgaaatgattgaagtttctttatttggaatcgtcttaggtctaattcctattactttggctggattattcgtaactgcttatttacaatacagacgtggtgatcagttggacttttgattaattaacatctcttttttttgactgacctccttcttgctttcatatgcgggaggtcgaattcagattgctgctcaattatttgcgaacagtggaattttgacacaatctaataaacaagagtgacatcacgctctgtaggatttgaacctacgacattgggttttggagacccacgttctaccgaactgaactaagagcgcttttcttgttttttataaaaaaacgaaaaggctagaaagaggacattctttaactcgaatcgattttgtacgtatatactatatca

SEQ ID NO: 63 - DNA, 680 bp, Brassica napus
gaccctcctcaacacactttctctctacaccaaaaacatcaacgaaacaagaagaagtcactagcttagctcaccaacactcgctatggctctgaagacggcttcgacgaacgggatgtggatgttggaggactgcaagagatcattcatggagatgaagtggaagaaagtgcaccgatacatcgttttcaagatcgaggagaaatccaagaaagtcgccgtggacaaggtcggtgccgccggtgagacctatcatgatctcgccgcttccttaccggaggacgactgtcgctacgcggtgtttgatttcgactacgtcaccgtcgataactgccgcatgagcaagctcttcttcatcacctggtccccggaggcgtcgaggataagggagaagatgatgtacgcgacgtcaaagagcggactgaggagagtgttggaaggcattcactatgagcttcaagccaccgacccaaccgagatgggattcgacaaaatccaggaccggaccaaataaaatatttacattccgtttaaccggggttacatcggtagtgacgtgatcattaacagacgttaattatctattaatgttagttattgttctttagtgcttgacttggtatgtgagactttcctttctttccttttgttgacaagagttatcatctattaccgtttct

SEQ ID NO: 64 - DNA, 585 bp, Brassica napus
gaaagagattgttctctctcctctcctagagagagagttctctcgaactttcttcctcttttatctctaaaaaatctttcagagagggtagggatcgttttgagagtgagaaagatcatcgtttttgttctctgaaaccgaccgactccggtgttttcgcctctctttagacgatggacagctggctcttgactctgggttgcagtggcttcctcagctccattttgtcggcttttgactcagtgtcacgcctctttgatgtcgacggccgttgttcaccggggttatcctggtttgtttgagttttcttcaccagcttcggctccgctgacggcggagatctgtcacctctcttctatagcgccggtggaagatggttgactctcatcttgtcacgtgtttcagaatgcaggacattcgtatacgtggtgggaaactatcagttttttctttgacggtacataagcctaaaatggttttgtattattgcccattttgttgggccttttgtttttacgtaggacagggtctagttaggccttgatctatttaacgaaaaaaaaaaaaaaaaaaaaaaaaaaaaaa

SEQ ID NO: 65 - DNA, 501 bp, Brassica napus
gggaagcctagtgggcttcggatgttgggtttgtggcctcgggtttttggggcgccgggttcggtttaatgtttgtttgggctttgacccataacgtttctttagtttgccctttgggcctttgtgatgtttggtttctgggctttgacccatcaataataataatagatggaaaaaaaaaaaatgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatggggtaaaaaaaaaaaaaaaaaaaaaaaaaaaaaaaaaaaaaaaaaaaaaaaaaaaattaagggaacccaaaaaaaaaaaaaaaaaaaaaaaaaaattttgggggccgccccgccccaaaaaaattttttaaaaatttttttttgggggggggggcccaaggggaaaaaaaacagggctatttttttt

SEQ ID NO: 66 - DNA, 613 bp, Brassica napus
gggagaagagcttatccagaaaccaaacttcaaatgaatgtctgaaggctagatccttcaattcgaagaagatggctcttctctgtttcaattctctcgctcctctctcttcttcatcttctccaaggcttcaactttcgtctttagcttcaccagttttcatcgttaaacccaacacggtcgagtcaaagaacagagtctcactcagtgcttacatcttgagcaacagccatggaagagcagcaattgtgagagcagctgcttctggcgtggacggagctgagcccgaggaaccacctaagactgttgtctcagtggacaaactaccattggaatcaaaagaagctaaagacaagctattgctggaacagaggatgaagatgaagctggccaagaagattaggctacgcaggaaacgtctggttcgtaagcgtaagttgaggaagaagggtcgatggcctccttccaagatgaagaaactcaagaatgtttagagatgattatcatgactctttgctctctttttttatttaactttttttttgtaatgttgtttgtataagcattgttttttggacaaaaaaaaaaaaaaaaaaaaaaaaaaaaacatgtc

SEQ ID NO: 67 - DNA, 718 bp, Brassica napus
ggagctcttcttcttcgtctaatcttgctgcgatccctctctcgctctcgctctcgctctctacagcgcaaagtgtgagacttttggcgggaaaatgtcggggaaaggagcgaagggtttgataatggggaaacccagtggtagcgagaaggacaaggacaagaagaaacaacccatcacccgttctgctcgagctggtcttcagttccctgttggaagggtgcataggctgttgaagacaaggtccactgctcacggaagagttggagcaactgcagctgtttacacagcagccatactggagtatctgaccgcagaagttttggagctggctggcaacgccagcaaggacctcaaggtgaaacgtatctccccgaggcacttgcagcttgcgattcgtggagatgaagagctcgatactctcatcaaaggaactatagctggtggtggtgtcatccctcacatccacaagtctctcatcaacaaatccgccaaggaatagattcttaccactctttgggccttgcttctctgtttctttaagtatctctctctctcgagagagagtttgtattttgataaaagaagacgctagagagctcttttgttatgcgtatgtatttgatgatttatctaaaactcattttctgactagaagcttgtaagcaaatcaatgtgttttttcactgaacatgaatcgtatttgcatttacatgat

SEQ ID NO: 68 - DNA, 495 bp, Brassica napus
  misc_feature (495)..(495): n is a, c, g, t or u
gtctctcacatgggacgcgtgggcggacgcgtgggcatgtcttctctgtctagtacttctcttgacctgtcttcttgctggacgccatcggagcttctcggcgtagtggtagaagaggcagaggagattgatggaggaggagttagcagaggtggtggtggctcgtgactcgtgtgcagaaccggcttgaagggtgaagaagaagaagctacggtgaggaagcagaagaagcgtgatggcggagcaggcgtgacgcgttgcttcatccgttacaccggttgttccagaagcagatcgagtcgccgcttcagacgtagcatccggagccagtggaggtgtctgtcgtgaagccggtggcggaaccggtggtggagtcggtggttgagcctgtgtatatatttgtgtgtatatgtgtatatattcataatttgtaatttaattaatagaaaaatatttttatgcatttttgatttatttttaatatatattttttan

SEQ ID NO: 69 - DNA, 681 bp, Brassica napus
ggttatcttctcagaactcgcgagtgaagtcttcttcttccgacaatgcttagcctctctatcacctcaccagggacagcggcgactttcctcagaggagctagctcggcgacttcaacctcgtcttcgttccatggcgtcagaatccagcacgaggtctccgctcgcgtgccggcggcgacggtttcatcgtcgtcacgtaaacaaatgggggtgaggatgtcgaaaagagaggcggagctgaaagatataagggggaagacgacggaggagctgaacgaggaggtgattgaccttaaaggagagctcttcatgctccgtctccagaaatcggcgaggaacgagttcaagtctagcgactttcgccgcatgaagaaacaagtggcgagaatactgacggttagaagagagagggagatagaggaagggatagggaagaggttgtctaggaagcttgacaggcagtggaagaagagcattgtagtcagaccacctccttctctcaagaagcttcaagaggaggaagctgctgaagaggcagctgaagctgccaaatctgcttgaatgccccagccatttgattggtcggtctcttcttcttgtttctttgggatgttattgtctctctgttgttgcttttttggtttataaactctcctcatcaagtagtttgcatatgaa

SEQ ID NO: 70 - DNA, 573 bp, Brassica napus
ggaatcaccaccgttggatcgaaatcttccgatccggtcacacgcttgaatcaatggagagagaagcacgagatcaaatccctcccaacgcgccgctgcgttcgggaaacgcaaccatcggcgaccaaagctgatctacataaaggacacgtgaaattgagatgatgaagccatccctctaagcacggcttgtggaacacgtgtccacactccagcttcctcacctcctctccttcatgcagctccgacagacacacgacgcagtctgaaccgccttcgccggcgctccggtacgaggagagccggttcaggctcagctgatcggcgaggacgatgatgttggcgagtcccgaggctatgatggagccgtcgtcgagggggagattagaagctggatcgcagggagggaggaggaaagagcggagacggctgaggaatacggcgaggagagagaggagcatgtgagggagtgaatcggaagagacgtcactgagctgaccttgtagccccatcttcttcttttcttcttcggattatttgcttagctcgtttttttttttttttttttttttt

SEQ ID NO: 71 - DNA, 726 bp, Brassica napus
attcccgcttcatcttctgtgcttttaataatttagcgctgagtttcttcctctctagccacttcctctctctctctctccctctctctcttgtgccctttttgactaattgctctctagctttgaagaagaagaagagaaagagatcctggggttagggttcgaaggccatgtctgctgctcaggaggacttgttgttgacgacaagacaccagatgctgatggagaatcacgagagcctgaagcaagactacgcgagcctcgagcaaaggtttaagcttgtggaagatttgaatgcgatgatgaagccgcgtcttcagaatcagagccagtctaaggagctgcgcttgctcaaggatctcgagagggagaggaaggagaaggaagagttcaagaagatggtcgaggtccttgaggccacgaacagtgaactgatgctcaagaacgaggagcttttgactgcgttggagaaacaagaggatgaggggaagaggcaggaggagaagtttgatgagttagcgagggtatgtgatgagcttggaaaggagtgtttgcatttaaaatcactctatgatgatcctaagagactcaacggtgggcaaggaaagagcaatgtgggtttggaagatgatgtctttgtggtgggtgagagtcataatgctgtgaagaacaacaacaacaacacagtggctgatgcgattgtgctcagtgacgagagtgatgctg

SEQ ID NO: 72 - DNA, 598 bp, Brassica napus
ggtctctgagcttcttcttccacttccttgagcttccttttctacaagagaattaaacattttttgttcgtttaaactcttgataatggactcgtattacgtcaggtttgtggaaaacgacgagccgcactttctagagtcttgctctctttgccgcaaaaccctttctcttaactcggatattttcatgtacagaggagacatggcattttgtagccaagagtgtaggcaagaacagattgagtctgatggaaagaaagccaagaggtggagaaaagagtcgtcgtcgtccagatccaaaaactccgtcgccggcaaatgtgtacggtcggaaacactcgtcgtgtcttagttattacaacatcttcacgagaagtgatctttttgtttgtggttttcttggtttcagtttaaagaaaacaaaataataatggtaaatcaattctaaattcatgtttttccattttcaatgggcttatcacgaaatgtgttagcccattatgacccatttgataagatcatgggctcattttttgaagatcttaatacactattttggattggtacctaaaaaaaaaaaaaaaaaaaaaaaaaaaaa

SEQ ID NO: 73 - DNA, 902 bp, Brassica napus
tcgctctaaatacattcttaaatatttttaagaatcttgtcagtaattaagatttccaaaagttggccgaaatgaatgtctattttctcagtctcattcttacagtcattttagtgatttcattcaacactgaagcacttagtcctcactactacgaccattcatgtccacaagccgaccaaatagtcaccaatgcagtcaagaaagccatgtcaaatgacaaaactgtccctgccgcgctcttgaggatgcatttccacgattgctttgttaggggatgtgatgcgtcagtgctgttagactcgaagggaaagaacaaagcagagaaagatggacctcctaacatctcactccatgcgttttatgtgattgacaatgcaaagaaggcattagaagagcaatgtcctggtgtcgtctcgtgtgcggatatcgtttcactcgctgcaagagatgctgtcgctctctctggagggcctacatgggaagtgccaaaagggagaaaagatggcagagtatcaaaggcaatagaaacaagacaactaccagctcctactttcaacatctctcagctgcaacaaagctttggccaaagaggcctttctatgcacgatcttgttgttctctctggaggacacacacttggattcgcccactgctcatcgttccaaaataggattaacaacttcagcacacaaaaacaggttgacccaacattaaacccatcatttgcggccagcttgaaaggaatttgcccagcccataacaaggctaagaacgctggagcgactatggacgctagcacgacatcattcgacaatatatattacaagatgcttatgcaaggcaaatcgctattctcatcagaccaagcacttctcacaacaccttctacaaagaaactt

SEQ ID NO: 74 - DNA, 1026 bp, Brassica napus
  misc_feature (860)..(860): n is a, c, g, t or u
tggtggctgatcgcagtggtatacgcctactatagtgaaagctggtacgcctgcaggtaccggtccggaattcccgggtcgacccacgcgtccgccagatcgttgttttcacggcggggctgagagagtacgcttctttggtgctggataagttggatccggagcggcgcgtgatctcgcggagcttttatagggacgcgtgtagcgagatcgatgggaggttggtgaaggatttagggtttgtgatgagagatttgaggcgcgtggtgattgttgatgataatccgaacgcttacgcgcttcagccagagaatgcgtttccgatcaaaccgttcaacgatgatttgagcgatgttgagctaaagaaggtcggtgagttttttgatggagattgtgagaagtgtgaagacatgagagttgctctgaaggagtttgtcggaagagaggaatgatttttggttaagttgattagttgatttgattaattagaggatttgattatgaaaggcattgaaccaatagctttgtttgttcaattgtcttgtgtaaaacctcctcgtttgaaaaaagcaatttgttcatttgttttgattagtgttttctccgtggctgaacaaaaggaggtctaatgaaggttttgaaatttaaaaaaaaaaaaaaaaagggcgggcgctctagaggatccaagcttacgtacgcgtgcatgcgacgtcatagctcttctatagtgtcacctaaaatcaattcacttgccgtcgttttacaacgtcgtgactgggaaaacccttggcgtacccaacttaatcgccttgcagcacatcccccctttgccaggtgggcgtaataccgaagagccccgnaccgatcggcctttccaacagttggcccaacctggaatgccgaatgggacccgcccctgtaccggggcaattaaccgccggcgggttgtgggggttccccccaaggtaaccgctacccttggccaaggcccttaggccccggtccttttggctttttttccttctt

SEQ ID NO: 75 - DNA, 602 bp, Brassica napus
gacaaaagagttggacacgacaagctgttcctaacaccagcgagataaaacagcgaagtctttaacaaaaagaaaatggtggaagtgtggtggcctctgttagcagcagcagtccctgcgctaatcggaggacaagcactgaggataaagaagagacgcggcgaagaagagaggatcaagagtgctcggggaagagagaaaagctctgacgagatctttgtctgcgaaagagtttgcacttccaaaagaatgctgaagaaagttggagctttctctaaagatcctattcccgacacttgtgttactgtctgcggcgtatctgagcttgacgcttgctctgatgcttgtgctcgaaccgtctgtgtcaaccagcaccaagttgctaactggaatgacatttgtcttaggaggtgtcagagtgagtgtcttaagctctcttcttcttcttctcgctcttcttgatatgttttttaagagtttgaatctctcttttgtgttgtttaagtttgagtctgaacgctatgattcgttccttttgaatctgatttttttttttgtaagttaaataacattatgagcaaatctttggtgggttacattcc

SEQ ID NO: 76 - DNA, 442 bp, Brassica napus
gatccctctaagcaccgcttgtggaacacgtgtccacactccagcttcctcacctcctctccttcatgcagcttcgacagacacacgacgcagtctgaaccgtcttcgcctgcgctccggtgcgaggagagccggttcaggctcagctgatcggcgaggacgattgtgttggcgagtcccgaggctatgatggagccgtcgtcgagggggagattagaagcggaatcgcagggagggaggaggaaagagcggagacggctgaggaatacggcgaggagagagaggagcatgagagggagtgaatcggaagagacgtcgctgagctgaccttgtagccccatcttcttttcttcttcgtatcatttgcttagctcatttccagtatatatatatagctttcttggttaaaaaaaaaaaaaaaaaaaaaaaaaaaaaacatgtc

SEQ ID NO: 77 - DNA, 719 bp, Brassica napus
tttgattttgtgtttgactggacggttctcaagtatcaacaatcacaacctggaaacccttcacctcgtgccaatgatggtggtgttgggactagctctggattcaaccctgcagtcagcaatgctgagaaacgtccagacttcccaaatcagaggacgaatctagatcttaacctgaagcagaagacaaagaacgtaaacgaatctgcaactgcaaaggacaagctggtacctggatcgttcttaggtcgatcagaaggatcatcatcaagaagagttgtggactcaagtagtcgtgaaccgttcagtggcgggagtgacaatgcaaactatgaatctgcactcaaaggcattgatagcttgcggatcaatagcaatgcaggagatgagactgcagcgttgccacaatcaaatggagacgatactactcctcctgtagaacccaaagcaaagctgtcagactgattcgtagatgaaagctttagaacataaccggtattcacttttgttgattgcttcacccttcttgaaggagtttgaaggttgtctatgattcttctgaagaaaggctttaaagttagagatttgatataagggagggataaaaaatattttttataccacagtgcttaaatatatgattcatttgtcttctccgttgtatagagaaaaaccttcgtattttaagttccttctatctgataattatttcaaaagtt

SEQ ID NO: 78 - DNA, 789 bp, Brassica napus
tcggcacatgagagccagtggaagtagaagcgcatgaggagattgaagaggaagcgcagaaagatgagacagagatctaagtagtctttgtcttgctctatcttctaatctctggtggttgctttctagtttcttgaaccgtttatgttttgtgttttttacattcaagttttgaacaatcatgtgtagaacatgttatttgcttactcccttttattatctacaaagcagaggtttaatattaggcttctattttataaaatttaagtgcaacaccaaaaaaaaaaaaaaaaaactcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttgccaacagttgcgcagcctgaatggcgaatggcaaattgtaagcgttaatatttgtgtgaaattcgcgataaattttttgtgaatcagctcattttttaaccaatagaggcgaaagtgggcacaatccgtttgagtcgaaagagtagaccgagatacgggtcagtggtactcgcgagttgaaccagagtcggctatgagcgacctggcctccgaccgagaaagtgcgaaaaacgcgatcccgggcgagcgcgccgtccggaccgtcccccccatccagtgtgtgggtgcgtgagcgccgaaccccagcacggaag

SEQ ID NO: 79 - DNA, 572 bp, Brassica napus
ggaaaagcaaaaaaaaaaagatcacaaaaaaaaaacttaagaaggtaagcaaaaatggcgagaagaagtcaagaagaagaagaagaaaaggagaattttcctcttattacaacaaaaacagtcgaatacttacaaccagtaatgcgtcgagagctactccgcaaatttccagacaactctgcttttggattcgactacgcacagagctctctctggtctcctctattgcctcgaaactacgcaagcccttcagatctagactcggacactttcgtttgtaggaaccttgggctcggagagtttctggaaagcaagaagaagatgaagatctcaatgaaaaagaaaaacaaaaagaataaagttagtgaaactagacatgtcttcgatcaagagtgacgattctcctaaagttggctgcttttctcttcccaccaggggatgggatggtttactaaaggtagcttcaaaacacttcaagaaatcgaaaaagaagagagacccagtcgctgatgtcaagcttctcaacttctgcaaatgctgagagttcaatataactaccacttcaatgtaaa

SEQ ID NO: 80 - DNA, 653 bp, Brassica napus
atcaacgcagagtacgcggggagatcggaatcaggttatttggggattgggagagagagagagagaggatggcgtcggggtggggaataacggggaacaaagggagatgttacgatttctggatggatttcagcgagtgtatgtctcactgcagagagcccaaagattgctctcttcttcgcgaagactacctcgagtgtcttcaccattccaaagagttccaacgaagaaacaggatttacaaagaggaacagcgtaaactaagagctgcttcaaggaaaggtgaagaaactggcgatgctactcatactcatcactgatcaaagattttttctccttctactcctccaagtttgcatctctcgcatcgcagctgagaagaagaataaggaagattctatatgttgtgtgtcaaactccattttgaatttttttcagtttgcttctgcaatgaatattctgtctttttttgccttctctattttacatttgaactgagttgtaatatgggcttttagccttcactcaccaaaagttcttcctcatggcgataatttaaaacagactagtggatggaataatgaaatggtccatcgactttctctttgtcgtctcttagagaaaaccaatttttgcaaaaaaaaaaaaaaaaa

SEQ ID NO: 81 - DNA, 800 bp, Brassica napus
  misc_feature (553)..(553): n is a, c, g, t or u
atggacgcctgcggaccggtccggaattccgggtcgacccacgcgtccgcacagcgatcgatgccgtcgacgcagcccttaaccgtcgcagcgaagtccctacgaagccggatcctctccagatccggatccacatccgccggcgcgagccgatgggccactccaggccacgaggagcgtcccaagggcttcttcatgaaccgcactcctccgccgccggggcaatcgcggaaatgggaggattgggagcttccctgctacgtcacgagcttcctcacgatcgtcatcctcggcgtggggctcaacgcgaagcctgatctctcgatcgagacttgggctcatcagaaggccctggagcgactcgaggcggagaggctcgccggagattcttccgattgagaggatcagatgtttgatgctttggatctttgaatcagtatcttttgtttttgttgataatgatggaataagtatcattacaagtttaaaacttggtcacttttgatttgctaggtgttgatcaacatctgaaatttgattgcttttttcgaanaaaaaaaaaaaaaagggcggccgctctagaggatccaagcttacgtacgcgtgcatgcgacgtcatagctcttctataaggtcacctaaattcaattcactggccgtcgttttacaacgtcgtgactggggaaaccctggcgttaccccacttaatcgccttgcagcacatccccccttcgccagctggcgtaatagcgaagaggcccccaccgatcgcccttcccacagttgcgcagcctgaatgg

SEQ ID NO: 82 - DNA, 469 bp, Brassica napus
gaaagcaaagagcttctgcgatctttcatcctcctcggtttcgtatctaatctgatttgaggggaaagagatgggtttcataatggagttcgcggagaatctggtgctgaggctgatggaggatccggaggtgagagacaggaaagcgagggagcacatatatgagatgcacgagaggtgcaagaagattaaggagatgtgggctttgcctattcgtccttatggtttctggacttttgagcgtcacaacgctcagcttcgttgggatcctcagattagccaagttgctggtcgtaggacccttatgatgatctccttcaggaccatccttcctcttcttcatcctcaaactaatcatatacaatggcagaagatgtattgtgatgatgtatggatagatgcatatgtctctctttcaaattcccgattgtactccattcgtggcttttttttttctttcaggtgtctt

SEQ ID NO: 83 - DNA, 569 bp, Brassica napus
ggttgaagagaaagagagagatggagatggctcgagcgtggtcgaagatgatgacggtgatgatcttgatgttgacgtcgacgatctcggcgaaagagcagctgagcactaaggagtgcgaggatctagggttcaccggcctggctctctgctccgattgccactcgctctctgaatacgtcaaggaccaagagttggtatctgactgcttgaaatgttgcgctgatgattctgaggattccatgagtaagattacttattcaggagctatattggaggtgtgtatgagaaagctggttttctatcctgagattgttggtttcattgaagaagagaaacaaaacttccctacccttaaagttgagtacgttttcaactcaccgcccaagttgatcatgctcgatggagatgatgaacgtagggaaactataagaatcgacaactggaaacgcgagtatctaactgcaggtatatgcgggagagggtcaagcctacttcatcaagttagtgtaatcactctaatcattttgtagggctgagtacctgtgtcagaagcactgtgattctaa

SEQ ID NO: 84 - DNA, 433 bp, Brassica napus
ggtctcagccgtcgtatcaccgccgtctcgtcgccgtctgccattttgagaagaaaatgggggacaagaggaagaagacgttcatgttcatccgtctagtctcagctgctggaacagggttcttctatgtgaagaggaagagcggcaagggcctttttgagaagcttgagttccgcaagtacgaccctcgtgtcaaccgccatgtcctcttcaccgagcagaagatgaagtgaccccatttcttttatttcttttctagtggtggttcaagttggggtttagacttttgtttgtaagtttcagtttgaggatcagagaaagtggatctcaggtctaagtacaagacctcaaatcatggttttcattttgttaaaccaatcatttttaatcattacattaagtactttgggtgtataagaagctcaagactttt

SEQ ID NO: 85 - DNA, 607 bp, Brassica napus
ggttcagcgaccatctagaggcaatcaagtttatctgtaaagacttctggtctgaggtcttcaagaagcagattgataatctcaagactaatcaccggggtacttttgtattgcaagacaacaagttcagatggctatctcgtgtctcaatcgacccttcgtctgagaaggaaaccgaagatccgtctactccaggggaaagcaaagcggcacaagcggtgagcatgtacctgtatttcccatgtggaatcataagaggtgtcctctccaacttggggataccttgtgctgtctctgcggacatatccagccttcccacttgctctttcgtgattcgggtcaaggcttgatactggcattagctttgagtcctcctcctcgatttcaaactccagctcggctttatggttttcaagaaagatagattctttgtgcccttacgcagacttcttaagctgcgacttctgtcaaaacacaacttttgaattctccaaaacttgctgtagaacattattttggatgccttaatgggcctatctctccctcaaaaaaaaaaaaaaaaaaaaaaaaaaaaaaaaaggaaaaaaaaaaaaaaaaaaaaaaaaaa

SEQ ID NO: 86 - DNA, 202 bp, Brassica napus
aggtttaggttaattaggtttaggttaaatatgttttggttaattagatttgggtttaatgtttttaattaggtttcttcgttatttgaagtactccgaaattaccgagtttgtttacaggtatccaacatttctaatgtcgcataacaaaatgatgttcttggcataaaaaaaaaaaaaaaaaaaaaaaaaaaaaacctgg

SEQ ID NO: 87 - DNA, 734 bp, Brassica napus
ggggtgagacaagaaaaatggtagttgcagcagcagcagcatccactccattttcactctcttccgccgttttcacgcgccgccgtagcttcagagtaagcgccactctttctcgggagccctctcctcttctccgagctgctcagcacaccgtggatagttacgtggagagtgggatggttgttggtttaggatctggagaagcttcagacttggctatacgttatttgggtcaccaacttcgttctggttctattcaaggtgttgttggtgtaccaatgtctgctcgaagtgcgagcgaagcagcaaagtatggagtccccttgaaacatttccgggatgattttcaggtagctctgcatccgtgctttgtctaaacatctatctttcagcttgggatgtaatgtttgcagtgtgactcttcatctatgtagattgattttgcattccatgatgctgatgctgtagaagagggtactcttgtctcagttataggaagacgtacaagtacaacacaggaagatgactatattctgctacaaaagtctattgtgaaagcagctgatgaggcagtcttcctgataaaggatgagcaatacaagtctggacttgaaggatctatccctgtcttagttcaatctctaaattggttagctgtagctgaggagatagatgacttgtacttaggagatgcagaggtgtggagaagagcttctgtgggagatgcagggcct

SEQ ID NO: 88 - DNA, 441 bp, Brassica napus
cggcacgagggacacctcgagtctccagtcaccacccctctgtctcctttgctttggttacaacctcccaccttttttctctcttccctagcctttttcctatatgctctctactctgattttcttccggtaaattctctctagtggaccaaaaatgggtgttgtaggtgtatgtgtcctctttggagtaacggtgaggaaacattttggggttttgtcaaagggttccggtggagtttgttctttggtgggttgagagaccagggtcgaaggtacatgggcaaacggtggggcaattgtccaccgtgacttttaaaatttcaatgtattttctttaaatttttagtgaattctccttgtatatagatagaataatctgttaataaattttattttcgcctggatcaaaaaaaaaaaaaaaaaaaaaaaaaaaaaaaaaaa

SEQ ID NO: 89 - DNA, 769 bp, Brassica napus
cgtctcttaagacgacgccgtatccacgtccaccactgcatcaaaaacaattctcttctgaagagattacaccttatccaacttgagtttgcaataggagaagccattgttttccgtatcttagacctgtatactgagcttcatctctttcaatggcttccttgtcagccacttctctaagcttcaaagctccctccacacaatcaaccagaatctctcaggtgttaaggaaagcctcgacttttcagtctatctcctttggtcgtttccagtcttcgaagagccttcgtcttcaaatcagttgtgcagcaaaaccagagacagtacagaaagttagtgacattgtgaaggaacagttagctttatccgctgacactgcgctcactgctgagtccaaattctctgctcttggcgcagattctcttgacacggtggagatagtgatggcattggaggaaaagtttaacataagcgtggaggaagctgatgctcagaacattacgacgattcaagaggcggctgatttgattgaggatcttgttcagaagaaacctgcggcctaagcttcttaaactctcccggctttcttttcctttagacctttcttggtttgagaacatttaattagaccgtatgtcattttgtttttcttcttctggaagttgagattggatattgactatgagaaaactttgattggtacgttgagattgattcattgggaatgattggtcttttatgaagaatatagaaatgtgttatgtgtcac

SEQ ID NO: 90 - DNA, 669 bp, Brassica napus
gaaccgtttcttctctctctgtctctctctctctctccggcagctgtggaaccatgtggtcggcgacgctcgctttcccttcctttggggcttcatcaacttctctaccaagctacaaaataccaagacttccgaggatccaagcttcgctatctaattaccctctcgccagcaaaatcatggtcagaaacttaccgttttccacaagtgaagactttctgcaaaaggagttttcagcttttggagagatagctgaagttaagcttgtcaaagatgagtcgatcaagagatcaaaaggttatgcctttattcaatacatgtctcaagacgatgcctttctagccatagagaccatggaccgtaggatgtacaatgggaggatgatttatatagacattgcgaagccagggaagcgtgacttccaacaacagccgaggacttctggtccccctgagaagctccaagtgccagaagaatcagctagtaacgaggtcgctgactgctggtattaattgttaggaccgagttcagtaaactgtgtctggatttgcatagaaaacaatgatcaaatatgaacacatgttgtttttcactactgataaacaatcaccgacacggttccaatcacatctctatcagtttcgacaaccaaaaaaaaaaaaaaaaaaa

SEQ ID NO: 91 - DNA, 648 bp, Brassica napus
gctagcgaaattcacttgagccgctctccgaaagcattttcgatcccagtcttaagaaagaaaaatcaatgtcgacggtcggagagcttgcttgcagctacgctgttatgatcctcgaggacgagggtatttctatcacggccgacaagatcgctacgttgatcaaatccgctggtgttagttgcgagtcatactggccaatgctattcgccaaaatggcggagaaacgtaacgttactgacctcatcatgaacgttggtgctggtggcggaggtggtgccccagtttcagctgctgccccagctgccggtggtggtgctgcagctcctgcacctgccgctgaggagaagaagaaggaagaagtggcagaagagagtgatggtgatttgggtttcggcttgttcgactaagcaagaagcaattgcttttgtttttttcttgttattagttggttgttttatccaattgagacgcgccatgttttgtttagttgctacttcgtttcctcaagttttggttgttttcttgaatcaccctgtttgtgtatctcaagtctaccgagcaaaaccatcggggagtttgacattttctttatatttccttctttgacagctaatattggtgtgagatttataaatcatgaaatcg

SEQ ID NO: 92 - DNA, 660 bp, Brassica napus
gagatttctcatttcgtcagatcggagccacacaccacagcgatcgatgccgtcgacgcagcccttaaccgtcgcagcgaagtccctacgaagccggatcctctccagatccggatccacatccgccggcgcgagccgatgggccactccaggccacgaggagcgtcccaagggcttcttcatgaaccgcactcctccgccgccggggcaatcgcggaaatgggaggattgggagcttccctgctacgtcacgagcttcctcacgatcgtcatcctcggcgtggggctcaacgcgaagcctgatctctcgatcgagacttgggctcatcagaaggccctggagcgactcgaggcggagaggctcgccggagattcttccgattgagaggatcagatgtttgatgctttggatctttgaatcagtatcttttgtttttgttgataatgatggaataagtatcattacaagtttaaaacttggtcacttttgatttgctaggtgttgatcaacatctgaaatttgattgcttttttcgaatgattttgattcgggtattgtttgtttcagtattggcctttgctatcctgtgcttcttgatgaagttcatagccagtttaaatccttttttctagcaaaaaaaaaaaaaaaaaaaaaaaaaa

SEQ ID NO: 93 - DNA, 621 bp, Brassica napus
cacgatattagtggctcgaatctcccaatcaaactcatcttcctcaccggtgccgcatctctattccacggatttggttttttgaagatggcatcgaagttggtacaagttcagtcaaaggcgtgtgaggcttcaaagtttgtggctaagcatggaacttcctattacaggcagttgcttgagaagaacaagcattacatccaggagcctgccaccgtggagaagtgccaagagttgtctaagcagcttctctacacccgtcttgctagcattcccaaacgctatgagactttctggaaggaagtagactacgcaaagaacttgtggaagaacagaagcgatctgaaggtagaagatgcaggaattgctgcattgtttggtctcgaatgctttgcgtggtactgcgcaggagaaatcgccggaagaggattcaccttcaccggctattacccttgaaagagaggaacaataacttgaacaagattgtgtaatctgaatcaattttttttttacaagtttttccctgaaacggaaacagagtatttggatctgagaaaatttccattttgctaaaactctctttgataagtttgagattaataaagaacaaaaaattccaga

SEQ ID NO: 94 - DNA, 521 bp, Brassica napus
gtcatttcaactctgcaactccatccacatcctcagcagtttttaccaaattgccatcgaaaccgaccagagctcacaggagaatatggtatggccaggtgcattggctgagatcaaacgacatggcttcttcgaccagctacaaactcctgacttgtcggatcattaccagacccggaccgagatcagttttggatcttaccggtttatctgatcttaatggtaatggcgacgattttaaagaagacaaaggaggagaagctgaagatgaaatgacaaatagagagaatggacataatagcgacaaagaagtagaagatgggtcttgtggcgggtatgttgatggaagcatgagtttttacgatgtggggatgatgatggaacatgtgttagatcacgatgacgatgaagaagaagaagatggttggtgtttggtctgaaaaagaagaaagtaatgattatgtcatgtctctctcacgtggatcagcttttttttttttttttcgcatattcaagtgaat

SEQ ID NO: 95 - DNA, 573 bp, Brassica napus
aatcggagatcagatcgacctcctggtttgaacttcggctcgccgggatcgacggcggcgaactcgagcagcgtctcgttgaacgcgacctcctcctccgccgtcgtcacgaatccggaggtgctgccgagctcggctatcgccgagctgaaggagaagcttctgcggcgaatcgcgactctgcagatgagcgtcgcggcgaggaagcagaagaggagggcgctgaagagaggtcggacggatcgtttcatctggatctcaaatcgaatctgtaatggtgactagtgagctcgacgacgactccattgtgaagtcaacgagagggagacgacattgttgctttaatgaaagatttggcaacccctaaaatggtgatctgagcaaggaggttagtagatccgtcgacgattcaaactaaaaattaaataaatcttttctttttctggtagatgacgcagcaccctccatgttgttgtgttccccctttacgctttgtagctagagtagagagataaagagtgtgtgtgtgtagcttgtttaattcttttctttcattaataaacttcttttaac

SEQ ID NO: 96 - DNA, 961 bp, Brassica napus
gaaggtcacggcgagacgagccgtttatcattacgataggtgtcaagtggaagtgcagtgatgtatgcagctgaggcatcctaacagaccggtagacttgaacctggccgggtcgtgcctccggcgctgttactttgaagaaattagagtgctcaaagcaagcctacgctctgtatacattagcatgggataacatcataggatttcgatcctattgtgttggccttcgggatcggagtaatgattaacagggacagtcgggggcattcgtatttcatagtcagaggtgaaattcttggatttatgaaagacgaacaactgcggaagcatttgccaaggatgttttcattaatcaagaacgaaagttgggggctcgaagacgatcagataccgtcctagtctcaaccataaacgatgccgaccagggatcagcggatgttgcttttaggactccgctggcaccttatgagaaatcaaagtttttgggttccggggggagtatggtcgcaaggctgaaacttaaaggaattgacggaagggcaccaccaggagtggagcctgcggcttaatttgactcaacacggggcgaccccaggtcaggcgggatcacccgctgagtttaagcatatcaataagcggaggagaatctgagttcggcgacttgacgatgagggcgaagtggaagaagaagcggatgaggagactgaagaggaagcgcaggaagatgagacagcgatccaagtagtctctgtgtctccatgcctatccataactctctgaaccttttatgttttgtgtttttttacgtttaagttggaacaatcgtttgtagaacatgttttggatcaactccctttttatttatctacacagacagagatttatcttatttttcttggatctgtttattttggagattgtgagtgcaagtaacaattttctagaggcttgataaatgatc

SEQ ID NO: 97 - DNA, 462 bp, Brassica napus
ccggtaattggggtccgtaaaattgtttcaacactaaaaccaattttattcggaattttcaaatgaacaatacaaccggtttaattgaggattcgagaagcactatagtgaaccagttcggcttacagtggagcagagagagtaatgggaagattggcctctctgtatataaaaatcaagaaaaaagaaaaaaattgaagaatactaaccggaattccatttttttgtttctcttttctttacagagtatacttttaacatgatacaaaattatttgctacatttctttggagagaattttaataagactgatatttacgtgtatgtgtgatctatgtgtgttttgcaaacttgtgcttttgtttttggtaatgaacatgccaaccgtttgtaatgtttaagattctgataatctgatttatattttttgattatatttgggaatcggaagaagcattgtgt

SEQ ID NO: 98 - DNA, 716 bp, Brassica napus
gagaatagtcaaatcctctcctgagaacagcacacagggcaagacaaacaacaactaagcaagatgcttgagtttatcatgttaacccctcaataagatgcaaactaattttttttattttccaaaaaaaaaaacctagacctatccagtgaaatcacggtatggaactccatcaagtttagccagcttattgcaataactcatctgttgtcttgcatgctacacactgaactcttgcatatcctcacttcctaagcgtcaaaagttcactcaaaacacaaaaacctgtatcgattgaaaaagctgtaagagctagcatcctcatctcaactccatctcctcacatagccacgtaatcctcctctatcatgatgagcgagagatatcgagaggaggaatgagaggtacgggatcagagatctccgcgactccgtcgtccttaaggctgattcgaaccaatgaggactccgttcatcgcagcttcttgtgtcgcagcttttataggtgcagagagaccgacttgcagagggataagatgaatcaatctcagatcgtgctcgacgtgggttgatggtattaagatgtttgttaaagcgatgaattaataaccattaacgtttcgaaggattagaaaggaaggcgtaaagacgcaatcgaagcttcttacagatattcttcaagactattcaactcacgcatcctctagaaactccatt

SEQ ID NO: 99 - DNA, 182 bp, Brassica napus
ggatgatagatatagatgaactcattgatgatctaatagtgagagggatctttttttttcttcttcttaatgtctttttgagtctcttgagagtacatttggttcatgtcttgtttcatttccaagtgcaatgaagtgaaaaaatgttctcaaaaaaaaaaaaaaaaaaaaaaaaaaaaaaa

SEQ ID NO: 100 - DNA, 478 bp, Brassica napus
gactcgattctcactatcgtcaccaccatcactctctctctctcttctgttggaatttctccttatggccattaacacgagcttcattgttgaaattgcaggtcgtaaaaaaattttcagatctggtgcagcaaagacagaggttctggttttgtagaacaatttaaaatatagaagagatggctggagaggaagacggaggagataccgccggagaggaagacggaggagacgcggcttgagaagaagattgaggagacacggccggagagaaagatggaggagatactgccggagagtatataatattaattttatctataatttaatttttattttttttaattaatttttttagaatagccacggatatgtagtgttaaaccgtggttatacatagctatgataaatctttggcaaaaccgtcgctatctgatccacaaaaatgtagtggcacacgaatagccacggttttttt

SEQ ID NO: 101 - DNA, 750 bp, Brassica napus
agggactcttctctgctcatctccaccctcaatcttgaatcccttaatcccaaaatggcgtcagagaagaagctctcgaaccctatgagggacatcaaggtccagaagctcgttctcaacatctctgttggtgagagtggtgatcgtctcactcgtgcctccaaggtgttggaacagctcagtggtcaaacacctgtcttctccaaggcgaggtacactgtgaggtctttcggaatcaggcgtaacgagaagattgcttgctacgtcaccgtgaggggagacaaggcgatgcagcttcttgagagcggattgaaagtgaaggagtacgagctcctgaggaggaacttcagtgacactggctgctttgggttcggtatccaggagcacattgatcttggaatcaagtacgatccttccactggtatctatggaatggacttctatgttgttcttgaacgcccggggtaccgtgttgctcgtcgccgtaggtgcaagactcgcgttgggattcaacatagagtcacaaaggatgatgccatgaagtggttccaagtgaagtacgaaggtgttatcctcaacaagtctcagaacatcactggttgaagagtttgtttctattttgttttgtggtgttagctcttgtctcagaacatcgccttgggatttcttgtttttttgtatcttttttttaacttcagttggtttagaatctgagtaccctcgagatatcaacaatttttagaggtttta

SEQ ID NO: 102 - DNA, 717 bp, Brassica napus
  misc_feature (573)..(576): n is a, c, g, t or u
acggaccctgcggaccggtccggaattccgggtcgagccacgcgtccgaagctcttcaattctcgacaaaaccagtgggcttggattgaaagatttcaagcaacagattttcttcctgcaaactccacaaacctttttcatgatcccataagttgtccaatgttgggcattatgactccaggaaagagcagaaaccatttaagtgcttgtgcaataaggaggagaagagtgcattcgaactcggatacgtacgtactgctagaggcaggacaagatgaacagtttgtgtcagaagatgagctcaaggctaagctcaaaggctggctcgagaactggcccgtagaatctttgccaccggacctggcaaggttcgataaccttgatgaagccgttgatttcttggttaaagctgtatgtgaactagaggtttatggagaagtgggttctgttcaatggtatcaagttcgtcttgagtaattaataaatgtattgtgtttctcctatttaataagcacatggttctgtaagaaaaatggagaaatttgctaaaatagaacaaaaattgactactannnnananaannnnnnnnnnaaaaaaaaaaaaaaaaaaaaaaaaaaaaaaaaaaaaaaaaaaaaaaaaaaaaanaaaannggggggcggccgctctagaggatccaagcttacgtacgcgggcatgcgacgtcatagctcttctt

SEQ ID NO: 103 - DNA, 735 bp, Brassica napus
gaatcgagagaaaagcgagagaacgtgacggcgaggaagaagaagggagaatgtggttcaccggcggcggaggaggaggactgaggaagctctgtagagcctcggcgactgttttcgagaacgagatgagttgtaactctctgctagtgagatacatgtcgagagagcgagctgtgaacgtaaggaagataaacccaaaggtctcgatccaagaagctcacatcatctccagctctctctacgaagtcttcaagaaacacggacctctctccgttcccaacacttggctccgcgctcaggaagctggggtaagtggattgaacagcaagactcatatgaagctgatgttgaaatggatgagagggaggaagatgctcaagttaatctgcaaccaagttggttcctccaagaagttctttcacaccgttttacctgacgatgatattcaacaagagacaccacttgctgcaactcctgctgccaccgagaacaaaaagcaaacctccaagagaagaagcaaataaaagattgttatttgcctttcccatgatgaaccgtgtgagcaaatctgtgtggacaatctcttgttttgatctgtttttagtttcagagaaaacattatagtttgctctggtgtagaaagtatgtgggcttctctgtttcctttgaataaaagaaacaacagtttacattgcagaagtaacatcaacatcacattggatacatcaaaactcg

SEQ ID NO: 104 - DNA, 396 bp, Brassica napus
tgtagctggttccctccgaagtttccctcaggatagctggagctcggaaacgagttctatcgggtaaagccaatgattagaggcatcggggacgatggtgaagagtggtatctcaatggaccagctgaaagctctgtggcactctgaggtccacaatgagcagaagtgggcagctaacatgaagcttgtgagagcacttggggtgtttgcaggaggaatcttcctcatgcgtggctttggggatctcatggctgtctgaacaaacctgggaggagaacatttcatcgtttttttcgatttttgtattttctgaagtgtctgctctctccacagagtttgatggtttggtttatgattccaccattgtgatacagatacatctcagttcaagatttg

SEQ ID NO: 105 - DNA, 621 bp, Brassica napus
gaggggagaagataagcaaaatggcgacttggataacgttacaccctccttatctacgcttcatgccatcatcgccctcgtcgcctgattcatcctcgttgcgtcggcatcgccattctctgtttaaaccatttcgatgcgctccacttcagcaacaagaacgccagctcgagcagtcacaggggggcgaagaagaagaggatgatgaggctgcgataatgtgtgaggactgtaacgggaaaggatggttgctctgcgatttctgtaaagggcagaaaactaatgtcaaatccgagaacaagaaaatctaccgtcgctgtcccacttgtagagctgtgggatatgtgttatgcaaaaggtgtaaagtgttcaagtgcgtcacgtttcctaatccagaagacggcgacgagcttttgttctaagctatccttgtcctatctttagtgataggcccttttgtgtttcactttcaggttcctttccttttcctatttgttgttgttaatatataactccccaagctcaaaccaatttagtcatatgtataagcacattttgtaattgttaaacattaatctgataagtgattgaactgccaaaaaaaaaaaaaaaaaaaaaaaa

SEQ ID NO: 106 - DNA, 541 bp, Brassica napus
tggtagaaattgactgaagttgatcgagatggggaatgagacgaagggcaatggagcttcaagcttaggaggaggaggagggtttagggcaaagatggagcactatgtgtacagtggagaaaagaagcacgttttggcagggatcgggatattcaccgtcatcttcggcatcccttggtatcttatgaatcgaggatcaaacaaccatcggtctcaccaagattacatggaaaaggctgataaagctagaaaagcccgtctctcttcatctccatccccatcctctgacaagtagtccattttcagactctagtcagatacatgccgtgaatgatccactcactcagggttttccatttccatacaaacataactcagcaatgtctgagccagtgtggtttatttagttcagttacgtatcattccactgtactccagtttctatattgaataaataaaagtttttctcaaacgcatcaaggccggatcacaaatattaaacccgggggggcccgcctcgtccagcaaccttttattttta

SEQ ID NO: 107 - DNA, 1086 bp, Brassica napus
  misc_feature (699)..(705): n is a, c, g, t or u
ggcagacccactcgataaccctactaaagggaacaaaagctggagctccaccgcggtggcggccgctctagaactagtggatcccccgggctgcaggctcctcctctccctcctccacgtcgccacaacctcctccgccgagatttgctctccttatccgccgcatcgacgctacttctaacgcaatctctcccgtttctagctccgtcgccggcatccgccgccgaggacgaagagtacgtgaaagatacgtcggcggtgatctccaaagttcggacgacgctctcgatggagaggacagatccgaatgtggcggatgcggtggcggagctgagagaagtgtcgaactcgtgggttgctaagtacaggaaggagaaagctcttctcgggaaagcgtcgttcagggatatttactcggcgttgaatgctgtgtctggacattatgtgagttttggtccgacggctccgatcccggcgaagaggaaggcgaggattcttgaagagatggagactgctgagaaagctctctctagaggaagataaatttacagtgagacctaaccatttcttcttcttcttctccagttccttgtcgatgaagttttctttttacctttaaatgcttatatgagaagagaaacttgaaattgctcgttgtttttgtaaaatacttcatgataatttacttaattatcattnnnnnnnannnnnnnnnnnnnnnnnnnnnnnnnnnnnncctcgagggggggcccggaacccaaattcgccccttatagggagtcgaattaacaattcaactggcccggcggttttaaccaacggccgggaattggagaaaaaaccccttggcgttttacccccaactttaaattcggcctttggcaagacaacattccccccctttttccgccaaagcttgtgggctaaaaataacaacaaaaagggccccccggacacccgatatccgcccctctttccccacaacacaggttggccccgcacaccccccggaaaatggggcaaaaagagtgccaaaaaatttgtggaaggcccgcgccaaaaaaaaatttttgggttaaaaaaaaa

SEQ ID NO: 108 - DNA, 525 bp, Brassica napus
gatcccttggcgtgtccctttatattggataatccatcttctctccatcttctcttttgctcgacctccttcttcgtttgattcctcctccaatctcagtaactccatcttttatttcacttctgatctcgatttcttttttcgtttgattcgtttgattccttctctgcagttacgattgtttctcctctcgaaccatcttcttcaacgcgtttgactcttcttctctgtatctgagttagcaatgaagtcaggaggatcatgattgaacccaaatgcagcagcttacgcaccactctccaaaagagaaggtgattcttcaaagcatgttggtgctgatgcagccacacgtgacgttcagcaccaacaaccctatggttatggagtccaagggaagggaagttctccaggcgctcacatgagggatgatgattcggagactgataaggagatggagttccttttggccaactccatctccgtgatgtttgtcagatgagtctatatgtgatgtttacttagcca

SEQ ID NO: 109 - DNA, 516 bp, Brassica napus
gacagatctatctctcacctatcttcttctctttctttcatcaccaccaccaccaccaccaccaccaccctcccatccgccatagctcccatccgctatctcaccaccatcatcagttctcagatccgacgccaccaccaccgccgtctcgtcataatcaccactcagctctctgatttcgttctctctaatttgagaaagaagcacgaggcgctcaccagtggcagtgcttttaccaaatctaaaagtctggtgtttgggctattaaatctgctggagttaaagtgatagagaacagactaccaattgattcttgccgtattagaggtccagttatcaaaggatttggccgtggctctaaatgactcggaatcccacagtgatggatttttaagcgagaagggataaccaaatttcactaggttatgaagatgtggtgtggagatggcagaggaagctaggggtggtggtagagttggagccgagcaagaacaaaaagagaaaggtattcgtgag

SEQ ID NO: 110 - DNA, 538 bp, Brassica napus
ggagactaattaaaaagaagccaccgagaatctctctctcttcgtcgcccgacgagttttcgaagaatctccgtcagtttggaaagaaagagaaatgccacacagaacaaggcctttgacgggactcttgcttttcactggaatcaacgctgttttggtgcaaaccattactcctgtctatgacttcgtctgcttcttgccttactgggagagaaggagagagagaatccggaaggagcgtgaagcggtggctggatcattaaacagaacatcctcagcacaggataatactcaagcatctgttggttgattgaactcagtatcgaagacagtcgcaatgtttcctctgtgtaatcaactgtttaccctctaaagcactgaataattgagagcctgcagctaccgagccgtttttgtaatggcccatataaaggcccaaattgggcatgttgtctttaagttgctgcatatggcccattagtatcgagtattgtcaagttcagaagtcgcaatctatatcataaactcaacactacgc

SEQ ID NO: 111 - DNA, 736 bp, Brassica napus
tgcggaccggtcggaattcagggtcgaacacgcgtccggtggtgagtccgaggaggagaaagagttttgaatcgaaatatggctgggagagcttcgatccctgctcgcaactctgccctcatcgccatgatcgccgatgaggacaccgttgttgggttcttgatggctggagttggtaatgttgacatcaggaggaagactaattacctcatcgtcgactcaaagacaactgtgaggcaaattgaggatgctttcaaggaattctcatcgagagatgatattgctatcatcctcatcagccaatatgttgccaatatgatcaggttcttggtggatagctacaacaagccagttcctgcaatcctggagatcccttccaaggaccatccctatgatcctgctcatgactctgttctctcccgtgtcaagtacctcttctctgctgaatctgtttctcagcgttaagcatatacacattctgcaagctcgcctgagcttttatatcatttgacattcttgtatttctcagttttcaataatgtgtatctcttggtttttgtttggaaatggatgttatatcagtaagaagcaatgataacaccatctggttctttttgcattgaataaagagagtgagtcattgtgtattcagcagactttaaagatttaagtgaagactccatctattcaaaaaaaaaaaaaaaaaaaaaaaaagggcggccgctctagaggatcc

SEQ ID NO: 112 - DNA, 746 bp, Brassica napus
ggggactcgcgagcttcttcgtctctccctcttctggtgcgatctatctctatctctctctctctctatctgcgcgcgggtgtgagacttttggcgggaaaatgtcggggaaaggagcgaaaggattgataatggggaaacctagcggtagcgagaaggacaaggacaagaagaaacaacccatcacccgttctgctcgagctggtcttcagttccctgtaggaagggtgcatcgactgttgaagactaggtccactgctcacggaagggttggagcaactgcagctgtttacactgcagcgatattggagtatctgaccgcagaagttttggagctggctggtaacgcgagcaaggacctcaaggtgaaacgtatctcccccaggcacttgcagcttgcgattcgcggagatgaagagctagatactctcatcaaaggaactatagctggtggtggtgtcatccctcacatccacaagtctctcatcaacaaatccgccaaggaatagattcttaccactctttgtttctctgtttcctaagtatttcgagagagagtctgtgtttcataaagaagacgctagagtgctcttttgtaggagtcgtttgttattgttccatcagcgtactgtgttagagacgcttgttgatttatctatctttatgcatatgtattcgatgattatctaaaccccatttctgacttaccttctgggtttgttcctacccaaagttgcatatcatcg

SEQ ID NO: 113 - DNA, 797 bp, Brassica napus
cggcacgagggagagagagagagagagagagagaaaatgtcgacgcttctgcaatcgctcatcgatccgaagaagaacttcctcgctcgtatgcatatgaaagccgtatccactcgccttcgtagatacggtctcaggtacgatgatctgtttgatcagtatgaaagtatggacattaaggaagcgctgaacagattgcccagggaggtcgtcgacgctcgtaaccagcgtctcaagcgtgccatggacctctccatgaagcacgagtaccttcccaaggatcttcaggcggtgcagaccccattccgtggctatcttaaggagatgctggctctcgttgaaagggaaagaaaggaacgtgacgccttgggagctctaccactctaccagcgcacactcccttaagcacatccttttttccctcttcttttgataaactctacttctctgcataaaatctcctctccagatggattgaattttgatattttggaaatcacatttctgaacagagctgtttacttcctgaaaaaaaaagcacaagtcttgtgatacttttgcctaaatgggatgatatgtttgcttgttacattttttcacttttgctggaatgaataaaatcgacgacccaacactgagtctggttcagtgactctcattttctcatgttaaaaaaaaaaaaaaaaaaaattgactcttgtaccaaaaataattatcaatgggtgtcccaaaaaccttgatgacttttcttctccttgtcatattaggagtttccctattccactccattgtgttta

SEQ ID NO: 114 - DNA, 471 bp, Brassica napus
cggcacgaggctttaactcaaattcacctcaattatcgattccttaatcactcaccattctctctcattgctgtgaagaagataatggtgctctctgacttcactggagtcggtgttggattcgggttcggcgttggctgtggatttggcgttggctggggttttggaggaatgcctttgaatgttttaggcgttggtgtaggtggaggttgcggagtgggtttaggcctcgggtggggttttggaactgcctttggtagtcactaccgatcatctaggcttacctttcaaggcgtcgagctagagaaggagggtaaagttgaaaacatgtccaaaaacgcttagcagcctcgaaacttcccatcccaaatcactgagaataactccgctcggatttattgtattaagaaaaacatttttcttatttctcaataagaatcatatttcaaataaaaaaaaaaaaaaaaaaaa

SEQ ID NO: 115 - DNA, 982 bp, Brassica napus
  misc_feature (830)..(830): n is a, c, g, t or u
gtactgtagctccactcgtattaaccctactaaagggaacaaaagctggagctccaccgcggtggcggccgctctagaactagtggatcccccgggctgcaggtacgtacacggagcacgctcgccggaaaacagtgacggccatggacgttgtctacgctctcaagaggcagggtcggactctgtatggattcggcggttgattcgattgtttcagtttcagatcttgtaaaatctttttcagaatcaatggcaaaatatgcatctttttctaaaaaaaaaaaaaaaaaactcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaacccttaagggagccccccgatttaaaagcttgacggggaangcccggcgaacgtggggaagaaaagaaaaggaagaaaagcgaaaggagccgggcccctaggggcgccttgccaaatttttacggtcaaccctttgcccgtaaacaaccaaaacccggccggggcttaagtggcgccgttaccagggccccgt

SEQ ID NO: 116 - DNA, 443 bp, Brassica napus
ggccgcacttgctttgcttctcctcagtgtttgaattttagatatcgtctacttgaattcaagatgcagaacgaagagggtcaggtcacggaactctacattcctaggaaatgctctgctactaaccggatgatcacatcaaaggatcatgcctctgtgcaactcaacattggtcatttagatgctgatggcatctacaccggtcaattcactaccttggctctctgcggttttgttcgtgcccagggagacgctgacagtggggtggacaggctgtggcagaagaagaaggtcgaagccaaacaaatctaaagagcctgattaagctgtccttttgctttgttctgaatgttgttatttaattcaaaaatgatcatcggacatactgaagtaatgatgatatttgcttgactcttggttttaatgcttcttgtgttctgatt

SEQ ID NO: 117 - DNA, 692 bp, Brassica napus
ggaggcagccagggcttccatttcatcatgcacagctccttgtgcgagcttattctcctctaccaccaaacctatcaacagttcttcttcttcttcttcctcctcagtaaggctctcctctcggtttcttggaactcgggttgtgaagctcagtatccgactcggaccctccaatgggtctagagccacttgctggttcaagttcggaaagaacggcgtcgatgctgagaatgctggaatctatggcagccaatcacgtgatgatttcgatagagacgatgtcgaacagtatttcaactacatggggatgcttgcggttgaaggtacctatgataagatggaggctcttctaaaccaaaacattcatcctgtagatattcttctgatgttagcagcctcagaaggagacaagcctaagatagaggagctcctcagagccggcgctgactacactgttaaggacgctgatggaagaaccgctcttgacagagccagcagtgaggagatccgtgatttgatccttggatctctcactcaaaaggcttgacaagatctcctctagcttcttcttacagagaaggtgatgtctgttttttggtaccttggaatcttagtcgtttcaatgttctcttacttttacgtaatgggtccgaaacctaagtgtgcttcaacctttaaagtaagatgaatct

SEQ ID NO: 118 - DNA, 512 bp, Brassica napus
aaaatgaaaagtactctaacaattgtgagtggaagaaattttaacacaggaaacagctatgattttaagtttcttcaagagcaaagctaggatccaaatttggttttttgagcagaaagatttgaggattgaaggaagaatcactgggtttgacgaatacatgaacctagtgttggatgaagctgaagaagtgagcatcaagaagaacaccagaaaacaacttggaaggattctacttaaaggagacaacataacgctgatgatgaacacgggaaagtgatgactattctctcaagaaacactctcaaggattagtatatccatagcttttgctaagtatatgttgagttgagctcttcttcttcttcagttaactgacaatgccttcttcgttatcgtgtaatattgcaatttagtaagctagtaacaaggtttgagaaattatggagtttggtggaatcttaaggatggtattgcctctttttggttaatggaaattactattttcggga

SEQ ID NO: 119 - DNA, 1018 bp, Brassica napus
  misc_feature (1018)..(1018): n is a, c, g, t or u
ggtgctccaaaaacatggtgacccttatctctcctcgttctctcttccctcctcttagtagtaaacccacttcaaaatcctcgtctttctccaaacccgtctcttcccaatctcccaagttcgctaaaccggaatctccagctccccataaacttgttaaccgatgccaaacaggcttgtgcagttgggaggaggaggagcatgatgatgggtggattgctcatgtctggtttaatgctttcagaagccaatcttccaacatcagcatttgctttgacttcagtcttcagggagtacattgatacatttgatggatactccttcaaataccctcagaactggatccaagtccgaggagctggtgctgacatcttctttagagaccctatcgtcctcgacgagaacctctcggttgaattttcttcgccttcttcctccaaatacaagtcacttgaggacttgggaccacctgaagaagcaggaaagaaagtacttagacagtacttgactgagtttatgtccaccagactcggtgtcaagcgcgagtccaacatcttgacaacttcctccagagttgcagatgatggtaaactctactaccaagtcgaggtgaacataaagtcatacgcgaacaacaacgagctagcagtgatgccgcaagatagagtggctcgtttggaatgggacaggcgttacctggcggtcctaggagttgagaacaatagactctactcactcagactccaaacacctgagaaagttttcctggaagaggagaaagatctaagaagagttatggattctttcagggtcgagaagatttagagagagagagagagagagaagctcacaattcttcttcatggctttctttttccccagaaaagaaaaaagaaaaaaaatcttgttcttcctcccattgtatttttgaaaggcttattaagctaatctaaaggctttgttgtaatgcttttctacagttatcagattcattccttgacggcaggagatggttcan

SEQ ID NO: 120 - DNA, 600 bp, Brassica napus
ggaagtcttattcttcggtcgggggcgggtgggagaggaggaggaggaagaagacggcgacgatgggagagggttgggggttctacaaagaatacgtcgccggaatgaatgctggcctcgccacagtcgccgtcggccaccctttcgacaccgtcaaggtgaaacttcagaaacataacacagatgtggaaggtctcagatacaaaaacggtctgcattgtgcttcaaggatcctccaaaccgaaggagtgaaaggactttacagaggagcaacatcttcgtttcttggaatggcctttgaaagctcactgatgtttggaatatactcccaggctaagctcttcttgcggggaagtttaccagatgatgggccacgaccagagataattgttccatctgctatgtttggtggagctattattagttttgtattatgtccaacagagctagtcaagtgtagaatgcagatccaaggcacagattctttggttcctaacttccgtagatacaccagtcctctcgattgtgctgttcagaccgttaaaaacgaaggggtaacaggtatttttcgcggtggctcagcgacattattgagagaat

SEQ ID NO: 121 - DNA, 446 bp, Brassica napus
ggatcgtcaacgcgaatcagtcaccgaagacagaagagagatgaggttgttcgatccgtggccagtgttcttcaagagggagtggaaacgttgctggccgtttctcaccggattcgccgttaccggcgtcctcatcaccaagctcacggctggattcaccgaggaagacaccaagaactccaagttcgtccaacaacacaggcggtgaacatgagaaagggaaagtcggctttcaaaactattctaataacatagaagagaggattcttgatactgttcgtctctcttttccttttttttctacttttgttctccccgtatgtttgaagtggattttgattcctccctttctccccatacatttaaataaatcttacagtcctgaatgattgaatccaccaacaatagattcgagcaataaaaagaatgatgagatatggtttccc

SEQ ID NO: 122 - DNA, 638 bp, Brassica napus
tgaaggatgtaaccgtgtcatctgcggtaggaatattcatggcgtattttggaggagaaagaggggcaaaaaatgagaagtactctagcaattgtgagaggacaacaatttgagacaggaaagagctatgatcatgactggactcaggaagtacacgagcaaggtcatccgcaaggactccgccaagaagactgcgaagaagtcgagagtcaagtgtttcatcaaggtcgttaactaccagcatctgatgcctactcgttacacgttggatgtagatctgaaggaagtggcgactctcgaggctctggcttctaaggacaagaaggtggcggctcttaaggaggctaaggctaagctcgaggagaggttcaagactgggaagaacaggtggttcttcaccaagctcaggttctgagaaccgagtttctttacagtctacttttgtagttctagaaatgaatgtggatcgtgttttggatggtttaagacttttggtttgaaattgattctgggttttgtctttattacttgtcaattttaatcatttgtagctgcgagtgtctgctttgattatgtctggagaaacattttcatttcattttgcttccgatctttatatttcagtggaatttaccgac

SEQ ID NO: 123 - DNA, 547 bp, Brassica napus
ggagagagagagagagagagatccgattctgatcagagatttgtagattgttcgaagaagatgaacactgacatcactgccttggagaagccccagtaccccgtcgttgatcggaatcctccattcacaaaagtcgtcggaaacttcagcgtcctcgattacctccgtttctccaccatcaccggcgtttccgtcaccgtcggctatctatcagggattaagcctggtatcaagggaccgtcaatggtgacgggaggactgatcggactcatgggtgggttcatgtatgcgtatcagaactcggcggggaggctcatgggtttcttccctaacgagggcgaggtcgctagctaccagaagcgtggttggttccccaaatgagtctagggtttttgctggatctgggttcgatttcgaaaagaagtctcttttttttttatttgctactccttttgtttgaggataatgtttgcctctgtttgaggataatgtttgcctctgtttgttaaaaccttttggtatcttctttgtagtgtagtttgtgtcc

SEQ ID NO: 124 - DNA, 306 bp, Brassica napus
cttctctcatcttctctcgtgttctccgtcgaggagaaacatcgacggcgacggagcttcgtcttggaggaagagcggcgccgcctcgtttcgtcacgcttcgccgcctcgtttcgtcacgcttcgcctcgtttcgtcacgcttcgcctcaggcatttgacatagctttccttcaaagttcattgtcaattgtatcatttgtttctatgcttttggaattgacggtacttatttattgatgcaaagtctttctgttggatcagttgtatgtgccaacctttttttttaagaaattcccattggagc

SEQ ID NO: 125 - DNA, 695 bp, Brassica napus
gctctcttcctttacggacccaaatctaactctcttttgtctctctctccttctgtctctgtgttcgagattcagttgagatgatttcctttgttggtagagctctgttcgtctccgtctttcttctctccgcatggcaagagttcaatgaatttggggataatggaggccgagcagctaaatctctaagaccaaagttcaatgcttttgttagtcacgtaacaactcatactggccagcaattgccaccggtcgatacgaagcttcttgttgctgctgctatagccttaaagggtattggtggacttttgttcgtttctggcagctccttgagagcatatctcctacttcttcatcaagctgttgccaccccgattctttacgatttctacaactacgatgttgagagaaaggaatttagccaactgttttccaagtttactcagagcgtggcactcgttggtgcactactcttctttattggaatgaagaactcgaggagacatggtaggcaactccggaggaaggctccaaaagctaaagcaaactgaagatagaggaatgttttcatcgttttctctttttcagcttttgtagcacaatctgttctgtattttgagatttctgttttggaatccagacatcggtttaaagatgtatgcgatgaggcaaaaatctgcaaatcattttgcttt

SEQ ID NO: 126 - DNA, 952 bp, Brassica napus
  misc_feature (746)..(746): n is a, c, g, t or u
tgtatggctgatacccactcgtattaaccctactaaagggaacaaaagctggagctccaccgcggtggcggccgctctagaactagtggatcccccgggctgcaggctcacaacaatcctaaactcaaagctcatgctgtcaaagtttttaagatgacatgtgaaacagcaatacagctgagggagaaaggcaaagtggctgtgcctgacacaaccctccaatacctgggctctgtccatctcaaaagtggagttcttgatcctcactttgaggtggtgaaagaggctttggtgaggacactgaaagaagggttgggggagaagtacaatgaagaagtggaaggagcttggtctcaagcttatgatcacttggctttagccattaaggccgagatgaaacaagaaaactcacagaaaccctgaatatcatttgggtatatgctcatagatggtgaatctgtgtgcacaagtataatatcaaatcatctttgtgaaatttatctcaactacttttggttctttcttatacagagctacatgacactcaataaattaacataatacataaaagaagaaaaaaactcgagggggggcccggtacccaattcgccctatagcgagtcgtattacaattcactggccgtcgttttacaacgtagtgactgggaaaaccctggcgttacccaacttaatcgccttggcacacatccccctttcgccagctggcgtaataacgaagaggcccngaaccgatcgccctttccaaacattgcgcagccctgaatggcgaatgggcaatttggaagcgttaaaattttgttaaaaattccgcgttaaaatttttggtaaaacaacttcatttttttaacccaataaggcccaaaatcggcaaaaatcccttattaatccaaaagaaatggcccccngatttgggtttgaaaggtttgtccat

SEQ ID NO: 127 - DNA, 648 bp, Brassica napus; identical to SEQ ID NO: 91

SEQ ID NO: 128 - DNA, 450 bp, Brassica napus
gtcaacgcgaatcagtcagtcaccagagagagaagagagaagagagatgaggttgctcgatccatggccagtgttcttcaagagggagtggaaacgttgctggccgtttctcaccggattcgccataaccggcgtcctcatcaccaagctcaccgctggattcaccgaggaagacgccaagaactccaagtttgtccaacaacacaggcggtgaacatgtagaagctgagaaagggaaagtcagctttcaaaactattctaataacatagaagagaggattggattcttgatactactgttcgtctctttttttttttttttataataactcttgaacagtgcaagttcaacttttgttctccccatatgtttgaagtggattttgattgctccctttcttttggtgtgttcccatacatttaaataaatcttacagtcctgaatgattg

SEQ ID NO: 129 - DNA, 500 bp, Brassica napus
ggtctcgtttcaattctgtgagcgatggcatggagagggaacatatcgaagtctctcaaagagctcaggatccttctctgtcaatcttctcccgctagcgcttccaccaggactttcgtggagaagaattacaaagaattgaagtctctaaaccctaagtttcccttcctgatccgcgaatgtagcgggatccagcctcagatgtgggctagatatgatatgggagtggagaggtgtgtgaacttggatgggatgagcgagacacagtttctcaagtctcttgaagaccttgtgaaagctggaggagctacaaaagcctgaggctgtttctttctttctttttttttttttttttttttttatctagatgatctcgaataagctaattaactgcaaacactcaattttacatttcctgtcattctatttactttgcaactctctggtgtcttatcgcctttcgatttgaattgtttcttatttgtgtgtgtgtgtaatat

SEQ ID NO: 130 - DNA, 662 bp, Brassica napus
gacgtttccttcattgtcttattctctcttcccctttataaaaaaacatcccattatctacatagattagtgaaatctctaatcgtctcataaccaaacaaaaccttccaattataattctctttgttatttaatggctgaggaattcgatgaatctgaggttgtattctccgaagatttcaacttcaagagagacgacgagaatgaaaaccacatgttcggtgtcaaggagatgaagaagacgagccggatcatcaataggacggagttatcaagatcgcttccggtgaatgttcccgataacatgtttcggaggaggtacctcgggaaagaggaggatgaatactccggcggtggaggagagatggttccgccgcatgtcatcgtcggacggaggatccaaggaggagaaatggcgttttcggtttgcacaggtagtggaagaactctcaaggggagagatctgagccgggttcggaactcggttctcaagttaaccggttttttggaggcttgaattaaacctattactagtactactacccggttttgccatttgatttatcttcctattattcttacaatttacggaaaaatttaccagtgttgttttattttaagtaaagctgggaaaaatgtgaaacaaaaaaaaaaaaaaaaaa

SEQ ID NO: 131 - DNA, 1033 bp, Brassica napus
  misc_feature (609)..(615): n is a, c, g, t or u
ggactgaacccgtctatacgactactatagggaaagctgtacgcctgcaggtaccggtccggaattcccgggtcgacccacgcgtccggcaaacagcagccttgtcggcgttttctcagcctcgatttcctccgccggcgagcaatttcgtcgatggagaggcggagagattatgtcttaaccggagaatcagagactgttctgtggtggcgcgagcagggcccagtacgagtagctacttgctcgcgttcgctattcccgctactcttatcgccgccactgttttcacttcagccaaaatcgctgataagctcgacgatgacttcctcgaggatattgcgttgaatcaagcgatgaaagcagcagaagatggtgagaatgctgaagggggaagctcgctggatgatttgattaaggagcctgtgcttcaacgaacccgtaaacggccgaagcgtgaagtttaagcaaacacacttacgcttcttccgttcaatgatccgatgactgtgtaataatatgttgtgctttgtattaaggattgtagcctttttggtcacatttatagttgtggttactaaaaaatatatataatttgattatttaaaatcnnnnnnnannnnnnnaannnnnnnnnnnnnnnnnaaaaaagggcggccgctctagaggatccaagcttacgtacgcgtgcatgcgacgtcatagctcttctatagtgtcacctaaattcaattcactggccgtcgntttacaacgtcgtgactgggaaaacccttggcgtacccaacttaatcgccctgcagcacattccccttttgccaagctggcgtaataacgaaagaggccccgaaccggatcgcccctttccaaacaggtttgcgcaaaccctgaaatggcgaaattggggaccccccccccttttaaccgggggcaatttaaacccccccgccgggtttttgggtgggttacccccccacaacggggacccggtttaccttttggccaggggcccccttaaggcccccgctttcctttt

SEQ ID NO: 132 - DNA, 1023 bp, Brassica napus
  misc_feature (652)..(708): n is a, c, g, t or u
cggttgctgatacgcagtggtatacgcctcactatagtgaaagctggtacgcctgcaggtaccggtccggaattcccgggtcgacccacgcgtccgcggacgcgtgggaacgaaatggcgacggcgttgaagaggaacaggaagaagagaggccacgtcagcgccggtcacggacgtatcgggaagcaccgcaagcatccaggaggtcgcggtaacgcgggaggtatgcatcaccaccgtatcctcttcgacaagtaccatccaggctacttcggaaaggtggggatgaggtacttccacaagctccgcaacaagttctactgccccatcgtcaacctcgacaagctctggtctctcgtccccgaggacgtcaaggccaagtccaccaaagacaaggtgcccatgatcgatgtgacgcagcacggcttctttaaggttcttgggaaaggtcatttgcctgagggcaagcctttcgttgtcaaggctaagcttatctccaagactgctgagaagaagatcaaggaggctggtggcgccgtcgtcctcactgcttaagttagttcgagtttctcttttaattattgtctttttgtatcgttttttgttggactcaaagtttgttgcgttttggaatgagagatcttttaatcannnnnnnnnnnnnnnnnnnnnnnnnnnnnnnnnnnnnnnnnnnnnnnnnnnnnnnnnggagcggccgctctagaggatccaagcttacgtacgcctgcatgccacgtcattactcttctatagtgtcacctaaattaaattccttggcccgcgttttaccaacgtcgggaatgggaaaacccttgtggttacccaactttattcgccttttgtgaacattgccaattttggccgggctgggtgattaagaaaaagggcccccccgcatgggcttttccaaatttttcctccctccagggggaagtgccctctccgtagggggccctttaaagcgggtgtgtgtgggttcccccctggagatttttttttggg

SEQ ID NO: 133 - DNA, 582 bp, Brassica napus
ggaaaaatctcgccgccgttaaagaagaaaggagaagaagatgggagtattcacattcgtttgcaaaagcaaaggcggagaatggaccgctaagcagcacgagggagacctcgaagcctccgcttcttccacctacgatctccagcgcaagctcgttcagactgctctctccgccgattcctccggtggcgttcagtcttccttctccctcgtctcccctacctccgccgtctttcaggtgatcatcggcggtggatctggaggaggatttgctgccggaggtggtgcagcagctggaggaggcggtagtggaggtgaatccgctgcggccgcaaaggaggaagagaagaagaaggaagaatctgaagaggaggaaggcgactttggatttgatctctttggttaagtcaagtgacaaaacataaaactctttccaaggtggtgtttagtttctgtggttgttgatctcagattctttattttggtttttcttttcgaaacggttttgagattgagctctatgattttgcaatgtcagaaatatatgtttggtttctggttattatgaaacttatttagttt

SEQ ID NO: 134 - DNA, 513 bp, Brassica napus
gggcaacactcgatactaatggaaggttcattggtcgagacccggtcaatactggaagctggttcggttgtgccaccgggaagaaggatcccatcaggtgagctatggggaggcaatccagcaaggtttataagaacgttaactaacgaagaaaccttggagatttcgaaactcgctgtagccatcaaccatttgagcggagattacttctctgagttcttgccttactccactgtctacttagaggtagagaagttcaagaagtcccttggtatcgctgtttagaagtctctctagaagctttcttttgtgggtttcttgtgcttatgcctttccctattgattgcaataagtagctgaagaacatcaaaaacattcaaatgtttctctcttttttttttttgtttctttgtaaactgatattggctggatattgcctgagtgattgataatggcgagaacttcttgttttgttgtctatggacaagctaagatttgcagcataaataaaca

SEQ ID NO: 135 - DNA, 19 bp, Artificial Sequence
  primer
gcgtcccaagggcttcttc

SEQ ID NO: 136 - DNA, 21 bp, Artificial Sequence
  primer
gctcccaatcctcccatttcc

SEQ ID NO: 137 - DNA, 21 bp, Artificial Sequence
  primer
accgaggaagacaccaagaac

SEQ ID NO: 138 - DNA, 25 bp, Artificial Sequence
  primer
gagagacgaacagtatcaagaatcc

SEQ ID NO: 139 - DNA, 20 bp, Artificial Sequence
  primer
acagattgcccagggaggtc

SEQ ID NO: 140 - DNA, 22 bp, Artificial Sequence
  primer
ggtactcgtgcttcatggagag

SEQ ID NO: 141 - DNA, 24 bp, Artificial Sequence
  primer
gggagatcggaatcaggttatttg

SEQ ID NO: 142 - DNA, 25 bp, Artificial Sequence
  primer
atccatccagaaatcgtaacatctc

SEQ ID NO: 143 - DNA, 22 bp, Artificial Sequence
  primer
gcaccatcgtcaaccactacat

SEQ ID NO: 144 - DNA, 19 bp, Artificial Sequence
  primer
gtccactcctgcggttcct

SEQ ID NO: 145 - DNA, 25 bp, Artificial Sequence
  primer
acacaaatccataccttccagtgaa

SEQ ID NO: 146 - DNA, 20 bp, Artificial Sequence
  primer
acccaaagcaggctgcatag

SEQ ID NO: 147 - DNA, 550 bp, Brassica napus
aaaaaaaaaaaggtggcgtcacacggccctcctctggcccgtacaaaaaccatcattggccttggaacaacccatcggcaacagggaaattctatatcttctcccgaattggaaaaaaatggttcggtcttccggaaaccggtccaccaaactcttggcaaaaaaaaaaatgcttatcctcatggtggtcctcaaccctgcggttaaaaccaccatctggtacaacctcaacctgggcgaaatcgtcaccaccatcccaaccatgggttccaaggtcaaaacggtggaatacaaaaccttcagctccaccgccgggaatgtcggggttcagaacaaaatccgcccatgggggaggcttaactcccaaaacacccaagggctgatctccgggggggacagcaacgaccgtgaccgggtggggaaacctaaaaacaacctccacaaaaggctcaacaaaaataattggaaaaatgctgggcttctggtcttggctaacaaccaaaatctcccaaacgccatgaacgccgctgaaataacggacaaactgggc

SEQ ID NO: 148 - DNA, 485 bp, Brassica napus
gactgtgtgttctctaagttgcagcgacaacatcagaagaagcgagaatgcctaagcaaatccacgagatcaaggacttccttctgacggcgaggaggaaggatgcgaggtcagtgaagatcaagagaagcaaggatattgttaagttcaaggtcaggtgctcgaggtacctctacacactttgcgtcttcgaccaagagaaggctgataagcttaagcagtctcttcctccaggtttgagtgtgcaagacctttgaacatctcttcaagtgtcaatgggaccgacctgtgttgaagttttaagatacctttttatttatttggagattatagtttttgaatcaagactcttcttctcttcttttcaagacctggatttgtattttgcctaatgtggttagtaacaagaagcattcacattaattttctatatggttttgatttattccaaaaaaaaaaaaaaaaaaaaaaaaaaaaaacatgtc

SEQ ID NO: 149 - DNA, 630 bp, Brassica napus
ggagtcttcaagccaagatggtggaggttcaaccgatgtgatgggactatactcaacagatgcaagtgcacctgtgaagacaaccacaagtgttgactctctcttatcagagctgtctgatagctccagaggtacatatcatcaatctcaaaccacaaaaggaccaaactcaaaggaagctctggagaaagatgctcttgttagacagatgggtgtgaacccaacaagtcagaaccctacactgttcaaagatctcctcggctagcagaaccattctccaaaccaaaccatccattgactcacataatgatcatcatcatcaacatcgcccccacgttcccgtatttggttaatgcgttgtggaatttgttggtattgcttctgatacacattctcaagtctcaacatcgccacattcgcatatatttctgatgcttggggattagtagcgtttctgattttaagaacaggctttttgttgtatctatgttccttggagatttttttttgttttcaatggtttctcttttttgttttttttggagtatacagtgaatggttaccgtacggctgttggattttggagcttctcttgtttgtgtctatagaagaacatttggcaaaaaaaaa

SEQ ID NO: 150 - DNA, 714 bp, Brassica napus
gaacaacaacaacaacaacaagcgtgtttggactgcttcatctctcttctcctttcccacgtaattctcctaaaatcaactctctctaggtttcaatcaagccatgaaccttctccctctcttctaggcctctccaccctctctctctctaattccctttgatcggccgacggagaaagccaagtccgaaactctataatgtctgtctccgagctcaaagaacgtcacgccgtcgctacagagaccgtcaacaacctccgcgataggcttcgacagagacgcctccagctcctcgacaccgatgtggctaagtactcggcggcgcaaggtcgttctccggtgaaattcggagccacggatctggtttgttgccgtactcttcagggacacacggggaaggtttattcgttagactggacgccggaaaggaaccggattgttagtgcatctcaagatgggaggttaatagtgtggaatgctctaacgagtcagaagactcacgctattaaactcccttgtgcatgggttatgacttgtgccttttctcccaatggccagacggttgcgtgtggtgggttagacagtgtgtgttctatcttcagtcttagctccactgcggataaggatgggactgtaccggtttcgaggatgcttaggggccacagggggtatgtttcgtgctgtcagtatgtcccaaatgaggatgctcgtctg

SEQ ID NO: 151 - DNA, 375 bp, Brassica napus
tcttcttctgcccaatcttcatggcatcgtagagcttcctctttttacgggacatcatcaatgtcggcatatcagcagtatcttgggcaatttgagctgcatcaggaactgatgattcttcaccatcacctccttccaccacaggtgctgctgctacatcatctactgcacttcccgtcacttcttttttcagctcctcatggtattgcttctccagctcagccatcttcttcttggttttagcagcttcagcttcctcagcgcggctcataacaccagcgtacaacaagttctggggatcttcaagatcttctttgccaacacctggcaacggaagaacatcgttcctggctgctgcttgtagccttttgatgg

SEQ ID NO: 152 - DNA, 759 bp, Brassica napus
gctttgctgttctcttacatggaatccaaacaacattaatgggtcaatgaacaaacttacatgacttaacttcataactccaccaagtccccaaccaattgcatacagaaagtgttagaaaaaggctttacaaagttctgaaaaaaaatctcattgctctcttttctttccttttgtaaacatcaagatgactcatccttgagcctgtacaaggtgatttcttgctgcttgaagtaacggcgatcctcttcgtcaaccagcacaagattcagataatacttgacactgaatttgttgtttatgttcttatgcgttggcgtcagatcgtaaggtgtcaagaacagtcttacaggtatcgactcgcctctaacgggagcaccatccattaactcaaacttggctaatgtttctgtctcgacgtgagtattagctcctgctcctgttgattctcgccgtctgatctcaagatccatattctttatcttgattctcaccagaagaaagtatactttcccgaggataacatctttcaggtgatacttgcttttattgtactcaaactcgatatgcaggcagtcctcgattccaacttccatcttgatgctgttattgattggtggtaatggggaatagttacgcaccacaaagtcctggtactccacgatgcttccagcataaccacgagtaactgttactttcaggacataccttagccgaacattcacgccattgtatgtctcatatggcatctcaacggca

SEQ ID NO: 153 - DNA, 577 bp, Brassica napus
ctttctcacaagctcaaagagttgccaaaggtgcggtgggttcttcctgattcttaccttgatgtgaggaacaaggactatggaggggaacctttcattgatgggaaggctgttccttatgatcccaagtaccatgaggaatggataaggaacaatgcaagagcaaatgaaaggaacaggcgtaatgatcgtcctcgcaacttcgatagaagcagaaactttgagaggagaagagagaacatgtctggaggccctcctccccaacgtactcccatgggaggccctcctcctccacctcacatgggtggcgctgctcctcctccacctcaaatggggcagaactacgggggaccaccaccaccgcagaacaacatgggaggacagaggcctccaccaaactatggaggagcaccaccacagaacaacatgcgaggacagaggccacctgcaagctatggagggagcaccaccgcctagctacggaggaggaccaccacctaactacggaggaacaccaccgccacctaactatggaggaacagtaccccccgcaaaacagcatgggaggaggtccacc

SEQ ID NO: 154 - DNA, 408 bp, Brassica napus
attccacactcgattatgcccgttgctctaaagttcaaaacgacgactccagtgaagatgatgacagcgatgaagacagtgacgaggacgatcagcctcagtacagattccgtgttagaaccgtcggtgtgcgtcctgtgaagtgaaaaaaaaaggtgtagattcatagggacgtgtatggtttctttcattgttgttgttgcacactaagagccgtgaaaagagaaaaaccataagagagcagcagactgttcatgtcctgtgaagtagtgatgttatacatgttcgttcagaaaacacatgtataagtctagatgtgatgtcacagttgtatacaataggaccattatcaaatgaaatggtgccttcatctcaaaaaaaaaaaaaaaaaaaaaaaaaaaacatgtc

SEQ ID NO: 155 - DNA, 696 bp, Brassica napus
atcataaggctgtccagaggacttacgacagggagcaagatacctttcgccattggttcctcgtgcttccttgccttctcttagctcttctcattcacgaaaagtttacctttcttgaggtactgtggacgttttcgttgtacttggaggctgttgctatattacctcagcttgtcttgcttcaaaggactagaaacattgataacttgactgggcagtacatatttctccttggggggtacaggggattgtacatcctgaactggatctaccgttacttcactgagccacactttgttcactggataacatggatcgcggggcttgttcagacattgctctatgccgacttcttctattactatttcctaagctggaagaacaacaaaaagctccagttgccagcttaatttctcaatttgcgatgctcggaaacccttcggagattcagtttggtgcctggatacaatatctgccggaaagtttactagtttactctgttggtagttaatggatgagacgagcatagattatatgtaaactccagcgatctaacttgttgttacacatttttgaactttttcgtgtctcagcttttgttaagtgttactaaaatatttcgacaggacaaatcttgcaagtataactagtctactgtttccatatcaaaaaaaaaaaaaaaaaaaaaaaaaaaaa

SEQ ID NO: 156 - DNA, 287 bp, Brassica napus
ggctggggcatgcttttctacggtggatacaagctcttcaccggcggcaaaggagagaagcctgtcgaggctgcacaatgaatttatcactggggaaaagagtcttcttctcgtgcatttactttcactttcagcaccttgtgactggtcttgttatcaaagccagcaattacttgtttgtttttctttgacaataatatgcatcatttaatcaccttttgagatctcaatcatgtatccagtggatgaccaaaaaaaaaaaaaaaaaaaaaaaaaaaaaacatgtc

SEQ ID NO: 157 - DNA, 573 bp, Brassica napus
ggtgccgtctccaactccattctgaagagagcagtgatcgagggaaaatggagaaaggaaagggaagaaaggaggagatcgttaccagggagtacaccatcaacctccacaggcgcctccatagctgcacattcaagaagaaggcacccaatgcgatcaaagagataaggaagtttgcattgaaggcaatgagaaccaaggacgtcagagtagatgtcaagctcaacaagcagatttggagcagaggtatccgtggtcctcctaggagagtcagagtccgcgttgcccgtaagaggaacgatgatgaagatgccaaggaggagttttactctctcgtcactgtcgctgagattccagccgaaggattgtctggtttgggcaccaaggtcatcgatgaagacgagtgaaactcacatattgttgcagctttttgggttgaatctttagttgaaagacataaactgaaagacttatgcttgttttaccatttgggcaatctatttcgtatctttgaaagttgagagctaagtttttatccaaaaaaaaaaaaaaaaaaaaaaaaaaaaacatgtc

SEQ ID NO: 158 - DNA, 590 bp, Brassica napus
ggaagaatctcaccagcgtcaacctctctaacaatgacttcaacggaacggttcctgattctttggcgagtcttaagcgtgtgcagtcgttgaatcttgctaataattctctttccggcgacattcctgatctgagcggtgtgtctagtttgcagcatattgatttgtcttacaacaatctcaatggacctataccgagttggcttcagaggttccctacctcgtcttatcaaggcgttggtggattctctctcgtgcagccgcctcccgacttagctcatcaggagcttaaacctcgtcagaaacctaaacctcacttcttgggtctaaccaagactgtgttcttgttgatagtcatcgctgtctccgttgtgctactagcggttttagtctttgtgttagctgtttgttacttgaggatgaagctaagccaaggcgatgggattgtaaccgacgccaagctgcagaagaaaggtggtatgtctcctgagaagttcgtgtcgaggatggaagacgtgaacaacaggttgtctttctttgaagggtgtaactactcgtttgatctcgaggatcttttgagagcttctgcg

SEQ ID NO: 159 - DNA, 739 bp, Brassica napus
gtttgtggctgaagatgagcatgagatgcgtgcgagaaaaatctactacactaagatcgacactaaggtcacttcaaacggacataaaactcctctttgaggctgcgtgtggagaggtatagcgtctgaggcttctgggagattatcatcatccccaactagaatcggtttcgttgagtttgtcatggctgaaagtgcagtaactactctcaattgcagcgsgcgtgcyytcttggcccttyacatataaggcccaaaaaacaaaccattttctcaaacattttgtgttacttggttatgaatccgaacgttgacgtgtttttgatggtttgatcacagggatattctgagagctcaccccagatgcgagggttttcccctttttccacaatcagaaaatatgaatcttgacgacagaggaggcaaggcgtggtgcgtgcggtggagatgtgagcatcgttgtgcggaggttctcatggctgmcccagtattgtttcctctattgtgaatatccaacagggysmaaatgtvsyttsrcycctcttaggtgtytgccaatacgagtatcattctccatgatgatgagttcctgaagtacttaaaagatcatcaagagaactttaggtyttgagagaatgtaacttatatttacgtatgtgagagctggaactcgargctttcttagcttatttgtttttttacacttttaatgttttgatttgatttaagtttcttatta

SEQ ID NO: 160 - DNA, 507 bp, Brassica napus
gggatcatgtagcacgttgggaggcgagtacatatgttaacagattcttattgcctggatatttggagagaagccatgagcagcttaaggaggagttaatgttggacttcgatccaaccaagtgcttcttgagtgtgttggagaaacggtttgggatgttgttgaagccgtggtatgaccatttaggaggtgacttgattggtttaacttggactaaacagaagtcaaagaaacgaaaacgagatgaagatgaaactgatccgaaagagattttaaaggcggtaggcgaaatgggaaaagggttggtgagagatatcttagttgctcaagtctccatgacgtttctaaaagcttagtttcaaatctcctagttctgaccgattagatatcaaaactgtttatcaatcttgatgagtacacatcatatgttttctacaataccagtcgaacacaatctttttataatatgttttctacaataccaatcaaacacaatctttcttttgg

SEQ ID NO: 161 - DNA, 617 bp, Brassica napus
gattccgtctcccccaattaaaaactcagagaaccctagaattcgcactcaccaccgattgagaaatgatgaagcgtctggtcccaacgttcaaccgcatcctggtgcagagagtcatccagcctgctaaaaccgagagcggcatcctcctcccagagaaagcctcctcgttgaactcgggcaaggtgatagcagtgggacctggttcaagggacaaagatgggaaattgattccggtctctgtcaaggaaggcgacaccgttcttcttccagagtacggcggtactcaggtcaagctcggcgagaaagagtaccatctcttccgtgatgaggacgtcttgggaacattgcacgaggattgagattgagaatgctcaacacaaacacgagcgtccttgtttgcgtttgtcttatggttgaaaacaagagtcatttttttatctttttatatgagggttggactttgttgattggaactgaaatttggacatcaatcataaatcatgcatctttgttattcccaaatctttgtggtttttccgtaatctggtgatcgaataaaagctcattttgaggggattgattcttcgttaaaaaaaaaaaaaaaaaaaaaaaaa

SEQ ID NO: 162 - DNA, 544 bp, Brassica napus
gagggttttagtttctctccgacgagatttgagatggcactaagaggagtatggcagctccagaagctcgtggtgagctactgtaattggggtggcagcagtagaggcatacgagcctttatggaatcagagttacctgctctgatcgagaagaatccgcagctcgaagtggtgaccgagctctcaaggggacagcatccttacctcaagggcatttacaggaatagaaatgaaagagtggtgtgtgtgaagaacatggatcctgatcaagtgcttctgaatgcaacgaggctgaggaactctcttggaaggaaagtggtgaagctgcggactaggcatgtgaccaagcaccccagtgttcaaggcacttggactaccgctgtcaaattctgacaacttttgcggtctttgttcttctaattctagctgtgatgttgcaaatatcaggattcttcctttgttacctatatttgcgataccaaattgtttgctacctctactgttctctctggttcctttttcttacaacaatgttctgtcttgt

SEQ ID NO: 163 - DNA, 741 bp, Brassica napus
gagagagaacgcttcacatgaaagaaattccaaaactgttgtccgagtcggttgctaattgcaaggatgtatacaaggacgtgatagtcacaatgaagaccttaatggctgagaaagaatctccgacagcaaagctactctttggtgtaactggaatctcaacaagccttctttcaactctagaatcacaattctcaatgataatggatggtcagaaagcgggtagcatcgtagattatcctttatatgatcaatgggaaacacttcgtgttagtctgaagaacacagccacaagcctgctgttagacgcacaagcaaaagatgaaattcttaactcccacgacaagggacaagaaacgggtgtaatggaggagaagctgaagtctgagctcagcatagtcaaggaaagatataacgagctggagaaagagttgtgtttggataaactgcttctaaaagcttcaagagagagccatgagaggctggaaaaggaagtacaattcctgaaggaagaaagagattcattagatgtagcagtctctcaatcaactcagaggttgagagtgattgcatctgataaagagaatgctttgaaagatcttaatgtggaagtgaagagaaggaaagagatggaggaagagattaagcaaatcagctttgctttctctagcaggcagaagtctcttatgtcttttcacaatgaaatcaaatccaaaatgcagaaactaacaacgcagaatc

SEQ ID NO: 164 - DNA, 637 bp, Brassica napus
ggaagcttctttcatcctcgtcctcttgagccgccatctctcctcctccaaactctcgatttctttctccaaagagtgtaggacaatgccgaagattaagactaacagagtaaagtacccagaaggttgggagttgatcgagccgactctccgtgagctcgacgccaagatgagacaagctgagatggatgaacatgatggcaagagaaagtgtgaagccttgtggccaatcttcaaactctctcatcagaggagtcgctatgtctacgacctttattaccgcagggacgagatttctaaagagctctacgagttctgcttggaccagggctacgcagaccgcagcctcattgccaaatggaaaaagtcgggatatgagcgattatgctgtttgcgctgcattcagccaagagaccacaactatggaacaacatgtgtatgccgtgttcccaaacacttgcgtgaagagaaagttgttgaatgcgttcactgcggttgtcaaggatgcgccagtggcgattaatctctacatgtgttttcttcacgattccccactgttgctgtattctatatcttgtcaaatctgatttgtgtacccttaaaaaaaccttaagaaactccctttcagaacctctac

SEQ ID NO: 165 - DNA, 608 bp, Brassica napus
gagagagaggacattgagtcttctttgattgactacaagaaagctttgtccatcttagagcgattggttgaaccagacagtcggcacatagccgaactaaacttccgcatatgcatatgtctagagactggatgtcaaactaaggaagcaataccttattgtcagaaggctgtgttaatctgcaaagctcggatggagaggctcaataatgaggtcaagggttcatctggatcagcaacttcctcagctgtctctgagaaagaagctgagatcagaaccttatctggtctcgcagaggatctagaaaagaagcttgaggatttgaaacaacaagcagagaacccaaagcaacttcttgctgagctaatgggcatggcatccgccaaggctgttgctactgctgccggtgaaatgagtagaggtgtcaaatggacgggccgtccaatggttgcccatgtccatatacaaacgggcttaatatggacaaacccattttttccattagttttcattggacaaaatgtggaagaccattaagaaaatggtctttattggttttgggctttatggacgtggactgtccaatggtcacaaaacctaaggtttat

SEQ ID NO: 166 - DNA, 609 bp, Brassica napus
taaatttaagtaactttggctctttctctcaatcccacaacaatcttatcatccccttatcaagccttgtgttaatcttaagaccagtcctagtattacagatcaagggagaacaaaaatatagtggatggctccacaagatataaaaagggttagggcatttttatgaagaaggtgcgtattcttggatggctgttgctaacttatccctagcctctgctgtctttgtccttatcagaaaggttctaggtgttacattctcgttggaatcctccgaggagtgaaatattgcaacaagtgcgttcttctgtgcactcgtcttgattccagcgtacagtagagcattaagaagcagcttcccgacatcgtttcggacaaggattgtgggtttagattcttttgtccccttgtcgacgccttctttgcattttatgcagagattcccagttcctttatctttccatgcacctttatctgttgggtcatttgacttgacataaagtttgcatttgacttcatggaccacagtaacacccttctcttcggtctttctgacagatgggctgctcggttgaggttcttcatcttcaccgtctgcatcttctgggg

SEQ ID NO: 167 - DNA, 843 bp, Brassica napus
agaacgaccaaaaattccgcaggaaatctgcgtggaaggcctttcagcacattgctgcttatgattccgctgtctcagaatggctatggaagcagactgagggacgtaggtgcttattttttatgtgatcctccttacactagatcgagatgtagtggcttccagttgtgttggataccagccgtcagtgtccttagatacggtgtggagcttgctgttgcagataagagtgatgaagctgtttacgtcggttctggtggggagataacaaaactaactaatattagagccgctgaagctgggcaccttcacgccgcaagtgttgagaatcgtaaagacagccagctccctctgtccataacttgatgggagaaacatacagttttcagctaggctgacacatttcagtttgacttctaatcatcagaccttcacgatttcatccatttttaataagcgtaactgtccaccagtcccagatttcgttgttgatggagtgagctcacttaccatcaatgtctgtgccggtcctcctgataatgagaatgaaccagctggtgaaacaactgcaggtggtgcatcggtcacgaacttgctgatcccgacgagtttaaagcaactaagaaggcacgccttacatagattgaagaggatagtcgtcagccacggcgtcggctcctagaataatacttgtgcatagttgttggcatgttgcttttattacatcatctggcgtaaacagtttacaactgactataaacccaatcatgtttggaaattcattatctttgtctacaagtcggtgaactattctattttgcgtaaaaaaaaaaaaaaaaaaaa

SEQ ID NO: 168 - DNA, 655 bp, Brassica napus
gactcaacagtctaacatctatctcacttctcagtctttactttttcacttcgagttctgcgaaatttcgaaagagaagatgagtaaaggcgcagcagcaagcggcacgagcaaaggtggaaaagggaagggaaaggcaaccaagtcagtcgtctcacgctcgtcaaaggccggtcttcagttccccgtgggaagaatcgccaggttcctcaaggcaggtaaatacgccgaccgtgttggttccggagctcctgtctatctcgcagccgttctagagtacctcgccgccgaggtgttggaggtagctggaaacgcagcaaggcataacaagaagactcgtattataccgagacacattcagcttgcggtgaggaacgatgaggagctgagtaatcttctgggatctgttacgatagctagtggtggagttttgccacacattcttcccaaccttttgcctgccaaggttggaaagaacaaaggagatgttggttctgcttctcaagagttctgagttcattttcattttctcccgttgtttttgttccttccttgtaataaatattatctgctttttgtttgcatgcacctattgttatggaacaattgttttgaataataaaaaaaaacacaaaaaaacaaaaaaaaaaaaaaa

SEQ ID NO: 169 - DNA, 763 bp, Brassica napus
cggcacgaggaatttatgcaccgaaataatagaatacacaagtccaacagaaactgctgtgtgcaatcttgcatctattgctttacctcgattcgttagggagaaggatgtccccttggactcacatccatctaagattgtaggcagtttgggctcaaagaatcgttactttgatttcgacaagctagcagaggtgactgcaaccgttactgttaatctcaataagataattgatgtgaatcactatcctatggagacggctaaaacttcaaacttgcgtcataggcctattggtattggtgtgcaaggtcttgcagatgcatttattcttctgggaatgccatttgattcccctgaggctcagcaactgaacaaagacatatttgaaaccatatactaccatgcacttaagtcatcttcggagattgctactaaggaaggtacatatgagacataccaaggaagtcctgtgagtaagggtattctacaacctgacatgtggaatgtgattccttcggatcgctgggactgggctgctctcagagatatgatttcgaagaatggaattagaaattctcttttagtagcaccaatgccaactgcttcaaccagccagattcttgggaataatgaatgttttgagccgtatacctcaaacatttatagtcgcagagtcttaagtggtgagttcgtagttgtgaacaagcatcttcttcacgacttgactgatatgggactgtggtctcctacgctcaaaaacaag

SEQ ID NO: 170 - DNA, 708 bp, Brassica napus
gggaacacaaagcatctcatgaacgctgtgaactctctcccggagttaaccgagaggaagcaggtgattgacaagcacaccaacatcgcaaccgcgctcttaggacagatcaaggagagatctatcgatgctttcactaagaaagagagtgacatgatgatgagaggcggaatcgacagagctgagcttatggctgctcttaaaggcaaaggtacaaagatggacaagctcaggttcgcaatcatgtacttgatctccacggaaaccataaaccaatcagaggttgagtccgtggaagcagcgctgaacgaagctggagctgacacaagcgcgtttcagtacgtgaagaagatcaaatccttgaacgtgtctctcgcagcttcagcgaactcagcaagcagaagcaacattgtagactgggccgagaagctttacgggcaatccataagcgcggtcactgcaggagtcaagaatctgttgtctagtgatcaacagttggcggtgactcgaaccgttgaagctttaacggaaggaaaaccgaacccagagttcgattcttaccttctgcttgacccaagagctccaaagcctggctcaggtggtggtagccacgtgaagggaccggtttagagaagcaatggtgttcatgatcggtggagggaactacgttgagtatgggagtttgcaggagctgacacaaagacagtt

SEQ ID NO: 171 - DNA, 689 bp, Brassica napus
gacctggttacaactacggtgttattctgattccagaaggtttgatcgattttattcccgaggttcaggagctgatcgcagaactgaatgaaattctggccaacgaggtggttgatgaaagtggactgtggaagaagaagctcaccgaacaatccctgatgctgtttgatcttctgcctgaagcgattcaggaacagctgatgctcgagagagacccacacggaaatgtccaggttgccaagattgagactgagaagatgcttattcaaatggttgaaactgaactggagaaaagaaagcaagctggttcatacaagggacagttcatgggacagtctcatttcttcgggtatgaaggaagatgcggtttgcctacaaattttgatgcaacctactgttacgcacttggttatggcgctggagttctcctcaacagtggaaaaaccggactgatttcttcggtcgggaacttggctgctccagttgaagaatggactgtaggtgggactgctctcacagccttgatggatgtcgagaggagacacggcaagttcaagcctgtgatcaagaaagcaatggtggaacttgaaggtgcgccgtttaagaaattcgcatcgctgcgtgaggagtgggctttgaagaaccgatacatcagccctggtccaatccaattcactggacctggttcg

SEQ ID NO: 172 - DNA, 910 bp, Brassica napus
tcctrgcacttacccaagcaaactcccatcatcatcatcgcttgatggtacacaacataatgtagtgacatatacaacttttctcttcagtagtacattcatggaagacagcagacttggactcaaagcctaaacctaacgaagargccggttcarccataccatgtcgaaagataacaaagcagagtggttgtaggagttaatgttgcagagattgttggcctccttgctgaactgaaccttggtgttgcctcttccgarctgtttgaccaagaaacagtccaggacaacgaaggtgagccattaagccctgcttcatccaccctgcacgtttctccctgtttttggccagtgagatccagaaaacggttcttgagctccacagaagaacattcagtccattggttttgtcctggaaccttgtaagcrccagcatagaattcatttccaagcatgtcctgtatcattccacggrrccgaacaagagtgttgggctgcrcccacttgatacttgacaaatcaagaattggaaccttcaagagctttcgaatgtgttgtttcagggacgaggataatctatctccacggccaggttccaggtacacagataagtcattctttgtgtaacgacggaaggaatcaaagggagaaagaggatcgagtctggragaagctctttttatgaagtcgtgaaccttcttcttcttctccaatatctgcaccaacaagtaatccaattaccaaatttcaaatcgaaatttcgaaaactctgagaaaacaaagtttcgaaatttcttttctctctacaaatctacgccacaaagaagaaggaagaagcgtagagacagaaccaccagacgtattcgttgcacaagatgaaattttgggggagaaacaaaagttgcagagc

SEQ ID NO: 173 - DNA, 688 bp, Brassica napus
atttgacctttgaaactatcggtaatggcagatcagagcaagaacgacactcctgagatcttcgagctcaacaatggatcaatgcatgtcaagatctcaaattatggtgccaccatcacctccttgtttgtccccgacaagaacgggaaatcggctgatgtagttctcggattcgactccgtggatccgtacgtgaaagggcttgcaccctactttggttgcatagttggccgtgtagcaaatcggatcaaagagggcaagtttagtctcaatggagtcgactacactcttcccatcaacaagcctcccaacagcctccacggtggtaacaaaggtttcgataagaagatctgggacgttgctggacacaagaaagacggtgataaacctttcatcattttcaaatatcacagcgccgatggagaagaaggttaccccggtgcggctgccgtcatggcgacatacactctcacgtcagccacgaccatgagacttgacatggaggcagttccggagaacaaagacactcccatcagcttagctcagcatacatactggaacttagcgggtcacgcctcaggaaacattcttgaccataggatccagatttgggggtctcatgtcactcctgtggatcagttcacagttcctacaggggagatcttgccggtaaagggaactcctttcg

SEQ ID NO: 174 - DNA, 390 bp, Brassica napus
gtccggaaaagccttatgattgacccggtaacagaagaggaaggatgtgaagatggaagtttcatgatgacgtgcatgccatctcggagtgaaatcactcagctgactatcactggtgaatggacaacgcctaacattaacgaggcaatgcagctatgcgtggatgcttgttcaaaacttggagagatcatgcgtgattgtctgaaacaggctgcctcagcttccgatgaatgaacatactctcaagaaactatggatcctcagccattggtatgaaaccggttaagatgtggtttaggtaaaattgagtttatactaaactggagtggtgtagtttaaaaccgatgtaatttgctatgtcaaatttgatggaactagagatatttaaag

SEQ ID NO: 175 - DNA, 732 bp, Brassica napus
ggcggcagtcattgttggagctgtcgccggtaaacgccctgcttttccatgcgcacatcgcatgcccagaccgtgaacccatgatatgcatttcatccactcggaggcggtgggacagcccatgcccatgtgcctcgaagccgtggtgtgtcgtcaaacaaagaaacaaaagcgagacgcttaagcctccctctgcttcttcttctcctcgcgaccggcgctcttgcgctccaggatagactcgcggtccttgtcgagcttgagcttggtgatgacgaccttggagggagcgatgggggtgggaaccgactggccgttgctcttctcgcggacgatgccgttgacatgaatgcagtacttgaggcggtagacggaggagaccttgccctcacgtcccttgttagaaccgcggacaacggtgatctcgtcatccttacggatgggaatgctgcgcactccgtgcttctcgcggagctccttactcaaaggcgcgctcatgatgacccgacgcacactactcggcgcactgaagtgcgcctttcgcgatttgcggcgagagctcgcgaccattgactggaccttggtcatttttgctgtgggatgtgcgggttgtcgggttagtagacaaagcggacgcgtgggtcgacccgggaattccggaccggtacctgcaggcgtaccagctttccctatagtgagtcgtattagagcttggcgtaatcatggtcatag

SEQ ID NO: 176 - DNA, 500 bp, Brassica napus
tctggagggacatcgaagaaggaggaagtgaaacgccttcctgggggaaaactaaaaaagaaagatagacaagaagttattattgaaaaggttgtgcgcaacaagcgcaagtctatcaccatcgtgaaaggactagaactgtttgggataaaactcagtgacgcgtctaaaaagtttgggaagaagtttgctacaggagcgtcagttgtcaagggaccaactgaaaaggagcaaattgatgttcaaggagatataatctatgacatcgttgaattcattacagacacttggcccgatgtacctgaaagatccattttcttcattgaagatgggaagaaagttcaagctggttgatcaccacctctggcctgtttttttttgggcatggcaggttcacgtgtttggaggaaacgctacgagataataacttgcaacgtaacatatagttgaattatctctgtttatgacatttaaaatgtgctttgaaaaaaaaaaaaaaa

SEQ ID NO: 177 - DNA, 635 bp, Brassica napus
gacacacagtctctttctctcgctcatctccgccaccgggaaacaagtaccacgtcgtcagggtttcacggcggagagtatcagtacctgatcggagaagcgtcatcatctcctaagtagattattcaatggcaaccagtcagtacactttcccttcttcacgtgcatatatttatcgcatctgtatttgacgatttctctcaatctcgtactctctccgcagtggctaggtcgtttctgcaggcgatatctaaggacgaggcggtggctcctccgctcagagttgttcagatcgaaggactggctgtgttaaagataatcaaacattgcaaggagtttgctccaacgctcgtcacagggcagcttcttggacttgatgttggtagtgtgcttgaagtcaccaattgttttccttttccagtgagagatgacgatgaagagattgaagctgatggtgctaactatcagcttgagatgatgaggtgtttgagggaggttaatgttgacaacaacactgttggctggtatcaatccacagtacttggatcttatcagactgtagagctgattgagaccttcatgaactacccaggagaacatcaagaggtgtgtgtgtattatatacgatccctcta

SEQ ID NO: 178 - DNA, 741 bp, Brassica napus
ggtcgtcgtcgtcactctctcctcccttcttcctcagtcttccaaacttttcagagaaggcctcaaaaaattcaaaatctccatttcttattatatattaaaaaaaatgataaatccgttgtctctcctctgatgtgcatttgcgttgctcatctgagaaaccctagatttcgaaatcaaaatctagcttctttctctctttttgaaatttcatcctctcagtagactgtaacagtattaggaaagagctactttctctctctttgctaatttgggcaattagggtttcataaagtttcaatctttcaagtaattcaaaaggtcttagatgcatggacgggagagaagcaatggcatttccaggctctcactactatctccagagaggaggaggaggagccttcacgaatctctcaccttcccaggtcgcgagtggacttcacgcgccgccggggatgaggcctatgcctaaccctaacgttcaccaccctctggctaacaatccgggacctcagttccccatgtctgaccacagactctctgacttcggacacagcattcacatggggatgtcttcttctgttgcggtggagcagcagccgccgccaatgatggagacgccgatggttaagaagaaacgtggacggccgaggaagtatgctcctgaaggtcaagtctccttagggctttctcccatgccttcttcttcttctgcgagtaacaaagctaaggactcttctg

SEQ ID NO: 179 - DNA, 568 bp, Brassica napus
gagggcgtaaaaggaaggagacgaagccgtacaaagagggctcttccaccatctccgtactatctgaatcttctactccctcgaagatggagaagtatgaccgtgtggtgaagaagaaggaggagaggacgattgatgcgaacgagattcgtatcactagcatgggcagggcacggaactacatcacctacgccatgactcttcttcaagaaaaagggtcgactgaagttgtgttcaaggcaatgggaggagctatcaacaagactgtgaacattgtggagctgattaagagaaggatccctggtcttcatcagaacacatctattggatccaccgatataacagacacatgggaacctaaagaggaaggccttctacctattgagaccacaaggcatgtttccatgataaccattatcctatccacgaaagagcttaatacatcctctgttgggtatcagtgcccaattcctattgagatggtgaagcctttaggcgatatcgactatgaaggaggagagggttcacctggtggcagaggcagaggaaggggaagaggaagagggag

SEQ ID NO: 180 - DNA, 619 bp, Brassica napus
agtcttcattagcgagaaaaaatccaatcgttagagcctccaggcatctcggccctccacctgggtttaactctgtcccctctaagctggtaaaggaggaagcacctggtccggatatgtctggcaacaatccacaggttgatgattacagctggttggatggataccaaggccaatcttctcgaggcaccggtttcaacggttctttgaattacgcatctcctgggaaaccagagcacatgggtaccggcaatggattgaacgggcctgccacctttccgttccctggaaaacaagctccatcatcacagacacaagcagaatttccttatgctcagcatcctcagagagacaacgatcaatcagctcaactccccgatcaaaatcaaggacagcccagttggtcgagtcgtcgctttgtgtgaaatcacaatggaagagtaacacggtgagtgtctgctacctaactccaacttagcctcgttctgttgaagtctctcttgttggagtgtttgtagccttggtttcttgtggggcgcttcctacctgacagattcaggacacagcttcatgttttgtgaaggatatgtatggtaacaagaatgaatggtttctggga

SEQ ID NO: 181 - DNA, 465 bp, Brassica napus
caagtgtgggattgatagtagagaagtgccgttggaggcaagattctttacagagaacatgactataaaaattggcaagtcctgtgacatgggtcatagatggacggtttcaaaatctgtatcagattgcgctgaggccctgattggtgcctattacgtcagcggtggattgactgcggctcttcacatgatgaaatggcttggctttgacgttgaatttgacagagaactagtgaatgaagccatcaatagagtgtctctacggtgttacatccctaaagacgatgagcttacggagttggagacaaagatccgacgtgaattctcttcaaagttccttttgaaagaggctatcacacactcatctgttcatgaatcctactcctacgagaagattggagtttctaggccattccgtgcttgattttctaatcacccgacatcctctttaacacctacgaaaaa

SEQ ID NO: 182 - DNA, 555 bp, Brassica napus
agcagtgctatcaacgcagagtacgcgggtttgtctctagcaaaacttaaatacattttctcccgtgaaataatgatggctcactttgtgtttttttttgtgtgtgttgatttgcaggacatgggtttcagtctattcgagtaaggcagtaatgcttgttggcttatgtttttgtaatccttttccattttatgttgttgtttcttagtttgtactggatgggttatctttttgacatttctgcaattctcaagagatttttagttaaattcattatcctcgtgtctcttctccaaattcttgccttttttcagatcaacattcacataactaccatccttgccaaaatatgattttctaaaaccacaattattgtgaatggtgaataaatttgatgttggcgattcagttcaattcgtttacactaacaatctcgctagaacttcagattattattctgaaatctgcatttacagattaaccatcctagctaaatacgatttaacaccaaaaaaaaaaaaaaaaaaaaaaaaaaaaaaagtactctgcgttgat

SEQ ID NO: 183 - DNA, 520 bp, Brassica napus
gatactgaatccaccggagctttggacttgtctaagatcagagagatgaggcttgttccttctgactctacccagttggatactctgtttgatgtgttctgtgaatgtgctgagctcaatcctgaaccaattgaagaagaagaagaagaagaagaagaagaagaacataactgggtgtttagtgctgatcagatggttgttggtggaggagcggaagaagacggcgaatggccaatatctcagagcccgacgagtgtaattggtctatccattggagatgaagggggtcttgctcagcctatgctcgagctccagatcaatgatcagaggtttgacgacgctgaagagatggttcatgaaagtgagaccaaagatcactaatctgttcacaaatgtattactccaaaacagagttgtttcttatgttaaaacagtgtcgtctctctgttcttcaaagctgagctgtgtgctttgttaaatatgcgtttagacttggaaggtagattttgctcatcttgtt

SEQ ID NO: 184 - DNA, 606 bp, Brassica napus
gcatattgagagaggtattgcagatggtttgttaataagctgtgtatcatcatgttcaaatctatgggcattgataatggatgctggaactggcttcacaaaccaagtctacgaactatctcctgtcttcttgcataaggaatggataatggaacagtgggagaagaattactacataagttccattgctggtgcagccaatggaagctctttagttgtcatgtcaaaaggcacggcgtatacacagcagtcttacaaagtaagcgattcattcccattcaagtggataaacaagaagtggagagaaggcttccatgtaacctctatggcaacagctggaagcagatggggtgttgttatgtctcgcaactctggctacagcgagcaggtggttgaactggattttctgtatccgagtgaaggtgtccacaggcgttgggatggtggattcagaattacatcaacagcagcaacgaccgatcaagctgctctaattctaagcatcccacggcgtagactggtcgatgagacacaagagacattgcgtacctctcaattcccaagcacacatgtcaagaaaaaaaaaaaaaaaaaaaaaaaaaaaaa

SEQ ID NO: 185 - DNA, 686 bp, Brassica napus
ggttttgcatcttctactccttccgtttgcttcttcttcttcttctagatctgatctccaatttcacccccaaggtttgaaaagtctttgtcactttattatacaaaatgggtcgtggaaacagctgtggtggaggtcaaagctcactcaactatctttttggtggaggtgatgctcctcctgctgagccaaccaacaggactgctgcacctgccccggcagctgtgactacaacagcagctactagtactactgttgagcctgctaagatcaacaaggagatccctgctggtatcaaaactcctgtcaacaactacgcccgtgctgaaggacagaacaccggaaacttcatcactgtacgtttggctgaaatgaaatgctcactctctaaaacgacattgtaatgctttctatctctgcaggaccgtccttccaccaaggttcatgcagctcctggaggtggatcatctcttgattacctcttcgctggtcgcaagtaaaaaaacacacattgtgtgtaatgcaaaatgcgtatccattgcgtttctgttctcaattttaaaactgtttgatgtgaacctctaaatgataagaagtcggtttctttgacaaccctatcttaatcgtatatctcgtaattttctgaaagaaaaaaaaaaaaaaaaaaaaaaaaaaaaaaacatgtc

SEQ ID NO: 186 - DNA, 395 bp, Brassica napus
aagcatgggagtttataatgaagatgcctatggagcctgatgagaaagtctggtcgacgttgcttggagcttgtagggttcatggggacgttcagttggcagagattgcggctcgaaaggttgtttgttataacacggaagattcagctgctcttgttctattgtcaaacgcgtacagagaagctgggaatattgaagctgagttgagcgtaaggaatgtgatgaactctcaagccatgagaaaagaacctggcctgagctggattaccattggaggtcaaatccacaagttttgctctggggatcaccaccatccccagaaggatgatatatacagagttttgaatgatttgatggaaaaaaaaaaaaaaaaaaaaaaaaaaaaaaaacatgtc

SEQ ID NO: 187 - DNA, 786 bp, Brassica napus
  misc_feature (645)..(645): n is a, c, g, or t
cccacgcgtccggtgccgcggcgacccaatcaattataagaccattaaagtcataatctttcagacaatctctgtctctctctctcttctggtagtcttcgattacgaaagttcatctcttttcagagatacgatgtcgacaccagcgaggaagaggctgatgagggatttcaggaggttgcagcaagatcctcctgctggtatcagtggtgctcctcaggacaacaacatcttgctctggaacgctgttatcttcggtccagatgatacgccttgggatggaggtacttttaagttgactcttcaatttacagaggattatcccaacaaaccacctactgttcgttttgtttctcgcatgtttcacccaaacatctatgcagatgggagtatctgtcttgatatcttacagaaccagtggagtcccatttatgatgtcgcagcgattctcacatctatccagtcactcctctgtgatcccaacccaaactcgcctgcaaactcagaagctgctcgcttgttcagcgagaacaagcgtgagtacaacagacgtgttaaggacattgtcgaacagagttggaccgctgactaataagtcacccccgagtttgtaacatttgaagaagtgaaggagaggcctatcanattggttaaaaaaagtgttgtttcatgcctccggttcagttaatgtatcatgtaatgtaattgtcggatgaacctaaatgtatttgaatgataagacatcttgcttgcttttaagttgtgtgcgtaatgtaactatttaag

SEQ ID NO: 188 - DNA, 775 bp, Brassica napus
cctatcagtaaccttttacaccagtttcactgatcaataacaaagagaaattaatgttgtgaaccccagaaagaaagaaaaaacgctgaatccaaacacaagctaaaccaaacaacatgtatataaaaatgctaaaccaaactatacacagattacaaccaaaaggtctttgtgtgtgttttcccgttagaatcatctcatccttccatccaaaagtacaaacattcatgtaattaacttaagagaatgatgccttcagcttcacacatctctctttttctctcatccaagactcaaacgatccaatggtttgatgatgttagtctgcctacgcggagttccaatctcacaagcattcactctcgctgcgaatctcagcgagcagagagactcaccggttgaagaagattcaggagcaatgttcacaaacatcagagtcttcgagtcaccacctaagcaaggctggagaagataagtcagcttccagttttggaatggaacatgatcatctttctttgctaaggcgaagatgacatcttcaagagacgacaagcttttgttgattgcttgagctcttttaagtctatctctacttgatgcacgttttggacaaacgctgactttctgttagattaattatgtccaacactcttcgctcttgctgttcaccgctcttgtttatacctgtcattctcaatctgaaccttactgtctttctttatactgtttcttcatcttttcctcctactccgactgcctttcacattgccgacag

SEQ ID NO: 189 - DNA, 890 bp, Brassica napus
aatctggtacgcctgcaggtaccgtgtccggattcccgggtcgacccacgcgtccgcccacgcgtccgcccacgcgtccgcttttctgcaagccaagccttacaaggaccatcctaccattgcaagatgaccttaaggttgtccaggcaaagtttgcaaaaactgaagtagtctgctatatgtttggatcaacggtttccaaggaagctcgagctatggttctacatggtgctgaatactgttttatctcactgcttttggctgtgaagttcaagacgagggattggattcacgtgatcattggaatagagtcatgaaagggcatactggagataatatgttggagtggctttgttaaaatacaattatggtcctatccagttttgaagccaaatctgcaagtttgaattcacctcgcatccgctccaaggtaaagaaataacgtaagtattattatatcttactgcaaagtaaattttcctgaataacatgcatgcataccctggtaaaattgtgaaaaactcaacagaaccaatagaaattgtccatagttagataatctggatatatgaactttttcacacactaatagaatatctggttgtcctttccggtgatcattgcaacatttgttaccgtttatcagacgagacccaggaccaacttgaggaaggagacaaatataatccactcgaagtccctgagtgtccaaaactgtgagaaaaaatctaatcatagttgataaatttggtttcttcagattaatgtcaatggacagtaaatgtatgaattgcacctgattacacttgcttgagaaggtcattgggtcagaatcaagtggcagacttataattgtcgaatgacattacgcttattagttattaactata

SEQ ID NO: 190 - DNA, 305 bp, Brassica napus
ggcagaggaagaaggcgaagaaagcagcaggtggtgaggatcttatggacggagattatgatttcaaagaagattacgggaagaagaacaaagatacagccatggatgaaggagatggtttccagattgatgctaaaataccaatggctgcacttcttgatttgcctgaagaatgagtgatcttaaacaaacaatcgttttgttctttgtgtttatttatgtgctactaatcatacatgttgcttcatcaagcgattttgattctacgtaaaaaaaaaaaaaaaaaaaaaaaaaaaaaacttgtc

SEQ ID NO: 191 - DNA, 822 bp, Brassica napus
  misc_feature (15)..(15): n is a, c, g, or t
ttatttatttattgnttgttantaattttattggattgagagtaaaggggtgtggtgatttttggatgtganattattctttagttaggatagccgacttttgttacaaaatgtatgaactctgacatacaaaatctctgggattaattaatgccttcaattcaaaattacagacatcaaaaaaagaaagatggtaggaggatcaaagggaacaaaatgagattcaaacttaacagctgattcagttttcattcatcgattgggttaggaggtggaggagctaaaatgatgttcaatgtccaagcgtgcgttcaaactacttctttagcaaatacattccatctcgaaactcaagtttctcttcagataccaaccctgacagaaagctctgtagctgttgcagtgacttgtcgtacgatggatccgctacacagaacatctttagagtgttatggattcgctctagtgccatgcttccaaaatttgtgagcattcccatgatgaatttctcgtatattgtcatttctttgcgaagctggtcttccacagatgcgattgatctttccactctcttcatcacctcctaaaagctcctcactttcgtttttccccgaatcagtcatggattcaactagcgtgtacacgttcccatttgggtctcttcctcttggttctctcaaaactcccttgcttatccacaagtttactcttctgtttaatacatcaattggtatccccttggctgcacccagatctatcgagttccaacttttcttttcttgaaactgctgataatggctgcatgtgggcacgagactgtaa

SEQ ID NO: 192 - DNA, 470 bp, Brassica napus
ggcggccaagaaaatggtgcggcgtcttgtataccggtcgcgtcacagctacgccaccaaatcgaaccagcaccgtatcgtcaaaactccaggaggtaaattggtgtatcaaaccaccaagaagagagccagtggtcccaaatgccctgttacaggcaagcgtatccatggaattcctcacttgaggcccacggaatataagaggtctaggttatcgagaaacagaaggactgtgaaccgtgcttatggtggagttttgtctgcttcagctgtcagggaaaggatcgtgcgagcattccttgttgaagagcagaagatcgtgaagaaagttttgaaactccaaaaggctaaggagaaagtagctaccaaggcttgagggttttacgttttttttattttggccttgaaaggatacatctttttcactgtctcgtctctgacattatgaattacacatatttttacttgtc

SEQ ID NO: 193 - DNA, 662 bp, Brassica napus
  misc_feature (598)..(599): n is a, c, g, or t
taatgaaacagattgaaatttgtcaggaagagaacaggattctggacagaatgcacagacaaaaggttgcagaagttgaaaagtttacacagactgtgcgagagctagaagaagctgttcttgctggtggcgcagctgcaaatgcagtgcgagattaccagaggaaattccaagaaatgaatgaagagaggagagtccttgaccgggagctggcccgtgcaaaggtaagcgcaagcagggttgcaactgtagtggcaaatgagtggaaagatggtagcgacaaagtgatgcctgtgaagcaatggctcgaagaacggagatttctgcagggagaaatgcaacagctacgtgacaaacttgccatagctgaccgagctgcaaaatcagaagctcagctgaaggagaaatttcaactgcggcttaaggtcttagaagagagtttaagagggcctccaagcggtggcaacagaacgacacctggaggaagaagtatcagcaacgggccttcacgaagacaatcacttggcggagccgatgtcataccaaaactgacctcaaatggattcttctccaagagaacaccgagttcccagttcanntctttgagttctagtgcaagtacagtgctgaagcatgcnagnggaacatccaaatcattcgac

SEQ ID NO: 194 - DNA, 480 bp, Brassica napus
  misc_feature (449)..(450): n is a, c, g, or t
ttgccgctattacacagtcttatctggatgcattgaataatggtgctgtgccaacaataacctcagcttggcagaatgtggaagaaactgagtgtcgaagagcatatgattctggtatagaagcttatttggctgcctttgaccaatcaaaagctcctgaagaaggtgcactgagggaagaacatgaagaagcagttcgaaaagccttggctatgtttaacgccaatgctgtaggggctggtatagcaagaaaaagatacgaggatcttcttcacaagggcttgaagaaaaaatttgcggattacaaaagaaatgcattcatggaggcagatttgcggtgcaggagtgctatccaaagtatggagaagcagctgagagcagcttgccatgcctctaatgcgaatatggataatgttgtcaaggttctagaagctcgtttgtcagcctannaagcatcatgccatggcccagggaaatggc

SEQ ID NO: 195 - DNA, 577 bp, Brassica napus
cacgaattggtggattcaagactccataacttcaaagatgccgtctcttccaacaaaactttcttggatgaacacgtctcagccgtaaacaatctggctaaggacgcaaaaaggaaatgggagacattttccatgcaagctgagaatgacgccaaagaaggtgctgatttctcggctgcaaagcattgtcgaatggagttactgcttcagcaatctgtgggtcatgcggagtcagcttttaagcactgcaagacgactcatgaatcattaaaggtgatgaacagtaagcaagtcgcagatgtctcttcacttgttaagtgtgcttgtgataataacgaggagcacgacaccgaagtggattctgcaaggactgcggctgagaaagatgtagccaaaaacagcgatgagataatccagcaaattgatcgcatgtcggaggacgagaaagtgtctttatcacaaatattggacaatgtcaagacacatgaaaaatctcttgaaagtttccaacaggatcattgttgccaagccagatgcattgaggataaagctcaagaaacttttcaacaaagataca

SEQ ID NO: 196 - DNA, 511 bp, Brassica napus
aaaacaaagatgttaaagtggtaatcgtagacagtatcacgtttcattttcgtcaggactttgatgatttagctcataggacacgagtgcttagtgaaatggctttgaagtttatgaagcttgtcaaaaagttctctcttgctgttgtgttattaaaccaggtgactacaaagtacaccgaaggttcgtttcagttagctctggtgttaggcgatagctggtctcattcatgcaccaactgagtgattctgtattggaacggtgatgagcgttatgcgtatatcgataagtccccttcacttccttcggcttccgcttcgtacactgtgactggtagaggtctaagaaattcctcatcaagtagtaagtgtgtcaagatgatgtaaagacataatacaagcatatgagtaagctttctatggtttcctttgctgcgtgtcaaaatgatgtaaagacataacataagcacatgagtaagctttctttcgtttagttttgattttgattgaag

SEQ ID NO: 197 - DNA, 655 bp, Brassica napus
gatcgcaatggcagacatcgaatgcttaagtatactccagaacacatgcattgccttgctatgttctggggtcctcttgtcccacccaacacaggctttgtcgctttccaaaacttgtcaaccaatcagacaggatttaggataacagcaacctcggtcgtacttgactataatcaccaaacccatattgcaaagaaaatcaagctggttgggcacccgtgcaagatcaagaagaatactgcatttataaaagacatgttcacttctgaccttgaaatagctcgatgtgaaggttcatctgtccggacagttagtggcattagaggacaagttaaaaaggctgggaaaaacatgctcgataacaatgcccaagaagggattgcaaggtgtacctttgaagatcaaataaaaatgagcgacatagtcttcttaagggcttggaccccagtggaagttccacaattttacaaccctctaactacagccttgcagccccgcgataagacctggacagggatgaaaacgtttagggaactccgtagcgagcataacattcctattccagtgaataaggattcactctacaagccgatcgaaaggaagacaaagaagttcaacccattggtgattccaaagaaactacaagcagagttac

SEQ ID NO: 198 - DNA, 681 bp, Brassica napus
gtagcttcttgacgtcaccaaatcgctaaccctttttgatgttcgaactctcggacacatacacttcgggttctcactaatcccaatcgcataactatattcgtatatttgagatgggagataaagaggagttcgatgagggtgagattgagtacactagctacgcaggcgagcatcacttgcctttgattatgtctctcgtggatcaggaactcagcgagccttactccatcttcacttaccgctacttcgtctacctctggcctcagctctgcttcctggcctttcacaaaggtaaatgcataggaaccgtagtgtgtaagatgggggagcataggcagagttacagagggtacatcgccatgttggttgttatcaaaccgtatcgtggtcgaggcttagccacagagcttgtaacaagatcaataaaagtgatgatggaatctggctgtgaagaggtaacgttggaggcagaagtgtcaaacaaaggagcgttggctctatatgggagactcgggttcatagaagccaaacggctacaccactattacttgaatgggatggatgcttttcgccttaagcttttgttcccaaggcctcatgttcctcaaatggtttccgaggatcaaaacgagccggagcatgagattttccccaagcctcgtgactgaaatgacatga

SEQ ID NO: 199 - DNA, 573 bp, Brassica napus
gatgaccaaacaagcaaagattgtagcaatgacgtgtactcatgcggcattaaagaggagagattttcttaagctgggattcaagtatgataacttactgatggaagaaagtgctcagattttggaaatcgagactttcataccaatgttacttcagaggcaagaagatggccattcacgactcaagcgctgtatattgattggtgatcaccaccagctgcctcccgtggtgaagaacatggctttccagaaatacagtcacatggatcagagtctgtttacgaggtttgttcgtcttggtattccatatatcgagcttaatgctcaaggaagagcgaggccaagcttagccaaactctacaactggagatacagagacttgggagacctttccattgttaaggaagcacccatctttcatagagcaaatgctggtttctcatatgagtatcagttgattaatgtgcctgactaacaagggaaaggagagtcaacaccatctccatggttttatcaaaatcaaggagaagctgaatatatcgtcagtgtctatataatacatggagattgcta

SEQ ID NO: 200 - DNA, 552 bp, Brassica napus
  misc_feature (510)..(510): n is a, c, g, or t
ggtggcgtgacagctccaggaccagtctctgttgcgcagctcccaaaactcaaagacaacgttgccaactccatgttcttctgttaggttcgaagaagccatctctaataatgtgcagaagtctgtttttccccaaacttggctcttagtagtaatcggcttttataactttcaccttaggatctctctcggtttgaaaggcaacgaaactaaaggcttgttacgtgttttttttttgtgacttctcgctagccatgctgccttttggggattgaaatatggttttaccccgtaactctctctatcagggttgtttctagacgacttattgtatatctctaagctttaggaagtgatgctattgttacaatagctcttgctgccttaacaaaactaaaacccaaacaaactacatatccctactcaaagttgagaggtattgtagccttgtaggcagtacttgtaagttgtaacgtttagttaaccaccaatgatgctttcaaatatccnagataaaaaaaaaaaaaaaaaaaaaaaaaaaaaaaaaaaaaa

SEQ ID NO: 201 - DNA, 659 bp, Brassica napus
ggataaccctgagaagaaggaagatcaggtcgaggccgttaccgatgttaaatcaaatcaggagaaagcgttggaagatgcaaatgaagctaaaaaggaaggaaacaaactgtttggagatggtctttacgaagatgcattgtcaaagtatgagcttgcgttacactttgctcaggagtttcctgaatctgtggaccttcgatctatcagccattcaaacagagctatatgttatctcaaattgggcaaatatgcagaagcaatcaaggaaagcacaaaagcgatagagctgaatccgagctacactaaagccttggttcgtcgtgcagaagctcatgagaagcttgaacattttgaagacgccctcactgacttgaagaagatattagaactggatccttcaaaccgtcaggctagaaaatcgattcggcgtttggaaccattggctgctgacaagcaagaaaagatgaaagaagaagccatagcaaagttgaaggagatgggaaacacgatattgggaaaatttgggatgagtgtagacaacttcaaggctgttaaagatccaaacactggctcttactctttttctttccaaaactagtcaaccacattcctcatggttttgcatcaaccattttcttaggtttctcggggtgata

SEQ ID NO: 202 - DNA, 683 bp, Brassica napus
ggatcttctctgagctaagagaaaacaaaataacggagacgaaaagagtgagaagcggcgatgttggcgaagagcaagatgtgtctcccccgttactcccttttcttgatcttcttctctctcatttccttccaaggtttcgcaaagaagacaggagatgtgtctgaattgcagatcggagttaagtttaagccaaaaacgtgtgaatttaaagctcacaagggtgataggatcaaggtgcactacagggggaaacttactgatggaactgtattcgattcgagctttgagaggggtgatccgttcgagtttgaactaggaagtggtcaggttatcgaaggttgggatcaaggattgctaggtgcgtgtgtaggagagaagaggaagttaaagatacctgctaaacttggatatggggaacaaggctctcctccaactatccccggtggtgcaacactgatatttgatactgagcttattgcggtgaatggaaaaccaactgttggaaaagaagaagatgaagacgaagacgaagacgacacatctggagatgacgaactgtaactggatctccatgaatgaaacgaagttctatgggcctttttttcgttctttgaatgctttagtttggcttcttccttataaattgttattccttaaaaaaaaaaaaaaaaaaaaaaaaa

SEQ ID NO: 203 - DNA, 527 bp, Brassica napus
ggtccaaaactcgtagggaagagaggcaggtgcgagagagagagggggaaaccctatattcgatctgctccgttcgttttctcgaatcgaaggcgatatggtgttctacttcaaggcccgaccagatgctggagactacaccatcttcatggggcttgataagttcgagaacgaggagctcatcaagtacggcttccccgaagacgtctgctgatgcaggtttcacgttgataaaatgtcgtcggctcatgtctacttgaggcttcatagaggccaggggtttgatgacattagcgaaggtgtgcttgaggactgtgctcagcttgttaaggctaactccattcaagtttcgttttagaaggagggcacatgtaggctatatactgattttttaccgaatggtgtgagtggtattatgtgccagggaacaaggtgaacaacgttgatgttgtgtacactccctggtccaacttgaagaaaaccgcctccatggatgctggtcaagttggtttccacaattccaagat

SEQ ID NO: 204 - DNA, 345 bp, Brassica napus
agttctcctccgccacaccaataccaaatcctcgcgaaaatggtagtctctgaatatccagaagcggctcgccgcgtcggtgatgaagtgcgggaagggcaaagtctggctcgatccgaatgaatctcaagacatctccatggccaactctcgtcaaaacatcagggaagcttgtgaaggacggtttcatcatcaggaaaccaaccaagatccactctcgctccagagcacgtcgtatgaagattgccaagatgaagggtcgtcactctggatcggtaagaggaagggtacccgtgaagctaggttgcctactaaggtactctggatgcgcaagatgcgtgtgct

SEQ ID NO: 205 - DNA, 605 bp, Brassica napus
ggggtttggtaacgttgcgttttattcttacatccgatgagggcatcggtgttgtcctctttacagaactcaacctcgctttcacgatctgcaaaagtggtgtttcttttctcatttctttacttcccatgtcgttatctcttcacctccctgtggttcaatgtatttcttttaattcaggattaattgttaatcacattaaacccagttaggattaattgttattcctttgtttccatattgatttattgtatctttctctctggaatgcttaatgctttcatgatgagttttactctctgagatttttggttaacgttagggatattttttctttattaatttgactaagataattcatcctaataggatggtctctgcatatttactttgaatgttatttctgttaaactagatggactttcgatataaaaccaataggtgacaagtgaaatgacttatctatgttatatattactaagaattctttttaatatccgatgtgggacactttgtgtgtaatacgcctctcgagatgatggctctttgagcgtcaatctcggaatgcttggacaaggatcgatgggtcaactttggaccatatc

SEQ ID NO: 206 - DNA, 733 bp, Brassica napus
attgtttgtgcgcattcttctctttgcttccaaaaagttattgtttataggatactgagtcatctctactgaaccatacaaagaacatatatgaatacgtaaaataaagaaaacattaaacagaaagaaatatgtttaaatgccgctataaaacgaatttttcttcgttgtatgctttgattagatgatctcagtaaagaaatcttcggtgaatcttgttttttgtcctcattataatcaatgatcatcactccaggtctctgaggctctcacttgtcaaccagagcttcttctggacaatgagtataacattgtccgaatctgaggttgcctttgtatcaactgcttcccaatgcaaagccttgagatacttcttcaccagatcaactacggattgtgtgtcccttatgagtatgaacccacttggacgaagtatacgatccatctccaacagcaaatcctctgcgctgcaacctctcttcttgatatcagaaatgatgtcccaagcatggagtaaatcataagtcctagggtaagttgagaatgcttcacaccagctatgaacagcacccatcaggcctctgtcgtatataagtttaagcgtgttgggcccgtcttcaggaacaacattcataacccaaacatctttgtcattcagagcagcagcaaaagaacccatgtttgctttcatgtccattatgttcctcacagtgtctgattgaattgcaggact

SEQ ID NO: 207 - DNA, 534 bp, Brassica napus
attgggtggcattgtcagataacaacaacataacaggctttgtagacgaacaatctcttgaaaacctgagagtaacaggctttgtagattcatcatctgctttttgtatatcttctgtttgagatccattattagtcttggaagaagctgcctctgcaatgggagttgaactctctgtaacaataacgccaccgtctttgtctgcttcaagcagaggaggagacatatcttctcctaaaagagaaatagatgtttcatactgaattgaacaagccctagttgtagcatcttgtggttgcaacgcactgttcgtcttggatgaagctgtttcggcaatgggagtcgagctttctatcacagtaacaccaccggtgtcagcttcaggcaaaggagagagatcttttctttttctgagactccattgcttcaagagcatcactcttcagctcatccagctgtgctttggcaatgcttagctccaaattttcctcgtacttaggaagatattctttcctaatcccaagcatgttgttc

SEQ ID NO: 208 - DNA, 769 bp, Brassica napus
gctatgaggtaacgcacacacagcaagggtaggtagagtaaaccatggtcgcaccagctcactggctcacctgcgttatatatatgcataatgctctctgtttttctcacaactcagattcaatcctctgaagactttttgaaacaaaagggcaattcatcatcattataactcttaaggctaatagaagaacttgataaaccttctttgaaggacagcaccagcagaacgttaccgacttgttttctctcatttcttcagggatcttttacatgaaatcatcttcgagctcttgaatagacttgttgcttgaagcaatgtctttgttggatgtcatcttgtcagttaccattaatcccttgtaactcctcatgtctgcttgcctctccttctccagcctctctatatcttcacgtttcttcttctccctcaggtgttctttcctctcagctctttcagcagcattcactgcttccctctcagctctcagatcaggggttctctcaacttttgttttgttcaacctgttaattatgtcattaactcgcttctccactctgatagtccggaccatctttgaattgtggaagccaacttgaccaacatccatggaggcagtcttcttcaagttggaccacggagtgtacacaacatcgacgttgttcaccttattggcctttatgcagcctcaagtaaacgtgagctgacgacatcttatcaacgtgaaaccagacgtcttcggggaagccgtacttgatgagctcctcga

SEQ ID NO: 209 - DNA, 785 bp, Brassica napus
  misc_feature (646)..(646): n is a, c, g, or t
gactcaaacgaggccctaacgtcggcgacggcgaccatgagaacctccatggctcttctcctccgtactctctcgtcgaaagctcgcacttttcatcctcattctccatctctctcttgggctcgatgttatcctctgttaccagctccccaatcgcgagacatcactacttcgcggcgatcctacgcagcgatcgcgtcggcgcaatctcccttccggtccaacctccttcgaatcataagaaacgagatcgactatcaatcggaatatgctcctcctcatcagccagcgactgagttcaaatcattctctgttgaggatcgtcccggtgagcaatgcattgtgatgaaagggaaatttggagaagacgaaaccatcaaaatggaagcaactatgttcgatgggtttatggctgttccaagaaccggtttagatgcttcagggagcgatgtccgtcttcacgttagcctgctcgtcgacatctccaaggctgatgggagtgaagacatggagttcctctgttccgtctggcctaaccgtatcgaaattcaaaacctttacatgcttagacgtgataaaatcactggtcagccttacatgggaccaaagttcgggagtttgaagtatgattttcagacggcgattnaagagtttttgcgagtaagagggatagactcggagctttgctttttcttgcatgaatatatgatgaataaagataggattgagctcattcaatggttgagaaagctaattcattcatcgcaaagtaaattttcccattg

SEQ ID NO: 210 - DNA, 644 bp, Brassica napus
gggttatgaagtgttgaagagaatctcggaggtaatgagccggtttgatagagcgaggcttgaggaactcagtggagagttctatactgtgatacctcatgattttggtttcaagaaaatgagtcagtttgttatagacactccccaaaagttgaaacagaaaatagaaatggttgaagcattaggtgaaatcgaactcgcgacaaagttgttgtctatcgaccctggattgatggatgatcctttatattatcactaccagcaacttaattgtggattgacgccagcaggagccgattcagaagagttctctatggttgccaattacatggagaacactcatgcaaaaacgcattcgggatacacagttgagatttctcagctatttagagcttcaagagctggtgaagctgatcgattccaacagttctcaagttcgaagaataggatgctactttggcatgggtcacgtctcactaactgggctggtattttatctcaaggtcttcggatagctcctcctgaagcgccagtaactggttacatgtttgggaaaggggtttactttgccgacatgttctcccagagtgcaaactattgctacgctaacagcggttgctaatgacggcgtcctgctcctctgc

SEQ ID NO: 211 - DNA, 716 bp, Brassica napus
ggagaagagatggtgtctggttcagggatatgcgcaaagcgtgtcgtggtcgacgcgaggcaccacatgctaggccgtcttgcttcgatcatagccaaggagctgctcaacggacagaaggttgtggtggtccggtgcgaagagatatgtctgtcaggtggattggtccgacagaaaatgaagtacatgaggttcctcaggaaacgcatgaacactaaaccttctcacggtcctattcacttcagggctccctctaagatcttctggcgtaccgttcgtgggatgattccacacaagaccaagcgtggtgctgctgctcttgcccgcttgaaggtttttgaaggagtcccacctccttacgacaaggtcaagagaatggtcattcctgatgctctcaaggttttgaggctccaagctggtcacaagtactgcttgttgggccgtctctcttctgaggttggctggaaccattgcgacactattaaggagcttgaggttaaaagaaaggagaagtcacaggctgtgtatgagcgcaagaagcaacttatcaagcttaggaccaaggccgagaaggttgcggaggagaaacttggatcccagcttgatgttcttgcaccgatcaagtactagaaacccacatctatcttttggtttaaacctttctccgtttttacctttgaattttgtttttttatttgaatttcttaaccttgtag

SEQ ID NO: 212 - DNA, 455 bp, Brassica napus
gaaaccgggatcgatctaactccacagacacagtcgtcgaagagaaaggttgaatcgagagaagaggatgggtggaggaatggagacgaacaagaacaagttcatcgaggattggggatccgcgagagagaatctggagcataacttccgctggacgcgtcggaacttcgctctcatcggaatcttcggcatcgccctccccatcatcgtctacaagggcatcgtcaaagactttcatatgcaagacgaagatgcaggcagaccacacagaaagttcctatgagcttgtttgcaacaacattctcttctgcaacatgtctcagcagtttggaattttcaagaagctgatgctagaacccacctttctttcttttttgcattttattattcagaataaaagaacatctcaaggataatacttgctgtttccaaaagtttgttttgattgggcaagt

SEQ ID NO: 213 - DNA, 750 bp, Brassica napus
agttgtcaaaagcaggctggtaccggtccggtattcccgggatatcgtcgacccacgcgtccgcggacgcgtgggtgaagcggaggattcagcgcagcttaatgggttttcttcagctgttggtttgatgagtcagctaagaggacaacaacaacaacaacagcagcagtcaatgatgatgatgccattgtcaaggtctacaagtgatggctacgatgcatctggtggagaagacagtgatcaggttccttctagaaacatccacaaatcaagatgagaaagtataattaagtttttgagctttataagggtttgagaggcattctttgataaaatgctgtggtcacattcaagaaaagtcaacattcttagtcaaatgtttaaaagagactcttacagtgtctgccaagagctcttaggtgaaggcatcaggaagagttgtggattctgaagttttattatttttatttcaaggtaatgagaacagtttgaacattagggattgaccagaactttcatctcttgttttgtaaggaaagatcttgttttgtttttttttcatgaaaagaatgtcaagttgcacaattcatatgttgttatctcaaattcttgcaaacctcctgttttctgtaagaaagtgactacaaacaattcacgatcgagattacaccttataaaaattggagaagttgagaaaaaaaaaaaaaaaaaaaaaaaaaaagggcggccgctctagagtatccctcgagg

SEQ ID NO: 214 - DNA, 721 bp, Brassica napus
ggtcgactcagtcggagatttagggtagttccaacggcgactcgcgtcgcagcgtcgtttaggctggcaatctcgctcacggcggtgagagagatgaatcacgggcaacagtctggcgaggcgaagcatgaggatgatgctgttcttacagagttccttgggtctctgatggattacactcctactattcctgatgatctagtggagcactacttggctaagagtgggtttcagtgccctgatgtaagattaataaggctagttgctgtggctacacaaaagtttgttgcagatgttgccagcgatgctcttcagcactgcaaggctagaccagctccagttgttaaagacaaaaaacagcaaaaggataaacgtttaatattgacaatggaagacctttcaaaagctctgcgtgagtatggagtgaacgtgaagcatccagaatattttgctgatagcccttcaaccggaattgatctagaaacgaaccgcaagccttgaccatatttgcctagtaaggatgatcgtgtgtgagagatccctggttttatgttagtgtttcagtgaaaacaaaactctctctcttccctagtacatgtatattctttttttttatttgtttcttgactcgtgtctctcttgataataatatttctgagttcttggtatgtattctaatacgcttgtgtaacaggtccctttggatgatccctccaaaaaa

SEQ ID NO: 215 - DNA, 527 bp, Brassica napus
ggtcatttatataaactgcgacatatagagagagagagggcgagagagcactggagcatcatcccaggaagaaaatggaggaagcaaagggaccagtgaagcacgttctgcttgccaagttcaaagatgacgtaaccccagagaagatcgatgaacttatcaaaggttacgctaatctcgtcaatctcatcgaacctatgaaagctttccactggggaaaagatgtgagcatagagaatctgcatcaagttcaccaacatgttcctaggcagcttggataaagtgctggtcatagactacaagcctacctctgttcaaattcatcatcttgtgatctttctttgcaataaagtatgtttctcttttgatgtaatggcctgcgttatttctgtcaatctctttctttttttttgttgtatcagaccaagtgtgtaaacgatcatgagggtttgcttgttttcttttatgattttgagtaacttttgtggtccaaaaaaaaaaaaaaaaaaaaaaaaaaaaaacatgtc

SEQ ID NO: 216 - DNA, 532 bp, Brassica napus
ggagcaattggagcggtttttggaaatgcagaagtttagtgacacatcagtagggatgtggtcgaaaccgacagtaaggaggaagacaaagattgtctgcaccgttggtccttctactaacacacgagaaatgatatggaaactagctgaagctgggatgaatgttgcaaggatgaacatgtctcatggggatcatgcttctcataagaaggttattgatttggtcaaagagtacaatgcacaatctaaagataacaccattgctatcatgcttgataccaagggtccagaagttaggagtggagatttaccccagccgattatgttagaccctggtcaagagtttacttttacaattgagagaggagtcagcacaccaacttgtgtaagtgttaactatgatgattttgtcaacgatgtggaggcgggagacatgctccttgttgatggtggtatgatgtcgtttatggtgaagtctaagactaaagagactgtcatatgtgaagttgttgatggtggagagctaaagtca

SEQ ID NO: 217 - DNA, 502 bp, Brassica napus
  misc_feature (444)..(446): n is a, c, g, or t
cttccctactagtgccagtggctccggtggcagcgccactgtgacaccaggcacaactaatccaaaaggcagctcaacaagcaccacgcttcctggtggcaatggcaacagtccttattcaggaacctcctcaaccaatggagtttttggaaacaatagcacagggattaacccggactacacgacagagagcagcgcgtttgctctcaggaactcaggcacatttctcacctgccttctcttgatcgctttgagtggattctcttctttcttgatgctctaagaatattattgttctatggttttgttggtgcacttattgtctcaaggacattgagtctagcttagtgataatctctgtctgtctgtttcgagtctctctctctgtgtgttatattcttgttgtaagaagacttcagcttatgctgtcactcttacctcttnnnacggtttctggtagattttatcttagcgannnaaattatatataaatgatcttatc

SEQ ID NO: 218 - DNA, 433 bp, Brassica napus
cttgaagttcagtgatgttgatgaggtgataaagagggcgaacgacactaggtacgggctagccgcaggggttttcacaaagagcctggacactgcaaatagggtgtcgagggctttgaaagctggcaccgtttgggtcaactgtttcgatgtctttgacgcagccatcccctttggtggttacaagatgagcggcaacggcagagagaaaggtatttacagtctcaacaactacttgcaggtcaaggcagtcgtcactccccttaataaccctgcatggatctgatcaaaacaaagactcttcttaaaagcctaaataataaaaataaggccatggtttgttacttggaaagattgtgaacactgtgtgtgtgatcaagttctcactgtttgtttttctcatgttacataaggttacattgatccgtttact

SEQ ID NO: 219 - DNA, 605 bp, Brassica napus
ggagaatccagctaggaaggagaataaaaaatcagggaagaaggattctccggttactactactactactcaaaccactgagtgtgaagagagtagtaatgtgaatcagaacagtagcccgggcctaccaagctatatgcaagctactaaatccgcaaaagcaaagctgaggctgcaaggctcttcttcccctaagcaacaagtgactgagaaagccactagacgttactcgctaccatcttcaggtaataacgcaaaagtcacttctgactctcctaaaacaacaagattctcaaactcgggtggcaaaaccgggaagaagactgagaaacctcttcttaacgggaagacaactccggtagagtggaaaagatgattaaatgattatgtggtgaaagaaaagaagaccattattgatcactctacaatatttcaccatctatcttcctagttagtgtttgtgggttgtattgagaatgtacatcatcatatgtttgtttttgtgggagtttcagtttctgttttcatcatacattattttaactgcaaaattagatgtttatgatatgtaatgatactcaactcttaaaaaaaaaaattatatt

SEQ ID NO: 220 - DNA, 725 bp, Brassica napus
ggcacaaccgaagactcccaccatctctcctccaggcttctgcttcttcgtctctgaaactagtttgattcagccatggctaatgcggcgtcaggtatggcagtccatgatgactgcaagctcaagtttctcgaactcaaggctaaaaggactttccgtttcatcgtttacaagatcgaggagcagcagaaacaagtggttgttgagaagctcggcgagcctggtcaatcccatgatgactttgcagctagtcttcctgctgatgaatgccgttacgcagttttcgattttgatttcgtcactgctgagagttgccaaaagagcaagatcttcttcgtcgcatggtctccggacacagcaagagttagaagcaagatgatctacgcgagctccaaggacaggttcaagagagagcttgatggaattcaagtagagcttcaagctaccgatccaactgagatggatctcgatgttttcaaaagccgagccaactgaggaatacatcctccataaaagaattggttgaatccaactttctcctactattactgaattgatatatgcttgttctttaagtttgctctgaaggcaccaccaaaccaaaacaacttcatttcattggtctttgtcttcctatgtcttttatatttttttgggtccttgcagtgtttgcttaaaacttaagagattggcttgtactcttttaccattgtgtaagaaaaccc

SEQ ID NO: 221 - DNA, 696 bp, Brassica napus
gagcttagaccagttcccccacgaagctcctcgtcgtcgtcgtcgtcctgagcttgtaaccaccgatggagatcgatgattcaggtgatgttctcgatctcactagctgccagctccacagtctcgactcagtcgagttacctccgacgctaattgagctggaccttacggcgaaccgtttatcggaattggactcgaggatcgctcacctctcgatgctcaagaagctttctcttcgccaaaacctaatcgaagactccgccgtcgagcctctctctcgctgggacgccttgtgcgatctcgaggagctgattctcagagataacaagctagcgaaagtaccagacgtcagcatattctcaaggcttttggttttcgacgtatcgttcaatgagatcacttcgttggaagggatatcaaaggcctctagcacgttgaaggagctctatgtgtctaagaatgaagttaacaagattatggagattgaacacttgcacgacttgcagattcttgagctggggtctaatagattgcgggtaatggagaatatggagaacttgacaaagttagaggaactgtggctgggaagaaaccgtatcaaagttgtgaacttgtgtgggctcagatgtgttaaaaagattagcttgcagagtaatcggttgacctctatgaaaggatttgaggactgtgttgctc

SEQ ID NO: 222 - DNA, 332 bp, Brassica napus
gatcggtgggaggtatgagggcttctatttacaatgcgatgcctttggctggagttgagaagcttgttgcgttcatgaaagagtttcaggctaggcatacttgatttcaatcttcttcgttgttgagtgagttttagctttgaatgcttatggtttgagtttttgttttcatcttcatctactttgaatccagttgtgtgcggaactgtttcagtaataatcttttagcgtatgctaactgagaacttctagagagattaaaatctttgaataaataaaaaaaaaaaaaaaaaaaaaaaaaaaaaaaaaaaaaaaaaaaaaaaaaggcttgt

SEQ ID NO: 223 - DNA, 756 bp, Brassica napus
cggcacgaggatttgattgtctctcatcatgtataaaaccacatcttcctccctccttcgagccgctccttctcgctctccactcctctcctcccgatcgtctctgactcaatcctccgcatccgcatctccttccccgccttcgcctcttctcggccgtagatctttcgccacttcctcacccgctttccgatctcttcctcgatggagccactgccttcactccagaccatctccctttcgcctcagctctcagatcagagccgtctctcccggtttagaccgcctcgagaggaacttctcttccatggcgtcggagcatcccttcaaggggatcttcactactcttcctaagcctggaggtggcgagttcgggaagttctacagcttgcctgctctcaacgatccaaggattgataaattgccttactctatcaggattcttctcgaatccgccattcgtaactgtgataacttccaagtcaccaaggaagatgttgagaagattatcgactgggaaaagactgctcctaaacaagtcgagattcccttcaagccagctcgtgttcttcttcaggactttactggagtccccgctgttgttgaccttgcttgtatgcgtgatgctatgaacaagcttggcagtgattccaacaaaatcaaccctttggttccggtggatcttgtgattgaccattctgttcaagttgatgttgctcgatctgagaatgcagtgcaagcaaacatggagcttga

SEQ ID NO: 224 - DNA, 414 bp, Brassica napus
gaactcagagattacgcactcctttcagctaatcacagccgtcggaagaaaaaggagagaagcaaatcagccatgggaaaagtgcacggatccttggcacgtgccggtaaggtgagaggacagacaccgaaggtagcgaagcaagataagaagaagaaacccagaggacgtgctcacaagcgtcttcagcacaatcgccgtttcgttaccgccgttgttggtttcggtaagaagagaggacccaactcttctgagaagtagaagattgatgatatttgcatgaaagagatgcttctttcaaagctctattgctctttttgtaacttgatctttctccgtttgttttagtcatgtatctttggttcttaactctatatgttttgattccatgctatgaaatacgatcttagtttt

SEQ ID NO: 225 - DNA, 526 bp, Brassica napus
aagcttttttttttttttttttttttttttttgatacttttggcttatatatagtcgcagagataaagcttacagagatgtctctcgagcggccgcccgggcaggtcttttttttttttttttttttttttttgtttttaactgcgaatcaatggataaaacaaaaggggggatacccttatggcaaacatgtcttcttcatggacagaatcaaacataaggggagaggaaggagatagaacaaaacatcaaacgactcttggccttctttttttttttatgtttgggtaacaaaaaaatctcactctcatgtctcaagcaactgcctggactgggggtccatttgctctgttgttgaatccaccaccaccgggacctcctcctcttccacgacctccccttccacgtccacgccgaggagcatcatcataatcatcacggccctgaggaggaccctcataaccaccaccacgcccctgatgaccatcgtaaccaccacgcctctgaggaccatcataataacccc

SEQ ID NO: 226 - DNA, 495 bp, Brassica napus
acaaaggaagaatgcctcaactgaaagaattcatcaaggtcattcaacaagaacactactcctacaaagatcccatagtggagttcctggcatgtgtctatgtcaattatgacttagacggagctcaaaagaagatgaaagagtgtgaagaggtgattgtgaatgatccattcctaggaaagcgagttgatgatggaaactattcaactgtgccactgaaagatgagtatctagaaaatgcccgcctctatatctacgaacctatagcagaatacatcagagaatagacatgggggtgcttgctgagaagctgaatctgaactatgaggaggcagagagatggattgtgaaccttatacgtacctcaaagcttgatgcccagattgattccgagtctggaccgtaatcatggagcctactcagcccaacgtgcatgagcagttggttaatcacaccaaagccctatcagaaagacttaccagttaatgactcacc

SEQ ID NO: 227 - DNA, 671 bp, Brassica napus
cgagatgttgatatgaagcgtgactttgcttttgtcgattttagcgacccccgagatgctgatgatgcaaggtaccgcttagacggaagggacgttgatggtagccgcatcgttgttgaatttgctaaagggacaccgcgtggttctggttctggttatggttctcgtgagatatcttcaagcagaggaggacctcctcctccaggaagcggacgctgcttcaactgtggtcttgatggacactgggctagagactgcactgctggtgactggaagaacaaatgttatcgatgtggcgatagaggtcacattgagaggaactgcaagaacagtcccaagaagctcaagcgggaccaaagttactccagatcaccagttaggtctccatctcctcctcgtcgtagtagaagaaaccgcagcagaagcagaagcagaagcccaagctacagtcgctcaagatccccggttaggactgaaaaggctcggactcctgaaccagctgtgaggagcaggagccctgagccaacggttgttaactcacctccactcaaggatagaaaccatagcctaagtcctaatgaaaagagcccaatgcctgagaagaacggtcaaagccctaaagatcaagagaacggcaatggaaccaatggacaagaccacagtcctagaga

SEQ ID NO: 228 - DNA, 369 bp, Brassica napus
  misc_feature (162)..(163): n is a, c, g, or t
cggggacctcgtccttgtagcctctctctcagattttaaaaggagcttttgcttttcttcgttcccaaggcaattcagagagtcaacatgatgtctggtgatgaagctgctgctcctgtcgttgttcctgcggttgctgaggcagctgctgccattccagannacatggatctgttgactgcattggagttgacgctaaggaaagctcgtgctcacggtggtgtggttcgtggtctccatgaaagcgctaagcttattgagaagcgttctgctcagctttgtgtcttggctgangnctgcaaccagcctgattacgttaagcttgtcaaagctctttgcgctgatcacaacatcaacttgcttactgtt

SEQ ID NO: 229 - DNA, 550 bp, Brassica napus
gccgcatagtcgtctccatctcctcctcggggtagcttcttctctaaaccaaaaggggtagatgtccgacgaggaaggtgaagaagaggcgtttgtctcacaaaacagctcgtcgcgacaaattcgaagtcaaaggcgacgatttagtgtacactgagctgcgtaaaccagagacggagacaaagccttgggagcttaacgaggatttgcctgggatgggtcaattctactgcttgcattgcgatcggtatttctccaaggcgtctgtgcgagatgatcatttcaagacgaagaaacataagaagcgtgtgaagataatgaatggaccagcgccacactcgcaactcgatgcagatttagccgctggaatgggaatgccggacaatggtccaaagctcatggctgcttgagtttacttcagtttagtttggtttggtctcgtttctcattagtctacagtttatggaacctagattgttgttggtagagatggtccttagtagttaaactatataaatcctaaattaatgatatgcgtttttttgttctt

SEQ ID NO: 230 - DNA, 597 bp, Brassica napus
cggggagaaaaaaaagaaagagatggccggtggtcgtggatcgtcgaaatcaactgcacccaaaccgaggaagagggttgaagctgaatccaaacccgaaacaaccaccaacaacaacatcaacaccttgcttcgcgctaaagatggcagcgcctttgctaaatgtgaaggatgcaacaagaacgtagctgttgcgcttatcagtatgcacaattgcagcctcgacgctaaaattcgagtcaatctcgaagcacaagttgttgagacacaaaccgaggctaagaagaaacctgctgagaggaagaagtcaacatctgatgaacctaatttaaagagactcaggaagtccaaggatgagaataagaagaagagctcttcctccaacaagcccaagcgacctcttacagccttcttcatcttcatggctgatttccgcaaaactttcaaagaagagaatcctgacgctggcgttaaggatgttgcaaagcagggtggtgaaaagtggaagtctttggatgaggaagagaagaaagtttatttggagaaagctgctgaactaaaggctgaatacaacaagtcactggagagcagcaacga

SEQ ID NO: 231 - DNA, 583 bp, Brassica napus
agggagaagaagatccaaatcgatccgttgaaaagtcgggatttcgaatttgtgagctatgtcgaatgctgtggacgcgacggggaacccgatccctacttctgcggtgttgactgcatcggcgaagcatataggattaagatgtatgcctgagaacgttgcgtttctcaaatgcaagaagaacgatccaaaccctgagaaatgtcttgaaaagggtcgtgacgtcactcgctgtgtccttggcttgctgaaggatcttcaccaaaagtgccaaaaggagatggatgactacgttgggtgtatgtattactacacaaacgagtttgatctctgtaggaaagagcaagaagccttcgagaaagtgtgtcccctgaaatgagaatcacaagctaatttgtaatctcctattcattaaaatgagcttatttactctcttccttttgttgtcgtcaccattttttactctcaagtgctttcgtagcgacttctcaaagacagccaaatgactcagttttcattgtttgattgttctttaaccaggcacagaatctgttttatgtgcttatcttttgccatacttgac

SEQ ID NO: 232 - DNA, 743 bp, Brassica napus
ccgggctaggggggaggcatatggtgttctcaggcgtttatcctgctgatggatctgattttgaagcacttactcatgccatagagaaactgacgtgtaacgatgcaagtgtctcggtggctaaggaaacaagcgcagctcttgggatgggattcagatgtggcttcctcggtttgcttcacatggatgtatttcatcaacggcttgaacaagagtacggtacacaagtgatctccacgatccctaccgttccttacaccttcgaatactcagatgggagcaagttgcaagtgcagaatcctgctgcattaccttcaaaccccaaatacagagtcacagcttcttgggagccaacggtgatcgccacaatcatccttcctagtgagtacgtaggagctgtgatcaacctctgctctgaccgaagaggccagcagctagagtacacatttatagacgcgcaacgtgttttcttgaagtaccagttacctttgagggaggtagttgttgatttctacgatgagttgaaaagcctaacatcaggttacgcttctttcgactacgaggatgcagagtatcagaaatctgatcttgtgaagctggacatcctcttgaacggtcaggcagttgatgcgttagcaactattgttcacaaccagaaagcttaccgcgtgggtaaagagcttgttgagaagcttaagaacttcatagagaggcagatgtttgaagtaatgatacaagctgct

SEQ ID NO: 233 - DNA, 613 bp, Brassica napus
ggacggtgaggacgaagtggtggtttttagatctgtttcgagcggaagcggtggatgtcgtgatgagcagcttcgggtatagattggcatggagggaaacacggcgagtcaagattggccagtggagatcgcttgccccggcgagatccacagctccagcaaggtccacagagaaatcagaaacagaactaaagctgatacaaaaaaacattcgttatgacaacaatgatgataatgaagttatgggatccacattcgtcttttactcgagcatcctctttgagaaacgccaaatgaaagactgtgaaaggatcatctatacaatgtacacgcatgttggtgactaagtttgcagcatcttagaaacaatgaaatagccgatttccagtggaaaaaggttgaagaaccaaaagagttcacacttgagctgttctttgaggcttattgcactggagaacaatgagattgccggtttggagaacccttactaccataacactgtcttcaccaagacatctcacatgattgataaagataagcttatcctcgagatggccattgggtccaggagtaacaagagaagctgaagtatgatgaaaggaagccaaagtga

SEQ ID NO: 234 - DNA, 602 bp, Brassica napus
aaggtttgataaatgtggagtctagctcaaccgagattgaatctgagttgccagagagtttcattgttgatatgcaagacgaggaaggaagagttatcaacagtatttctgccaaacttgcatcggttgggagtggcgtgtatgagtactatacatgggcaaagcttggtgaaaagattagctttgttcctcgggattctaggggtaacgcggagaagaagatgttgttttatccgaaagagcttcgtgcggtagtggcaaatgatggttgtcaagctgctgtttccccgtttgctggtcgacctggtctttacatacaaggatccgtctctccccctcttcccggtgtcaacattaaggtctctgcaggaaaagacagccatatttcttcacttaagaaaggtgaagtagctgtcgaaacaagcacatcgccagatggctcttttgttgctgggcctttgtatgatgatataccatatgacaccgatgcttcaaagcctggctaccatattaaaagactaggaccatattccttctcctgccagaagcttggccagatctctgtccgtgtttcttcaaagggacacgccgagaactcaatc

SEQ ID NO: 235 - DNA, 513 bp, Brassica napus
cagcttcactgttccttcttgttgtgcgtaggcttggcgttgtttactccaccgtgctatctttgtgctgctaatcagtatagtatattctttgcatactgctatctttgtgctaatccatttcagtaaacgggaaagtggaggagtaatgattggttccatcaatcttattatttcctttgttttggcttgggtttctttcgcattgcccctccctcgagacgaaaatgggtgccttcaggtttcaccagtaccaggttgtcggaagagctctcccgacagagaaggatgttcaacccaagatttacaggatgaagctctgggccacgaacgaggttcgtgccaagtccaagttctggtacttcttgaggaagctcaagaaggtcaagaagagtaacggtcagatgctcgccatcaacgagatctttgagaagaacccgacaacgatcaagaactttggaatctggttgaggtaccagagcagaactgggtaccacaacatgtacaaggagt

SEQ ID NO: 236 - DNA, 123 bp, Brassica napus
atgtcttatctctgaacttgaacttagtttctagtggttcgcagtacttttgttttgtcaaggtacaatgatgttttgatgatttcaaggaaccaatgcgtttaatctattgtcagaattgcg

SEQ ID NO: 237 - DNA, 208 bp, Brassica napus
gtcagttggttcactgagacccaaccaaatgacgatgacatccatgacgaggttgctgaaatcatcaaggaggatctgtggcccaacccgctgacatacttcaacaatgatgctgatgaagtggaatttgagggagatgaagatgatgaggaaaatgatgaaggctctgatgaagacgatgaggagggggacggtgaagatgatgagc

SEQ ID NO: 238 - DNA, 721 bp, Brassica napus
cactccaatctactcccgcatcatttcactctacgcggaacagaacgagcttcagtttgctgttcctggaggtctaattggagttggaacaacaatggacccaactcttactcgtgctgatcgtttggttggtcaagtccttggtgaaatcggttcccttcctgatgtctttgttgaactcgaggtgaacttctttcttctacggcgtctgttgggagtgagaacaaagggatcagagaagcaagggaaagtgtcaaagctaacaaagggagagattctgatgctcaacatcggttccatgtcgactggagccaaagttgtgggagttaagaacgatctggctaagttgcaacttaccgctcctgtatgcacgagcagaggagagaaagtggcactaagtaggcgtgtggagaagcattggcgtttgattggttggggtcagattcaagctggaaccaccattgaagtgcctccttcacctttctgaggtgtgttgtcttaattttcatgttggttttttttttggagagtacctatctgcggatatacatttgttactagttttactcttgttttgtgcttttctttcttagatgtttattgcatttttgtctcattttcattaacttctctgtttaattatactctgggaatttcagtatggccggtgaagtttacagaaacgagttaaaaaaatttaaaaaaaaaaaaaaaaaaaaaa

SEQ ID NO: 239 - DNA, 260 bp, Brassica napus
  misc_feature (235)..(237): n is a, c, g, or t
tgtgtgtatcttctctgtcaagcccatgtcggagaagctagttgagtgagtccctttggttctgttctcacagcaatcaatcctaaagttaatgtctctgtgagcttagggattaaagtggttggctttgagagatggaggatattgggttggttaaacaaggttggaaatggatgcagtctcagaaacatatgtgctcgaacgcttgttctgcggcgcggcgttttgtggagannntnngggagcttgtggagcgtcac

SEQ ID NO: 240 - DNA, 663 bp, Brassica napus
ctaccagttcaatgtccctacctttggccctggtgttgtcttcgacgtcgactacaccgttcgccaggagcagttcaggttcttcaccgaggctctaagggttaacaagttgaaaggctatgtggagatgatggttacggaagctcaggattttttctcgaaatggggagagagtggtgaagtggatctaaagactgagctagagcggctcatcatcttaaccgcaagcagatgtttactaggtcgtgaagttcgtgaccagctttttgatgacgtgtccgctttgttccacgaccttgacaacggcatgcttcccatcagcgttctcttcccttacctcccaattccagctcaccgtcgcagggaccgtgcacgtgaaaagctctccgagatttttacaaagatcattgggtcgagaaaacgctccgggaaatgcgagaacgacatgttgcagtgcttcatcgagtcgaagtacaaagacgggaggcagacgacggagtccgaggttaccggtctgctcatcgccgctctctttgcaggacagcacacgagctccatcacttccacgtggacaggtgcgtacctgatgaagtacaaagagtacttctccgctgccttggatgagcagaggaagctgatggagaaacacggagacaagatcga

SEQ ID NO: 241 - DNA, 759 bp, Brassica napus
aagctcgaggtgcagattgaatcagttgataatggagagtgaaacgaaaccaaccttcagcaaatcaacaggccttccacgtaagcgtttctacagagcaagagctcatagcaacccactcagtgactctcacttccccattcccatttctccatctcatgtggacttctcgcttcacttccccaagttctttggagccaacaacgaagaagtctccaagaaggctgagttcgcagatatcggatgcggctttggtggccttctcatctcccttgcaacgctcttccctgatacgctcatgatcggcatggagctgagggataaagtgactgaatacgtcaaggaacggatcttagccctgaggaagacgagctcggggggacagtatgagaacgtctctgtcgttaggacgaactcgatgaagtacattccgaattactttgagaaaggacagctttccaagatgtttttcctcttccccgatccgcatttcaaggagaagaatcacaggaggagagtgatcagcgttgatttgcttgatgagtatgcttacgttcttagagctggtgggattatatacacgatcactgatgttgaggagcttggagagtggatgaggtcttgcttggagaaacatccgatgtttgagtctttgacgcaagatgagcttgattcggatcctgtcgttgagcttctgtgcagtgctactgaggaagggcagaaagttgctaggaatggaggacagactttcagagccgt

SEQ ID NO: 242 - DNA, 733 bp, Brassica napus
gtgttattgttttacatgacattgtattatgacagatggaacacctcccaaacacaaataaaacaataaaagtctacttcagcttcttcttggggcggaggttgtttgtgtgaccacacttcttcttgcggcagttagttgccctagggtgaagacgggcatagcacttgcggcagatcatcttgtcacagttgtacttcatggcaaggatacggagagtgggctcaatgacaccaccacggagacgcagcaccaagtgaagagtagattcggacgcgtgggtcgacccgggaattccggaccggtacctgcaggcgtaccagctttccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacacctgattgcccttcaccgcctggccctgaaagagttgcagcaaggcgtccacgctggtttgccccagcaggcgaaaatcctgtttgaaggtggttaacggccggataaaa

SEQ ID NO: 243 - DNA, 460 bp, Brassica napus
ttgaggactcggtaccaaaagcaatcatgcatttcctggtaaaccacacaaaacgtgagctgcacaatgtcttcattaggaagctttacagggagaacttgtttgaggaaatgctccaagagacagatgagatagcagttaagaggaaacgcactcaagcgactctacacgttcttcagcaagcttacaggacactagacgagttgcctttggaagcagaatcagtgcacaatcatggtacggatacaacaggtgtgtcaaaatatctggacttgccaacttcttcttcgaagtataccacgagcagtagttcatacactgcatctccgggtacaggaagaagatctagaagagcagtagatcaacaccaaaacggatacggtttctaacatgatccgaaaataattatatctgactcgtgataactgataaacgcattttattcatttaatggctctca

SEQ ID NO: 244 - DNA, 394 bp, Brassica napus
ggctccctgtaaaccctaatcgcattctcggcaggaaacatctctgagagattatcattgaaatcaaaggaggacgaacatgtatcttcagtgctatatcaacgagaagggcgagaaggtttacaccactaagaaggaatcaccgctcgggtcagcaactgaatctgcccatccagcccggttctcccctgatgataaatattcgaaggagagagttttgttgaagaagcgttttggtctcctgccgatccagggcgcacctgtcaagtactgaaagggcttagcttctcttgttttttttctttttttttgttactgactatgttgtctattaatgaaattatgaaaccatcattttacgtctccaaaaaaaaaaaaaaaaaaaaaaaaaaaa

SEQ ID NO: 245 - DNA, 757 bp, Brassica napus
atggacgcctgcggaccggtccggaattccgggtcgagaacgcgtccgtgatttcttgtttctccttcaagtcatccagagtccacttcatcaccaccaaagctggaatggcagagcagataaagtttgaccgttgcttgtacccacatttccggttctacatgagggaagtgagaacggtggtttactctcagtttgttggaatcttacaagagcgtgacggttgatgccatggcaaaggcctttggtgtttcagtggacttcattgatcaggagctgtcatgcttcactgcaagatagtcaaagttgcaggtgttttggagacaaagaatgcattttaccaggcaacaatcaagcaaggggatttctagctaaacaggatccagaagctgtctcaagtcatcatcactttagttagttgcagatattaatctttgcaaggcataaaagtgttggatggtttggaacaaaaggctaaggctagtggtgcttctcacagaggagtttgtcaatgattacatattcccttgtcttagagctggtgccatatatgaacgaaaaaccttgcttgtcttagagattatgttggaggcaatggtagatgtagtatcagaggttggagctgatgccgtttcacatgggtggtactggctaaggaaatgatgaccaggttcggttagagctcatcttcttctcgttgaatccctgaacttaaagttgtggcaccatggagagaatgggaaatcctaggcaggga

SEQ ID NO: 246 - DNA, 710 bp, Brassica napus
ggctacaatcagccaccgccaccgtatgttccaccggttccttaccagcatggtggtggctatatggctcctccacctggcgtgcaaggctatgctcagcaacaaggtgcttatcagagtagaggtgggtaccagccacgtggaacgagtgggagattcccgtcggatccttacccggttcagtcacggggaggccaccgtgacaacagaggaggaggttattctggtggttacaaccaacaccatcatcaacaatatgagcagcatgggcacggtgaaggtgggtctgagcattataacccacgagagtatggtggtcagcagcatcaccgtcagcatcagcaaggtggtgatcgcagaggaagtcatcatcgtggtcgttattaagcagagtgacgttctcgtatgaatcatcatcaggtgtagtgttgagcttagtttcatatggtttgttgtatcatttagaatttgagagcctgctcttggtttctttggtttataagggagataagagcagctgaggaatactataggtggtggggggttacattagtttgcttgcaacaatttttatttttatttgtattgtatctttgttttaaaaacatttgtttatctttgaataaactaaacagtgtggttatttgaattttaatgattttgaagttaattttctaaaaaaaagaaaaaaaaaaaaaaaaaaaaaaaaa

SEQ ID NO: 247 - DNA, 508 bp, Brassica napus
tcagcaaagggcaaagcagcgaaaggcaaagcaaactctacacctgcctccaagggcgaggagagcgacgcagagtcagaatctgaagagacaccaaaggcgccagaaccagcaacaaaagggaaaccagtgggttcaggcaagtcgcaggcgaagtccggtaagaagaggaagcgatgagtctggcctcaggaaagactttctccttatgtaattcaaggtggaaagctaagtacaatggcggtggtgttggtggttttggatcgatcggtcttaacgtcgtctgggaattacaacttacaactgttgtctttgtttttaagacaagaagcgtagtagtagtagtgagtggtttttctttttttttttgcacttttaggttcgattttcagcggatttttctttagtcgcccaaataatttaatctttgtgaatttagagagttgtagaggcctcaaagacaaatttgccgaatgtattttattcaattgtaaaaggatttgttctg

SEQ ID NO: 248 - DNA, 697 bp, Brassica napus
ggccgctctcacttttttttttattttctctctcgcagtcgcagccctactccttgatcaacaatggaattttggggagctgaagtaaaggctgggatgcctcttaaagtgaggcctcaagtagaccatcttatccacttgtctcaggcaacaattgatggagccaaaaagggagaatcaggtctcttgtacgtgaatattgattggaataaatatgtcattggaacactctctcaagaccacattcctcagattagctttgacctcgtcttcgagcaggagtttgagctctctcactctctgtctcaaggaagtgtccactttgttggctacaaatctcccaacatcgatcaagaggactacccttctgattctgaagagggggaggtgctggcagttcctgctacttctaatgtagtcaaggctgactcgaaaccaaaggccaagcctgctgaagtgaagcctgagtcagacgaggatgaggatgatgagtctgaggaggaggaggatgattctgaagatgatggacccgaccctgagaaagtgatggaggttgacgaggatgaagaagaggactctgaagaagaggaggcccctaagcagcctgcgccaagcaacaagaggacggcgactgcatctgtttccaaaacacctgtccctgcaaagaagacgaaaacagcagtagcagccactcctc

SEQ ID NO: 249 - DNA, 485 bp, Brassica napus; identical to SEQ ID NO: 148

SEQ ID NO: 250 - DNA, 736 bp, Brassica napus
  misc_feature (557)..(557): n is a, c, g, or t
gaactctagggttcccaaagccgtcaccaacgaacaccaacgcgatggggcgaagacctgcgagatgttaccgtcagatcaaggggaagccatacccgaaatcgcgatactgccgtggtgtccccgaccccaagatccgtatctacgacgtcgggatgaagaagaaaggagtcgacgagttcccattctgcgtccacctcgtctcatgggagaaagaaaacgtctccagcgaagctctcgaggccgcgcgtatcgcttgcaacaagtacatggtcaaatcagctggaaaagatgctttccatttgaggattagggttcatcctttccatgttctgaggatcaacaagatgctttcgtgcgctggggctgataggcttcagactggtatgagaggtgcttttggtaaggctttggggacttgtgctagggtggcgattgggcaggtgcttttgtctgtgaggtgtaaggataaccatggggctcatgctcaggaggcgcttaggagggctaagtttaagttccctggtcgtcagaagattattgttagcaggaaatgnggattcaccaagttcaaccgtgctgattacactaggctgagacagtccaagaggattgttcctgatggtgtcaatgctaagttcttatcgaaccatggtcgtttggctaaccgtcaacctggaagtgcgttcatatcagccaccagcgattaagagtgaagaaatgatttgtggttgta

SEQ ID NO: 251 - DNA, 508 bp, Brassica napus
gatctctcccttcccacaatctctcctgaaaccctaaatcaaaaagtcgagaacaatggcgtcgttcgctcgccgagctatgagcctagctcagattccatctgtagcacgtgcccccgcctctgtctgtcagcgtcgtggtctcgccagcgccgctgatcaccatggatctaccaaggttgacttctggaaacagcccacgaacccaggaaactggaaagaggagcattttgtgcttatttcgctctccggctggggcatgcttttctacggtggatacaagctcttcaccggcggcaaaggagagaagcctgccgaggctgcgcaatgaatttatcactgagggaaaagagttttcttctcgtgcatttactttcactttcagcaccttgtgactggtcttgttatcaaagccagcaattacttgtttgttttctttgacaataatatggatcgtttaatcaccttttgagatctcaatcatgtatccagtggatgattctgttcc

SEQ ID NO: 252 - DNA, 411 bp, Brassica napus
ggaggtggaagcagcaaggcacagaatggctctgtagatgagagcaagactccgactggttctgtagatgagagcaagacaccgagtgggtctgcggatggaagcaagtctgagaatggctctgcagcaggtggggaagatgatgatggcttcaagacgattacaagccgtaggaacagaagaggaaacggagacaagaaaaacagtggaactcctaaggtcaaagcttgatataatctcctctcctctgcaaacacccaacaagtacaagaaagagttacagttttcaagcatttgatagaaaattaattgttttgaagagaagggaaactttgagataatttttcgacattacagatttacatctctttacttaaaaaaaaaaaaaaaaaaaaaaaaaaaaaacatgtc

SEQ ID NO: 253 - DNA, 769 bp, Brassica napus
gaagccaaaacacaaaagggtcgatttgcttaacactcgaactttccgaccgaatcgatggcgaattcagatacggtgatgctcgtgatcagatcctctcggccacagtttcgccacaactgtgacaagatcgccttcgtagttcacgcttccttcatcgcctccggttacaagctcgttgccactggaaggccggcgttcgccgaagacgcactttcttcctctccttcccaaggcgaggttgggatcgaggggtggaacgagttcgaggagtacgcgtttgtgtatgcgaagaaaggatcgaagaagattctggtcaagtgtttggcgatagatgataagcttcttgttgatgctatagctgaaggtggaaaagaagagcctgctcatctcgagattgaagttgggaactatgttgctgagtcgagagaggaaggagactacgatgcagagtttaagaatctggggaagttagtcactgatttgcagaatcagattctgtacaaggttgctgaaggccttaaacctgttcctcctaggacccagtccagttcggagacaaatgaagagcgtgagtctggatattacgttagaaggcctgttccgttaggtcctcagattcacccatcaggggtggtagttcctccgatcccaggcccaggttacagcgatctcatccctggcccaggtgctggagtatacccgtcagggtggtttggcgatgaagctgctgtagacgacgatctcctgtcctgagtgtgatctcact

SEQ ID NO: 254 - DNA, 213 bp, Brassica napus
ggaggaaggcgactttggatttgatctctttggttaagtcaagtgacaaaacataaaactctttccaaggtggtgtttagtttctgtggttgttgatctcagattctttattttggtttttcttttcgaaacggttttgagattgagctctatgattttgcaatgtcagaaatatatgtttggtttctggttattatgaaacttatttagttt

SEQ ID NO: 255 - DNA, 434 bp, Brassica napus
atcaacgcagagtacgcggtggcttgtatttaaataaccatgtggataatgataatcattgataataatcccgtaattatcaatcagccttccattatatggtgtccaaatatcaaaaccaaaaagacgttttgacaatgcaaatagtgaagtgtcaaacacgaccaaagagagggttaaactgttaaagccaagtatttgcatcgtccttcaaaaactttcaagacgtgggagagggacaacttgggaacaataagaagatgcatataattaatctctctcccagaaagaagagattaacaaaagaggcaatagatgtatgtacatgggattttagctccacggtatgtaataactccgggaatgttatgaactcataagaagtaacctaaaaaaaaaaaaaaaaaaaaaaaaaaaaagtactctgcgttgat

SEQ ID NO: 256 - DNA, 630 bp, Brassica napus
ggcattgaacagggtcctcaggttacacatattctttgcattataaggagttttttattatatctttttttttgtcttttttttttttgttgattggttttgtgtagattgattcgaggcaattcgagaaagtgatgaggtacataaggtcaggtgttgaaagcaatgctactcttgaatgtggtggtgatcagattgggaacaaaggttacttcatccaacctacagtcttttctaacgttaaggactatatgcttatcgctcaagatgagattttcggtccagtccaatcaatcttgaagttcagggatgtggatgaggtgataaagagggctaacgagacgaggtacgggctagccgcaggggtattcacaaagagtctggacacagcaaatagggtctcgagggctttgaaagctggcaccgtttgggttaactgtttcgacgtctttgacgcagccattccctttggtggttacaagatgagcggtaatggtagagagaaaggtatttacagtctcaacaactacttgcagatcaaggcagttgtcactgctcttaataatcctgcttggatatgatggagctgtagtttgtttcttgcataaatgttcaaaataaaattaacaa

SEQ ID NO: 257 - DNA, 614 bp, Brassica napus
ggtgctttcgccgcgccagagttcagagagagagagaatggcgtcatcatcggcgagaggagagctggagaagatggggatcgatcagctgaaagcgttgaaagagcaagcggatctggaagtgaatctcctccaggacagtctcaacaacatccacacagccaacgctcgcctcgaatccgccgcgggagctctaaacgatctctctcttcgacctcaaggcaagaagatgcttgtaccactcactgcgtctctgtacgtgcctgggacacttgacgaagctgataaggttctagttgatatcggcactggttacttcatcgagaaaacaatggaggatggaaaagactattgtcagaggaagatcaacttgttgaaatccaactatgaacaactctttgaggtgttggccaagaagaagagcgtggcagatgaagccgggatggtcttgcagggtaaagttcgacagttgcaagctgcaaccacgtcgtgaaaattcattcattctctcgtggcttttactttttttgacgttttgtgactctgcacatttgttttgtctgaacagtttggtaacgaatcttatgatctcaaatcgggatatgttttgtgtt

SEQ ID NO: 258 - DNA, 521 bp, Brassica napus
tccgccgcacagatccaggacgggacagttgtctatctctaattctctatctctctgcgaaaatggtgacggccaagaaaacgaagaagtcacacgagggcatcaacagtaggttggctcttgtgatgaagagtggtaaatacactcttggttacaagtccgtcctcaagtctctccgtggctccaaagggaagctaattctcatctcctcgaattgcccaccgttgaggagatcggagattgagtactacgcaatgctcgctaaagttggtgttcaccactacaatggaaacaacgttgatttgggaacagcttgtgggaagtacttccgtgtttcttgtctgagcatcgttgatcctggtgattctgacatcatcaagagcattcctggtgaccagtgataccattttcgattttgttttccccctacttccatggatattctcaaattttatgagcaaaagatactgtgtttggtgttttattacttttacattatgaaccggataggaaaatataat

SEQ ID NO: 259 - DNA, 586 bp, Brassica napus
gacagcagatctctctctcccagctcctcccaatctccaacagggagattcgaagatggtgtccgacgctagcaagaagaaggctctccagaagaaagccgccgccgccgccaaaagaggcggtaaagccgccgccgccgccgccgcttcgaaatccgccgccacttcctcaaacggagttgacgccttgtcgagcggcgtcgatgctatccatatatcggaccggacctgtaccggaatcctctgctctcatccacagtccagagatattcgtattgaatctttgtctgttacgtttcatggttacgacctgattgttgattcgatgctggagcttaactacgggagacgctacggtctcctcgggctgaacggatgtgggaagtctactcttttaacttcgattggactcagggagctcccgattcctgatcacatggatatctaccatctctcccacgagattgaggccactgacatgacttcccttgaggctgtgatgagctgtgacgaggagaggttgaagttggagaaggagattgagactcttgttgaacaggatgatggaggcggagagaggcttgac

SEQ ID NO: 260 - DNA, 470 bp, Brassica napus
ggagtctgcatcttctctctcaatcagtttacatctcacgactgacttaacgccatggcagacgatgaggttgttgatccaaagaagtaccttgaggagtcttgcaatccaaaatgcgtgaagccactactcgagtatcaggcgtgtgtcaagagaatccaaggcgatgactctggccacaagcactgcactggccagtacttcgactactggcagtgcattgacaaatgtgttgcacccaagctctttacaaaactcaaatgaaacaaggcgaagctggagtcgacattgtctttgagtttaagggatgtataaaacgcagttgatgttggaacatttttgttaactgcaaacctgattatgccaaattgctttgctgctcggttttgtttctatcgcttttatgttttttttttcctacagacattgttattgtccttcttttgaagtaataaaatgtctaataacag

SEQ ID NO: 261 - DNA, 519 bp, Brassica napus
gactcatcactcgtcttcgaaatatctctctaacagcgataatggcggaatcaccggcgcagagtggtgcggagctggttcctcagcaagctgaatcggatctggcatctctcgtattcgaaatgtcacagcaagttcagatggggatggagaatatgctcaagttggtcaatgagatcgatcaaaactctgttggaataaaggaagagattgagaaatcaaaggattatgcaatggagaagaaaagggttctcgaggaagagaaagaacagttccagaaagctgcttacactatccttgacatgttaagcaaccgaagctagatgtagatgcggaaggggatgcatgctctatagaaagaaagaaaaacttcatcaggtaaagaaataatataaaatgtccaaaaaagattcactggcttggttttgtatttagctttgacgttgctattaccataaacttttaacacggagtggtgaatttaaaaaaaaaaaaaaaaaaaaaaaaaaaaaacatgtc

SEQ ID NO: 262 - DNA, 690 bp, Brassica napus
gatcttttggttaattcagaagattaacctgtgagactgtctctgcggccgaagcgagaatctccgatcagcgatgaatgcccccgaccgatacgaacgattcgtcgtccctgaaggcaccaaaaaggtttcgtatgagagggacacgaagatcatcaacgctgcttccttcacgattgagagggaagaccataccattggcaacatcgtccgcatgcaacttcaccgcgacgagaatgtgttgtttgctggataccaactgcctcatcctctcaagtacaagatcatagtcaggattcacaccacaagccagtcttctcctatgcaagcatacaatcaggctatcaatgaccttgacaaggagcttgactttctcaagagccaattcgaggcagaggttgccaagttctcgaatccgtactaacaaccaggcggcataaggagctcagagttctccagtggcacttggagaaacgtgtcaccattttccttatctaagcagacgctgcttgttttgcttgtataaattagaaacaaatcttcctgtgattcgttctatgttttggatattatttccggccgctagtatttggattaccttctctctctctctctagtctctaccacttttttctaccaaaaaacaaacgcttgtgaaatgtctctgaagagtttgtaaaaaaaaaaaaa

SEQ ID NO: 263 - DNA, 517 bp, Brassica napus
gggagctcacagagacatcgggggtgactattcttctcttcttcgcaatttcaaaaccctccgagaacgtttgagatcaggatgtccgtcgaggaagacgccaccgttagggagccactcgatctgattcgattaagtatcgaagagagaatctacgtcaagctccgatccgaccgtgaactccgcggcaagctacacgcttttgatcagcatttgaatatgattctgggtgatgttgaagaggttatcacgactgtagaaatcgacgacgagacatacgaagagatcgttcgtacttcgaaaagaaaggttccgtttctatttgtgagaggagatggagtgatcttggtgtcgccgcctttgaggactacttgacttcagatacagtgttcttccgtattggctgagatcacaaatatggagctgcctcttttaatgattccttagcttctgatttgtatttgtgtgttttgttttttcaaacattttaactgaatgtagctgagatattgttatt

SEQ ID NO: 264 - DNA, 813 bp, Brassica napus
gcacgaggggagaccgtggaaaggagtactcatgtttggtcctcctggaacaggtaaaactctgttggcaaaagcggttgcaacagaatgtggtaccaccttcttcaacgtctcttctgctacattagcttcaaaatggcgcggggagagtgagcggatggtgagatgtttgtttgatcttgccagggcatatgctccaagcacaatttttattgacgagatcgactctctttgcaattctcgtgggggttctggagagcatgagtcgtcaagaagggtgaaatcggaactcctagtccaagtagatggagtgagcaatacagcgacaaatgaagatggaagtcgcaaaatagtgatggtgttagcagctaccaatttcccatgggacattgatgaagctctcaggaggaggctggaaaaacgtatatacatcccacttccagatttagaaagtcgcaaggctcttatcaatatcaatctgagaacagttgaggtggcgagtgatgtaaacattgaagatgtggctaagagaacggaaggatacagtggagacgatctgacaaacgtgtgcagggacgcctcaatgaacgggatgaggcggaagatagcagggaaaacgagggatgagataaagaacatgtcaaaggacgatatctccaacgatcctgtcgctatgtgtgatttcgaagaggccattaagaaagtgcaacccagtgtgtcttcctccgatatcgagaaacacgagaagtggttctctgagtttggatctgcttaaacccttggaaccaaccactttttctcagtattagtctc

SEQ ID NO: 265 - DNA, 417 bp, Brassica napus
caagccgagctcttgcaaagatgtcgtttgaggagaaagatctgacttgggatatctctggtcttggtccctttgagcttgaagctcttcaggactgggagtacaagttcaacagcaagtatgccactgttggcactgtcaaagtgaccgtttcagaagctgaaactgcatctgtgtctgaacccgcagagactgatgacggagacgctcatgtaaccaagcaagagccgacagttgtggataagaatcttgagactcctgctgagagtgatgtgaagaacgagtagattctgtttccatgatttactctgatgatgttgaagtttcaatgatataatctttgtttcattacctcaaaaaccaagtttgctggttaataaagtatttataaaatgctcgatcacatggtgtttgaat

SEQ ID NO: 266 - DNA, 363 bp, Brassica napus
cggcacgaggaattcacgagattaaggacttccttctgacagcaaggaggaaagatgctcgatccgtgaagatcaagagaagcaaagacattgttaagttcaaggtcagatgctcgaagtacctctacactctctgcgtctttgaccaagagaaagccgacaagcttaagcagtctcttcctccaggtttgagcgtgcaagacctttgaagatgaagaagcctttggtgagtgatgaatctttgtgccattctatatttttcttcttctaagttttgtgtcgttcagttatgcctagactttttgtggattggttcagtttttaatatcagattatgttctcgctaaaaaaaaaaaaaaaaaa

SEQ ID NO: 267 - DNA, 481 bp, Brassica napus
gaacagatccgacaatgcctaagcaaatccacgagatcaaggacttccttctgacagcaagaaggaaagatgcacgatcagtgaagatcaagagaagcaaagacattgtcaagttcaaggtcagatgctcaaagtacctctacactctctgcgtctttgaccaagagaaagccgacaagcttaagcagtctcttcctccaggtttgagcgtgcaagacctttgaagatgaagaagcctttggtgattgatgaatctttataccattctatatgtttcgtcttctaagttttgtgttgttcagttatgcctagactttttgtggattgattcagtttttaatatcagattatgttctaaaaaaaaaaaaaaaaaaaaaaaaaaaaaaaaaaaacaaaaaaaaaaaaaaaaaaaaaaaaaaaaaacatgtcggccgaaaaaaaaaaaaaaaaaaaaccaaaaaaaaaaaaaaaaaaaaaaaaa

SEQ ID NO: 268 - DNA, 590 bp, Brassica napus
gttaggaactccgtttaagcctttcaatcaacttcttggggtgttcccagctgcaagtgcccatgctcttccagagcgttataggaccttgatgactgatcccaactcacccatcattgatttttacccaactgattttgaagttgatatgaatggaaagcgttattcctggcagggtattgctaagctgcctttcattgatgaaaaacgccttctggaagctgtttccgaagtggaattcactttaacggatgaagagaagcgtagaaacagcatgatgtgtgacatgctattcatcgcaacgtctcatcgccttgctgaactcatcttttcccttgacaaccattgtcggcaactgagtgaacgggagagactagactataaggctaggattagacctgagctcagtgatggtatgaacggctacttgactccctgttcaggggagactcaccctcctgtgtttaggtccccagtggagggtaaggaggacatcttggacaaccaagtcatttgttgcatttacagacttccggatgcacatgaacacaaaatcagacctccttccaggtgtcatcttccccaaaaag

SEQ ID NO: 269 - DNA, 453 bp, Brassica napus
ccccgcgtccgctgacattcatctgaagagaagaaatgaagcacaacaatgtcatccccaatggtcatttcaagaagcactggcagaactatgtcaagacttggttcaaccagcctgccaggaaaaccaggagaagagttgcgagacaaaagaaggctgtgaagatcttcccccgtcccactgctggacctcttcgccctgttgtgcatggtcagactctcaagtacaacatgaaggtccgaaccggtaaaggtttcactcttgaagagctcaaggctgctggaatccccaagaagttggcaccaaccattggtatctctgttgaccatcgccgcaagaacagatctctcgagggtcttcagtccaatgtccacaggctcaaaacctacaaggctaagttggtcatcttcccgcgtcgtgccaggaaggtcaagcctgtgattctctgttgaa

SEQ ID NO: 270 - DNA, 606 bp, Brassica napus
gttgcttcctttcgagttgatcatttgcagttctgggagttggtggctgagcttgtagtcgactcttctccacgtgcaagagatagagctgtcagagcagtggaattttggggtctaagcaagggagccataagctctttgtatgcaataatgttctcctccaaaccaatcccttcgctgcaacgtgctgcatacattgttttgtctaccgaacctatatccaggctggccatagttgctgatgggaatgtgtcacccagtgatgagtctctcagtgaccaggactccagcaacgttggcttgccatctgaagaaaagctgcagctaagagatgaaatatcttgtatgggtgagaagttaaattatgaactccttgatacagatttaactgcaccagacagggtgcaaacattcctgggatggtctttgttactctcgcatgttaactctttaccttcactaacacaaggacgagagagactggtgcagtatatagagagaactgcaaatcctttgatattagatagtctttttcagcatattcctttggaacaatacatgggcacagagcttgaagaaaaaagatggagatattccatctgaatt

SEQ ID NO: 271 - DNA, 520 bp, Brassica napus
agagtgacgtcttccttcttcagctctctctctctctctctctccttggaatcattcaatggcggagacagagtcaccgagctctgttcgtaaagttgttgttcatctgagagctactggaggcgctcctattctcaaacaatctaaattcaagatcccaggaactgataaatttgctaaagtgattgactttttaagaagacagcttcattctgactcattgtttgtctatgtgaacagtgctttctcgccaaatcctgatgaatcagtaaacgatctatacaataactttggctttgatggtaaactggtggttaactatgcgttttcaatggcgtggggataaatccgtatatgctactacagtctacagtgttctcaggccgtggtcttgatggatgctctggtgactttcagatggaagttgctgtacataatgtagcaatggacttgatatttttcactctacatacatgtaaataaagaccacatcaaagttctaaaaactagatcttcaatt

SEQ ID NO: 272 - DNA, 716 bp, Brassica napus
atagagtttcataccataattgttaccctaatgactccattttattcatagatttcgattctatcgaatacaaacgagaccgattgatttgattctaagattaagaaaataagggaaactaacccatagagtaagaaggtgtaggcaagttaatgacagtggtcttcactttggtcatcaaactgatgctatttgtcacaccttgtgatcctattccactgtccttaagcccctggaaagggaagtggtccggtccacgcgctggtgcagagttgatctgaaccgttcctgtctccattgcatcactgatcagcattgccttgttgatgtcctttgtgaatacacatccctggaggccaaagttactagcattgcaatgattaatgccttcttcaacagaattgatcctcaagacgggtaagacaggaccaaacggttcctcccaagcaatcctcatgtccggtttgacattgtccaaaagcaacggccaaatcaagttaccttgtcttttatactcttggcagaacgttgctcctttatccttagcatccatcaccaatgaaattagctgatgattcagacaccaccgctgtaatatcacagttctcctcgggtggtcccaccgtgagtttagccactttagctttcactttctcaacaagctcattcgccactgattccatcactaggactaccttaactgcagtacatctctg

SEQ ID NO: 273 - DNA, 657 bp, Brassica napus
ggattttaaccttttcaggtttcttccgacgatttctcaaacagacaaaaccaaatcaaacctctggccatgctcagatccagcctcgcttcgttaatcgtcgatgtcaacttacggcgcacgttacgtccacgcgcatccttctctcaccctgcgcatattagccgttcctcacgccgcgttcttccttccctgtactcctcccacacacctctaaggtttcggattcagacatgtaaccaccgccgcctctcctccttctcgtctccctctgtgaaacacggaccatcttcgtctacccgatcaagcctcgcgggtcaagatgacgcattcacggcggctcagaacagcttcaacggtcataaccacgacgacggcaacgagaactcttcaccgccgcaaccctcttccaaagttcttacattgcccaccgtattaaccattgctcgtgtagctgccgtaccacttctcgtcgcaaccttttacgctgatagttcgtggggaacgacggcgactacaagcatcttcattgcagcagccattacagattggcttgacggctatcttgcacgaaagatgaagttaggttctgcgtttggtgcctttctggatccagttgctgataagcttatggttgcagctacattgatcttactc

SEQ ID NO: 274 - DNA, 145 bp, Brassica napus
tggcgtgctcaattaggagtaacgctttccggtgttacaccgaaacggtgcgtttaactccccgacaacaacaatgcggccgcgtaaagcctactccttcctctttcctctcctttaattcctctaccatcctcgttcacactct

SEQ ID NO: 275 - DNA, 864 bp, Brassica napus
cggcacgagggctccttcctagattcctcaaagctccccattttcaatttttcagctatggcggttttcaaaaccctagctttcctgttcgttttcagcttcgaaactttggcggctgctgcagatgcaaattcctcgttttcctttgatgggtttgtgaaatctccgagctttcacaagaatattgctctgtttggagattctaagctcgtcagtggtggttcgtcgattcagctgactggctcagtgagtcggagccaagggcgagtcatttacatgaaacccatcaaactctcccaagctactactaaagggagaagctttccaggttcattctcgactagtttctcgttcaccatgccctccaaagagattgggagtgtcctggcttttgtaatggttccaagtggtttggatcttaggctgtttggtagaaaggataacgcttcctcaggtctaggtttcttaatgcataaagagattgttgctgtcgagtttggtatctccaagagaggaaaccgtgtgggggtcttagttggtagacctgaagctgctaaaattagaaacttatcatcttttgatggccatttcaacggagagaagagattgaattgttggattgattacgaggcgagctcaaagagagtagaggttcgtttgagtgtttctactgccttaaagcctgtagatccgttcgtgtcttactcagtagacttggctaagatatgggaagacaagaagttcatggtgggtttgacctcttcaaatggaaactcttccaagccacattatctccactcatggagtttcaagctgagacatccctcggtgaggattcactcgcagccgcttgatccaaacgca

SEQ ID NO: 276 - DNA, 746 bp, Brassica napus
ggacgcctgcggaccggtccggaattcccgggtcgacccacgcgtccgcagtaaaacgtgaggacgaaacctcttatctttgcaactacagacatggctggttcttctctaccttcatctctcctcactcaaggaggacctgctttactattctcttcttcgaatcaagtctcagaagctaagtgtcaacttgtggttacatctggaaaacgctcacttgttcgttgcttggctaagaagaaaatcagttttgtggatcagattcttgattacatcgaaggtggtcctaagcttaggaaatggtatggagcgcctgagcttttgccaaaggacggatcatcaagtgaagaagacgatgaagcagaggaacaagaagaagatgctcttgatggagtcaaagacactgtttttgtaacggacggagacagtgacgttggtcagatgattatactgcaactgattgtgaaaggcactcgtgttaaagcacttgtcaaggacaagagaaaggctatggaagcttttggagcttatgttgagttgacgtctggagatgcaagtgacgagcgtttcttaaggaaggcttttagaggcgttggtgcaatcatatccccaactgagggtttcttatcaaatgtcaagagtttaaaaggtgtgaaacatgtagttctcttgtctcagttgtctgtttatgaaagcagcggtggaattgaagctatgatgaatagcaaagctaaaaaattagctga

SEQ ID NO: 277 - DNA, 679 bp, Brassica napus
gcagttggtacttgcagggagtgttcttacatcggaacattcaacgagaacaagtgcttaagaaactgtggagaggcttctcagagatggtaccatgtccccaggtcatggctgaaaccaacaggcaatctattggttgtctttgaggaatggggaggagatccaaacggaatctcactggtcagaagagaagtggacactgtctgtgcagatatctatgagtggcaatcaacgcttgtgaactatcagttgcatgcttctggaaaagttaataaaccactgcatcccaaagtgcatttacaatgtgggcctggacagaagatcaccacagtgaagtttgctagctttgggacacctcaaggaacttgtggtagctaccgtcaaggaagctgtcacgctcatcactcctatgatgctttcaacagactttgtgttgggcagaactggtgttctgtaactgtagcaccagcgatgtttggtggtgatccttgtccaaacgtgatgaagaaactcgctgtagaagcggtttgcgcttaaaccagatgcagcagcaattgaaactgtagaagtggttacttcgcattcttacaggtttttctgatagtattagtatgttgttgtggctattttacaagcggtggaagtgtacaaaagggaacaacacaccgttcaaacaatt

SEQ ID NO: 278 - DNA, 187 bp, Brassica napus
atagaccagacatgccctctgttgtgtccatgtttggtcataaggccaatgactttcctcctcccaagcatcctgcgtttacgtccggaaggaagacacacgtcaagaatgacgaccgtccggtgcttaccccagtggagaaaatagcaattctgttaatgacattactctcactgatgttcatgag

SEQ ID NO: 279 - DNA, 506 bp, Brassica napus
gaaaccaaaaaaagatccattctcttcgatctcaatcaaccttacgtatctttccccaagtttgcctcggggaagaagaagagagaagttagattgtcaatggagccaaagcaaggaaacgtctcagccatgggagttggagtttccgcggcagcgactgcagctttaacggctgtgatgagcaacctgacaatggcattcgtggatgagagattgtccacaaaagacctccgtttaagatggccgccgcgtccgtttattctgttggatctgatggcgcaaggagtgaagttttgaagctctaaggcatatgaaagagattcctttacttaccattaacgatggctacaatcccatgaccggggataaatgattgatgatgaatcttttttgtttgggaatcttttttttttttttttgaaacacaactgtttgggaatcaattttttcttttgctaataaataaaaattaggatgaaaaaaaaaaaaaaaaaaaaaaaaaaaaa

SEQ ID NO: 280 - DNA, 285 bp, Brassica napus
  misc_feature (15)..(15): n is a, c, g, or t
ttcgccttcatcttntnnnncttctgtttttcgccttcatcttctccttctcgcatagaattttggttgagcttcttcttcttctcctaaacctcagaaagcttcacaacaatacaaacgtgatgggatctcttgtccgtgaatgggttgggtttcagcaatttcaaaattgattgaattcttcgccaaattgaagcaaaaggacatgaactcattgacagttgttgttatgggaaaaggcggtgtaggaaaatcatccaccgtcaactctcttattggtgaaca

SEQ ID NO: 281 - DNA, 704 bp, Brassica napus
gaaggagcagaatctcacgacctcagtctatctgcaatggagcgtaactcctcagacgacgacgagcatttgatccctcagaacgacactcgcagccgccacctagaagatccgccatcctcctcctcctcctccgccacaaacggcggatcatctttccagatcgaagacattctgtcgcggaggaagatctcgctcaacaagcgttacctcctcgccgccgtctcgctaactatctcgatcggtctcatcttcttattcactgatctccgtcagcaattctccagtttcaaggtggatccgctctggagtcgcgtcaaagaatccgagctccgcgcgctctatctcctcaggcagcaacaggtcgagcttctctctatctggaacggtacgacgagtgcgaactcttctgtgagagtcgccgtcgcgaagcacatctcactgaataaggagatccaagaggcgcttctgtcgccacacaaaaccgggaactactccggctcggtggctaacttgtacttgtacgagagatgcgtgaaggtggatcagaagttgtcagatcggaaaacagtagagtgggaaaccgagatcggacaagtttctgttcgctatatgcctctcgggacaaatgagcaaccacttgatttgcctagagatgcacatgttctttcgccgcgttgctggatcgggttctttgtgatccc

SEQ ID NO: 282 - DNA, 329 bp, Brassica napus
tcaacaggtcaaaaagttcctaacggacaaaccagagtcactctttctttaatcctttctgcattcactacggttcttgcctctcttctacttatgtgatcactattgactgagtcctcagaaacttgttacctcctaacggttcttgagattcttgatttctattggggttttggtttgttcatattcttagttatcatgtggatttgttgtgtagcataattcactgtagtgttctttaagactctggtgtgctttgtagtatttatgagattatatcattgttagtatacaaaaaaaaaaaaaaaaaaaaaaaaaaaaaacatgtc

SEQ ID NO: 283 - DNA, 705 bp, Brassica napus
gtccgcgcaagagattccgaagaaacataagtacaagagagagaaaagtgtgaagctggagagagaaaagctgctagagtgagagtgagacaagaaaaggaacatgctgttggaaacagaattttggaatggaccggctgctacacaagttttgcagctgcggtttggacatagctgcaacagttatcttttttttttttagaaacagctgtgatcaagtcaattttttcaggcatccagatgattattatcttcttgaagttgactgtttacgagtctctttaacctctgtagttactgtgaagacagattgaaaactcacttccaaactcaaatgaacagcaatgctctctattcagagatcatgtggagagatcccagagctttttacacatatttctatcatggccgctggaactggaagcactgcttttttcactgtgatttgtagctttgcttcaaggcgtgtacccttttgcgcaaacaagttcttcaacactggacttggtttcagtttggtcatactgctttgggctgtggatagactcgggaggtgattacttagcgcatacttcttccatgggttgtggcttctaggtaagcaacaagaaaaagagtaatttctgaggaagcaaaagccacaaccaagttctttatattcttataggacacatctttgctcctatgtaaactaaatatgtttct

SEQ ID NO: 284 - DNA, 570 bp, Brassica napus
gaaaacagatagcattagaaccaaaagtaatggcgacatctgttgcgactctatcttctcctccaccagtttctttacctctctcatcatctcgctcttccttattctccaactgcttcacagtaacgacccgaccaaagacccgttctttggtagccattcgaccggagcagaggagaaaggcactcacttgcaatgccctgtttggtctcggtgtccctgagcttgccgtgattgctggcatcgctgcgttgcttttcggacccaagaagcttcctgagattggtaaaagtattggcaagaccgtcaaaagctttcaacaggctgcaaaagagtttgagtctgagcttaagacggagcctgaagaaactgttgctgactcatctccggtggcaatgagcaacaaggcagaggagaaaactgaggtttcgtcaagctccaaggagaatgtatgaagaatgatgaatgagactttaggctttggtttgtttttttcatgtagctacaagggtttgtgaaatgagtttagatcaaatactctttgttgaaagaagcagagtagtcttcccg

SEQ ID NO: 285 - DNA, 933 bp, Brassica napus
gcaatacagacacttcttagatgcaaatatgtacccaatgctcagttacacttcctcgcctctctttcacacaaaagctcgaagaagaagaagagcatccatggatgcaatcaaatgtagctccacctttctccctcgcaccaatctcaacactctcttctctaaccacgcgtttcttaaatctcgcacgcccaatttcaaccgtcgagccgcgcagccactgtccatctccgccaaggacacgagaatcaccgaacccgaaaacgtcgccgtagcggagaaacctcctctatcaaagaggttcctcgtctccgacggactcccttccccgttcggcgccaccgtgagagacaacggcgtcaatttctccgtctactcttcaaactccgtctccgccaccatctgcttgatctccctctccgatctccgtcagaacaaagtgacggaggagattgagctcgatccatcgacgaatagaacgggccacgtgtggcacgtgttcttgaaagggagcttcgaagatatgctgtatggttatagattcgatgggaagttttctcctcaagaaggtcattactatgactcctccaatatcttattggatccttacgccaaggcaattattagtagggatgagtttggagttttggaagctgatgataattgttggcctcaaatggcgtgtatggtgcccactcttggtgaagagtttgattgggaaggggatatgcctttgaagctttcacagaaggatcttgtcatatatgaaatgcatgtgcgaggtttcacaaggcatgaatctagcgacatcgagttcccgggcacgtaccagggtgttgcggagaagcttgaccacttgcaggagcttgggatcaattgcattgagttaatgcatgtcacgagttaatgagctggagtattacagctacaatc

SEQ ID NO: 286 - DNA, 753 bp, Brassica napus
gttctctatgatgtcgaggagaagaatgcttttgttaagagtaccgttgatgtttccccgcgtcttcagctccgagctcttcataatgttaaggcgcaacaaggagaggttgctatggaggcaaatctagctgaacctggctattctctcgagctttcatcacctgttcctattggctatccaagagcgactcttaagttcccccttggcgaagtttcagtacaagagaaagaagtggaggaggaggagaagagcaagaggatagtatctgttaacggagtcctcaaacgtcaagctatgaacggtgtttgttctgctctgtacacagatgaagagttgaggctaagatatgcttataaggataaagcattgtctttcatcccaaccatctcccctcctttcaatgctgtttcatttgcattcaagcgccagtttagcccctcagataagttgagctactggtacaactttgattcaaacatgtggagtgcagtgtacaaacgcacatatggtaaagactacaagttcaaagctggctatgattctgatgtacgccttggctgggcatctctatgggttggggatgaagatggaaaagtgaaaacgacgccaatgaagatgaaagtgcagttcatactccaagttccacaagatgacatcaaaacctcagtcttgatgttccgagtcaagaaaagatgggacctttgagtgaataatctttgaatcacataaaatctcaatttacttctctct

SEQ ID NO: 287 - DNA, 740 bp, Brassica napus
tagacttcttatattataactcgacaacttgttcttgctaatagaagaagtccattacaatcactaccaaagattataagggaaaacaccaaagggattacataaactactcatcaaatatcatctttttcttttgtctttttcttggacatcactgcaaaaatggtctgaaaccatttttcgtctgaaactttacctttcgtttggcttattttcactcggagcgattagcgaacctctcaaccttgttcgtcgttgacattgatcccaaccatggtctctccaagcccacagctcacctccacaagcatctccggctcactgtaatgagtcaccgcctgcacaatcgcacgcgccctacgagctgggtcaccgctcttgaagataccggaacccacgaagaccccgtcgcatccaagctgcatcatgagagccgcgtcagccggaccatcgactccacccacggcgaactgaccgacggcaagacgcccgacctgctcgcgctgccaccaccagatcgcaaggagcagctaccttctcgcccacagtgaagacctcgacgtcatccatgctccttacagccctgatggtccccataacagacctcccatgcctcaccgcctccaccgcgttcccacccccagccccccgcttccgcctaatcacgccgcacctacgcggatccccgtcgagactccccccccctccccccacccccaccaccgccccccgccccaccg

SEQ ID NO: 288 - DNA, 1032 bp, Brassica napus
gccttaggacggccaagatggtcggagccaatcagaactacttaacggagcttgaaaacagggtgtcttggcagctggaccaagcctctctcaaggagctcatgataccttcgtttagccacacgtgcgggactttgctcgacgttgagctcgtgactcgtctcgtgaagaagttcgctggtttggatagtgaaggtgttaaaaccggtgcggctcttgtcaaggtggccaagcttgttgactcttacctcgctgaagctgctattgacggtggtttgactctaccggagcttatatcactagccgaagctctgcctagccatgctcgtaccacagaggatggcttgtaccgcgccattgatacatatctcaaggcacatcctcaagtgatgaagcaagaaaggaaagaactgtgtagactcattgatagcagaaagctatcaccagaagcagctcttcatgcagctcagaacgatcgtttacccgtgagatcaatcatcagagtgttgttcactgagcagacaaagctaagccgtcacgttgactggagtccatctggctcacattcctccgagccaggctctggaccacagtgcttgtctaaacgcgagatgaatgtccagcaagcggagataaagagacttcgagatgatgtgttgaggctgcagagtgagtgtggtgcaatgcatatgcagatggagaggcttatggagaagaagagtagtggtggtgggagtaaagggtttttcaggtggaagaggcttgggttaggaccttctattagaggaagtgttggcgttgaaaagatgatgaacggtgaggaagaagtaggtgataatggtgaggagttggagccaaggactcctgggaatatgaagacaaggcttgtcaaaggaagaacaacaccttcaaggtggagaaaatccatgtcataattaatgtttgcattaatggaaacaatacaataagaagtctttttgtatacacaaacacgctatagaacaaatgcaagttatgaaaaaaaaaaaaaaa

SEQ ID NO: 289 - DNA, 849 bp, Brassica napus
gtgtatttgcattccgttcatggctgttctgtttctctatcttctcagtctcctcatggctggccattgtagtgccacatggtgtgtgtgcaaaacagggctgagtgactcagtgctacaaaggacactagactacgcatgtggaaatggagctgactgtaacccaactcacccaaaagggtcttgcttcaaccctgacaatgttagagctcattgtagctatgctgtcaatagcttctttcagaagaagagtcaagcttctgagtcttgtaatttcactggtaccgccactcctacaacccaggatcccagctattcaggatgtccattcccttctagcgttagtgtcactggaggtggtggcagcaccactggaggtggtggcagcaccactggaggtggtggcagcaccactggaggtggtggcagcaccactggaggtggtagcagcagcaccgtgacgccaggcaaaagcagtccaaaaggaaacagccctatcacaacatttcccggcggaaacagtccatacactggcagtccattcactggcacaccaaccactggactgctaggaggcaatatcactgatgctaccggaaccgggttaaaccccgattactcaaacgaaaacagcggtttcgtgctccgtaattccaacacttggttcctctgcttgtgttctgtcttgatgatgatctgaagatgatgaatggacttcctctctagtttactatccaaatatgctatgttcttgttttattttggacttctgtacgttcttatcaattttagggaaacaattaacaatgtaaaactgaatcgtttcgggacgaatttggtttctatttg

SEQ ID NO: 290 - DNA, 783 bp, Brassica napus
ggtgaataaatatctattttttgaatccacactttagcaatgatatatttaacagctgataaacccctttcaaaactaattaaaattaagaagaaaaaaacattgaacgtatttttcaactaaacttcaacaaactatctaaacgaattcaaattcagaccaacaattttgaaaatcgatcaatttaaaccccacctcgaaaaaatcaatcagacaaaggacctggtgaaccagatgaacgagaaacctcctcaatgagcctcgtgacttcaggcattgaaggacgactatctggatactgagcagtgcagcttataccaatcttcaaaagcctgatcatattctcattatccccatcttgataccttgtgagctctggatcaaacacatctgaagtagattgttgttcagtgatcgacgaaacccatctcggtaaatctactccctcttcgcttaactgctgatgcgttggagatttccctgtgagtagttccagaatgagaacaccaaagctgtaaacatcagccttttgagagattctacgagcatcggttacttcaggagcacggtaaccatcaatgcggttaggcgtagacgtaggactgatcataggcgcaagacagtaatcagagaccttagcctcgtaggattcagacaagagaatgttagaagacttaatgttcccatgagacgttgtcgcgtcacgtgaatgaagataactaatagctctcgccgctcctaaagctataccagctctcgtttcccaattcaacggacttctac

SEQ ID NO: 291 - DNA, 455 bp, Brassica napus
atcaacgcagagtacttttttttttttttttttttttttttttttttaatgtttcttaaagcttttggttagtagtggtgtacgctaatgaatgacctggtgggattatggctcggctcttatttgattaaaaacattccttcgttgactgctgtgatgcaaaggagttctctggcatctcatacgaccccattccactttttttcccttcgaacctccaaaaagctatacttctgattttttaccttcaaaagagtcatgttcgtcttttggctattgaacttttagtaaccttctaataatatttgtttctgcaggacattgtggacgacagaatctaacattgtctgagcaatgaggaaggtgataccttatgcagacgaatgtctggtttgtgctcgaacaaacaagtggaagctctacaatagttttaatcccgcgtactctgcgttgat

SEQ ID NO: 292 - DNA, 733 bp, Brassica napus
tgaatgcacgcgtcgtaagcttggctcctctagagtcgacccacgcgtccgacacagtccagtcctgaagcgagttttgtccgtgttgtgaaaggctggtacttgattcttctctacttgtgtcctccatcctgtcttagtatgataacttgacaaacctacatacaagtgcctcctgtgcggtaaaaatgtctctgctttgctcttcatctcgtgtagctccccccttgccacagaccaaacagtcaagtccacgttttctgaaactcaaaacacgtaccttaactctatgctcttccatcagctacactaatgtcaggaagcacatcagttacatgttcgcagaatcacataattttggtaggctaacaaagagtcagatactagttagtccggagtcttttacagagaacagcacgatagatatggattgggaagatcaagaggacgtggaggataccggttcaccatgggaaggttcggttatgtaccggagaaatgcttcagtcacacacgttgaatactgcaccaccttagagaggcttgggttggggaagctgtccactgaggtctcgaagaagagggcttcttcgatgggacttagagtgacaaaagatgtcaaggattttccggacgggacacccgttcaagtttcggttgatgtcataaggaagaagaagaagctgagactcgatgggattgtcaggaccgttatcacacttggctgcaacag

SEQ ID NO: 293 - DNA, 622 bp, Brassica napus
gtctcccttcttcgtctttcaaccatacgaagcttttggtagaaaagcatatacacctttcttaatcatccaaactccaaagagttccttaggacctgatggaacaatgtgtggctccttaagataaaaagatccgtctttagtttggatttggatctgagctaagttctcattcatcatctttgttggtggaatggcgtcagaggatgtgagtgttgttgcctattcaaagaaagaggaagagagtgttgatgttgacagttccaaggacaagaacagacctgagagtactcttgacttatctaccgaggaaactaataacgtcaatgataaagttcctgtaaaggaagtagacctccctgaggctggtggtacttcagtgaagagtaaaacagctaatgtcaagaaaaagacagggaccttctctcgtagcccgaggttcctttctcagagctcttcattcggagctcatgctaagagcattgatgcaacaccaacaaaggctaatcctaaacctgtcgttgcaaacggatccaaaccaaaggtcactctctcttcaaacaacggtgtttctgccaagcgcaacagtcttgtatctgcgcccatcagaaagcaaactatg

SEQ ID NO: 294 - DNA, 667 bp, Brassica napus
ggagagagagagagagagagagagaaagagagtaatactttgaagatgagtgcgagaagaagcaacgcagaagggaagaggagtttaatagaaatgagtgaggaagaggagcgagaagaagatgatgatgatacttttgttgaagaagacggagaagaagaagaggaggcttcagagaagaagcagaaaggtaaagctacaagtagtagtggagtttgttgtcaggtggagagatgtactgcggatatgagcagagccaaacagtaccacaaacgacacaaagtctgtgagtttcatgccaaagctcctgttgttcggatctctggtggttaccaacgtttctgccaacaatgtagcaggtttcacgagctccgtgagtttgatgaagccaagcggagttgcaggagacgcttagctggacacaatgagagaaggagaaaaagcacaaatgaataaaacgatggtgatgaaaggtctcaaaaacatgtttctgaatgttaatgaaataggctttgcttgctcacttctgtcaagtctctttttaagctcctagtaatcttatgtgtctctctctgtttatcaatactgtaatgcaatcaaaagctatatgctaaacctaagacatcaataaatggcttaagcaaaaaaaaaaaaaaaaaaaaaaaaa

SEQ ID NO: 295 - DNA, 751 bp, Brassica napus
tggatattaatgaaatagaggctctttatataaggattacaagtatagaaaaaagaaaattatccgaaacctaatacaacatgaaataggaaaagatctaaaacagataaatagaaaacgtcctaagtctaaagtcggccaagggataggccgactctctctcctcttggacgccgattctctctcctcttggacacggctgggccttgtcttggttttggctatgggacatccacttaatgatttataactaaacaagtaatgaatttaatgattgaattcattgtcccacaagaaaatcaagaaagatcaaaaacaagaaaatcgagaaaacaagattcccaaccacatgtaaccaacaacatctcacatgagccaaactccaaaacagagccccacaatcgccgtcacaagaacattgtttcgtgtaaccgtaacggcaccatttttatcatcagcaacctcaccgtcatcatctgacggagcatcagcgcttggtccgtcagcgtcttcatcagcctccgcgttcttaggcttatgcttacttgatttaggtgctggagcagacgccttaacggccttatagatctctctcggcaacaaaactttatctatcttgtaaataatcaacggctcttggtcctttaacgtccctgttaacttcgccgtcacaacgtccgtctcaagcgtcacttcctctccgtcgttctgaaccgtgaagtcaaacttgttgttaccctcagtcgccaac

SEQ ID NO: 296 - DNA, 411 bp, Brassica napus
acctccttcatcaaaccatcctacgtccagaatctcacatcttcatttccccacatcactctcacgccattcaccacggctcaacaaagaattcaccaccagaactcgtttcttggctcctttctgctcctctctcccttctccttcttctactcccgcctccaaagaagaggccatcctccaagccaagacttgcctctcttcttgtctcctcaaacccctcaacaaccccaagctcgcttccccaaagctcaagaaactcaaacagccccggttccgtctcgagatccccatcctagacgacgactcgccttcctctctctcccagctcgcgttttcgatctttagcgacatgcccatctcgagaagaggctccaactccgtcaagctcctcttcctctggccagaccc

SEQ ID NO: 297 - DNA, 652 bp, Brassica napus
  misc_feature (647)..(647): n is a, c, g, or t
gaaggttcagagagtgaagcgaaagtgtgcaaatggcttcttcagacgtagctcttttctcttctatgattcaatttcgttcgtcttcgtccatccgtaatgctcttccaacaatcttcaatctctcttcaccagccgcgagttcgtgcgcaatcaaatcgacccagattcttaaccggcgtttgagagccaaatcgacgaatttatctctttcttcgttgccacggagagggttcgtatgcagagctgcggagtacaagttcccggatcccatacccgaattcgccgaagctgagactaagaaatttagggaccatatgacaaagaaactatcaaagagggatctttttgaagactctgttgatgagattgtcggagtctgcactgagatcttcgagacgttcttgcgcagcgagtatggaggacctggaacactcttggtcgtacccttcatcgacatggctgatactctcaatgaaagggactgcctggaggtccacaagccgcacgtgcagctatcaaatgggctcaggatcatgttgacaaagattggaaggatggaccggcaccgattagtagcacccgaacctcaaatcatcaaaatatagttcagtactcatgtatcagaggatatattgtctttgtanactct

SEQ ID NO: 298 - DNA, 680 bp, Brassica napus
  misc_feature (648)..(652): n is a, c, g, or t
gatcgcagctgtagctccaactttgcaaggcgagtggatcaaggtggagcagaaaggaggaaatacactaagtgacagaagctcacacggcatagctgtggtcggggataagctctacgctttcggtggcgagttcaataccaacattgccatcgacaacgaccttcacgtctttgacctcaacactcaaaattggtcaatcgctccggccaagggatctggatcagacatccctaaaagctgcttcggcgtccgcatggtggccgtgggaactaagctatatgtctacggaggccgcaatcagacggaagggaacaacgacttttactcgtacgatacggtgaagaacgagtggaagcgtctgacggttctcggcgaagagggaggacccgaggcgcgtactttccattcgatggcttcggatgaaaaccatgtgtatgtatttggtgggacgagccaaggcgatggtgttacgccgaacccccatcggttcagaacggtcaaggcctataacgtttctgaagggaaatgggctcagctacctgatcctggagacaacttcgagaaaagaggaggagcagggttccatgttgtgcaagggaagatttgggtggtgtatgggtttgcgagttcgcttacgactgatgnnnnnagtgactatgagtctgacaaagttcaat

SEQ ID NO: 299 - DNA, 666 bp, Brassica napus
ctctctctctcactgttgaatgcttgttcttctctctatcatcgaactgaatcaaaacactgagtagtgggttctctctggaaaaagattcgatttgtttgaaaagacagacggatgatgacacttaactcactctctccatcggctgaatccatatctctttgcttcttggatacctccaggttcacccaaacccccaaacttccaggtgggtgtagtttgaggaagaggaatcaagggagaagttttggaaaaggagtcgtcaagtgttccgtgaaagtgcagcagcagcaacctcctccggcatggcctgggagagctattcccgagccgcctcgtcaatcttgggatggacctaaacccatctccatcgttggatctactggttctattggcactcagacattggatattgtggctgagaatcctgacaaatttagagtcgtggctctagctgctggttcgaatgttgctctccttgctgatcaggtaaagagatttaaacctgcattggttgctgttagaaaagagtcattggtcgatgagcttaaagatgctttagccggtttggactataaacctgagatcattcctggggagcaaggagtcattgaggttgcccgtcaccctgacgctgtaactgttgttacaggtatagtaggttgc

SEQ ID NO: 300 - DNA, 522 bp, Brassica napus
ggaacgcgatcaaactttcctttctctctctcgaatcctctcttccacgaaaaggaaaggcgaatcgtcggcgacgtctcggagcagcaaccagcagcaagggcggtttctctctcctcgtcgacgatcactcccatgacgctccagccgaagctgaaaccgattcatggcttagggacactaaaattcggttatttggtgaattctcatcttcctaaaaccagaaccgatgtctctctccggcgtagcgctatcgtcgcatctgccattaccggcgcttcaggaactgaaactgccgatttgctagagacagtgaaagtctcggacttgaaaggaaacgagattccgatctctgatttatggaaggataggaaagccgtggttgcatttgctcgtcatttcgggtgttgaactaaattgatcagcatgttcatacacttcgttttcattacattctgttttgaagaaaaaaaaattaaagtcttgagtttttgttcattttgcagttggctgagaaagaaa

SEQ ID NO: 301 - DNA, 620 bp, Brassica napus
atccaaaaacatgattgagagcctctccgttccaaccttccgtgttggttacccagtgaacactgatgcactcgacgcactctacgagaaggtgaagccaaagggtgtgacaatgactgctttattggctaaagctgcagggatggctcttgctcagcatcctgtggtgaacgcaagctgcaaagatgggaagagttttagttacaatagtaacattaacgttgccgttgctgttgccatcaatggcgggttgattacgcctgttctacaagatgctgataagttggatttgtacttgttatctcaaaaatggaaagagctggtggggaaagctagaagcaagcaattgcaacctcatgaatacaactctggaactttcactttatcgaatctcggtatgtttggagtggatagatttgatgctattcttcctccaggacagggtgctataatggccgttggagtttcgaaaccgactgtagttgctgataaggatgggttcttcagtgtgaaaaacaaaatgctggtgaatgtgacagcagatcatcgtattgtctacgggagctgattttggtgctttccttcaaacttttgcaaagatcgttgagaatccagatagc

SEQ ID NO: 302 - DNA, 656 bp, Brassica napus
aaaactcaaacgctcttgactttctgaagaagcaacagagagagagatttaatggcggccatgaactcgagtgttctcacctgcagctacgcaatctctggtgctagctcagtagagctaaaccagaaagtgggtttggtgaattcatcggttgggtttggccagaagaaacagacggttcctgtgatcaaagcagctcaacgagttggtggtggtgatgatgttaatggaagaagatccgccatgatgttcttagctgctacactcttctccactgctgctgtctctgcttctgctaatgctagcgtcttcgatgaatacctcgagaagagcacagccaacaaagaactgaatgataagaagagattggcaacaagtggagcaaactttgcgagagcattcaccgttcagttcggaagctgcaagttccctgagaatttcacaggatgtcaagatcttgccaagcaaaagaaagtgccatttatctcagaagattggcattggaatgcgaaggcaaagacaagttcaagtgtggttccaatgttttctggaaatggtgatcatgtttgtatccattcttatgtaaacccaacaatgtatctatctctctgttcatcaaagttctcatatccatttgaatctatcgtgttaaaact

SEQ ID NO: 303 - DNA, 661 bp, Brassica napus
gcgtccggtgagaaacttcttggtacaggtgcccgctctaatgcctacactgatttccaccatacagtgtttcatatccattctccaacgcacacaaaggactctgaggacgacctctttccatcggtgctcgacgccttgaatgagatagcttttcacccaaaattcctctcttctcgagttgagaaagaaaggcgggccatactttcagaacttcagatgatgaataccattgagtatcgtgttgactgccagttgttgcaacatctacattctgagaacaagttgggtaaaaggttccctattggattggaagagcagattaagaagtgggatgttgacaaaatcaggaaattccacgagcgatggtacttcccagcgaatgccacgttatatattgtgggagacattgacaacatacctcggattgtccacaatattgaagctgtgtttggaaaaactggtttggataatgaggcaacacccacttcttctacgcctggtgccttcggtgccatggctaattttcttgtcccgaagctaccagctggtcttggcggaaccttttcccaagagagaacaaatactgctgatcaatccaagatcattaaaagagaaagacatgcaattcgtcctccggtggagcataattggtcgcttc

SEQ ID NO: 304 - DNA, 867 bp, Brassica napus
cgctctctctctctctctccagatatttctttcttgcatccaaagcaaatcttcatcttcttcttcaaactctcttgagaatctcccagataacagcaaaccaggagaaaattaaaaggttaattaatattatctgaaactatgagttgtaatggttgtcgtgttctccggaaaggttgcagcgagacttgcatcctccgtccctgtatccagtggatcgaatcagctgaagctcaaggccacgccaccgtctttgtagccaaattcttcggccgtgctggcctcatgtcgttcatctcctccgtaccggaatctcagtgccctgctttgtttcagtctttgctctacgaagcttgcggaagaactgtgaatccggtgaacggcgccgtcgggttgctgtggacggggaactggagtgtctgtcaagcggcggttgagacggtgctccgcggcggttctttgaaacctatccctgagttgcttacacgtgacggcggattcccgtcgccgacttccgacgaagcatccgagatttgcatggagatgatgaatgattccggtgatcgggatgtctactaccaccactccagattctcgagctctaggtccagagcgacggcttctccgcccaaacggagacgattagcgtcagagcaacaacaagaacgaccgtcgtcggagctagatttctctctaatccctacttttccggttaaaacgacaccgtttaaggaggaaacgcgtcgaccagaaacaccgtactcggaggaatccgttacaacggcgccgtttctggataacatcgccagtgaacggtttgtacgcggcgggggagaaacgacaaagttgctcaaccttttcgcttga

SEQ ID NO: 305 - DNA, 720 bp, Brassica napus
ggtgacctcggtttcaacccaatctcctctgtcttstggagcttaatcatcaacttagcgtttagctatgttacacttccggtatagtaaacaaaccttaattaacccttcaaatttagtgcaagtaattattatttatatgaaattgatataactatgttgtgaatggttaatatggtaaatagagttggttgccatcaccattactgcctgtctatatcgacaacatacacaaagccaagcatgggagtgattcaagcacctatgataccgaaggaaggtatgtcccatttaatctggagaacatatttagcaagtacgcactaacggctccgaataagttaacattcaaagagatgtggaacttaaccgaggggaaccgaatggcaatcgatccttttggatggttagtaccaaacacattcttggttaaataaccgttaaaatcaaattaaataggtctgatttgtgtattggttatggcaggctttcgaacaaagttgaatggatactactctatattcttgcaaaggatgaggaagggtttgtgtccaaagaagctgtgagaggttgttttgatggcagtttatttgagtattgtgctaaaaagaataaagagaaggctaattctcgcaagcaagactaagrgtttggtttatcttttgtatgaataagtatgcagtattgtatttggagcctactagtattattacgctctcg

SEQ ID NO: 306 - DNA, 671 bp, Brassica napus
ggcttctccgttgtcttcttccaccgtcgtctcacaccggattctatctctccaccatccgcctcttaaccgcagcttagtcttcgtaaaacccaaattacccttcagccgaaccgtttccagagatcttcgcatgcgcttgcattctgaaaccacgaactatggcgataaggaacatataccttcttcaatcgattcaaagttgaaccctttggaaggagctggatcaaagaatctcgagaaagtagttgcaacgatcctgatactagctcaagtgtggtctccattgcccttgtttggtctagactctgcttacatctcacctgcagaggcggttctttactctccggacactaaatttccaaggagtggggaacttgcactaagaagagccattcctgctaatcccaacatgaagactatacaggcttcgttggaagatatatcgtatcttctgaggatccctcaaagaaagccttatggtaccatggagagcaatgtgaagaaagctctaaaggtggctatagacgataaagattcaatattggctagtataccagcagacttgagagacaagggctcagaactttatgcatctctggttgacggcaagggtggacttcaagcacttattgaatcaattaggtttctagatcctgataaagtgtcca

SEQ ID NO: 307 - DNA, 763 bp, Brassica napus
cttcgaatatcgctaatttgaaattttgagaatgttacaatttgagtttgctataagtttttggattggtcttaattagttactaatctcaaggtttacaaaccgtccgtttcatcataacttttaacacaaatggtatatttgactatgtttgtaataacacatgcctctttgattttattctttcccggccctagtagtcatcttcgtcgtcttcttcatttcttgatcggaaaaagtttctggccacggctgaagcggccccaaaaaacatgaagagaactaggaggtccaggccaccaccgccaccaactgccaccgcaggaccaggggcaaagtaagaaaacggtgaccatccccaaccgccgtagccatatccatagccaccgattaaaggtggtgcaacctgaggattcacgtagatgttggtcctgtaaagagaaatctacgttaaggatataattgaactacgccaatttatatttgtgtttgcatttacaatataaaaaaattaacaaataatacaatcatcagtataacattttctgtttttgtgtataatttatgtataacatgtttgatacataaagctaaagtttgtgagttttaaaaaaaaagtttgtgagtttatttggagaaaaactacacaaccaatcaaccaatcaacaaaatacaagggccttaaagtttaataataatgggctaaaatgtggacacgagatctgtaaggcccatttatgaagcctcttttatttgaagattt

SEQ ID NO: 308 - DNA, 632 bp, Brassica napus
gtggattccgtatcttgctactgcagagtagattcaggtctcaagaccgttgtagaagctagaaagttcgttcctggctcaaagctttgtatccaacccgacatcaaccccaatgctcatcgtcgcagcaagaactctaagcgagagaggacacgaatccaacctccgcttctccctggcctccccgacgacctagccgtcgcttgcctcatccgtgtcccgcgtgcagagcataggaaactcaggcttgtgtgtaagagatggtacatcactaaaggttatgtctccgagacagagtctggcaaaagacttgctcaggtggtgagtgatccgagcttgacgaagtcaggggtttactggagctggaacaaggcttcggcgtctttcgagaacgagttatcagaagaagcaagcgatgttgagaaggctcgtaaagtgtgggagatcagtgagaagctcgttgggttggcttaatgacacttggaatctcttctttctgatgagatcatgttgtgccaaatgatgaaatacctagaggatgttctggatttttagatgtctgattctgttgagtgtagatctgtttggcttgtagccccataataaagaaaataaaagtggccagcgaaaaa

SEQ ID NO: 309 - DNA, 703 bp, Brassica napus
ggaccttctcttccaccatcaaatccttcgtcacaccttccctctccctcgcaacccatcgattctctctttctcccccgaatctcaacctccttcgcgtcccctcctctccaccatctctccgtcgaggttttcgcagtggtcgtatcgcagcaatggcttcttctgctcccggatcggttaacaaaccagaggaagagtggcgtgcgattctctctcccgagcagttcagaatcctgaggcagaaaggcactgaatatccagggacaggagaatacaacaaagtattcactgacgggatctatagctgtgcagggtgtgaaactcctctgtacaaatccgccaccaagttcgactctggctgtggctggcctgctttctttgacggcattcctggcgctataaaccgaactgcggatccagatgggaggagaatagagatcacgtgtgcagcgtgtggaggacatctggggcatgttttcaaaggagaaggtttccctactcctaccgatgagcgacattgtgtcaacagtgtctccctcaagttcgcacccgggaacgcagccttgtaataataatctacttgtattgtgcgttaaaaaaatagtgtgtgttgattattgtgtcccacccgctggctcgcactgtgtttcgatctgactgttagtagtaataaggttgttgaattaacgttttgagactc

SEQ ID NO: 310 - DNA, 121 bp, Brassica napus
tagatgtctgaaaacgcttagcgggctaaactgagggttaccttaaaggctgcaacgggtttagtaaagagatagcgtaacactaacccgctattacccgaagggggcacggaacaaacta

SEQ ID NO: 311 - DNA, 401 bp, Brassica napus
atttgcagtttcttcttcttctagatattttttttttataaaatgtctaatttagattattctaatctatttagtgaaccctgatttctttgcgcgagttttggttagctttgatgggtttttaataatgcttctgatcaaatcattttcgtttgaaggagatacttttatggttaaaggatgtaaatgtggataatgtcattaatcagtgattgtctcaagaaggaagatccatagacgaaactgttcgtttttgtataatgtgtaggtttgtttatacgtccatctgttttgataaagaagagagttagtctatgtcttatatatgaatttacgtttctagctttctcttagtgaacattgtatgtatggtctctaatctatcatttcatgtttgatcg

SEQ ID NO: 312 - DNA, 340 bp, Brassica napus
atcaaccgtcttcttcaacaagatgaaaggtgaggtgactattactgctatcttgaagaggtcaaatcaggactcaggagatgagggggaaagaggctgtgttcagtctttaaaagtctatgaggtcttttggtttcccgaagaagactcaaccaaaccaggacacgcaacaactctgtacatatgaagacgaagcgcactaaacagatgagaagaactgaagaagtcggttttgtagactgacataaagtgtgtgtttatgtctatctttagctctctcttactcatgttcgttatatatatatatataccatatcagcaagcagctgaaaagtttatt

SEQ ID NO: 313 - DNA, 730 bp, Brassica napus
tgaggaccggtccggaattcccgggtcgacccacgcgtccgaaaagtttatctttcacgtaagactcgaagaaaatggcgagcactgtgatgactacactgcctcagttcaatggtcttcgagccagcaagatctctgcagctccagttcaaggcctggtttgtaattttcattttttctgtcttttgaaatgtgatttgatattttctatacacttacgaaacttacaaacattaaaaatatatagaaattttaggggttcaatttcgtccttgaacttgatttttatgtgtttaattgatcatatttgacaagagactttgtaatatagtatgaagttgttattacattacaacgataattttagtttaaaattcccaagttttagttaatgttatcctactatataaaagggactaaattcaaggcttttgagactatccacctcagctaaaatattctcatcaataaaatattagaaatagatgtcatttgaaagataattaactaatatatgacttttgatatttctagaaatttcaaatttgtaactgctaatttattaatagaatcatatatttatattgaattatgttctatttttaatcattagatttcaaaggtaaataaaatattaatttataatatatatatatagtttataactttatattgtagttatgaataaagagaaaataaccggagagggtgaaaaaattcatgtaaaatt

SEQ ID NO: 314 - DNA, 582 bp, Brassica napus
gaagatgccgtgccttaacatctccaccaacgttagcctcgacggcgtcgatacctcttccatcctctccgaagctacctcctccgtcgctaaaatcatcggcaagcctgagaactatgtgatgattgtgttgaaaggatcagtgccaatggcatttggtggcaccgaggaccctgccgcttatggtgaacttgtctccatcggtggccttaaccccgatgtcaacaagcagctcagcgctgcggtctccgccatccttgagactaagctctctgtccccaagtctcgcttcttcctcaagttttacgacaccaaggcccatcaaagtcaagaatatgcacaatgtttacatgctttacaccagcagtactagatcatgattgaatagagaacaaaaacatcttcttgtgccatcctgaaaggtcacttcttttatcgactggtcactttatgcagggatccttctttggttggaacggatctactttctaggtttcgtcgcaggaaataaagttaaagtgatccctcgccaatgtatgaacgtgtatgatattattaagtattcaagcttctctcatcgtc

SEQ ID NO: 315 - DNA, 731 bp, Brassica napus
  misc_feature (27)..(28): n is a, c, g, or t
ggaccactgtaaagagaccaggattgnntttagttcgtgaaacttnttaggtgtagatctgaaaatcaatgcttgaatgacagttgaatcaaacttattaggtgaagacatgaaaatcaaacataaagtgatatggatcaatttcatatatatatgtgtatattgtgtttacgcgtgtgtctctcttcctcattccctgactttattttttaagaccaagcataaagaggacaagcttgaagtaagcttcacgaaatgcgagtctaagaaaggtgagcttgaaactgttccatggagggtatcgagcttcaacatttcaattagagtagacgcatagctcttttcgacaagaacgtaatctacagagatacaagcttgtccactgcaagaaccccacttccctccagagattctcttcacaaccgacttcatgtccttggagacagagtggtgatcaactatggtgggacattttccaccaagctccaatgtcactggagtcaaatggtctgcagctgcagccattatgatttttccaatctttggactcccagtgaagaagatcttgtcccattgatgcttcaagaggatagtggcgacatcaggtcctccttcaataactttgatggctttgttgtcgagataagacgggattgtcttggcaaggaaggcagatgcgttagggcttaattcagatgcttnnaagcacacggtatttctgctgatatgcc

SEQ ID NO: 316 - DNA, 370 bp, Brassica napus
gagtatgagagcaatgcaacgattgtgaaagttgatacagatgatgagtacgagtttgcacgcgacatgcaggttcggggtttgcctacattgttattcatcagtcctgacccaagcaaagatgcaatccggacagaagggctaattccaattcagatgatgcgggatatcatcgacaacgacatgtgaagtcttgtcctgaagaaacactttctttctttaggattgaaatagtttatttgactttttgaggatgcggtaacaacgtcatcatgtttcataagaaaatgtaaacgagatgaatgtagtaatctaagaatttatatgtatccagtgttagtttcggttaaaccttttaagctcgatgatt

SEQ ID NO: 317 - DNA, 489 bp, Brassica napus
ctctttccgcttagacaaatcaccgcttctgttctctatcctccaaccgccggatacaaccgtcgcggcatggcgtcccgcggcgattctgtttcgtttaagtccctagcggctcgtcagacttcgatcagtagtgatgacgatggttactgtactctcgtagactttgcaggtaatggaggaggagggagagaaggtacgactgtaggagatgatcttgtggtgttgctttaccacttgcaacatgcctgcaaaagaatagcttctcttgtcgcttctcctttcaactcttcacttggaaagctctctgtcaactcctctactggctctgaccgagatgctcctaagccgcttgatattgtctcgaacgatatcatcttgtcatctctcagaaactcagggaaagtagcagtaatggcatctgaagaagacgactctcccacttggataaaagatgatggtccttacgttgtggttgtagatccttta

SEQ ID NO: 318 - DNA, 731 bp, Brassica napus
aagagtcgttgcttatgggacacacgtcgacaaagtctttctctcctcaaaggaggaaccgaacctcttcttgatcaagccgttgatctaccaccaccggaggaggaggattacgatccggcaccggctttttgacctgttcggctcaggaggaggaggagggcgagagagggaagatcggagaaggagatccggcagtcgtcgaactcgctttcctcgtcggaggatgatgctaggtctacgggtacgacggtggagatgcggttagagctctcgaagaagtggtcgtcgacgtcatcttctttaacttcgcctaaccccacccaattcatcttctagtgtatgttaaagcctcagttccacccagaaaacccaaatccatctctacgtcgatacccttctccaatggaaccagaaaatgaatcttacggaaactaaagaagctgatgaagtaatgaagaggcatattgaagattctcttgcgatattgcctcctataaggacttgttacagcttgcagtcaagtgatcaaatcagtttaatcgatgttggaagtggtgcaagcctttctgggctggttctagcaattgcttgtccagattggagagttactctactggagtctatcaataagcttgtgttttcttggagcacgtcgttactttcactaggctaacaaatgttaaaatcgttagggggcagagcagagtggggatgtgatctagtgcaga

SEQ ID NO: 319 - DNA, 770 bp, Brassica napus
  misc_feature (11)..(11): n is a, c, g, or t
ctaaaagctanataaaagttgtaaagaagcaaaacacatggttttgtttcaaagctaaagataaaacaattatcttatgaatacatggttaaaacttttgatggaactaagtaataagaaaatgactgaactaaagacggcttaagtctagctctgtttcacacagatactcctttctgttcccccaaaagaactggactcatcaaaccaaaccggtgtaaccgacagtgtctaaaagcactgctttgatttgttcaggcaccacatcatggccttcttggcattgctcagcgcggaaaacatccaaagcaaacccgttaaagcagctaacaacagcttgttgaacatccaaccctaaattatccaaagctctcatggtcgaaagcaaaagaccaggtcgtcgtccacagaacatgtgaatgttcactgccttcccttcccttaacttaacctcaattcttgcttgctcgcctctagggcttgggaaggaagaagaagggcacaactcttccttaacacggtaacttagcgtttgcgtggtcggtgttaacggattcaagcttgaagaactcggtggagcagtagactcaagttcggtatgaagatcgttgattctctgtaaaagctctttaagataatcaatagcatcaccaagtatagatgctctatccattttgctgatcttgggaacgaccgatctaagcatgtaaagcctgtcgttaagcttcttccttcttcttctctcagccattagattcttagcaggcatg

SEQ ID NO: 320 - DNA, 484 bp, Brassica napus
tgttatgaagatctagggacaggacaggtgtttcagatagcatttcgacacaccaaattcacttatatatatatatatatatatatatgatcactgaatataaagcacaacaacgacaaccatccgtggagtacatcacataaagcgtaattaacacaattatcatcaaatatatattttctttttgtaacaccaatgatttgcatgaaaccgttaactccgaagtctcattatggtgatgccgctaagagtcgcaggcaacaacagcagcacttgccaagacatgaccaaaatccatcgcctttggacttcgtttccactgcggctaccacaggatcaccagcagccgcatccctggtcggataaccaatcggcggcggacttgtgtagggcccctgtggcttttccactacgactgaagattgattcatatttgattcagtgataagtgagggaataaggattatgaagaggagagagtgtg

SEQ ID NO: 321 - DNA, 721 bp, Brassica napus
ggggatcgttctatctcattctctctctcacctcataaaactcactagtccaacatcaaccaaggtataacccacgaaaagaaaaaaaattcacttagataaaccatatcttcatcatggcttcaataacctcagccaccgtcgcaatcccatctttcaccggtctcaaatccacatcctccaaaccctcaaccgtcgtcaaaatccccaccgtggcggcagcatcaacgaagctcaccgtgaagtcatcactaaaggacttcggagtcgctgccgtagcggctgcagcttccattgctttggctggaaacgccatggcaatagatgttctcttgggatcaggagatggggcgttagcctttgtccccaacgacttcacaatagccaaaggtgagaagattgtgttcacgaacaatgctggattcccacacaacgttgtgttcgatgaggacgagatcccaagtggtgttgacgctagcaaaatctcgatggacgagcaagatttactcaatggtccgggagagacgtacgaggttgctttgactgagcctgggacttacagcttttattgtgcgccgcatcagggtgctggtatggttggtaaagttaccgttaactaataaaatgtgactgtaatgtgacagagaatctctgagtcaggtagaggcttttgtttcagtcttacatagggtgttttgaaccgaaatggacgtgatatgct

SEQ ID NO: 322 - DNA, 755 bp, Brassica napus
atactggagctcccgcggtggcggccgcatcagactaggagatccccgggctgcaggtcttcggcacgagggaagaaacattataagaaagagaacatctcaacgtaacatcctctaaatttgataatagcaatatatacacaagtcagcttcaggatgagccagtgtgttccaaactaccacatcgatgatactccggcggccaccgtccgctccactaaagctgcagatatccccacgctagactatgaggtagccgagctgacgtggcacaacgggcaactagggttgcacggcttatgtccaccgcgggtgcctgctctgtcaaaattctccactggcgccggtggaacgttggagtcaatagtggaccaagctactcgcttccctaaccctaagcccaccgatgagctcgtcccatcgttccaccaccgctattccagggtccgcatggacgcgcttgtccccgagcagcagagccagcccgccaccggcgtgggctcgtgtagcgatggtcatcccatggatggtgggaaacgagccagagtggcgcccgagtggagcgcgagcaggagccagcgcctgaccattgacacctacggcttcacctcaacatcgctggatgataactccagctccggcgggaagcctttccccaaaaccaccaacatcgacgatcatgactccgtctgcccacagccgccacaggtggaggaagaagaagaagagagcaaacgggaggaaaatcatcagct

SEQ ID NO: 323 - DNA, 648 bp, Brassica napus
gggtcttgctgtggcaatgataaagctatgggagaagagcgcgatcgcgaagatagcaatcaaacgcggtgtggagaacacacggaacgagagaatggctgaagagctcaagaggcttacgcgcggcgtactcgtttcgagaaacaaagagtacattgtcttctacagaggcaacgacttcatgcctcccgcggtttcagaggcgttgagggagaggcaaaaggagataacggaggttcttcagaacaaagaggatcaactccgggaaacggcttcagcaagagtcacacctgtatcgcaaggcaaggggattaaaacgccgttgctcgctggaactctcgctgagactatagctgcaagttcaagatgggcgccagaggcaagcagtgttgatgtagaggagttgaagagagaatcagcttctattaagagagctgcgttgatcagagatcttgacttgagacttctttatgcaaagcaaaagctgagaaaagctgagaaggctttagctaaagtgcagaaggatcttgacccatctgatctcccaactgactcagagatcatcacagaagaagagagacttcttttccgcagaatcggtttgagtatggatccatttctcctcgtaggaagaagagaagtgtttga

SEQ ID NO: 324 - DNA, 729 bp, Brassica napus
atggatctacaatctcacttaaatcgtcttccaatggcttagaacctccatctcctgtctggctgaataatgtttctcttccaatgatgaacaacgagtgtttatgaccatagagaattgctgcaatgttaccaacaaatgagagttttgggcgcttacccaccttgaatcatctcacttcttaacacatcttctctccsttrttctakaaragcgagaaraattactaacattctasgtcwskcctctwattccggacttarrataascatwaaaagtgttgtcagatacgaatcacctctcatatctttagccaaaccctcaatgacatcatatatctcatcactctgaggatgctcttgatctctagacacaaaacaatttacattacctgcaacttcaatcccactacgaccaacttctttccttaaacccttttctctcattcctctcctcaacctatcaacactcgtccatttctgttcctcagcatacatattcgagagcaaaacctgatatcctgaaaagttcttccctttatccactttagctaacttctctgaaacagtttcagcaagctcaatctcaccgtgtactctacaagctcccaggaccgaaccccatagctcagcaatgtttccttcttcaccaagccctttaacaaactcgtaggcttcatcaacgcggcctactctccctaacatgtctgtaacgcagcaatagtgctcc

SEQ ID NO: 325 - DNA, 832 bp, Brassica napus
caaacatggacagatctatctttctttctctagcactagcctcactcttggtcggagtcgtctcagctcgtgactggaacatcttgaaccagctcaaatcactcggtatttcatccccaagccaacccggtcttgttacttcggggctatcgaccaacctgaaacggtactgcgaaagctggaggttcaacgtggaagtacacaacatcagaaacttcgacgtggtgcctcaggagtgcgtgtctcacgtccagaattacatgacgtcatctcagtacgaggatgacgttgagagagccgtcgatgaggtcatccttcatttcgggagcatgtgttgtagcaagagtaagtgcgatggcatggacgcttggatctttgatattgatgacacgcttctctctaccattccttaccacaagagcaacggcttcttcggaggagagaaattgaactctactaaattcgaggattgggtaaggaagaggaaagcaccaccagtgccacacatggttaatttgtaccatgagatcagagaaagaggaatcaagatcttcttgatctcttctcggaaagaatatctcagatctgccaccgttgacaacctgatccaagccggttactatggctggtccaacctcatcctcagggggctagaagatgaacaaaaagaagtcaaacaatacaagtcagagaagaggacatggctaacaagtcttggttacagagtttggggagtgatggcagaccaatggagcagctttgcaggttgtcctcttcccaagagaaccttcaagctccctaactccatctactatgtcgcctaatcaaaaca

SEQ ID NO: 326 - DNA, 724 bp, Brassica napus
tcgcatccaatgttgagaaatggggagactgcagattggattggtacattcgaaggtcataaaggtgcagtgtggagttcttgccttgataacaatgcgttacgtgctgcatctgcatcagcagacttctcagcgaaactctgggatgcgttaacaggggatgtgcttcattcttttgagcacaagcatatcgttcgagcatgtgctttctcagaggatacaaaacggttgctcacaggagggtttgagaagattctccgtgtttttgacctgaaccggttggatgcacctcctacggaagtggataagtctcctggttctgtcagaactctcacatggcttcacagtgatcaaaccatactaagttcttgcactgatattggtggtgtaaggctatgggatgtgagaagtgggaagattatgcagacattagagactaagtctcctgtcaccagtgctgaagtgagccaagatgggcgatacattactactgctgatgggtcaaccgttaagttctgggacgctaaccattttggactggtgaagagttatgagatgccatgcaacattgagtctgcgtcgttggagccaaaatctggtgagaagtttgttgctggtggtgaggatatgtgggtccgtgtgtttgatttctacactggcgaggagattgggtgcaacaagggacatcatggtccagtacactgcgtgaggttttcaccaacgg

SEQ ID NO: 327 - DNA, 703 bp, Brassica napus
  misc_feature (577)..(577): n is a, c, g, or t
acacgtcagcacgtgccaccgacgatctagacacaagacttctacgagccatgatcaccaagggaatcatcgagccaatctctgacgtgtatgtcaacttggtcaacgcagatttcacagccaagcagagaggtctgagaatctcagaggaacgtgtgctcttggacgggtcaccagagaacccgttggagacaataactgtccagctaggcaacgtggagtccaaatttgcgagctcgttgtctgagtcaggagaggtgaaagtggaaggaagggttaaagatggagtgccgcatttgacgaaggttggatcgtttgaggttgatgtgactcttgaaggtagtattatactgtgcaggcaggcggatcaaccgggtatgatagggacggtggggagtatacttggagagtctaacgtgaatgtgaactttatgagcgttggaagaatcgcaccgaggaagcaagctattatggcgattggtgtagatgatcagccgagtaaggacactcttaagaagaattggggagatccctgcggtgggaaggagtttgttttcctcaaggtctagtttggggnggggggggggtggtgaattatggaatcctgaaaggngaggagttcctatgaaagggttttggagtttggttggggttaaggcaataatgttatcttatggctttataacaaagttttgataaaaaa

SEQ ID NO: 328 - DNA, 825 bp, Brassica napus
  misc_feature (9)..(10): n is a, c, g, or t
ggaaatttnnggggaggatagggaggtaaaaattttagtatgattaattttttttttgttataacaataaggggagatgcggtctatgtatctttttgtcccaatccaaagattgaagtacagttacaaattaaaatagttagagtattacacagcaaacaaaacagcgaatctgttctaatcttcctcagcgtcgatgcttgttcggaagaggtagtcttcgccttcaagttcatcttgatacatgtttattggtggatctttatgtttttcatcctcttctttctcatcaatacgtctctttccctttcttagtatatagaagaaataatggaatccatcgccaaaagtgttgtactccatggaccaattaaacttgggatccataaacaaaggacgcctgaaatgcggctggccaaaagtaattgaaatgaaaatcccatctggtttcagaacccggtgaactccatcaagagtagccatgactttgctaacggtttcttgacggggattccatggatcaccagcatccacgaacagtacttcctctagttctagatcgcgggcggccgctctagaggatccaagcttacgtacgcgtgcatgcgacgtcatagctcttctatagtgtcacctaaattcaattcactggccgtcgttttacaacgtcctgactgggaaaaccctgccgttacccaacttaatcggcttgcagcacatgcccctttcgccagctggcgtaatagagaagaggcccccaccgatcccccttcccaacattgcgcaacctgaatggcaaatggacccccccgtaaccgcgcattc

SEQ ID NO: 329 - DNA, 673 bp, Brassica napus
cagaaggtggtcatcgtgcggtgcgaggagatttgcctctccggtggacttgtccgacagaaaatgaagtacatgaggttccttcgcaagcgtatgaacactaagccttctcacggccccattcacttccgtgctccctccaaaatcttctggcgcactgttcgcggtatgattccacacaaaacgaagcgtggagctgctgcgcttgcacgcatgaaggttttcgaaggtgtgcctccaccatacgacaaggtcaagaggatggttatccccgatgctctcaaggtgttgaggcttcaagctggtcacaagtactgtctgttgggtcgcctttcttctgaagtcgggtggaaccactacgacaccatcaaggagctggagacgaagaggaaggagagatctcaagtggtttacgagagaaagaaacaacttaacaaacttagagccaaggctgagaaggttgctgaagagaagctcggtgcacagctcgaaattcttgcacctgttaagtattgaagagaagatcatcatcatcactagttgtgttctctgtcttcgcatgttttttgaaccgacccattatactttatcattacttagggtgttgtatgagattttgtcctgatattatctttcttgagttccaagaaaaaaaaaaaaaaaaaaaaaaaaa

SEQ ID NO: 330 - DNA, 546 bp, Brassica napus
ggatatccgcctcagatggttcctggaatgaggccaggcttttttgggccgatgatacagcaaggcccacgaccaggtggaaggcggccaggtgatgggcccatgcaccatcagcctcagcaaccaatgccttttatgcagtcacagatgatgccaagaggacgtgggtaccgttacccacctggtagaaacatgccagacggtctaatgccaggaggaagggttccatttccttatgactctaatggagtgcctcttggtcagcatttgcccgccggtgcattggcttcttcccttgcttcttcccttgctcaagcttcccctgctcagcagagaactcttctgggtgagagtctatacccattagtggacctgatagagcacgagaacgctgccaaagtgacgggtatgcttttggagatggatcaggccgaagttttgcatctgctcgagttcccggaagctctaaatgctaaagtttcagaggcattggatgtgttgagaaacgtgaatcagccaacgcaggtgagtgaaggcaagagtgga

SEQ ID NO: 331 - DNA, 790 bp, Brassica napus
agaaattttaatactttaatagaaatttcaggaagagtgaagtaacttgtactagaaacagaatcaaaagctctctagcagacaaaacaacaaagaatggaacaaacaatcatctcttcaagcttctgcgactgggtactcgaaaactacatctctccctgtaagtttcctgtagacaccgaccattgtctcaagcttgtactcagtgttgttcctctccttaggctccaaaaacaccttcatgatcttggtgccatcaacacggtatctagtacgctttccaacaatctcagcagggtaagcgacatcctcaagcatggcttcatggacggaggtgagagtcctgttgcgtggtctctgaacagctgagcccttcttagggggacgcatgattctcctggtggcaacaaagataacatctttgccactgaacttcttctctagctctctgacaagacgaggatggatcttgcggaaggctttccttaatctgaagggaacgtagatcacaatagccttgcggttgccagaacaatccatctgaacagcttggttaatgtagagatctttcaagtcgcttttcaactcctggttggtgttttctaagtcaaagaaagcctgagcaacttcctgttcgaagtctgttggtgccacacccttgtccttgtggatcttgttctgagcagagaacatcttggcagattcagagaaacacgacgaagaaccctagaaggtggtgcaaaggtttactgcttggtgaagcaatatcatatacttgtgagtggttcag

SEQ ID NO: 332 - DNA, 588 bp, Brassica napus
  misc_feature (16)..(18): n is a, c, g, or t
gcgtccgtgaacaatnnnatgtcgagaggagaaacaaagagaagtcagtttcttattcttcttcttcgtcggaagacagtgacggccatatctcgacttccgaggacgctaaggtgagtccaaggattgatttgaccagtgactgcttaagaggtagctacgctaagatgaatagctccacgaaacttgctcctcctaaacagagacgtcaaggtgctggtggtggtaagatgaagaaacagagacgtcttgatgtcaaggagaagtcagtttcttcttcttcttcgtcggaagacagtggcggccatatctcgaaacttgctcctcctggtaaagtaggacctacttttaaagactttgccgggattaaaaaagtgttggatgagttggatcagtatattctgtcccctattctcaatcctccattgtttgggaagattggagcgaagccaccaagcgggattctattccatggaccacctggctgtggaaagactcgcttgncnnntgccgtcgccaacgaagctggtgttcccttttatcnnntttcagccacagaaattgtttctggtgtttctggtgcgtctg

SEQ ID NO: 333 - DNA, 648 bp, Brassica napus
gattccctctgcacaaagtgaaatctagggtttcagataaagattccttaagagatctccgatcaagatgagttacggacaagataggctcaggggcactgtcaagtggtttgacacccagaagggtttcggtttcatcactcccgacaatggtggcgacgatctattcgttcaccagtcctccatcagatctgagggattccgaagcctcgctgcggaggaacctgtcgagttccaggtcgagaacgacaacaacggccgtcccaaggcgatagaggtgtctggacccgacggcgctcctgttcaaggtagcagcggcggtggcccgtctggcggacgcggtggattcggcggtggtggaagaggtggaggtggaggtcgtgggtttggcggcggaagaggtggtggtggtcgaggaggaagcgactgttacaagtgtggtgagccaggtcacattgcgagagactgttctgaaggtggtggaggatacggaggagggagaggtggatacggcggtggtggaggtggcggaggaggaagctgctatagctgtggcgagtctgggcatttcgccagggattgcactagcggtggacgttaatcagatggagaagtattctcgcagcttattatctatttattgttcgc

SEQ ID NO: 334 - DNA, 818 bp, Brassica napus
tgactgggaaaaccctggcgatttggtttaccaaagcttgaaaaaattgaggcctatacatgtccaacggttgaagagattctcttctctctatgaaaaaacaagtttagggtccagctgttttaagttcttgaaacatgtaatgaaaaaaataatgtgttcgaaacagacaaatcttgggaactgaagattgttcacatttcaagcttgtcttggttttcgggttctggaagtttcattgatggcaaaaaccacaaagccatttatgtgaaatgtggtggtcctgtttagattgttatcattagatttagatctaatatgtataagctcagtccaagtgttccttcttttccttacatgaccagatatataacttttgtggtccttaagctccacttttatgtggtccttctcaaaatcttttagaaagaaaatctttcatacaacttttgctatgtttcaacgttttttgtcttttgataagtctggaatctcaagtactggggtctccttgagtttcttatgtctttattccaacaaaagaaaatatcaaatctttgtttatagattctgtttctaccgtaccattactgattaatgaaggttatgatgttcttaatctgtagtacttggatgagaacaagtctttgattctgaagatagttgagtctcaaaactcaggaaagctcagcgagtgtgccgagtaagattaaacatatctccttgattttcacttgtgtttaaaatatgatcataaggttaaatattttgttacaggaatcaggcaaggcttcaacgcaacctcatgtacttggctg

SEQ ID NO: 335 - DNA, 642 bp, Brassica napus
aggcacgagtctccttcttctccgccgccatctaacatcgaaatgacaacagttccaggacagctggtctgggagatcgtaaagagaaacaactgtttcttggtcaaacagttcggcagaggcaatgctaaggttcagttcagcaaggagaccaacaatctctgcaacctcaactcctacaagcactctggtttggccaacaagaagacagtgaccattcaggtagctgacaaggagcaatgcgtggtactcggaacaaccaagaccaagaagcaaaacaagcctaagctctctgttaacaagtctgtcctcaagaaggagttccctaggatggccaaagctgttgccaaccaggttgtggacaactactacaggcctgacttgaagaaggcagcacttgctaggctaagcgtgatcagcaagggccttggagtcgccaagtctggtcccaagaggaggaacaggcaggcttgaattgaagaatccgccattggatgaacatttctacttctgaaaagcttgttttgaaagtttcagacacttctttgtaggttaaagttttttttatggacggatgagtgttttgcttgaaacacctacattgttatccttttctcacaaaatgatattaaaaactatttt

SEQ ID NO: 336 - DNA, 123 bp, Brassica napus; identical to SEQ ID NO: 236

SEQ ID NO: 337 - DNA, 411 bp, Brassica napus
tggaaagaactctcttaaaagactgttgaagactaagaaatgtagaagcagtagtggtaactgcaagagtagtagtagcacaagcgttgtttgtgtgaatggagttccagggattgggaagacagagttagctcttgagttcgcttaccggtactctcagaggtataagatggttttgtgggtaggaggggaagctagatacttcagacagaacctcttgaacctgtcgtttagcttgggggttgatgtgagcgctgatgctgagaaggatagaggtaggctaaggagctttgacgagcaggagtttgaagctttcaagaggataaagagagagcttttcagagacatgccttatctgctcatcattgataatcttgagatagagaaagactggtgggaagggaaagatct

SEQ ID NO: 338 - DNA, 506 bp, Brassica napus
taaacaagaggaaaagaggacgaccacataagttcataagcgaagagcctaaacagaagactggtgtggctgctagtgatccaatgcttaataatgctgttgtggaaactcctaaagccaaagattcaacaatggttctgccctttgtaaagaagtcaccgtgttggaaggttcttgaatcaatggagatcttcaaagctatgccacagcgtccacatttcaatccactgctggagtgcgaggaagagtcacgtgaaggagacgccattggtgcaatggtgaagttcactggactgttagagaaagtcaataatattcaaggggacgattcagtgactgagataaacaggatcaaagagtgttttctcaagctagaggaacacgggttcgacgttaccgcaccttgttctcgggatagacaagctgctctccgtttaaagaaagccagacgtgggcgtttggaagagtttaaaagttgctggagagagagattccagagaatgata

SEQ ID NO: 339 - DNA, 770 bp, Brassica napus
  misc_feature (623)..(623): n is a, c, g, or t
ggatttgattcgattccaagtcagcaacgaatatctcatgctccggaaggacaaggattagtgttgatggcagctttgcaaatgtctcgtgaatggatcggtattcagcagttcccacctgctactcagtctaagttgatcgagatccttaacaagttcaaagaagaggatgtgagctcgctgacagtacttgtaatggggaaaggcggtgttggaaagtcatctactgttaattcagttataggcgagaaagctgctgcagtcagtactttccagtctgaaggactgagaccgtccttggtctctcgctcaaggtcgggttttacattaaacattatcgacactcctggtcttattgagggaggatacgtgaatgatcaagccgtcaaccttataaaaaggtttctcctgaacatgacgatagatgtgctgctatacgtagaccgtttggatgtgtatcgggtggatgacttggataagcaggtagtcacggctataaccgatgcatttggtaaagagatatggaagaagtctgctcttgtcctcagtcatgctcagttctctccaccggacgggttaaactatgatctctttgtctccagaagatccgatgctcttctganactgatccgtgctagcgctcagctgaagaacaggatattcaggattcacctattcctgtcattcttgttgagaacagcggacgatgtcataagaatgagagcgatgaaaagattcttccagacggaagtagttgggatccgaatctg

SEQ ID NO: 340 - DNA, 688 bp, Brassica napus
ggttttcagaggcagcagagcttcagagtagcagagaactctctagagcggagaccaaaagaagacagagtgtggtgggatgaagaggagataagcaggagaagagattatgatgatatgaatgtaacagaagaacccaacgagatagctgagatcaggagatacgaagaggtactacggagtgagagggaggaagaggaacgacaaaaagcgaagaataaaaagaagaaaaagaagatgcaacccgaactagtagaagggtatctagaagattatcctcctcgaagaaacaacaacgatagaagaggccggaatgttagaagccggtatgtgtccgactttgaaatgaatggtgcggattatgcacctcaacctaaaagacgcaaaaaaggagaggttgggctagcaaacatcttggaaaccattgtggacacactaagactcaaagaagaagtgtcggggctcttcttgaaaccggtttcgaagaaggatgctccagactattttgatatagtgaaacgcccgatggatctgtcgacaatcagggataaggtacggaagatagagtacagaaaccgcgagcagtttagacacgatgtgtggcagatccagctcaatgctcatctttataacaacaatgggcgtaacccagggataccgcctttggcagatcagtggctggagattt

SEQ ID NO: 341 - DNA, 500 bp, Brassica napus
caaggatcagagtacagatacctcagaaagaaacgaagcttggcgggacgtcaagccacaacgacgactctccaattttagctcatccaggggaactagtaatctgcaagaagaagaggaaagacagagaaaagtctgcgccgagaaccagaaccgccggctcaagcagcccggtgtcaccaccagcaatggtgggtcgtggtcttagaagtcctgtgtctggttcaggtacaagggagacaaggctagcacagcagcaaagatggccaaaccaaggcactcatccaaacaacagcggtgcggctggtgattcagtgggatgggctaatccggtgaagaggttaaggacagattctggtaaaagaaggccgagccatttatagacagattaagcctcctttatctttagtgcttagaggtgtgatatgtaatgtggcaattgaaagcaatgtagactcgcttgtaaacaaataataattctctagatctcctttcctggt

SEQ ID NO: 342 - DNA, 733 bp, Brassica napus
aagatatcgaatgtactaagaaagaggttcaagacaccaaactgggttaagcacaaggagccacgagaggttcacatgtatgtcgatatgcttcttcagcagttggaagaagtcggtaaagaagtgaaacagattctacctcaaggaactttccgaaagcacaagagatcagacagcaacggaagtaacactacaacctcatcacggagcactacactacatagcgataagatggcacggtcgaactcacagagagctcgaagtcagcttttcgagacgcatcttgcaaagctgttcaagcaaaaagtagaaatattcaccaaggttgaatttacccaggaatcagttgtaacgacaacagtgaagctgtgtctgaagagtttgcaagaatatgtgcggctccaaacgtttaacaggagcgggttccagcagattcagctagacattcagttcttaaaagctcctttgaaagaaacagtcgaggacgaagctgctattgacttcttgctcgacgaggtgatcgttgcggcttcagagaggtgtcttgatgtgactccattggaaccaccaatcttggacaaactcatacaagccaagctcgctaaatctaaggaacacaacaacaacaacaacaagaccaccgtttcctcttgaaattaataagccgtaactcagaacccaagtgtgtatctcatgattcttgaattttttttggtacatctgctcagagcta

SEQ ID NO: 343 - DNA, 310 bp, Brassica napus
cagatgcgaaaagtgtggatctggacgatctgtggcgcagattaaagctggttttgtggctcgtgttggtcgcaggaagacctgagaaattggtgggtgaagtgaaaactaaaagagcttgtttgttatggagttgagaaacttgtcttgttttttcgttttgttttagaatatgaggcataatctactttgatctccatattatgacttgaaaaaatcaaatatgggttaggttttaaacattcaactttgttagatccatgtggctcaattaaaggtcgaatcagtaaatccgtggagtttttcagcc

SEQ ID NO: 344 - DNA, 713 bp, Brassica napus
ggatcgggttttgtgagtttcgccaaggggaagagaaagatggttgtgaggctccgattatcgagactaggatgcaagaatcgaccgttttttagggtaatggcgacggatagcagatctccgagagacgggaagcacatcgaggtcttgggttacttcaatcccttgccaggccaggacggtggtaagagaatgggtctcaagttcgatcgaatcaagtactggttatctgttggtgctcaggcatcagacccagttcagcgtcttcttttcagatccggtttacttcctcctcctccaatggtggctatgggacgtaaaggcggggaaagagacactcgccctgttgatccaatgacaggtcgctttgtggatgcagagaagaaaacaccggttatcatcgataaccagcctaaggaagaagacaatgcacaagacaagagtccatgattcattagtctaaaggaagaagacgagagtccgtgaactattggcgttagcctttgtctttgtagcttcgaagttcagtttggcacttttgtgtaatgcacatttctagttaatcttggtttattttattcctttgcataattccccatgtttggtgtgtaaaacggcaaggcgtgttgaggtcttgagggagattcaggttataagtttgacgtttatgatgtgcttattaacactacctatatgagctattctttaatccc

SEQ ID NO: 345 - DNA, 493 bp, Brassica napus
ggtcgacgacgtgaggggactaattacacttctgcgttttcatcttaggctttataccaataaaaggggttagtttttatcactgcgatggtagataaagctcaagtcatcagcctctgtcgcaccctgcttcgagcggggcgtcagtaccctgactacaacatcagggagtacgctaagcgaaggaccttggatgggttccgcatgaacaagaatctcaccgaccattcaaaggtggaggaggcttatgttgaaggtaagaagcagctggaggttgtggagagggtggtcaaggtttacctggcgtatcctcccaagaccaagaacatcatgggactcaagcttcagtagtggatatgtatgtttgggcttttttcaatacccttaaaaagctatttgttattgtcattggtcatgtttcttagttcttttccaactatgagacattgctttgtgtgattctcgtgtaatatttgcgtcttgatggcttcat

SEQ ID NO: 346 - DNA, 817 bp, Brassica napus
cgacgccatccagacaagccctcaacgcttagccgagaccgcactagccgttaccctaagccgagttcaatccacgaagctctttgtctcgcgtctgacgcgtttcaagaccctcaagaagcgcgaggtcgaagccatcaaggactgcgtcgaggagatcaacgataccatcgaacgtttgaccaagtcggtccaggagatgaagctatgtggtagtgccaagaatcaagaacagtttgcgttccacatgagtaatgctcagacttggactagtgctgctttgactgatgagaacacttgctccgatgggttctcgggtcggtttatggatggacggatcaagaactcggttcgggctagaatcgttaacatgggccaggaaaccagcaacgcactgtccttgattaatgcatatggtaaaaagtactaattaaaatttagtacttaagaaatgttttattttatgtatctcaaatatatatagaatagtaatgtctggctaagagtttgatgtgatatatatatgtcgattttgttagattattttcttatgttatgtgttcaagtgtattttgagctataaaagttgaagaagatgtgctttttattgccgtaaaaaaaaaaaaaaaaaactcgagttttttttttttttttttgagattaatcttttagcttttttcattttagtaacaggaaaacgcagcgtaagggacaattattacacttttagataacataatcagaccattcggtcaaatacgatgtctggtccatggtcaaacctggaatcttatctacccttttcggattctccttc

SEQ ID NO: 347 - DNA, 534 bp, Brassica napus
gacagctgtccactttgttcgactctcgccggagccataaaaagtcgaccattcctcgcgccgtcgctgtttcttccacttcaaccaatggtgagcatctccttcgtcgggtttccggtttatgtgaaaccggaaacctccacgagtccttccgggtcatcgaggagttcgacagagaggagaaatcatcatctgatgcgtttcttcttctacgagaagctctcgggcttctcctacaagcttccggaaggagaaaagacattcaactagggagaaagatccaccagcttgtttctgagtcggctcggttaagcaacgacgatgttctctgcacacgtgtcatcaccatgtactccatgtgcggctctcctgatgattctcggtctgtgtttgatgtcttgcggaagaagaatctcttccagtggaacgctgtcatcagctcttacttcagaaacgagcttttaccacaacttttctagagatgtttgtgaagatgatcacagagagtgggtcttttaccggataacttcactt

SEQ ID NO: 348 - DNA, 577 bp, Brassica napus
cggacgcgttggcccacaactcttcagaggctgaggtatttaaatcatttagatttatccaagaacatgctctccggtggttttccggtttggatcggtgacatgaccggtttagtacatttggatgtctccagcaatatgttaaccggaaagctaccttcttcaataagtaacttgaggtctctaaaggctctaatcttgtctgataacaaactctccggcgagattccggagtctttggagtcttgcaaagagcttatggttgttcagctctttgcaatggcttcaccggtagcattccggatggtttatttgatcttggtttgcaagaaatggatttttcaggtaacggtttaaccggttcgatcccgagagggtcaagcaggctatttgagtcacttgtgaggcttgatctttcgcgtaatagtctcactggaaacatacctggtgaagtagggctattcagcaacttgagatacctcaacttgtcatggaaccatttcaacacaagagttcctccagaaataaagtttctacagaatctaacaattttggaatctaggaacagcgcgttgat

SEQ ID NO: 349 - DNA, 813 bp, Brassica napus
  misc_feature (716)..(716): n is a, c, g, or t
cggcacgagggcaattcacaacttaatttatctgctttagttgcagcaaagagcaagcaaactatcatgcaacaagatggttcttgttactaacaaagtagaaaaatagttgtagaggtgattattttagataaatccgttgtttcaatatttctataactaacacactcaagtttatctgctctcacattacattgagatatctgatattgcaggcctagtgaaatatggtggctttgctttaaacaactttcttgaagtgtcctcaaactctttaccatctgggagctccattggttcgtgtagcaatatagtttctcaaagaaggagactgagtaagaccatcaggtgcaacagtggtgttctgagttccattcttctgcttcatccttctccgtgctatgtatcctctgacccaaggagttacatcctcgttggtcttaatcatgatgaagaaaatgatgcggtagatgataatcatgctgagaatcacactcaggttaatccattttgatcggtgcaagttgatctggaagacgttctcaagaacgtattcacctggaatcttgaaagcactcccctggctgtcaaacatcaagcctctcagatcattctggtattgaccttgtagcgcccagaagtggaagctaatgtatgacatagggtaacgccagaaaggtttagggatgtcatttggaagcctgaagaatccaganaccagcatgaagatcccctggattccagctccaatgatgatacncatgagggagttgggaaaatgctagctatagccatcatcaagctctccacaac

SEQ ID NO: 350 - DNA, 638 bp, Brassica napus
caacggtaaaccgatttgcgagtctctcaatgttgtccagtatgtggacgaggcttggtctgacaagaacccattcttcccctctgatccttacgggaaagctcaggctaggttttgggctgatttcgtggacaagaagtttagtgatgcccagttcaaggtatggggaaagaaaggtgaggaacaagcagcaggggtgaaggagtttattgaggcagtgaagattcttgaagctgagcttggagacaaaccttactttggtggagatagctttggatatgtagacattgcacttattacattctacagttggttcggagcatacgagaagtttggtaacttcagcatcgaggcagagagtccaaaactgattgcttgggctaagaggtgtatggagaaagagagtgtgtctaagtctctccctgaccaagagaaaattgttgcgtacgccgctgagtttaggaagaataatctctgatctgaataaaaccttgtttgtgccttctgtttctgatgatgctgtgttctgtttttaactttggtttttgtgtgtttcctaataatgcgtgagggcaagcgtcagtctttgtgtggtggtttgtatggaccctaagtaatgcattttaaaataaaatcta

SEQ ID NO: 351 - DNA, 637 bp, Brassica napus
gaggagaattgatcgatcatcgattcgaatccctaattcttatcttcgattcattctttccatggcgcttcattgcaaagtttcgaacacagcagcgagcacttctctgcttggtggttcgacgaggctgcttcacttgcctaagtcttatcccattcactgcaacatggtttcaccaagtagtacattggggttaggtcctctcaagctgcacaacaaaggttctcgtctaaggccatgtcgagttaaacgggaagagaatagccaaaccacaaatgtggaaagcgtatcagtggatgaggacacgttgaagcaagatctcgagactgcgatcgaggaagaaaactatgtggaggcagctagaatccgggatagactaaaagagcttcaagaagacaacaaagcctctgttttgtctgccaacactcggttctaccaagctttcagaaatggagatttggctgcgatggaatcgctatggtccaaaaccggaaagccctgctgtgtccatccaggagctaaaggcataaccgggtatgatgacgtgatggagagctgggaagttgtgtggatgaactatgagtttccgttacttattgagctgaaagatgttgaggttcatgtccgaggggatgta

SEQ ID NO: 352 - DNA, 846 bp, Brassica napus
gaactagtctcgagttttttttttttttttttaagaccaaaaaaataatgagttaaatgaaagagtccaaacaacttttataggtagattttttaagactccttcccttttaatagtattgattttacagaaactcaaaagataaaaaaaaaaaaaaacacaagggaaggggattacaaacacatacaacacataaactaaagagagattattcaagaaagcttttgtttcttataataactaaagatcaaattaattaactaattaaacattcatcctcattggggttggttttgttttattgagctttctgaatatatatatatcatctcactttgacgaggactggttcaagggactccttccacaactaacaggctttggactattaccaacgactcgaggactcatctgaccaaacctgagttcacccactcgtggactaaacacttgagacaaactcctattattgagcggagttgaactcgaactcgtgaccttaggactactcgtgtgtctaaactctcctctgcttcgtgtaataacctcatcaagaatctctgagtctcctggatacgaaattgtcgatccaatctccgtttccgctgcgagattgaagggtaaagctggagcttcttcatactctgctgcgtccaagtcctgaagatgagctcctttgaagaatctagggaggatgtattgtctaacggggattaagaacatgatcattaaagggaacatgactcctgctattgggatccatgtgaggccaaagcagattaacagataaaccgtttggaaaatagtgaacattgcaatcgtcttgaatggaaccgtctcaacgaa

SEQ ID NO: 353 - DNA, 461 bp, Brassica napus
gagatacctaacgtaggattcaatcaatggcatcgatgaccatgacagcaacattcctcccggccgtcgcgaagcttccgtcagccaccagcggacgaaggatgtacgtagtcagagcctcaacgagcgagaacacaaccagcttagaagtcaagaccaaggaggaacagaccagcaccacaatgaggagggatctcatgttcaatgctgcagctgcggccgtttgttccttggctaaagcagccatggcggacgaggaggagcccaagcgtgggacggaggcggctaagaagaaatacgctcaggtttgcgtcacaatgcctaccgccaagatctgccgctactgattcaaccatccacctttcatgtccctctctctatctctctttctattgttcggtaaaatttctcatatttggtatgtaatcatatgtttgatcaaaatattataatcttcaact

(19) United States
(12) Patent Application Publication
(10) Pub. No.: US 2013/0191936 A1
(43) Pub. Date: Jul. 25, 2013

(54) GENE EXPRESSION SIGNATURE FOR THE SELECTION OF HIGH ENERGY USE EFFICIENT PLANTS

(71) Applicants: Marc De Block, Merelbeke (BE); Matthew Hannah, Gent (BE); Katrien Van Der Kelen, Ledeberg (BE); Frank Van Breusegem, Ledeberg (BE)

(72) Inventors: Marc De Block, Merelbeke (BE); Matthew Hannah, Gent (BE); Katrien Van Der Kelen, Ledeberg (BE); Frank Van Breusegem, Ledeberg (BE)

(73) Assignees: Bayer CorpScience NV, Diegem (BE); VIB vzw, Zwijnaarde (BE); Sint-Pietersnieuwstraat 25, Gent (BE)

(21) Appl. No.: 13/876,365

(22) PCT Filed: Sep. 26, 2011
(86) PCT No.: PCT/EP2011/004858

§ 371 Date: Mar. 27, 2013

Related U.S. Application Data
(60) Provisional application No. 61/388,366, filed on Sep. 30, 2010.
(60) Provisional application No. 61/419,022, filed on Dec. 2, 2010.

(30) Foreign Application Priority Data
Oct. 1, 2010 (EP) 10075588.3
Dec. 6, 2010 (EP) 10075750.9

(51) Int. Cl. C12Q 1/68

(52) U.S. Cl. CPC C12Q 1/6895
USPC 800/260; 506/9; 506/16; 800/298

(57) ABSTRACT

Means and methods are provided to produce abiotic stress tolerant with improved yield based on the specific identification of a gene expression signature in said plants out of a population of said plants.

### FIELD OF THE INVENTION

The present invention belongs to the field of agriculture more particularly to the field of molecular breeding. The invention provides gene expression signatures which are associated with the presence of high energy use efficient plants. These gene expression signatures are breeder tools which can be used for the selection and production of plants which possess a high energy use efficiency. The high energy use efficiency is reflected in a higher tolerance to abiotic stress and also in an increased vigor.

### Introduction

Abiotic stress is defined as the negative impact of non-living factors on the living organisms in a specific environment. The non-living variable must influence the environment beyond its normal range of variation to adversely affect the population performance or individual physiology of the organism in a significant way. Abiotic stress is essentially unavoidable. Abiotic stress affects animals, but plants are especially dependent on environmental factors, so it is particularly constraining. Abiotic stress is the most harmful factor concerning the growth and productivity of crops worldwide. Drought, temperature extremes, and saline soils are the most common abiotic stresses that plants encounter. Globally, approximately 22% of agricultural land is saline and areas under drought are already expanding and this is expected to increase further. Other crops are exposed to multiple stresses, and the manner in which a plant senses and responds to different environmental factors appears to be overlapping. The most obvious detriment concerning abiotic stress involves farming. It has been calculated that abiotic stress causes the most crop loss of any other factor and that most major crops are reduced in their yield by more than 50% from their potential yield. In addition, it has been speculated that this yield reduction will only worsen with the dramatic climate changes expected in the future. Because abiotic stress is widely considered a detrimental effect, the research on this branch of the issue is extensive. When a plant is subjected to abiotic stress, a number of genes is differently expressed, resulting in a changed level of several metabolites and proteins, some of which may be responsible for conferring a certain degree of protection to these stresses. Obviously, a key to progress towards breeding better crops under stress has been to understand the changes in cellular, biochemical and molecular machinery that occur in response to stress. The development of genetically engineered plants by the overexpression or downregulation of selected genes seems to be a viable option to hasten the breeding of “improved” plants but has thus far not generated a significant impact on the generation of crops with an enhanced tolerance to abiotic stress. It is a constant challenge for breeders to improve and to shorten the timelines of the breeding processes. One particular aspect is the ability to select suitable starting material for breeding comprising optimal agronomical traits such as abiotic stress tolerance. The present invention provides an expression signature profile which can be used as a breeder tool for the selection and production of abiotic stress tolerant plants.

### SUMMARY OF THE INVENTION

The invention relates to methods of finding a gene expression profile (or a gene expression signature which is equivalent wording) characteristic for a plant with a high energy use efficiency. In one embodiment the invention enables the artisan to correlate the gene expression profile of a plant with a high energy use efficiency.

The present invention provides a method for the production of a plant with a high energy use efficiency comprising i) providing a population of plants of the same plant species, ii) obtaining a nucleic acid sample from said plants, iii) determining a gene expression profile of said plants by quantifying the mRNA expression level (mRNA abundance or presence) of at least two genes from Table 1 or genes comprising at least 70% nucleic acid identity with the genes in Table 1 and/or at least 2 genes from Table 2 or genes comprising at least 70% nucleic acid identity with the genes in Table 2 and/or of at least two genes from Tables 25-28 or genes comprising at least 70% nucleic acid identity with the genes in Table 25-28, iv) identifying at least one plant having an at least increased 1.5 fold presence of at least two genes from Table 2 or genes comprising at least 70% nucleic acid identity with the genes in Table 2 with respect to the average expression level (mRNA abundance or presence) of those genes in the plants of said population and/or having an at least decreased 0.66 fold presence of at least two genes from Table 1 or genes comprising at least 70% nucleic acid identity with the genes in Table 1 with respect to the average expression level of those genes in the plants of said population and/or having an at least increased 2.0 fold presence of at least two genes from Tables 25, 26 27 and 28 or genes comprising at least 70% nucleic acid identity with the genes in Tables 25, 26 27 and 28 with respect to the average expression level of those genes of the plants in said population.

In a specific embodiment the population of plants consists of genetically identical plants.

In another specific embodiment the population of plants consists of doubled haploid plants.

In another specific embodiment the population of plants consists of plants which are produced by vegetative reproduction.

In yet another specific embodiment the population of plants consists of inbred plants.

In another embodiment the produced plant from the methods is further crossed with another plant.

In another specific embodiment the produced plant and which is further crossed with another plant are both inbred plants.

In another specific embodiment the produced high energy use efficiency plant is a Brassica oilseed rape, tomato, rice, wheat, cotton, corn or soybean plant.

In a specific embodiment the quantification of the mRNA expression level (i.e. determining the mRNA presence) in the methods is determined by microarray analysis.

In a specific embodiment the quantification of the mRNA expression level in the methods is determined by RT-PCR.

In another specific embodiment the invention provides for a method for producing a population of plants or seeds with a high energy use efficiency comprising selecting a population of plants according to any one of the previous methods.

In another embodiment the invention provides for a method for increasing harvest yield comprising the steps of producing a population of plants or seeds according to the previous method, growing said plants or seeds in a field and producing a harvest from said plants or seeds.

A method for producing a hybrid plant or hybrid seed with high energy use efficiency comprising selecting a population of plants with high energy use efficiency for at least one parent inbred plant, crossing plants of said population with another inbred plant, isolating hybrid seed from said cross, and optionally, grow hybrid plants from said seed.

In another embodiment the invention provides a kit comprising the necessary tools for carrying out the method of the invention.

In another embodiment the invention provides a method for obtaining a biological or chemical compound which is capable of generating a plant with high energy use efficiency comprising i) providing a population of plants of the same plant species, ii) treating a subset of the plants of said population with one or more biological or chemical compounds, iii) obtaining a nucleic acid sample from said treated and untreated plants, iv) determining a gene expression profile of said treated and untreated plants by quantifying the mRNA expression level (mRNA presence) of at least two genes from Table 1 or genes comprising at least 70% nucleic acid identity with the genes in Table 1 and/or at least 2 genes from Table 2 or genes comprising at least 70% nucleic acid identity with the genes in Table 2, and/or of at least two genes from Table 25-28 or genes comprising at least 70% nucleic acid identity with the genes in Table 25-28 iv) identifying a compound which results in an at least increased 1.5 fold presence of the mRNA of at least two genes from Table 2 or genes comprising at least 70% nucleic acid identity with the genes in Table 2 in a plant from said population with respect to the expression level of said genes untreated plants of said population and/or which results in an at least decreased 0.66 fold presence of the mRNA of at least two genes from Table 1 or genes comprising at least 70% nucleic acid identity with the genes in Table 1 in said plant from said population with respect to the expression level (mRNA presence) of said genes in untreated plants in said population and/or which results in an at least 2.0 fold presence of the mRNA of said at least two genes from Table 25-28 or genes comprising at least 70% nucleic acid identity with the genes in Table 25-28 in said plant from said population with respect to the average expression level (mRNA presence) of said genes untreated plants in said.

In another embodiment the invention provides a gene expression profile indicative for high energy use efficiency comprising the expression level of at least two genes from Table 1 or genes comprising at least 70% nucleic acid identity with the genes in Table 1 and/or at least 2 genes from Table 2 or genes comprising at least 70% nucleic acid identity with the genes in Table 2 and/or at least 2 genes from Table 25-28 or genes comprising at least 70% nucleic acid identity with the genes in Table 25-28.

In another embodiment the gene expression profile is used in any of the previous methods.

### DETAILED DESCRIPTION OF THE INVENTION

To facilitate the understanding of this invention a number of terms are defined below. Terms defined herein (unless otherwise specified) have meanings as commonly understood by a person of ordinary skill in the areas relevant to the present invention. As used in this specification and its appended claims, terms such as “a”, “an” and “the” are not intended to refer to only a singular entity, but include the general class of which a specific example may be used for illustration, unless the context dictates otherwise. The terminology herein is used to describe specific embodiments of the invention, but their usage does not delimit the invention, except as outlined in the claims.

In a first embodiment the invention provides for a technical method for the production of a plant with a high energy use efficiency comprising i) providing a population of plants of the same plant species, ii) obtaining a nucleic acid sample from said plants, iii) determining a gene expression profile by quantifying the mRNA expression level (mRNA presence) of at least two genes from Table 1 or genes comprising at least 70% nucleic acid identity with the genes in Table 1 and/or at least 2 genes from Table 2 or genes comprising at least 70% nucleic acid identity with the genes in Table 2 and/or of at least two genes from Tables 25, 26 27 and 28 (SEQ ID NO 147-353) or genes comprising at least 70% nucleic acid identity with the genes in Table 25, 26, 27 and 28, iv) identifying at least one plant having an at least increased 1.5 fold presence of the mRNA of at least two genes from Table 2 or genes comprising at least 70% nucleic acid identity with the genes in Table 2 with respect to the average expression level (mRNA presence) of said genes in the plants of said population and/or having an at least decreased 0.66 fold presence of the mRNA of at least two genes from Table 1 or genes comprising at least 70% nucleic acid identity with the genes in Table 1 with respect to the average expression level (mRNA presence) of said genes in the plants of said population and/or having at least increased 2.0 fold presence of at the mRNA of least two genes from Tables 25, 26 27 and 28 or genes comprising at least 70% nucleic acid identity with the genes in Tables 25, 26 27 and 28 with respect to the average expression level (mRNA presence) of said genes in the plants of said population.

The terms “increase,” “elevate,” “raise,” and grammatical equivalents when used in reference to the level of mRNA expression (presence) of a gene in a first nucleic sample relative to a second sample, mean that the quantity of the mRNA expression in the first sample is higher than in the second sample by an amount that is statistically significant using a statistical method of analysis. Thus, an “at least increased 1.5 or 2.0 fold presence” as used herein, corresponds to a fold change in expression level with respect to a control value that is equal to or higher than 1.5 or 2.0 respectively.

The terms “reduce,” “inhibit,” “diminish,” “suppress,” “decrease,” and grammatical equivalents when used in reference to the level of mRNA expression (presence) of a gene in a first nucleic sample relative to a second sample, mean that the quantity of the mRNA expression in the first sample is lower than in the second sample by an amount that is statistically significant using a statistical method of analysis. Thus, an “at least decreased 0.66 fold presence” as used herein, corresponds to a fold change in expression level with respect to a control value that is equal to or lower than 0.6667. This can also be said to be an at least a 1.5 fold reduction (i.e. a fold reduction that is equal to or higher than 1.5).

A “gene expression profile” includes but is not limited to gene expression profiles as generally understood in the art. A gene expression profile of high energy use efficient plants selected from a population of plants of the same species contains a number of genes differentially expressed in comparison to the average of energy use efficiency of the plants present in said population (see Table 1 for the genes which are downregulated in the high energy use efficient plants compared to the average energy use efficiency of the plants present in the population of plants of the same plant species and Table 2 for the genes which are upregulated in the high energy use efficient plants compared to the average energy use efficiency of the plants present in the population of plants of the same plant species). A gene that appears in a gene expression profile, whether by upregulation or downregulation is said to be a member of the gene expression profile. For example, at least 2, at least 3, at least 4, at least 5, at least 6, at least 7, at least 8, at least 9, at least 10 member genes can be selected from Table I for an optimum signature for a high energy use efficient plant and/or at least 2, at least 3, at least 4, at least 5, at least 6, at least 7, at least 8, at least 9, at least 10 member genes can be selected from Table 2 and/or at least 2, at least 3, at least 4, at least 5, at least 6, at least 7, at least 8, at least 9, at least 10 member genes can be selected from Tables 25-28 for an optimum signature for a high energy use efficient plant. A further refinement of the gene expression profile by the identification of coexpression networks is presented in the example section.

Thus in another embodiment the quantification of the mRNA expression profile can be carried out with at least 2 genes or genes comprising at least 70% nucleic acid identity with the genes in Table 3 or Table 4 or Table 5 or Table 6 or Table 7 and/or with at least 2 genes or genes comprising at least 70% nucleic acid identity with the genes in Table 8 or Table 9 or Table 10 or Table 11 or Table 12 or Table 13 or Table 14 or Table 15 or Table 16 or Table 17 or Table 18 or Table 19 or Table 20 or Table 21 and/or with at least two genes or genes comprising at least 70% nucleic acid identity with the genes in Table 25, 26, 27 and 28 Quantification of the mRNA expression profile can also be carried out with at least 3, at least 4, at least 5, at least 6, at least 7, at least 8, at least 9, at least 10 member genes or member genes with at least 70% nucleotide sequence identity with the above genes.

In yet another embodiment, quantification of the mRNA expression profile can be carried out with at least two genes or genes comprising at least 70% nucleic acid identity with the genes that have been identified in the coexpression networks of both the HV110 and the HV112 hybrids with respect to control line 115 (genes that were significantly upregulated by at least 2.0 fold), i.e. the genes comprising the nucleotide sequence of SEQ ID NO's: 148, 149, 150, 151, 153, 155, 157, 159, 160, 161, 162, 163, 164, 165, 166, 167, 168, 169, 170, 171, 174, 175, 176, 178, 180, 181, 182, 183, 184, 185, 188, 190, 191, 192, 193, 194, 195, 196, 197, 198, 199, 200, 201, 202, 204, 205, 207, 209, 210, 211, 212, 214, 216, 218, 221, 221, 222, 224, 226, 227, 228, 229, 233, 234, 235, 236, 237, 238, 240, 241, 242, 243, 246, 247, 249, 250, 251, 253, 254, 255, 256, 261, 262, 263, 266, 267, 269, 270, 323, 272, 273, 274, 275, 276, 277, 278, 279, 280, 283, 284, 285, 286, 287, 288, 289, 291, 292, 294, 295, 297, 298, 299, 300, 301, 302, 303, 305, 306, 307, 308, 309, 311, 312, 313, 315, 318, 319, 321, 322, 324. Quantification of the mRNA expression profile can also be carried out with at least 3, at least 4, at least 5, at least 6, at least 7, at least 8, at least 9, at least 10 member genes or member genes with at least 70% nucleotide sequence identity with the above genes.

While not intending to limit the invention to a particular explanation of the occurrence of a specific gene expression signature associated with high energy use efficient plants, it appears that several genes with mitochondrial function such as genes of the respiratory chain are transcriptionally upregulated in high energy efficient plant in addition to the upregulation of the transcription of a number of ribosomal genes and upregulation of transcription of genes involved in chloroplast function.

Thus, in even yet another embodiment, quantification of the mRNA expression profile can be carried out with at least two genes or genes comprising at least 70% nucleic acid identity with the genes that have been found to be significantly upregulated in HV110 or HV112 vs. control line 115 using the agilent (at least 1.5 fold) or combimatrix (at least 2.0 fold) array and that are involved in mitochondria, translation or chloroplasts, i.e. the genes comprising SEQ ID NO's 66, 69, 78, 80, 81, 82, 84, 87, 89, 90, 91, 92, 93, 96, 101, 104, 105, 107, 113, 116, 117, 119, 121, 122, 123, 127, 128, 129, 131, 132, 133, 134, 148, 157, 161, 162, 176, 177, 182, 192, 201, 207, 209, 211, 212, 224, 226, 228, 231, 235, 236, 238, 249, 250, 254, 258, 260, 266, 267, 269, 274, 276, 279, 280, 284, 286, 291, 292, 296, 297, 299, 300, 301, 302, 303, 306, 308, 309, 311, 313, 316, 321, 323, 324, 329, 330, 331, 335, 339, 343, 344, 353. Quantification of the mRNA expression profile can also be carried out with at least 3, at least 4, at least 5, at least 6, at least 7, at least 8, at least 9, at least 10 member genes or member genes with at least 70% nucleotide sequence identity with the above genes.

In an even further embodiment, quantification of the mRNA expression profile can be carried out with at least two genes or genes comprising at least 70% nucleic acid identity with the genes that have been found to be significantly upregulated (at least 2.0 fold) in both HV110 and HV112 vs. control line 115 and that are involved in mitochondria, translation or chloroplasts, i.e. the genes comprising SEQ ID NO's 148, 157, 161, 162, 176, 182, 192, 201, 207, 209, 211, 212, 224, 226, 228, 235, 236, 238, 249, 250, 254, 266, 267, 269, 274, 276, 279, 280, 284, 286, 292, 297, 299, 300, 301, 302, 303, 306, 308, 309, 311, 313, 321, 324. Quantification of the mRNA expression profile can also be carried out with at least 3, at least 4, at least 5, at least 6, at least 7, at least 8, at least 9, at least 10 member genes or member genes with at least 70% nucleotide sequence identity with the above genes.

In a further embodiment, quantification of the mRNA expression profile can be carried out with the above described genes that have been found to be significantly upregulated with respect to the control line by at least 2.0 fold, by at least 3.0 fold, by at least 4.0 fold, by at least 5.0 fold, by at least 10 fold, or by at least 25 fold (i.e. wherein the fold change in expression is equal to or higher than 2.0, 3.0, 4.0, 5.0, 10 or 25 respectively) and/or that have been found to be significantly downregulated by at least 1.5 fold, by at least 2.0 fold, or by at least 2.5 fold (i.e. wherein the fold change in expression is equal to or below 0.6667, 0.5 or 0.4 respectively).

The nucleic acid sample is obtained from the plant in a manner which allows further cultivation of said sampled individual plants, e.g. by isolating a tissue sample or explant from individual plants of said population. In one embodiment, the nucleic acid sample is obtained from a leaf. In a particular embodiment the nucleic acid sample is obtained from leaf 3 or leaf 4, at the 3- or 4 leaf stage.

“Expression level” as used herein, refers to the net mRNA presence or abundance, i.e. taking into account the rate of mRNA synthesis and the rate of mRNA degradation.

The average expression level (mRNA presence) of a gene in a population of plants can be determined by adding the expression levels of the individual plants and dividing that by the number of plants of the population, or by pooling the nucleic acid samples of all plants of the population and then determine the expression level of the gene in the pooled nucleic acid sample. A gene expression profile may be “determined,” without limitation, by means of DNA microarray analysis, PCR, quantitative RT-PCR, etc. These are referred to herein collectively as “nucleic-acid based: determinations or assays. Alternatively, methods as multiplexed immunofluorescence microscopy or flow cytometry may be used.

Gene expression profiles may be “compared” by any of a variety of statistical analytic procedures including, without limitation, the use of GeneSpring 7.2 software (Silicon Genetics, Redwood City, Calif.) according to the manufacturer's instructions.

The aforementioned methods for examining gene sets employ a number of well-known methods in molecular biology, to which references are made herein. A gene is a heritable chemical code resident in, for example, a cell, virus, or bacteriophage that an organism reads (decodes, decrypts, transcribes) as a template for ordering the structures of biomolecules that an organism synthesizes to impart regulated function to the organism. Chemically, a gene is a heteropolymer comprised of subunits (“nucleotides”) arranged in a specific sequence. In cells, such heteropolymers are deoxynucleic acids (“DNA”) or ribonucleic acids (“RNA”). DNA forms long strands. Characteristically, these strands occur in pairs. The first member of a pair is not identical in nucleotide sequence to the second strand, but complementary. The tendency of a first strand to bind in this way to a complementary second strand (the two strands are said to “anneal” or “hybridize”), together with the tendency of individual nucleotides to line up against a single strand in a complementarily ordered manner accounts for the replication of DNA. Experimentally, nucleotide sequences selected for their complementarity can be made to anneal to a strand of DNA containing one or more genes. A single such sequence can be employed to identify the presence of a particular gene by attaching itself to the gene. This so-called “probe” sequence is adapted to carry with it a “marker” that the investigator can readily detect as evidence that the probe struck a target.

Alternatively, such sequences can be delivered in pairs selected to hybridize with two specific sequences that bracket a gene sequence. A complementary strand of DNA then forms between the “primer pair.” In one well-known method, the “polymerase chain reaction” or “PCR,” the formation of complementary strands can be made to occur repeatedly in an exponential amplification. A specific nucleotide sequence so amplified is referred to herein as the “amplicon” of that sequence. “Quantitative PCR” or “qPCR” herein refers to a version of the method that allows the artisan not only to detect the presence of a specific nucleic acid sequence but also to quantify how many copies of the sequence are present in a sample, at least relative to a control. As used herein, “qRTPCR” may refer to “quantitative real-time PCR,” used interchangeably with “qPCR” as a technique for quantifying the amount of a specific DNA sequence in a sample. However, if the context so admits, the same abbreviation may refer to “quantitative reverse transcriptase PCR,” a method for determining the amount of messenger RNA present in a sample. Since the presence of a particular messenger RNA in a cell indicates that a specific gene is currently active (being expressed) in the cell, this quantitative technique finds use, for example, in gauging the level of expression of a gene. Collectively, the genes of an organism constitute its genome.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch (1970) J Mol. Biol. 48: 443-453) The computer-assisted sequence alignment above, can be conveniently performed using standard software program such as GAP which is part of the Wisconsin Package Version 10.1 (Genetics Computer Group, Madision, Wis., USA) using the default scoring matrix with a gap creation penalty of 50 and a gap extension penalty of 3. Sequences are indicated as “essentially similar” when such sequence have a sequence identity of at least about 75%, particularly at least about 80%, more particularly at least about 85%, quite particularly about 90%, especially about 95%, more especially about 100%, quite especially are identical. It is clear than when RNA sequences are the to be essentially similar or have a certain degree of sequence identity with DNA sequences, thymine (T) in the DNA sequence is considered equal to uracil (U) in the RNA sequence.

Thus, at least 70% nucleic acid identity, as used herein, refers to 70%-100%, 75%-100%, 80%-100%, 85%-100%, 90%-100%, 95%-100%, 96%-100%, 97-100%, 98%-100% or 99-100% nucleic acid sequence identity with respect to another nucleic acid sequence.

In another aspect, the invention is embodied in a kit useful for detecting the gene expression profile of the invention. To effectively detect a gene expression profile which is characteristic for a plant with a high energy use efficiency or a population of plants with a high energy efficiency the gene expression (mRNa presence) of at least two, at least three, at least four, at least five or more genes depicted in Table I and/or at least two, at least three, at least four, at least five or more genes depicted in Table II, and/or at least two, at least three, at least four, at least five or more genes depicted in Table 25-28 is measured. A kit to carry out a PCR analysis, preferably a multiplex PCR analysis such as a multiplex RT-PCR analysis comprises primers, buffers, polynucleotides and a thermostable DNA polymerase.

In another embodiment, the kit measures the expression level of at least 2 genes or genes comprising at least 70% nucleic acid identity with the genes in Table 3 or Table 4 or Table 5 or Table 6 or Table 7 and/or with at least 2 genes or genes comprising at least 70% nucleic acid identity with the genes in Table 8 or Table 9 or Table 10 or Table 11 or Table 12 or Table 13 or Table 14 or Table 15 or Table 16 or Table 17 or Table 18 or Table 19 or Table 20 or Table 21. The kit measures the expression level of at least 3, at least 4, at least 5, at least 6, at least 7, at least 8, at least 9, at least 10 member genes or member genes with at least 70% nucleotide sequence identity with the above genes. The kit can also measures the expression level of at least 3, at least 4, at least 5, at least 6, at least 7, at least 8, at least 9, at least 10 member genes or member genes with at least 70% nucleotide sequence identity with the above genes.

In yet another embodiment, the kit measures the expression level of at least two genes or genes comprising at least 70% nucleic acid identity with the genes that have been identified in the coexpression networks of both the HV110 and the HV112 hybrids with respect to control line 115 (genes that were significantly upregulated by at least 2.0 fold, as indicated above). The kit can also measures the expression level of at least 3, at least 4, at least 5, at least 6, at least 7, at least 8, at least 9, at least 10 member genes or member genes with at least 70% nucleotide sequence identity with the above genes.

In even yet another embodiment, the kit measures the expression level of at least two genes or genes comprising at least 70% nucleic acid identity with the genes that have been to be significantly upregulated in HV110 or HV112 vs control line 115 using the agilent (at least 1.5 fold) or combimatrix (at least 2.0 fold) array and that are involved in mitochondria, translation or chloroplasts (as indicated above). The kit can also measures the expression level of at least 3, at least 4, at least 5, at least 6, at least 7, at least 8, at least 9, at least 10 member genes or member genes with at least 70% nucleotide sequence identity with the above genes.

In an even further embodiment, the kit measures the expression level of at least two genes or genes comprising at least 70% nucleic acid identity with the genes that have been to be significantly upregulated (at least 2.0 fold) in both HV110 and HV112 vs control line 115 and that are involved in mitochondria, translation or chloroplasts (as indicated above). The kit can also measures the expression level of at least 3, at least 4, at least 5, at least 6, at least 7, at least 8, at least 9, at least 10 member genes or member genes with at least 70% nucleotide sequence identity with the above genes.

In another embodiment, the kit can measure the expression level the above described genes that have been found to be significantly upregulated with respect to the control line by at least 2.0 fold, by at least 3.0 fold, by at least 4.0 fold, by at least 5.0 fold, by at least 10 fold, or by at least 25 fold (i.e. wherein the fold change in expression is equal to or higher than 2.0, 3.0, 4.0, 5.0, 10 or 25 respectively) and/or that have been found to be significantly downregulated by at least 1.5 fold, by at least 2.0 fold, or by at least 2.5 fold (i.e. wherein the fold change in expression is equal to or below 0.6667, 0.5 or 0.4 respectively).

In a particular embodiment based on the identified gene expression profile it is possible to determine a corresponding protein expression profile. A protein expression profile can conveniently be detected by the use of specific antibodies directed against the differentially expressed protein products.

In a particular embodiment the starting population of plants is of the same plant species or of the same plant variety. In another particular embodiment the population of plants is genetically identical.

As used herein “a population of genetically identical plants” is a population of plants, wherein the individual plants are true breeding, i.e. show little or no variation at the genome nucleotide sequence level, at least for the genetic factors which are underlying the quantitative trait, particularly genetic factors underlying high energy use efficiency and low cellular respiration rate. Genetically uniform plants may be inbred plants but may also be a population of genetically identical plants such as doubled haploid plants. Doubled haploid plants are plants obtained by spontaneous or induced doubling of the haploid genome in haploid plant cell lines (which may be produced from gametes or precursor cells thereof such as microspores). Through the chromosome doubling, complete homozygous plants can be produced in one generation and all progeny plants of a selfed doubled haploid plant are substantially genetically identical (safe the rare mutations, deletions or genome rearrangements). Other genetically uniform plants are obtained by vegetal reproduction or multiplication such as e.g. in potato, sugarcane, trees including poplars or eucalyptus trees.

“Creating propagating material”, as used herein, relates to any means know in the art to produce further plants, plant parts or seeds and includes inter alia vegetative reproduction methods (e.g. air or ground layering, division, (bud) grafting, micropropagation, stolons or runners, storage organs such as bulbs, corms, tubers and rhizomes, striking or cutting, twin-scaling), sexual reproduction (crossing with another plant) and asexual reproduction (e.g. apomixis, somatic hybridization).

As used herein, “energy use efficiency (EUE)” is the quotient of the “energy content” and “cellular respiration”. High energy use efficiency can be achieved in plants when the energy content of the cells of the plant remains about equal to that of control plants, but when such energy content is achieved by a lower cellular respiration.

The energy use efficiency can be determined by determining the cellular respiration and determining the NAD(P)H content in the isolated sample and dividing the NAD(P)H content by the respiration to determine the energy use efficiency. The energy use efficiency can also be determined by measuring the ascorbate or ascorbic acid content of the plant or by measuring the respiratory chain complex I activity in said sample.

“Cellular respiration” refers to the use of oxygen as an electron acceptor and can conveniently be quantified by measuring the electron transport through the mitochondrial respiratory chain e.g. by measuring the capacity of the tissue sample to reduce 2,3,5 triphenyltetrazolium chloride (TTC). Although it is believed that for the purpose of the assays defined here, TTC is the most suited substrate, other indicator molecules, such as MTT (3-(4,5-dimethylthiazol-2-yl)-2,5 diphenyl-2H-tetrazolium), can be used to measure the electron flow in the mitochondrial electron transport chain (see Musser and Oseroff, 1994 Photochemistry and Photobiology 59, pp 621-626). TTC reduction occurs at the end of the mitochondrial respiratory chain at complex IV. Therefore, TTC reduction reflects the total electron flow through the mitochondrial respiratory chain, including the alternative oxidative respiratory pathway. The electrons enter the mitochondrial electron transport chain through complex I, complex II, and the internal and external alternative NAD(P)H dehydrogenases. A suitable TTC reduction assay has been described by De Block and De Brouwer, 2002 (Plant Physiol. Biochem. 40, 845-852).

The “energy content” of cells of a plant refers to the amount of molecules usually employed to store energy such as ATP, NADH and NADPH. The energy content of a sample can conveniently be determined by measuring the NAD(P)H content of the sample. A suitable assay has been described by Nakamura et al. 2003. (Quantification of intracellular NAD(P)H can monitor an imbalance of DNA single strand break repair in base excision repair deficient cells in real time. Nucl. Acids Res. 31, 17 e104).

Plants or subpopulations of plants should be selected wherein the energy use efficiency is at least as good as the energy use efficiency determined for the control plants, preferably is higher than the energy use efficiency of control plants. Although it is believed that there is no particular upper limit for energy use efficiency, it has been observed that subpopulations or plants can be obtained with an energy use efficiency which is about 5% to about 15%, particularly about 10% higher than the energy use efficiency of control plants. As used herein, control plants or control population are a population of plants which are genetically uniform but which have not been subjected to the reiterative selection for plants with a higher energy use efficiency.

Plants or subpopulations of plants can initially be selected for a cellular respiration which is lower than the cellular respiration determined for the control plants. Typically, plants with a high energy use efficiency have cellular respiration rate which is between 85 and 95% of the cellular respiration rate of control plants. It has been observed that it is usually feasible to subject a population with lower respiration rates to an additional cycle of selection yielding a population of plants with even lower respiration rates, wherein however the energy content level is also declined. Such selected population of plants have a yield potential which is not better than a population of unselected control plants and the yield may even be worse in particular circumstances. Selection of populations with too low cellular respiration, particularly when accompanied with a decline in energy content level is not beneficial. Respiration rates below 75% of the respiration rate of control plants, particularly combined with energy contents below 75% of the energy content of control plants should preferably be avoided.

It has been observed that selected populations with a high energy use efficiency are also characterized by an increased respiratory chain complex I activity compared to control plants and by an increased ascorbic acid content compared to control plants. These characteristics could serve as an alternative or supplementary marker to select plants or (sub)populations of plants with increased energy use efficiency. Ascorbate content can be quantified using the reflectometric ascorbic acid test from Merck (Darmstadt, Germany). Complex I activity can be quantified using the MitoProfile Dipstick Assay kit for complex I activity of MitoSciences (Eugene, Oreg., USA).

It has been observed that the selected subpopulation was more tolerant to adverse abiotic conditions than the unselected control plants. Accordingly, the invention also provides a method for producing a population of plants or seeds with increased tolerance to adverse abiotic conditions by selection plants or populations of plants according to the methods described herein. As used herein “adverse abiotic conditions” include drought, water deficiency, hypoxic or anoxic conditions, flooding, high or low suboptimal temperatures, high salinicity, low nutrient level, high ozone concentrations, high or low light concentrations and the like. It has also been observed that the selected plants have a higher yield (or have a yield improvement). Thus, the wording ‘a plant with a high energy use efficiency’ is equivalent to the wording ‘a plant tolerant to abiotic stress and having an improved yield’. It is understood that the tolerance to abiotic stress and improved yield is with respect to the average of the abiotic stress tolerance and yield of the plants of the population from which the plant was selected.

Interplanting, as used herein refers to the mixed planting of parent plants of which seeds and/or progeny plants are to be obtained.

In a particular embodiment the method for the production of a plant with a high energy use efficiency may be applied to both parent lines and if hybrid production involves male sterility necessitating the use of a maintainer line for maintaining the female parent.

The invention also provides selected plants or populations of plants with high energy use efficiency as can be obtained through the selection methods herein described. Such plants are characterized by a low cellular respiration (lower than the cellular respiration of control plants as herein defined) and at least one of the following characteristics: ascorbic acid higher than control plants; NAD(P)H content higher than control plants; respiratory chain complex I activity higher than control plants; and photorespiration lower than control plants.

The methods and means described herein are believed to be suitable for all plant cells and plants, gymnosperms and angiosperms, both dicotyledonous and monocotyledonous plant cells and plants including but not limited to Arabidopsis, alfalfa, barley, bean, corn or maize, cotton, flax, oat, pea, rape, rice, rye, safflower, sorghum, soybean, sunflower, tobacco and other Nicotiana species, including Nicotiana benthamiana, wheat, asparagus, beet, broccoli, cabbage, carrot, cauliflower, celery, cucumber, eggplant, lettuce, onion, oilseed rape, pepper, potato, pumpkin, radish, spinach, squash, tomato, zucchini, almond, apple, apricot, banana, blackberry, blueberry, cacao, cherry, coconut, cranberry, date, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut and watermelon Brassica vegetables, sugarcane, vegetables (including chicory, lettuce, tomato), Lemnaceae (including species from the genera Lemna, Wolffiella, Spirodela, Landoltia, Wolffia) and sugarbeet.

In yet another embodiment the invention provides for a method for obtaining a biological or chemical compound which is capable of generating a plant with high energy use efficiency comprising a) providing a population of plants of the same plant species, b) treating a subset of the plants of said population with a biological or chemical compound, c) obtaining a nucleic acid sample from said plants of said population, iv) determining a gene expression profile by quantifying the mRNA expression level (mRNA presence) of at least two genes from Table 1 or genes comprising at least 70% nucleic acid identity with the genes in Table 1 and/or at least 2 genes from Table 2 or genes comprising at least 70% nucleic acid identity with the genes in Table 2, and/or of at least two genes from Tables 25, 26 27 and 28 (SEQ ID NO 147-353) or genes comprising at least 70% nucleic acid identity with the genes in Table 25, 26, 27 and 28 and d) identifying a compound which when applied to a plant results in an at least increased 1.5 fold presence of at the mRNA of least two genes from Table 2 or genes comprising at least 70% nucleic acid identity with the genes in Table 2 in said plant from said population with respect to the expression level (mRNA presence) of said genes in untreated plants of said population and/or results in an at least decreased 0.66 fold presence of the mRNA of at least two genes from Table 1 or genes comprising at least 70% nucleic acid identity with the genes in Table 1 in said plant from said population with respect to the expression level (mRNA presence) of said genes in untreated plants of said population, and/or results in an at least increased 2.0 fold presence of the mRNA of at least two genes from Tables 25, 26 27 and 28 or genes comprising at least 70% nucleic acid identity with the genes in Tables 25, 26 27 and 28 in a plant from said population with respect to the expression level (mRNA presence) of said genes in untreated plants of said population.

In step (b) any biological or chemical compound may be contacted with the plants or plant parts. It is also envisaged that a plurality of different compounds can be contacted in parallel with plants or plant parts. Preferably each test compound is brought into physical contact with one or more individual plants. Contact can also be attained by various means, such as spraying, spotting, brushing, applying solutions or solids to the soil, to the gaseous phase around the plants or plant parts, dipping, etc. The test compounds may be solid, liquid, semi-solid or gaseous. The test compounds can be artificially synthesized compounds or natural compounds, such as proteins, protein fragments, volatile organic compounds, plant or animal or microorganism extracts, metabolites, sugars, fats or oils, microorganisms such as viruses, bacteria, fungi, etc. In a preferred embodiment the biological compound comprises or consists of one or more microorganisms, or one or more plant extracts or volatiles (e.g. plant headspace compositions). The microorganisms are preferably selected from the group consisting of: bacteria, fungi, mycorrhizae, nematodes and/or viruses. It is especially preferred and evident that the microorganisms are non-pathogenic to plants, or at least to the plant species used in the method. Especially preferred are bacteria which are non-pathogenic root colonizing bacteria and/or fungi, such as Mycorrhizae. Mixtures of two, tree or more compounds may also be applied to start with, and a mixture which shows an effect on priming can then be separated into components which are retested in the method. Using mixtures, also synergistically acting compounds can be identified, i.e. compounds which provide a stronger priming effect together than the sum of their individual priming effect. Preferably compositions are liquid or solid (e.g. powders) and can be applied to the soil, seeds or seedlings or to the aerial parts of the plant.

In another embodiment in the method for obtaining a biological or chemical compound, the quantification of the mRNA expression profile can be carried out with at least 2 genes or genes comprising at least 70% nucleic acid identity with the genes in Table 3 or Table 4 or Table 5 or Table 6 or Table 7 and/or with at least 2 genes or genes comprising at least 70% nucleic acid identity with the genes in Table 8 or Table 9 or Table 10 or Table 11 or Table 12 or Table 13 or Table 14 or Table 15 or Table 16 or Table 17 or Table 18 or Table 19 or Table 20 or Table 21.

In yet another embodiment, in the method for obtaining a biological or chemical compound, the quantification of the mRNA expression profile can be carried out with at least two genes or genes comprising at least 70% nucleic acid identity with the genes that have been identified in the coexpression networks of both the HV110 and the HV112 hybrids with respect to control line 115 (genes that were significantly upregulated by at least 2.0 fold, as indicated above).

In even yet another embodiment, in the method for obtaining a biological or chemical compound, the quantification of the mRNA expression profile can be carried out with at least two genes or genes comprising at least 70% nucleic acid identity with the genes that have been found to be significantly upregulated in HV110 or HV112 vs control line 115 using the agilent (at least 1.5 fold) or combimatrix (at least 2.0 fold) array and that are involved in mitochondria, translation or chloroplasts (as indicated above).

In an even further embodiment in the method for obtaining a biological or chemical compound, quantification of the mRNA expression profile can be carried out with at least two genes or genes comprising at least 70% nucleic acid identity with the genes that have been found to be significantly upregulated (at least 2.0 fold) in both HV110 and HV112 vs control line 115 and that are involved in mitochondria, translation or chloroplasts (as indicated above).

In another embodiment, in the method for obtaining a biological or chemical compound, quantification of the mRNA expression profile can be carried out with the above described genes that have been found to be significantly upregulated with respect to the control line by at least 2.0 fold, by at least 3.0 fold, by at least 4.0 fold, by at least 5.0 fold, by at least 10 fold, or by at least 25 fold (i.e. wherein the fold change in expression is equal to or higher than 2.0, 3.0, 4.0, 5.0, 10 or 25 respectively) and/or that have been found to be significantly downregulated by at least 1.5 fold, by at least 2.0 fold, or by at least 2.5 fold (i.e. wherein the fold change in expression is equal to or below 0.6667, 0.5 or 0.4 respectively).

In yet another embodiment the invention provides a gene expression profile indicative for high energy use efficiency in plants comprises the expression level of at least two genes from Table 1 or genes comprising at least 70% nucleic acid identity with the genes in Table 1 and/or at least 2 genes from Table 2 or genes comprising at least 70% nucleic acid identity with the genes in Table 2 and/or of at least two genes from Tables 25-28 (SEQ ID NO 147-353) or genes comprising at least 70% nucleic acid identity with the genes in Tables 25-28. In a particular embodiment the gene expression profile consists of at least 2, at least 3, at least 4, at least 5, at least 6, at least 7, at least 8, at least 9, at least 10 member genes or member genes with at least 70% nucleotide sequence identity selected from Table 1 and/or at least 2, at least 3, at least 4, at least 5, at least 6, at least 7, at least 8, at least 9, at least 10 member genes from Table 2 or member genes with at least 70% nucleotide sequence identity and/or 2 and/or of at least two genes from Tables 25-28 or genes comprising at least 70% nucleic acid identity with the genes in Tables 25-28. In another particular embodiment the gene expression profile indicative for high energy use efficiency comprises the expression level of at least 2 genes or genes comprising at least 70% nucleic acid identity with the genes in Table 3 or Table 4 or Table 5 or Table 6 or Table 7 and/or with at least 2 genes or genes comprising at least 70% nucleic acid identity with the genes in Table 8 or Table 9 or Table 10 or Table 11 or Table 12 or Table 13 or Table 14 or Table 15 or Table 16 or Table 17 or Table 18 or Table 19 or Table 20 or Table 21. In yet another embodiment, the gene expression profile consists of at least 2, at least 3, at least 4, at least 5, at least 6, at least 7, at least 8, at least 9, at least 10 member genes or member genes with at least 70% nucleotide sequence identity with the genes that have been identified in the coexpression networks of both the HV110 and the HV112 hybrids with respect to control line 115 (genes that were significantly upregulated by at least 2.0 fold, as indicated above). In even yet another embodiment, the gene expression profile consists of at least 2, at least 3, at least 4, at least 5, at least 6, at least 7, at least 8, at least 9, at least 10 member genes or member genes with at least 70% nucleotide sequence identity with the genes that have been found to be significantly upregulated in HV110 or HV112 vs control line 115 using the agilent (at least 1.5 fold) or combimatrix (at least 2.0 fold) array and that are involved in mitochondria, translation or chloroplasts (as indicated above). In an even further embodiment, the gene expression profile consists of at least 2, at least 3, at least 4, at least 5, at least 6, at least 7, at least 8, at least 9, at least 10 member genes or member genes with at least 70% nucleotide sequence identity with the genes that have been found to be significantly upregulated (at least 2.0 fold) in both HV110 and HV112 vs control line 115 and that are involved in mitochondria, translation or chloroplasts (as indicated above).

In another embodiment, the gene expression profile consists of the above described genes that have been found to be significantly upregulated with respect to the control line by at least 1.5 fold, by at least 2.0 fold, by at least 3.0 fold, by at least 4.0 fold, by at least 5.0 fold, by at least 10 fold, or by at least 25 fold (i.e. wherein the fold change in expression is equal to or higher than 2.0, 3.0, 4.0, 5.0, 10 or 25 respectively) and/or that have been found to be significantly downregulated by at least 1.5 fold, by at least 2.0 fold, or by at least 2.5 fold (i.e. wherein the fold change in expression is equal to or below 0.6667, 0.5 or 0.4 respectively).

In another embodiment the herein before defined gene expression profile is used for the production of a plant with a high energy use efficiency according to the methods described herein.

In yet another embodiment the gene expression profile is used in the method for obtaining a biological or chemical compound which is capable of generating a plant with a high energy use efficiency.

The following non-limiting Examples describe methods and means according to the invention. Unless stated otherwise in the Examples, all techniques are carried out according to protocols standard in the art.

### EXAMPLES

### 1. Selection and Characterization of Brassica napus Plants with High and Low Energy Use Efficiency

Selection of B. napus plants with high energy use efficiency (EUE) and low energy use efficiency (EUE) and yield was performed as described in Hauben et al. (2009) Proc Natl Acad Sci USA November 24; 106(47):20109-14 and in the examples 1 and 2 of the priority application EP09075284 (filed on 1 Jul., 2009), both of which references are incorporated herein by reference.

In short, starting with these selected individual plants with high or low EUE we performed multiple cycles of self-crossing and selection for EUE for the production of isogenic clones. Seeds of the low-EUE clone and the high-EUE clone were up-scaled. As described in Example 2 of the priority application EP09075284 (filed on 1 Jul., 2009) and schematically depicted in FIG. 9 in EP09075284, hereby incorporated by reference, B. napus hybrids were generated with elite parental lines of canola which were selected for high EUE. Two high EUE B. napus hybrid were generated and designated as HV110 and HV112.

We showed that stress testing in growth chambers and the greenhouse revealed that high-EUE B. napus plants have an enhanced tolerance to ozone (4 days, 400 ppb) and heat (10 days, 45° C.) compared with a control B. napus plant (Variety “Simon”) and compared with low-EUE plants. In field trials of three subsequent years it could be demonstrated that these high-EUE plants yield ˜8% higher (kg seeds/ha) than control plants, while these low-EUE plants yield ˜10% less than control plants. In fields with moderate drought stress, the line with the highest EUE had a 20% higher yield than that of the control, while the seed yield of the line with the highest respiration and lowest EUE dropped by 20%.

### 2. Transcript Profiling Between the High Energy Efficient Hybrids and Control Hybrid Plants of Brassica napus

The transcriptome of the high-EUE B. napus (also designated as the high vigor hybrid line HV110), showing lower respiration levels, was compared to the transcriptome of the B. napus control hybrid line (also designated as B. napus control line 115). This transcriptome analysis was carried out because it was shown that the genome of line HV110 has a decrease in global methylation, which pointed out to an effect on transcription of genes.

Leaf 4 was harvested from four trays, each containing 4 to 5 plants from line HV110 and control line 115. RNA was isolated from individual leafs and used in a pilot cDNA-AFLP experiment, which indicated differences on the transcript level between the HV110 and control line. For each line (3 replicas/line), RNA from leafs harvested from the same tray was pooled, resulting in 3 samples/line for hybridization to microarrays.

In the experiments we used the commercially available 44K array developed by Agilent. This 44K array is a transcriptome-wide Brassica napus microarray. One slide contains 4 identical 44K microarrays. Each microarrays contains 43,803 probes sourced from RefSeq, UniGene, TIGR Plant TA and TIGR Gene Indices.

As an RNA source for hybridization 3 replicas/line were used. The quality report showed an increase in number of signals above background based on absent/present calls. The percentage of probes with a signal above background was 70.26%.

With the microarray 44K data, probe filtering was followed by quantile normalization. Based on P/A calls, the intensities for 27,297 probes were retained and after removal of duplicated probes we ended up with intensities for 26,851 probes. Limma and qvalue packages for R Bioconducter were used for further analysis. Pairwise comparison (t-test) resulted in 865 transcripts with a p value lower than 0.001. After correction for false discovery, we found 174 transcripts to be significantly differential between line HV110 and the control line 115.

Out of 174 differential B. napus transcripts, 61 transcripts are at least more than 0.66 fold down-regulated and 73 transcripts are at least more than 1.5 times up-regulated in the HV110 line. Table 1 depicts the list of genes which are at least 0.66 fold downregulated. Table 2 depicts the list of genes which are at least 1.5 times upregulated.

TABLE 1
list of genes which are at least 0.66 times downregulated in a high EUE plant with
respect to the average of the expression of said gene in a population of plants which
belong to the same plant species. The B. napus Probe Id, as present on the
Agilent 44k microarray, is depicted in the first column. The sixth column is the
likely Arabidopsis thaliana homologue (the AGI codes are shown). The last column
describes the gene based on homology with other proteins found in nucleotide databases.

| | | Seq | | | | | |
|---|---|---|---|---|---|---|---|
| Probe | | ID | | | | | |
| Id | Name | No | FC | P.Value | Q.value | AGI-code | Annotation |
| 14489 | EV206820 | 1 | 0.359891 | 7.06E−05 | 0.034484 | AT1G50240.2 | FU (FUSED); protein serine/threonine kinase |
| 1998 | CX279803 | 2 | 0.387082 | 0.000807 | 0.016731 | AT5G41790.1 | CIP1 (COP1-INTERACTIVE PROTEIN 1) |
| 9288 | EV015321 | 3 | 0.414199 | 0.000139 | 0.038068 | AT1G15940.1 | T24D18.4; binding |
| 1778 | TA33205_3708 | 4 | 0.42834 | 9.68E−06 | 0.012302 | AT5G06043.1 | unknown protein |
| 5711 | EE473212 | 5 | 0.443661 | 0.000301 | 0.036199 | AT2G39260.1 | RNA binding/Armadillo-type fold (InterPro: IPR016024), |
| | | | | | | | MIF4G-like, type 3 (InterPro: IPR003890)/AtUPF2 homolog |
| | | | | | | | (nonsense mediated decay) |
| 9679 | CX190620 | 6 | 0.446002 | 8.47E−05 | 0.014795 | AT1G76810.1 | Putative translation initiation factor IF-2 |
| 15621 | TA33599_3708 | 7 | 0.450138 | 6.01E−05 | 0.033289 | AtMg00090.1 | Ribosomal protein S3, mitochondrial |
| 4948 | TA34467_3708 | 8 | 0.451921 | 0.000766 | 0.019406 | unknown | unknown |
| 2282 | CN730812 | 9 | 0.460531 | 8.10E−05 | 0.014163 | AT5G22760.1 | PHD finger family protein |
| 5440 | EV039574 | 10 | 0.472461 | 3.13E−05 | 0.030558 | AT5G52070.1 | agenet domain-containing protein |
| 10640 | CX194960 | 11 | 0.479429 | 0.000292 | 0.036433 | AT4G17330.1 | ATG2484-1 (Arabidopsis thaliana G2484-1 protein); RNA |
| | | | | | | | binding |
| 8108 | TC108098 | 12 | 0.479563 | 0.000143 | 0.025417 | AT3G16630.2/ | ATKINESIN-13A/KINESIN-13A; microtubule motor |
| | | | | | | AT3G16630.1 | |
| 4691 | TC78536 | 13 | 0.480597 | 0.000499 | 0.015375 | AT3G19780.1 | similar to unknown protein [Arabidopsis thaliana] |
| | | | | | | | (TAIR: AT1G33780.1) |
| 2183 | TC84863 | 14 | 0.483555 | 0.000297 | 0.013584 | AT1G73960.2/ | TAF2 (TBP-ASSOCIATED FACTOR 2); binding/ |
| | | | | | | AT1G73960.1 | metallopeptidase/zinc ion binding |
| 14986 | TC72091 | 15 | 0.488018 | 0.00014 | 0.027054 | ATCG00350.1 | psaA protein comprising the reaction center for photosystem |
| | | | | | | | I along with psaB protein |
| 4754 | TA32705_3708 | 16 | 0.493421 | 0.000209 | 0.013584 | ATCG00680.1 | encodes for CP47, subunit of the photosystem II reaction |
| | | | | | | | centre |
| 3149 | CX189248 | 17 | 0.498384 | 0.000277 | 0.015567 | AT1G21570.1 | Zinc finger CCCH domain-containing protein 7 |
| 16426 | EV009084 | 18 | 0.498643 | 0.000393 | 0.034844 | AT5G16780.1 | Encodes a protein belonging to SART-1 family |
| 1509 | TC104814 | 19 | 0.507582 | 6.46E−05 | 0.026577 | AT1G44910.2/ | similar to FF domain-containing protein/WW domain- |
| | | | | | | AT1G44910.1 | containing protein/putative PRE-mRNA-PROCESSING |
| | | | | | | | PROTEIN 40 |
| 9209 | EE463056 | 20 | 0.509626 | 9.11E−05 | 0.047934 | AT3G62900.1 | zinc ion binding |
| 4936 | EE493614 | 21 | 0.52039 | 2.58E−05 | 0.025121 | AT4G31880.1 | binding; putative uncharacterized protein |
| 12014 | TC94522 | 22 | 0.521316 | 0.000509 | 0.049497 | AT2G06210.1 | ELF8 (early flowering 8); binding/VIP6 (vernalization |
| | | | | | | | independence) |
| 3638 | EE474589 | 23 | 0.523448 | 0.000346 | 0.023365 | AT5G35950.1 | jacalin lectin family protein |
| 1414 | EV169901 | 24 | 0.52443 | 0.000517 | 0.026285 | AT4G32940.1 | GAMMA-VPE (Vacuolar processing enzyme gamma); |
| | | | | | | | cysteine-type endopeptidase |
| 13639 | EV039765 | 25 | 0.524489 | 0.000216 | 0.044441 | AT5G16270.1 | RAD21-3 |
| 555 | TA28643_3708 | 26 | 0.54603 | 0.000736 | 0.011364 | unknown | unknown |
| 12900 | TC91536 | 27 | 0.554747 | 0.000144 | 0.024643 | AT4G32850.6/ | nPAP (NUCLEAR POLY(A) POLYMERASE) |
| | | | | | | AT4G32850.2 | |
| 10533 | TC84862 | 28 | 0.555474 | 0.000306 | 0.047464 | unknown | unknown |
| 6006 | TC108230 | 29 | 0.556876 | 0.000223 | 0.014163 | AT1G13220.2 | LINC2 (LITTLE NUCLEI2); protein binding |
| 11419 | CX190245 | 30 | 0.559209 | 0.000939 | 0.028994 | AT4G28710.1 | XIH (Myosin-like protein XIH) |
| 4064 | EV168303 | 31 | 0.565255 | 0.000513 | 0.017208 | AT5G55300.1 | TOP1BETA (DNA TOPOISOMERASE 1 BETA); DNA |
| | | | | | | | topoisomerase type I |
| 6080 | EV036137 | 32 | 0.568792 | 0.00046 | 0.040766 | AT1G15940.1 | T24D18.4; binding |
| 16034 | ES901767 | 33 | 0.568793 | 0.000623 | 0.041055 | AT2G05170.1 | ATVPS11 (A th vacuolar protein sorting 11); transporter3 |
| 7789 | EV167944 | 34 | 0.572384 | 0.000302 | 0.015941 | AT1G77800.1 | PHD finger family protein/BEST Arabidopsis thaliana protein |
| | | | | | | | match is: ATX1 (ARABIDOPSIS HOMOLOGUE OF |
| | | | | | | | TRITHORAX); histone-lysine N-methyltransferase/ |
| | | | | | | | phosphatidylinositol-5-phosphate binding |
| | | | | | | | (TAIR: AT2G31650.1) |
| 2617 | CX194438 | 35 | 0.575749 | 0.000584 | 0.023753 | AT1G77460.1 | BEST Arabidopsis thaliana match C2 domain-containing |
| | | | | | | | protein/armadillo (about 20 repeats)/beta-catenin repeat |
| | | | | | | | family protein (AT1G44120) |
| 2375 | TC87724 | 36 | 0.577814 | 0.000511 | 0.014844 | AT4G33200.1 | XI-I (Myosin-like protein XI-I); motor |
| 492 | EE472605 | 37 | 0.580138 | 0.000967 | 0.014844 | AT3G33530.1 | transducin family protein/WD-40 repeat family protein |
| 1627 | DY001899 | 38 | 0.581232 | 0.000308 | 0.014795 | AT4G33620.1 | Ulp1 protease family protein |
| 4405 | EE483172 | 39 | 0.585316 | 0.000687 | 0.025403 | AT2G21440.1 | RNA recognition motif (RRM)-containing protein |
| 12531 | EV158622 | 40 | 0.585909 | 0.000639 | 0.026775 | AT1G13160.1 | SDA1 family protein |
| 10952 | EV152694 | 41 | 0.587161 | 0.000248 | 0.023911 | AT5G37830.1 | OXP1 (OXOPROLINASE 1); hydrolase |
| 2025 | TC84680 | 42 | 0.58754 | 0.000122 | 0.048067 | AT1G32490.1 | EMB2733/ESP3 (EMBRYO DEFECTIVE 2733); ATP- |
| | | | | | | | dependent RNA helicase |
| 41 | DY000956 | 43 | 0.59288 | 0.000818 | 0.012069 | AT5G20490.1 | type XI myosin protein family involved in root hair growth, |
| | | | | | | | trichome development, and organelle trafficking |
| 3899 | EE462563 | 44 | 0.595331 | 0.000445 | 0.013687 | AT5G46210.1 | Cullin4 |
| 7924 | EV100070 | 45 | 0.595589 | 0.000487 | 0.039355 | ATCG00720.1 | Encodes the cytochrome b(6) subunit of the cytochrome b6f |
| | | | | | | | complex |
| 7088 | TA29115_3708 | 46 | 0.597567 | 0.000441 | 0.037391 | AT5G46070.1 | GTP binding/GTPase |
| 1347 | DY015857 | 47 | 0.597874 | 0.000454 | 0.017035 | AT5G38560.1 | protein kinase family protein |
| 5774 | EV169612 | 48 | 0.601745 | 0.000171 | 0.015885 | AT3G54440.2/ | Beta Galactosidase-like protein |
| | | | | | | AT3G54440.1 | |
| 624 | TC82199 | 49 | 0.606962 | 0.000358 | 0.028994 | AT5G13010.1 | EMB3011 (embryo defective 3011); RNA helicase |
| 12090 | ES911471 | 50 | 0.609079 | 0.000236 | 0.043533 | AT3G23640.2/ | Alpha glucosidase-like protein |
| | | | | | | AT3G23640.1 | |
| 3636 | EV166070 | 51 | 0.614639 | 0.000306 | 0.017895 | AT2G41790.1 | peptidase M16 family protein/insulinase family protein |
| 1549 | TC95208 | 52 | 0.614875 | 0.000577 | 0.014403 | AT5G27030.1 | TPR3 (TOPLESS-RELATED 3) |
| 13507 | CX194577 | 53 | 0.61878 | 0.000775 | 0.02386 | AT1G15740.1 | leucine-rich repeat family protein |
| 5854 | ES912455 | 54 | 0.621061 | 0.000211 | 0.017033 | AT1G73960.2/ | TAF2 (TBP-ASSOCIATED FACTOR 2); binding |
| | | | | | | AT1G73960.1 | |
| 11345 | EV095656 | 55 | 0.629025 | 0.000364 | 0.031406 | AT3G47730.1 | ABC transporter A family member 2 |
| 2036 | TC90764 | 56 | 0.629339 | 0.000958 | 0.017294 | AT4G29440.2/ | unknown protein |
| | | | | | | AT4G29440.1 | |
| 9230 | ES910216 | 57 | 0.636673 | 0.000519 | 0.03577 | AT2G16860.1 | GCIP-interacting family protein/known CG methylation |
| | | | | | | | target (Tran et al., 2005 Curr. Biol. 26) |
| 5761 | EV209464 | 58 | 0.642771 | 0.000267 | 0.019226 | AT4G28490.1 | HAESA/LRR XI-16 (RECEPTOR-LIKE PROTEIN KINASE |
| | | | | | | | 5); ATP binding/kinase/protein serine/threonine kinase |
| 10583 | TA31855_3708 | 59 | 0.64719 | 0.000746 | 0.039323 | AT2G16470.1 | zinc finger (CCCH-type) family protein/GYF domain- |
| | | | | | | | containing protein |
| 5356 | TA31439_3708 | 60 | 0.66065 | 0.000709 | 0.017561 | AT4G03560.1 | ATTPC1 (TWO-PORE CHANNEL 1); calcium channel/ |
| | | | | | | | voltage-gated calcium channel |
| 10444 | EV178465 | 61 | 0.664836 | 0.000294 | 0.026109 | AT5G18830.2/ | Squamosa promoter-binding-like protein 7 |
| | | | | | | AT5G18830.1 | |

TABLE 2
list of genes which are at least 1.5 times upregulated in a high EUE plant with
respect to the average of the expression of said gene in a population of plants
which belong to the same plant species. The B. napus Probe Id, as present
on the Agilent 44k microarray, is depicted in the first column. The sixth column
is the likely Arabidopsis thaliana homologue (the AGI codes are shown). The
last column describes the gene based on homology with other proteins found in
nucleotide databases.

| Probe | | Seq | FC (110 | | | | | |
|---|---|---|---|---|---|---|---|---|
| Id | Name | ID No | vs. 115) | P.Value | Q.value | AGI | annotation | function |
| 1507 | TA28036_3708 | 62 | 6.151369 | 1.57E−07 | 0.020948 | ATCG00600.1 | Cytochrome b6-f complex, subunit V. | |
| | | | | | | | Disruption of homologous gene in | |
| | | | | | | | Chlamydomonas results in disruption of | |
| | | | | | | | cytochrome b6-f complex. | |
| 13879 | TA25835_3708 | 63 | 3.682052 | 0.000926477 | 0.033766 | AT4G34970.1 | actin binding | |
| 10581 | EE421604 | 64 | 3.199189 | 0.000118156 | 0.036512 | unknown | unknown | |
| 3938 | CX278105 | 65 | 3.168109 | 0.00075752 | 0.038863 | AT3G44200.1// | ATNEK6/IBO1/NEK6 (NIMA (Never in | |
| | | | | | | AT2G47650.1 | mitosis, gene A)-related 6); kinase//UXS4 | |
| | | | | | | | (UDP-XYLOSE SYNTHASE 4); catalytic | |
| 10019 | DW999632 | 66 | 3.065576 | 0.000180691 | 0.01978 | AT3G56910.1 | 50S ribosomal protein 5, chloroplastic | C |
| 12031 | TA22154_3708 | 67 | 3.060641 | 7.62E−05 | 0.030743 | AT1G52740.1 | HTA9; DNA binding | |
| 6707 | TA27587_3708 | 68 | 2.684381 | 0.000513556 | 0.017662 | unknown | unknown | |
| 5627 | TC97093 | 69 | 2.501035 | 1.55E−05 | 0.023796 | AT5G65220.1 | ribosomal protein L29 family protein | C |
| 9673 | EE436101 | 70 | 2.24658 | 0.000102907 | 0.039435 | AT1G15100.1 | RING-H2 zinc finger protein RHA2a | |
| 3170 | DY007779 | 71 | 2.204034 | 0.000676686 | 0.014163 | AT5G03060.1 | Putative uncharacterized protein | |
| | | | | | | | F15A17_90 | |
| 4372 | EE513191 | 72 | 2.188919 | 0.000608143 | 0.014646 | AT2G44670.1 | senescence-associated protein-related | |
| 3686 | ES902942 | 73 | 2.179193 | 0.000483703 | 0.015024 | AT5G42180.1 | Peroxidase 64 | |
| 9172 | EV091739 | 74 | 2.159295 | 0.000315197 | 0.017561 | AT5G45700.1 | NLI interacting factor (NIF) family protein | |
| 9189 | TA27889_3708 | 75 | 2.153614 | 2.63E−05 | 0.019532 | AT5G64816.2/ | Putative uncharacterized protein | |
| | | | | | | AT5G64816.1 | | |
| 2498 | DY003639 | 76 | 2.106356 | 0.00019152 | 0.012547 | AT1G15100.1 | RING-H2 zinc finger protein RHA2a | |
| 11496 | CX196079 | 77 | 2.080218 | 0.000172533 | 0.048054 | AT2G19470.1 | Putative casein kinase I | |
| 9867 | EV176241 | 78 | 2.070707 | 0.000188682 | 0.025573 | AT1G56045.1 | 60S ribosomal protein L41 | T |
| 10115 | EE453735 | 79 | 2.070494 | 0.000318886 | 0.036173 | AT4G15140.1 | similar to unknown [Populus trichocarpa] | |
| | | | | | | | (GB: ABK96081.1) | |
| 11642 | EE560078 | 80 | 2.059658 | 0.000132085 | 0.044602 | AT3G62790.1 | NDUFS5: 15 kDa subunit | M |
| 10819 | EV184671 | 81 | 2.037074 | 5.79E−05 | 0.019147 | AT2G42310.1 | NDU12-1; plant-specific subunit of | M |
| | | | | | | | complex I | |
| 1923 | CD821628 | 82 | 2.035021 | 0.000354489 | 0.035202 | AT1G67350.2/ | 11 kDa subunit of complex I | M |
| | | | | | | AT1G67350.1 | | |
| 11204 | EE458932 | 83 | 1.991268 | 0.000149493 | 0.037975 | AT1G05720.1 | selenoprotein family protein | |
| 9529 | CD812637 | 84 | 1.965957 | 0.000206008 | 0.027595 | AT3G06320/ | 50S ribosomal protein L33 | T |
| | | | | | | AT5G18790.1 | | |
| 788 | DY002151 | 85 | 1.924112 | 0.00033782 | 0.012069 | AT3G05000.1 | transport protein particle (TRAPP) | |
| | | | | | | | component Bet3 family protein | |
| 7619 | ES963374 | 86 | 1.920093 | 0.000460593 | 0.015941 | unknown | unknown | |
| 10402 | DW998509 | 87 | 1.909599 | 0.000714964 | 0.031239 | AT5G44520.1 | ribose 5-phosphate isomerase-related | C |
| 7391 | DY020522 | 88 | 1.8882 | 0.000258641 | 0.015189 | AT5G61750.1 | cupin family protein | |
| 9932 | TA27488_3708 | 89 | 1.88243 | 0.000370594 | 0.025573 | AT4G25050.1 | ACP4 (acyl carrier protein 4) | C |
| 5116 | EE541698 | 90 | 1.863489 | 0.00013695 | 0.013587 | AT4G20030.1 | RNA recognition motif (RRM)-containing | C |
| | | | | | | | protein | |
| 13795 | TA21968_3708 | 91 | 1.85915 | 0.000911199 | 0.021533 | AT5G47700.2/ | 60S acidic ribosomal protein P1 (RPP1C) | T |
| | | | | | | AT5G47700.1 | | |
| 3800 | EE468901 | 92 | 1.857317 | 0.000611184 | 0.023227 | AT2G42310.1 | NDU12-1; plant-specific subunit of | M |
| | | | | | | | complex I | |
| 2499 | DY001295 | 93 | 1.855851 | 0.000802145 | 0.022718 | AT4G29480.1 | mitochondrial ATP synthase g subunit | M |
| | | | | | | | family protein | |
| 12387 | TC100917 | 94 | 1.847804 | 0.000173141 | 0.020295 | unknown | unknown | |
| 6684 | TA34792_3708 | 95 | 1.832297 | 0.000908313 | 0.015885 | AT1G08280.1 | glycosyl transferase family 29 protein/ | |
| | | | | | | | sialyltransferase family protein | |
| 2644 | TC156798 | 96 | 1.827059 | 0.000548433 | 0.021614 | AT1G56045.1 | 60S ribosomal protein L41 | T |
| 11371 | TC98118 | 97 | 1.8216 | 0.000140671 | 0.020529 | AT4G03950.1 | glucose-6-phosphate/phosphate | |
| | | | | | | | translocator, putative | |
| 6862 | EL590475 | 98 | 1.818829 | 0.000804143 | 0.020771 | AT5G55940.1 | EMB2731 (EMBRYO DEFECTIVE 2731) | |
| 10132 | EV034165 | 99 | 1.818257 | 0.000826803 | 0.047174 | unknown | unknown | |
| 2368 | EE474143 | 100 | 1.815175 | 0.000142212 | 0.018517 | unknown | unknown | |
| 14557 | TA22213_3708 | 101 | 1.813612 | 0.000975621 | 0.026285 | AT4G18730.1 | RPL16B (ribosomal protein L16B) | T |
| 6784 | EV106558 | 102 | 1.813398 | 0.000884981 | 0.016022 | AT5G39210.1 | CRR7 (CHLORORESPIRATORY | |
| | | | | | | | REDUCTION 7) | |
| 14311 | TA27876_3708 | 103 | 1.807074 | 0.000100811 | 0.022976 | AT1G73940.1 | similar to unknown protein AT5G49410 | |
| 11951 | TC103797 | 104 | 1.801332 | 8.06E−05 | 0.032219 | AT5G08040.1 | TOM5 (mitochondrial import receptor | M |
| | | | | | | | subunit TOM5 homolog) | |
| 4660 | CN735121 | 105 | 1.793879 | 0.000613474 | 0.036421 | AT2G24395.1 | chaperone protein dnaJ-related | C |
| 5818 | TA23857_3708 | 106 | 1.79361 | 0.000432198 | 0.012825 | AT1G80890.1 | similar to unknown protein | |
| | | | | | | | (TAIR: AT1G16000.1) | |
| 2479 | EV169117 | 107 | 1.791825 | 0.000702804 | 0.012547 | AT1G03600.1 | photosystem II family protein | C |
| 2638 | CD822014 | 108 | 1.786305 | 0.000486176 | 0.029277 | AT5G25540.1 | CID6 (CTC-Interacting Domain 6); protein | |
| | | | | | | | binding | |
| 1216 | TC92592 | 109 | 1.78003 | 0.000489166 | 0.020464 | AT4G21470.1 | ATFMN/FHY (riboflavin kinase/FMN | |
| | | | | | | | hydrolase); FMN adenylyltransferase/ | |
| | | | | | | | riboflavin kinase | |
| 10441 | CD818969 | 110 | 1.771567 | 0.000254474 | 0.046179 | AT5G09225.1 | Putative uncharacterized protein | |
| 16224 | EV131396 | 111 | 1.76423 | 9.14E−05 | 0.032296 | AT4G02620.1 | (VACUOLAR ATPASE SUBUNIT F); | |
| | | | | | | | hydrogen ion transporting ATP synthase, | |
| | | | | | | | rotational mechanism | |
| 9604 | TA22152_3708 | 112 | 1.762849 | 0.000159349 | 0.030437 | AT1G52740.1 | HTA9; DNA binding | |
| 6889 | DY017585 | 113 | 1.758316 | 7.10E−05 | 0.016887 | AT4G32470.1 | ubiquinol-cytochrome C reductase | M |
| | | | | | | | complex 14 kDa protein; putative (QCR7- | |
| | | | | | | | 1) | |
| 16212 | DY023037 | 114 | 1.755392 | 0.000491622 | 0.04526 | AT1G27695.2/ | glycine-rich protein | |
| | | | | | | AT1G27695.1 | | |
| 8290 | EV169560 | 115 | 1.727391 | 0.000517663 | 0.014853 | AT3G53730.1 | histone H4 | |
| 11270 | TA23048_3708 | 116 | 1.721008 | 0.000280446 | 0.029072 | AT5G27700.1 | 40S ribosomal protein S21 (RPS21C) | T |
| 13950 | TA25994_3708 | 117 | 1.720911 | 0.000384894 | 0.036113 | AT1G50900.1 | unknown protein F8A12.12 | C |
| 12868 | CX281365 | 118 | 1.705674 | 0.000191271 | 0.021336 | AT4G30330.1/ | Small nuclear ribonucleoprotein | |
| | | | | | | AT2G18740.1 | homolog/small nuclear ribonucleoprotein | |
| | | | | | | | E, putative | |
| 138 | TA31407_3708 | 119 | 1.705189 | 0.000129071 | 0.019297 | AT4G15510.3/ | photosystem II reaction center PsbP | C |
| | | | | | | AT4G15510.1 | family protein | |
| 2156 | TA27971_3708 | 120 | 1.679557 | 0.000417144 | 0.015763 | AT2G33820.1 | ATMBAC1; L-histidine transmembrane | |
| | | | | | | | transporter/L-lysine transmembrane | |
| | | | | | | | transporter/L-ornithine transmembrane | |
| | | | | | | | transporter/arginine transmembrane | |
| | | | | | | | transporter/binding | |
| 2084 | TA24414_3708 | 121 | 1.672037 | 0.000192567 | 0.017035 | AT5G59613.1 | ATP 6 kDa; subunit of complex V | M |
| 7069 | TA21585_3708 | 122 | 1.640388 | 0.000416277 | 0.016573 | AT4G15000.1 | 60S ribosomal protein L27-3 | T |
| 4394 | TA21794_3708 | 123 | 1.634047 | 0.00072061 | 0.017889 | AT4G16450.1 | 20.9 kDa subunit of complex I | M |
| 11333 | TC101749 | 124 | 1.622678 | 0.00057502 | 0.035399 | AT3G06620.1 | protein kinase family protein | |
| 14049 | TA32534_3708 | 125 | 1.615252 | 0.000485263 | 0.03549 | AT4G14420.1 | lesion inducing protein-related | |
| 12061 | EV173428 | 126 | 1.605293 | 0.000498491 | 0.02878 | AT2G16060.1 | Non-symbiotic hemoglobin 2 | |
| 6328 | TA21968_3708 | 127 | 1.604449 | 0.0007473 | 0.0133 | AT1G01100.4/ | 60S acidic ribosomal protein P1 (RPP1A) | T |
| | | | | | | AT1G01100.2/ | | |
| | | | | | | AT1G01100.1 | | |
| 13094 | EV077787 | 128 | 1.584143 | 0.000464618 | 0.041934 | AT5G59613.1 | ATP 6 kDa; subunit of complex V | M |
| 13243 | TA29086_3708 | 129 | 1.572297 | 0.000848279 | 0.021533 | AT5G47890.1 | NADH-ubiquinone oxidoreductase B8 | M |
| | | | | | | | subunit, putative (NDUFA2: B8 subunit of | |
| | | | | | | | complex I) | |
| 13964 | DY007087 | 130 | 1.555607 | 0.000977598 | 0.029628 | AT2G34340.1 | similar to unknown protein [Arabidopsis | |
| | | | | | | | thaliana] (TAIR: AT1G29640.1) | |
| 1239 | EV177713 | 131 | 1.549724 | 0.000809426 | 0.021284 | AT4G31560.1 | HCF153, a 15-KDa protein involved in the | C |
| | | | | | | | biogenesis of the cytochrome b(6)f | |
| | | | | | | | complex. Associated with the thylakoid | |
| | | | | | | | membrane. | |
| 2708 | EV091750 | 132 | 1.549111 | 0.000577729 | 0.011364 | AT1G23290.1 | RPL27A (RIBOSOMAL PROTEIN L27A) | T |
| 3529 | TA21093_3708 | 133 | 1.536451 | 0.000966524 | 0.014646 | AT5G57290.3/ | 60S acidic ribosomal protein P3 (RPP3B) | T |
| | | | | | | AT5G57290.2/ | | |
| | | | | | | AT5G57290.1 | | |
| 4635 | TC105794 | 134 | 1.529567 | 0.00062427 | 0.015907 | AT5G63510.1 | mitochondrial gamma carbonic | M |
| | | | | | | | anhydrase-like protein (CAL1: carbonic | |
| | | | | | | | anhydrase like 1) | |

### 3. Identification of Transcriptional Networks in the High Vigor Brassica Hybrid

Subsequently, we used the lists of up- and down-regulated genes (Table I depicts the genes that are at least 0.66 times downregulated in HV110 with respect to the control hybrid line 115, Table II depicts the genes that are at least 1.5 times upregulated in HV110 with respect to the control hybrid line 115) that are differentially expressed in the HV110 line as input list for the web tool CORNET (http://bioinformatics.psb.ugent.be/cornet/). With this tool, co-expression patterns can be visualized. This co-expression is defined by calculating the Pearson correlation between gene expression profiles using precompiled publically available microarray gene expression data sets.

### 3.1 Transcription Networks for Downregulated Genes

The input list for CORNET after removal of doubles or Brassica IDs without an Arabidopsis homolog is 56 AGI (Arabidopsis Genome Initiative) codes. For 8 AGI codes no reliable probe sets were found according to the CDF file used by CORNET, which results in an input list of 48 AGI codes. The selected arrays (1488 exp in total) include arrays from abiotic stress (256 exp), AtGenExpress All (425 exp), development (135 exp), hormone treatment (140 exp), microarray compendium 2 (111 exp—no bias), stress (abiotic+biotic) (336 exp) and whole plant (85 exp). The selected databases for identification of protein-protein interaction include the Bar, IntAct and TAIR databases. We identified two networks with a Pearson correlation coefficient higher than 0.75. These networks are depicted in Tables 3 and 4.

TABLE 3
Network I: FC ≦ 0.66 (110vs115) and Pearson correlation coefficient > 0.75

| Systematic | | | |
|---|---|---|---|
| Name | AGI | Annotation | Pearson |
| TC72091 | ATCG00350 | psaA protein | 0.75 |
| TA32705_3708 | ATCG00680 | CP47 (subunit of the photosystem II reaction center) | 0.75 |
| EV100070 | ATCG00720 | cytochrome b(6) subunit | 0.75 |

TABLE 4
Network II: FC ≦ 0.66 (110vs115) and Pearson correlation coefficient > 0.75

| Systematic | | | |
|---|---|---|---|
| Name | AGI | Annotation | Pears. |
| EV158622 | AT1G13160.1 | SDA1 family protein | 0.75 |
| TC108230 | AT1G13220.2 | LINC2 | 0.75 |
| EV015321/ | AT1G15940.1 | binding | 0.75 |
| EV036137 | | | |
| TC84680 | AT1G32490.1 | ESP3 | 0.75 |
| TC104814 | AT1G44910.2/ | protein binding | 0.75 |
| | AT1G44910.1 | | |
| TC84863/ | AT1G73960.2/ | TAF2 | 0.75 |
| ES912455 | AT1G73960.1 | | |
| CX190620 | AT1G76810.1 | eukaryotic translation initiation factor 2 family protein | 0.75 |
| EV167944 | AT1G77800.1 | PHD finger family protein | 0.75 |
| TC94522 | AT2G06210.1 | ELF8 | 0.75 |
| ES910216 | AT2G16860.1 | GCIP-interacting family protein | 0.75 |
| EE483172 | AT2G21440.1 | RNA recognition motif | 0.75 |
| EE473212 | AT2G39260.1 | RNA binding | 0.75 |
| EV166070 | AT2G41790.1 | peptidase M16 fami;y protein | 0.75 |
| TC108098 | AT3G16630.1 | KINESIN 13A | 0.75 |
| EE472605 | AT3G33530.1 | transducin family protein | 0.75 |
| CX194960 | AT4G17330.1 | ATG2484-1 | 0.75 |
| EE493614 | AT4G31880.1 | unknown | 0.75 |
| TC91536 | AT4G32850.6/ | nPAP | 0.75 |
| | AT4G32850.2 | | |
| TC87724 | AT4G33200.1 | XI-I | 0.75 |
| DY001899 | AT4G33620.1 | Ulp1 protease family protein | 0.75 |
| TC82199 | AT5G13010.1 | EMB3011 | 0.75 |
| EV039765 | AT5G16270.1 | SYN4 | 0.75 |
| EV009084 | AT5G16780.1 | DOT2 | 0.75 |
| EV178465 | AT5G18830.2/ | SPL7 | 0.75 |
| | AT5G18830.1 | | |
| CN730812 | AT5G22760.1 | PHD finger family protein | 0.75 |
| DY015857 | AT5G38560.1 | protein kinase family protein | 0.75 |
| TA29115_3708 | AT5G46070.1 | GTP binding | 0.75 |
| EE462563 | AT5G46210.1 | CUL4 (CULLIN 4) | 0.75 |
| EV168303 | AT5G55300.1 | TOP1 ALPHA | 0.75 |

We identified two networks with a Pearson correlation coefficient >0.8. These networks are depicted in Tables 5 and 6.

TABLE 5
Network I: FC ≦ 0.66 (110vs115) and Pearson correlation coefficient > 0.8

| Systematic | | | |
|---|---|---|---|
| Name | AGI | Annotation | Pearson |
| TC72091 | ATCG00350.1 | psaA protein | 0.75 |
| TA32705_3708 | ATCG00680.1 | CP47 (subunit of the photosystem II reaction center) | 0.75 |
| EV100070 | ATCG00720.1 | cytochrome b(6) subunit | 0.75 |

TABLE 6
Network II: FC ≦ 0.66 (110vs115) and Pearson correlation coefficient > 0.8

| Systematic | | | |
|---|---|---|---|
| Name | AGI | Annotation | Pearson |
| EV158622 | AT1G13160.1 | SDA1 family protein | 0.8 |
| TC108230 | AT1G13220.2 | LINC2 | 0.8 |
| EV015321/ | AT1G15940.1 | binding | 0.8 |
| EV036137 | | | |
| TC84680 | AT1G32490.1 | ESP3 | 0.8 |
| TC104814 | AT1G44910.2/ | protein binding | 0.8 |
| | AT1G44910.1 | | |
| TC84863/ | AT1G73960.2/ | TAF2 | 0.8 |
| ES912455 | AT1G73960.1 | | |
| CX190620 | AT1G76810.1 | eukaryotic translation initiation factor 2 family protein | 0.8 |
| EV167944 | AT1G77800.1 | PHD finger family protein | 0.8 |
| TC94522 | AT2G06210.1 | ELF8 | 0.8 |
| ES910216 | AT2G16860.1 | GCIP-interacting family protein | 0.8 |
| EE483172 | AT2G21440.1 | RNA recognition motif | 0.8 |
| EE473212 | AT2G39260.1 | RNA binding | 0.8 |
| EE472605 | AT3G33530.1 | transducin family protein | 0.8 |
| CX194960 | AT4G17330.1 | ATG2484-1 | 0.8 |
| EE493614 | AT4G31880.1 | unknown | 0.8 |
| TC91536 | AT4G32850.6/ | nPAP | 0.8 |
| | AT4G32850.2 | | |
| TC87724 | AT4G33200.1 | XI-I | 0.8 |
| DY001899 | AT4G33620.1 | Ulp1 protease family protein | 0.8 |
| TC82199 | AT5G13010.1 | EMB3011 | 0.8 |
| EV039765 | AT5G16270.1 | SYN4 | 0.8 |
| EV009084 | AT5G16780.1 | DOT2 | 0.8 |
| EV178465 | AT5G18830.2/ | SPL7 | 0.8 |
| | AT5G18830.1 | | |
| CN730812 | AT5G22760.1 | PHD finger family protein | 0.8 |
| TA29115_3708 | AT5G46070.1 | GTP binding | 0.8 |
| EE462563 | AT5G46210.1 | CUL4 (CULLIN 4) | 0.8 |
| EV168303 | AT5G55300.1 | TOP1 ALPHA | 0.8 |

We identified one network with a Pearson correlation coefficient >0.9. This network is depicted in Table 7.

TABLE 7
Network I: FC ≦ 0.66 (110vs115) and Pearson correlation coefficient > 0.9

| Systematic Name | AGI | Annotation | Pearson |
|---|---|---|---|
| TC72091 | ATCG00350.1 | psaA protein | 0.9 |
| TA32705_3708 | ATCG00680.1 | CP47 (subunit of the photosystem II reaction center) | 0.9 |

3.2 Transcription Networks for upregulated Genes

The input list for CORNET after removal of doubles or Brassica IDs without an Arabidopsis homolog is 65 AGI codes. For 11 AGI codes no reliable probe sets were found according to the CDF file used by CORNET, which results in an input list of 54 AGI codes. The selected arrays (1488 exp in total) include arrays from abiotic stress (256 exp), AtGenExpress All (425 exp), development (135 exp), hormone treatment (140 exp), microarray compendium 2 (111 exp—no bias), stress (abiotic+biotic) (336 exp) and whole plant (85 exp). The selected databases for identification of protein-protein interaction include the Bar, IntAct and TAIR databases.

We identified five network with a Pearson correlation coefficient higher than 0.70. These networks are depicted in Tables 8, 9, 10 and 11.

TABLE 8
Network I + II: FC ≦ 1.5 (110vs115) and Pearson correlation coefficient > 0.7

| Systematic | | | |
|---|---|---|---|
| Name | AGI | Annotation | Pearson |
| TA21968_3708 | AT1G01100.4/ | 60S acidic ribosomal protein P1 (RPP1A) | 0.7 |
| | AT1G01100.2/ | | |
| | AT1G01100.1 | | |
| EV169117 | AT1G03600.1 | photosystem II family protein | 0.7 |
| EE458932 | AT1G05720.1 | selenoprotein family protein | 0.7 |
| TA34792_3708 | AT1G08280.1 | glycosyl transferase family 29 protein | 0.7 |
| TA25994_3708 | AT1G50900.1 | unknown | 0.7 |
| TA22154_3708/ | AT1G52740.1 | HTA9 | 0.7 |
| TA22152_3708 | | | |
| CD821628 | AT1G67350.2/ | 11 kDa subunit of complex I | 0.7 |
| | AT1G67350.1 | | |
| TA27876_3708 | AT1G73940.1 | unknown | 0.7 |
| CX281365 | AT2G18740.1 | small nuclear ribonucleoprotein E, putative | 0.7 |
| TA27971_3708 | AT2G33820.1 | MBAC1 | 0.7 |
| EV184671/ | AT2G42310.1 | NDU12-1; plant-specific subunit of complex I | 0.7 |
| EE468901 | | | |
| EE513191 | AT2G44670.1 | senescence-associated protein | 0.7 |
| DY002151 | AT3G05000.1 | transport protein particle | 0.7 |
| DW999632 | AT3G56910.1 | RSRP5 | 0.7 |
| EE560078 | AT3G62790.1 | NADH-ubiquinone oxidoreductase-related | 0.7 |
| EV131396 | AT4G02620.1 | vacuolar ATPase subunit F family protein | 0.7 |
| TA32534_3708 | AT4G14420.1 | lesion inducing protein-related | 0.7 |
| TA21585_3708 | AT4G15000.1 | 60Sribosomal protein L27 (RPL27) | 0.7 |
| TA31407_3708 | AT4G15510.3/ | photosystem II reaction centre PsbP family protein | 0.7 |
| | AT4G15510.1 | | |
| TA21794_3708 | AT4G16450.1 | 20.9 kDa subunit of complex I | 0.7 |
| EE541698 | AT4G20030.1 | RNA recognition motif | 0.7 |
| TA27488_3708 | AT4G25050.1 | ACP4 | 0.7 |
| DY001295 | AT4G29480.1 | mitochondrial ATP synthase g subunit family protein | 0.7 |
| CX281365 | AT4G30330.1 | small nuclear ribonucleoprotein E, putative | 0.7 |
| EV177713 | AT4G31560.1 | HCF | 0.7 |
| DY017585 | AT4G32470.1 | ubiquinol-cytochrome C reductase complex 14 kDa protein, | 0.7 |
| | | putative | |
| TC103797 | AT5G08040.1 | TOM5 | 0.7 |
| CD822014 | AT5G25540.1 | CID6 | 0.7 |
| TA23048_3708 | AT5G27700.1 | structural constituent of ribosome | 0.7 |
| EV106558 | AT5G39210.1 | CRR7 | 0.7 |
| DW998509 | AT5G44520.1 | ribose 5-phosphate isomerase-related | 0.7 |
| TA21968_3708 | AT5G47700.2/ | 60S acidic ribosomal protein P1 (RPP1C) | 0.7 |
| | AT5G47700.1 | | |
| TA29086_3708 | AT5G47890.1 | NADH-ubiquinone oxidoreductase B8 subunit, putative | 0.7 |
| EL590475 | AT5G55940.1 | emb2731 | 0.7 |
| TA21093_3708 | AT5G57290.3/ | 60S acidic ribosomal protein P3 (RPP3B) | 0.7 |
| | AT5G57290.2/ | | |
| | AT5G57290.1 | | |
| TC105794 | AT5G63510.1 | gamma CAL1 | 0.7 |
| TA27889_3708 | AT5G64816.2/ | unknown | 0.7 |
| | AT5G64816.1 | | |
| TC97093 | AT5G65220.1 | ribosomal protein L29 family protein | 0.7 |
| TA28036_3708 | ATCG00600.1 | Cytochrome b6-f complex, subunit V | 0.7 |

TABLE 9
Network III: FC ≧ 1.5 (110 vs 115) and Pearson
correlation coefficient > 0.7

| Systematic Name | AGI | Annotation | Pearson |
|---|---|---|---|
| TC98118 | AT4G03950.1 | glucose 6-phosphate | 0.7 |
| DY020522 | AT5G61750.1 | cupin family protein | 0.7 |

TABLE 10
Network IV: FC ≧ 1.5 (110 vs 115) and Pearson
correlation coefficient > 0.7

| Systematic Name | AGI | Annotation | Pearson |
|---|---|---|---|
| EE436101/ | AT1G15100.1 | RHA2A | 0.7 |
| DY003639 | | | |
| CX278105 | AT3G44200.1 | NEK6 (NIMA (NEVER | 0.7 |
| | | IN MITOSIS, GENE A)- | |
| | | RELATED 6)) | |
| TA25835_3708 | AT4G34970.1 | ADF9 | 0.7 |
| ES902942 | AT5G42180.1 | peroxidase 64 (PER64) | 0.7 |
| | | (P64) (PRXR4) | |

TABLE 11
Network V: FC ≧ 1.5 (110 vs 115) and Pearson
correlation coefficient > 0.7

| Systematic Name | AGI | Annotation | Pearson |
|---|---|---|---|
| EV169560 | AT3G53730.1 | histone H4 | 0.7 |
| EE453735 | AT4G15140.1 | unknown | 0.7 |
| EV091739 | AT5G45700.1 | NLI interacting factor | 0.7 |

We identified four networks with a Pearson correlation coefficient >0.75.

TABLE 12
Network I: FC ≧ 1.5 (110 vs 115) and Pearson
correlation coefficient > 0.75

| Systematic Name | AGI | Annotation | Pearson |
|---|---|---|---|
| EV169117 | AT1G03600.1 | photosystem II family | 0.75 |
| | | protein | |
| TA25994_3708 | AT1G50900.1 | unknown | 0.75 |
| DW999632 | AT3G56910.1 | RSRP5 | 0.75 |
| TA31407_3708 | AT4G15510.3/ | photosystem II reaction | 0.75 |
| | AT4G15510.1 | centre PsbP family protein | |
| EE541698 | AT4G20030.1 | RNA recognition motif | 0.75 |
| TA27488_3708 | AT4G25050.1 | ACP4 | 0.75 |
| EV177713 | AT4G31560.1 | HCF153 | 0.75 |
| EV106558 | AT5G39210.1 | CRR7 | 0.75 |
| DW998509 | AT5G44520.1 | ribose 5-phosphate | 0.75 |
| | | isomerase-related | |
| TC97093 | AT5G65220.1 | ribosomal protein L29 | 0.75 |
| | | family protein | |
| TA28036_3708 | ATCG00600.1 | Cytochrome b6-f complex, | 0.75 |
| | | subunit V | |

TABLE 13
Network III: FC ≧ 1.5 (110 vs 115) and Pearson
correlation coefficient > 0.75

| Systematic Name | AGI | Annotation | Pearson |
|---|---|---|---|
| TC98118 | AT4G03950.1 | glucose 6-phosphate | 0.75 |
| DY020522 | AT5G61750.1 | cupin family protein | 0.75 |

TABLE 14
Network IV: FC ≧ 1.5 (110 vs 115) and Pearson
correlation coefficient > 0.75

| Systematic Name | AGI | Annotation | Pearson |
|---|---|---|---|
| EE436101/ | AT1G15100.1 | RHA2A | 0.75 |
| DY003639 | | | |
| ES902942 | AT5G42180.1 | peroxidase 64 (PER64) | |
| | | (P64) (PRXR4) | 0.75 |

TABLE 15
Network II: FC ≧ 1.5 (110 vs 115) and Pearson
correlation coefficient > 0.75

| Systematic Name | AGI | Annotation | Pearson |
|---|---|---|---|
| TA21968_3708 | AT1G01100.4/ | 60S acidic ribosomal | 0.75 |
| | AT1G01100.2/ | protein P1 (RPP1A) | |
| | AT1G01100.1 | | |
| EE458932 | AT1G05720.1 | selenoprotein family | 0.75 |
| | | protein | |
| TA34792_3708 | AT1G08280.1 | glycosyl transferase | 0.75 |
| | | family 29 protein | |
| TA22154_3708/ | AT1G52740.1 | HTA9 | 0.75 |
| TA22152_3708 | | | |
| CD821628 | AT1G67350.2/ | 11 kDa subunit of | 0.75 |
| | AT1G67350.1 | complex I | |
| CD821628 | AT1G67350.2/ | 11 kDa subunit of | 0.75 |
| | AT1G67350.1 | complex I | |
| CX281365 | AT2G18740.1 | small nuclear ribonucleo- | 0.75 |
| | | protein E, putative | |
| EV184671/ | AT2G42310.1 | NDU12-1; plant-specific | 0.75 |
| EE468901 | | subunit of complex I | |
| DY002151 | AT3G05000.1 | transport protein particle | 0.75 |
| EE560078 | AT3G62790.1 | NADH-ubiquinone | 0.75 |
| | | oxidoreductase-related | |
| EV131396 | AT4G02620.1 | vacuolar ATPase subunit | 0.75 |
| | | F family protein | |
| TA32534_3708 | AT4G14420.1 | lesion inducing protein- | 0.75 |
| | | related | |
| TA21585_3708 | AT4G15000.1 | 60S ribosomal protein | 0.75 |
| | | L27 (RPL27) | |
| TA21794_3708 | AT4G16450.1 | 20.9 kDa subunit of | 0.75 |
| | | complex I | |
| DY001295 | AT4G29480.1 | mitochondrial ATP | 0.75 |
| | | synthase g subunit | |
| | | family protein | |
| CX281365 | AT4G30330.1 | small nuclear ribonucleo- | 0.75 |
| | | protein E, putative | |
| DY017585 | AT4G32470.1 | ubiquinol-cytochrome | 0.75 |
| | | C reductase complex | |
| | | 14 kDa protein, putative | |
| TC103797 | AT5G08040.1 | TOM5 | 0.75 |
| CD822014 | AT5G25540.1 | CID6 | 0.75 |
| TA23048_3708 | AT5G27700.1 | structural constituent of | 0.75 |
| | | ribosome | |
| TA21968_3708 | AT5G47700.2/ | 60S acidic ribosomal | 0.75 |
| | AT5G47700.1 | protein P1 (RPP1C) | |
| TA29086_3708 | AT5G47890.1 | NADH-ubiquinone | 0.75 |
| | | oxidoreductase B8 | |
| | | subunit, putative | |
| EL590475 | AT5G55940.1 | emb2731 | 0.75 |
| TA21093_3708 | AT5G57290.3/ | 60S acidic ribosomal | 0.75 |
| | AT5G57290.2/ | protein P3 (RPP3B) | |
| | AT5G57290.1 | | |
| TC105794 | AT5G63510.1 | gamma CAL1 | 0.75 |

We identified three networks with a Pearson correlation coefficient >0.80. These networks are depicted in Tables 16, 17 and 18.

TABLE 16
Network I: FC ≧ 1.5 (110 vs 115) and Pearson
correlation coefficient > 0.80

| Systematic Name | AGI | Annotation | Pearson |
|---|---|---|---|
| EV169117 | AT1G03600.1 | photosystem II family | 0.8 |
| | | protein | |
| TA25994_3708 | AT1G50900.1 | unknown | 0.8 |
| DW999632 | AT3G56910.1 | RSRP5 | 0.8 |
| TA31407_3708 | AT4G15510.3/ | photosystem II reaction | 0.8 |
| | AT4G15510.1 | centre PsbP family | |
| | | protein | |
| TA27488_3708 | AT4G25050.1 | ACP4 | 0.8 |
| EV177713 | AT4G31560.1 | HCF153 | 0.8 |
| EV106558 | AT5G39210.1 | CRR7 | 0.8 |
| DW998509 | AT5G44520.1 | ribose 5-phosphate | 0.8 |
| | | isomerase-related | |
| TC97093 | AT5G65220.1 | ribosomal protein L29 | 0.8 |
| | | family protein | |

TABLE 17
Network III: FC ≧ 1.5 (110 vs 115) and Pearson
correlation coefficient > 0.80

| Systematic Name | AGI | Annotation | Pearson |
|---|---|---|---|
| TC98118 | AT4G03950.1 | glucose 6-phosphate | 0.8 |
| DY020522 | AT5G61750.1 | cupin family protein | 0.8 |

TABLE 18
Network II: FC ≧ 1.5 (110 vs 115) and Pearson
correlation coefficient > 0.80

| Systematic Name | AGI | Annotation | Pearson |
|---|---|---|---|
| TA21968_3708 | AT1G01100.4/ | 60S acidic ribosomal | 0.8 |
| | AT1G01100.2/ | protein P1 (RPP1A) | |
| | AT1G01100.1 | | |
| EE458932 | AT1G05720.1 | selenoprotein family | 0.8 |
| | | protein | |
| TA22154_3708/ | AT1G52740.1 | HTA9 | 0.8 |
| TA22152_3708 | | | |
| CD821628 | AT1G67350.2/ | 11 kDa subunit of | 0.8 |
| | AT1G67350.1 | complex I | |
| TA27876_3708 | AT1G73940.1 | unknown | 0.8 |
| CX281365 | AT2G18740.1 | small nuclear ribonucleo- | 0.8 |
| | | protein E, putative | |
| EV184671/ | AT2G42310.1 | NDU12-1; plant-specific | 0.8 |
| EE468901 | | subunit of complex I | |
| DY002151 | AT3G05000.1 | transport protein particle | 0.8 |
| TA21585_3708 | AT4G15000.1 | 60S ribosomal protein | 0.8 |
| | | L27 (RPL27) | |
| TA21794_3708 | AT4G16450.1 | 20.9 kDa subunit of | 0.8 |
| | | complex I | |
| DY001295 | AT4G29480.1 | mitochondrial ATP | 0.8 |
| | | synthase g | |
| | | subunit family protein | |
| CX281365 | AT4G30330.1 | small nuclear ribonucleo- | 0.8 |
| | | protein E, putative | |
| DY017585 | AT4G32470.1 | ubiquinol-cytochrome | 0.8 |
| | | C reductase omplex | |
| | | 14 kDa protein, putative | |
| TC103797 | AT5G08040.1 | TOM5 | 0.8 |
| TA23048_3708 | AT5G27700.1 | structural constituent of | 0.8 |
| | | ribosome | |
| TA21968_3708 | AT5G47700.2/ | 60S acidic ribosomal | 0.8 |
| | AT5G47700.1 | protein P1 (RPP1C) | |
| TA29086_3708 | AT5G47890.1 | NADH-ubiquinone | 0.8 |
| | | oxidoreductase B8 | |
| | | subunit, putative | |
| EL590475 | AT5G55940.1 | emb2731 | 0.8 |
| TA21093_3708 | AT5G57290.3/ | 60S acidic ribosomal | 0.8 |
| | AT5G57290.2/ | protein P3 (RPP3B) | |
| | AT5G57290.1 | | |
| TC105794 | AT5G63510.1 | gamma CAL1 | 0.8 |

We identified three networks with a Pearson correlation coefficient >0.90. These networks are depicted in Tables 19, 20 and 21.

TABLE 19
Network I: FC ≧ 1.5 (110 vs 115) and Pearson
correlation coefficient > 0.90

| Systematic Name | AGI | Annotation | Pearson |
|---|---|---|---|
| EV169117 | AT1G03600.1 | photosystem II family | 0.9 |
| | | protein | |
| TA25994_3708 | AT1G50900.1 | unknown | 0.9 |
| DW999632 | AT3G56910.1 | RSRP5 | 0.9 |
| TA31407_3708 | AT4G15510.3/ | photosystem II reaction | 0.9 |
| | AT4G15510.1 | centre PsbP family | |
| | | protein | |
| TA27488_3708 | AT4G25050.1 | ACP4 | 0.9 |
| EV177713 | AT4G31560.1 | HCF153 | 0.9 |
| EV106558 | AT5G39210.1 | CRR7 | 0.9 |
| DW998509 | AT5G44520.1 | ribose 5-phosphate | 0.9 |
| | | isomerase-related | |
| TC97093 | AT5G65220.1 | ribosomal protein L29 | 0.9 |
| | | family protein | |

TABLE 20
Network IIa: FC ≧ 1.5 (110 vs 115) and Pearson
correlation coefficient > 0.90

| Systematic Name | AGI | Annotation | Pearson |
|---|---|---|---|
| TA21968_3708 | AT1G01100.4/ | 60S acidic ribosomal | 0.9 |
| | AT1G01100.2/ | protein P1 (RPP1A) | |
| | AT1G01100.1 | | |
| TA27876_3708 | AT1G73940.1 | unknown | 0.9 |
| TA21585_3708 | AT4G15000.1 | 60S ribosomal | 0.9 |
| | | protein L27 (RPL27) | |
| CX281365 | AT4G30330.1 | small nuclear ribonucleo- | 0.9 |
| | | protein E, putative | |
| TA23048_3708 | AT5G27700.1 | structural constituent of | 0.9 |
| | | ribosome | |
| TA21968_3708 | AT5G47700.2/ | 60S acidic ribosomal | 0.9 |
| | AT5G47700.1 | protein P1 (RPP1C) | |
| TA21093_3708 | AT5G57290.3/ | 60S acidic ribosomal | 0.9 |
| | AT5G57290.2/ | protein P3 (RPP3B) | |
| | AT5G57290.1 | | |

TABLE 21
Network IIb: FC ≧ 1.5 (110 vs 115) and Pearson
correlation coefficient > 0.90

| Systematic Name | AGI | Annotation | Pearson |
|---|---|---|---|
| CD821628 | AT1G67350.2/ | 11 kDa subunit of | 0.9 |
| | AT1G67350.1 | complex I | |
| DY017585 | AT4G32470.1 | ubiquinol-cytochrome | 0.9 |
| | | C reductase complex | |
| | | 14 kDa protein, putative | |

### 4. Development of a Quantitative RT-PCR for the Selection of High Energy Use Efficient Plants

RNA was extracted from the same leaf material used for transcript profiling as described in Example 2 to characterize expression characteristics of 4 genes. Specifically, 4 genes were selected from Table 2, i.e. the list of transcripts which are upregulated in the high vigor hybrid line HV110. These four genes, which are depicted in Table 22, encode subunits of the mitochondrial respiratory chain.

First-strand cDNA was prepared from 2.5 mg of total RNA, Superscript II RNaseH Reverse Transcriptase (Invitrogen) and a oligo(dT) 15 primer. Five microliters of a 1:12 diluted first-strand cDNA was used as a template in the subsequent PCR, which was performed on the iCycler iQ (BioRad, Hercules, Calif.) with 200 nM primers and Platinum SYBR green Supermix-UGD (2′) (Invitrogen) in a final volume of 25 ml per reaction, according to manufacturer's instructions. All PCRs were performed at least in triplicate. For each of the selected transcripts, the sequence of the Brassica napus cDNA or EST was used to design gene-specific primers with the Beacon Designer™ software. Two housekeeping genes (BAR and polypyrimidine tract binding protein (PTBP)) were used for normalization of the data.

TABLE 22
list of the 4 selected upregulated genes used to design a quantitative RT-PCR.

| Gene | IdProbe_Agilent | Description | AGI | Annotation |
|---|---|---|---|---|
| Gene 1 | EV184671 | gb\|0159533 Brassica napus etiolated | AT2G42310 | NDU12-1; plant- |
| | | seedlings (pSPORT1) Brassica napus | | specific subunit |
| | | cDNA, mRNA sequence [EV184671] | | of complex I |
| Gene 2 | TA24414_3708 | unknown | AT5G59613 | ATP 6 kDa; subunit |
| | | | | of complex V |
| Gene 3 | DY017585 | gb\|63JKCOT5_T3_005_A01_04JAN2005_015 | AT4G32470 | ubiquinol-cytochrome |
| | | 63JKCOT5 Brassica napus | | C reductase complex |
| | | cDNA 5′, mRNA sequence [DY017585] | | 14 kDa protein; putative |
| | | | | (QCR7-1) (complex III) |
| Gene 4 | EE560078 | gb\|BNZB_UP_053_D11_13MAY2004_089 | AT3G62790 | NDUFS5: 15 kDa |
| | | Brassica napus BNZB Brassica | | subunit (complex I) |
| | | napus cDNA 5′, mRNA sequence | | |
| | | [EE560078] | | |

Gene-specific primers used to quantify six selected transcripts are depicted in table 23.

TABLE 23
gene specific primers designed to carry out the Quantitative RT-PCR.

| | | Seq ID | | Seq ID |
|---|---|---|---|---|
| Transcript | Forward primer | No | Reverse primer | No |
| Gene 1 | 5′-GCGTCCCAAGGGCTTCTTC-3′ | 134 | 5′-GCTCCCAATCCTCCCATTTCC-3′ | 136 |
| Gene 2 | 5′-ACCGAGGAAGACACCAAGAAC-3′ | 137 | 5′-GAGAGACGAACAGTATCAAGAATCC-3′ | 138 |
| Gene 3 | 5′-ACAGATTGCCCAGGGAGGTC-3′ | 139 | 5′-GGTACTCGTGCTTCATGGAGAG-3′ | 140 |
| Gene 4 | 5′-GGGAGATCGGAATCAGGTTATTTG-3′ | 141 | 5′-ATCCATCCAGAAATCGTAACATCTC-3′ | 142 |
| BAR | 5′-GCACCATCGTCAACCACTACAT-3′ | 143 | 5′-GTCCACTCCTGCGGTTCCT-3′ | 144 |
| PTBP | 5′-ACACAAATCCATACCTTCCAGTGAA-3′ | 145 | 5′-ACCCAAAGCAGGCTGCATAG-3′ | 146 |

Table 24 shows the difference in expression level for the 4 genes between the high vigor hybrid line HV110 and the control hybrid line 115.

TABLE 24
summary of the results obtained from the Quantitative
RT-PCR for the 4 genes. The level of upregulation
for each of the 4 genes in HV110 with respect to the
control hybrid line 115 is depicted in column 2.

| Upregulated gene | Fold upregulation in HV110 |
|---|---|
| Gene 1 | 4.4 |
| Gene 2 | 3.8 |
| Gene 3 | 4.6 |
| Gene 4 | 5.75 |

### 5. Transcript Profiling in High Energy Efficient Hybrids and Control Hybrid Plants of Brassica napus and Identification of Transcriptional Networks in the High Vigor Brassica Hybrids

The transcriptomes of two high-EUE B. napus (also designated as the high vigor hybrid lines (HV110 and HV112), showing lower respiration levels, were compared to the transcriptome of the B. napus control hybrid line (also designated as B. napus control line 115).

Leaf 3 was harvested from five trays, each containing 4 to 5 plants from line HV110, HV112 and control line 115. For each line (3 replicas/line), RNA was isolated from leafs harvested from different trays and pooled, resulting in 3 samples/line for hybridization to microarrays.

The analysis was performed on a high density CombiMatrix 90K Brassica oligonucleotide array produced by the Plant Functional Genomics Center at the University of Verona. The estimated genome coverage of this array is 65% based on homology with Arabidopsis thaliana. This microarray contains 90,500 probes sourced from EST generated by the Brassica Genomics consortium (http://brassicagenomics.ca/ests/).

As an RNA source for hybridization 3 replicas/line were used. Probe filtering was followed by quantile normalization. The intensities for 55,994 probes were retained. Limma and qvalue packages for R Bioconducter were used for further analysis. Pairwise comparison (t-test) resulted in 603 transcripts with a q value lower than 0.05 between line HV110 and the control line 115 and 655 transcripts significantly differential between line HV112 and the control line 115.

Out of the 603 differential transcripts between line HV110 and control line 115, 582 are at least more than 2 fold upregulated and 21 are at least 2 fold downregulated. Out of the 655 transcripts differential between line HV112 and control line 115, 624 are at least 2 fold upregulated and 31 are at least 2 fold down-regulated.

The lists of 2 fold upregulated transcripts in line HV110 and line HV112 were used to build transcriptional networks. The input list for CORNET after removal of doubles or Brassica IDs without an Arabidopsis homolog for line HV110 is 485 AGI codes. For 55 AGI codes no reliable probe sets were found according to the CDF file used by CORNET, which results in an input list of 430 AGI codes. The selected experiments (1488 in total) include arrays from abiotic stress (256 exp), AtGenExpress All (425 exp), development (135 exp), hormone treatment (140 exp), microarray compendium 2 (111 exp—no bias), stress (abiotic+biotic) (336 exp) and whole plant (85 exp). The selected databases for identification of protein-protein interaction include the Bar, IntAct and TAIR databases. We can identify several networks with a Pearson correlation coefficient higher than 0.80. In the largest network, we can identify a cluster of coregulated genes enriched in mitochondrial genes linked to genes involved in translation. Another cluster of coregulated genes from the largest network is enriched in chloroplast-located proteins.

The input list for CORNET after removal of doubles or Brassica IDs without an Arabidopsis homolog for line HV112 is 514 AGI codes. For 63 AGI codes no reliable probe sets were found according to the CDF file used by CORNET, which results in an input list of 451 AGI codes. The selected experiments (1488 in total) include arrays from abiotic stress (256 exp), AtGenExpress All (425 exp), development (135 exp), hormone treatment (140 exp), microarray compendium 2 (111 exp—no bias), stress (abiotic+biotic) (336 exp) and whole plant (85 exp). The selected databases for identification of protein-protein interaction include the Bar, IntAct and TAIR databases. We can identify several networks with a Pearson correlation coefficient higher than 0.80. In the largest network, we can again identify a cluster of coregulated genes enriched in mitochondrial genes linked to genes involved in translation. Another cluster of coregulated genes from the largest network is enriched in chloroplast-located proteins.

TABLE 25
Mitochondrial network linked to genes involved in translation: FC ≧ 2 (110vs115) and Pearson correlation coefficient >0.8.

| | | FC | | | | | |
|---|---|---|---|---|---|---|---|
| | | (110 | | | | | |
| | | vs. | Q. | | | Seq | |
| Probe Id | Name | 115) | value | AGI | annotation | ID No | function |
| gi_32525687_NCBI_0_0_0_117_539\| | gi\|32525687\|gb\|CD843747.1\| | 165.4 | 4.00E−5 | AT3G62290.1 | ATARFA1E (ADP-RIBOSYLATION | 147 | |
| sense\|_161_195 | CD843747 | | | AT3G62290.2 | FACTOR A1E) | | |
| | | | | AT3G62290.3 | | | |
| Contig24_1883_final_0_0_0_62_268\| | gi\|83822180\|gb\|CX270403.1\| | 64.0 | 9.00E−05 | AT3G59540.1 | 60S ribosomal protein L38 (RPL38A) | 148 | T |
| sense\|_276_311 | CX270403 | | | AT2G43460.1 | | | |
| 49RDOATR_UP_046_H11_24OCT2004_081.ab1_PBI_0_0_0_15_254\| | gi\|150149702\|gb\|EE552105.1\| | 45.5 | 9.00E−05 | AT1G31730.1 | epsilon-adaptin, putative | 149 | |
| sense\|_222_256 | EE552105 | | | | | | |
| Contig2_8545_final_0_0_0_204_728\| | gi\|32517661\|gb\|CD835721.1\| | 42.8 | 9.00E−05 | AT4G34460.1 | AGB1 (GTP BINDING PROTEIN | 150 | |
| sense\|_615_649 | CD835721 | | | AT4G34460.2 | BETA 1) | | |
| | | | | AT4G34460.3 | | | |
| | | | | AT4G34460.4 | | | |
| Contig1_17115_final_0_0_0_2_430\|sense\|_81_115 | gi\|125943277\|gb\|EL592992.1\| | 39.9 | 1.00E−04 | AT5G14520.1 | pescadillo-related | 151 | |
| | EL592992 | | | | | | |
| Contig1_82325_remaining_0_0_0_283_1188\| | gi\|151201513\|gb\|EV114559.1\| | 35.6 | 1.00E−04 | AT5G53530.1 | vacuolar protein sorting-associated | 152 | |
| sense\|_997_1031 | EV114559 | | | | protein 26, putative | | |
| Contig4_1758_final_0_0_0_13_1137\| | gi\|95840952\|gb\|DY016483.1\| | 34.6 | 1.00E−04 | AT3G15000.1 | hypothetical protein | 153 | |
| sense\|_617_652 | DY016483 | | | | | | |
| Contig2_13142_final_0_0_0_30_398\| | gi\|150065154\|gb\|EE427893.1\| | 31.9 | 1.00E−04 | AT1G56090.1 | tetratricopeptide repeat (TPR)- | 154 | |
| sense\|_743_777 | EE427893 | | | | containing protein | | |
| Contig2_1039_final_0_0_0_136_780\| | gi\|126501180\|gb\|EE470903.1\| | 31.3 | 2.00E−04 | AT3G25040.1 | ER lumen protein retaining receptor, | 155 | |
| sense\|_788_822 | EE470903 | | | | putative/HDEL receptor, putative | | |
| Contig1_702_final_0_0_0_72_341\|sense\|_332_368 | gi\|83833667\|gb\|CX281890.1\| | 30.9 | 1.00E−04 | AT2G20820.1 | expressed protein | 156 | |
| | CX281890 | | | AT2G20820.2 | | | |
| Contig4_782_final_0_0_0_48_404\|sense\|_190_224 | gi\|119424582\|gb\|DY009984.1\| | 27.4 | 9.00E−05 | AT2G19740.1 | 60S ribosomal protein L31 (RPL31A) | 157 | T |
| | DY009984 | | | | | | |
| Contig1_7767_final_0_0_0_141_1091\| | gi\|95840104\|gb\|DY016080.1\| | 26.7 | 9.00E−05 | AT4G23740.1 | leucine-rich repeat transmembrane | 158 | |
| sense\|_957_991 | DY016080 | | | | protein kinase, putative | | |
| gi_21843860_NCBI\|senAnsen\|_184_220 | gi\|21843860\|gb\|BQ704441.1\| | 24.4 | 2.00E−04 | AT4G10610.1 | RBP37 (RNA-BINDING PROTEIN | 159 | |
| | BQ704441 | | | AT4G10610.2 | 37); RNA binding (CID12) | | |
| gi_32499613_NCBI_0_0_0_6_317\| | gi\|32499613\|gb\|CD817673.1\| | 22.6 | 2.00E−04 | AT1G63810.1 | nucleolar RNA-associated family | 160 | |
| sense\|_168_202 | CD817673 | | | | protein | | |
| Contig13_5494_final_0_0_0_90_383\| | gi\|95828809\|gb\|DW999284.1\| | 21.5 | 0.005 | AT1G14980.1 | CPN10 (CHAPERONIN 10) | 161 | M |
| sense\|_50_84 | DW999284 | | | | | | |
| Contig3_6771_final_0_0_0_34_390\|sense\|_8_46 | gi\|32512640\|gb\|CD830700.1\| | 21.4 | 6.00E−04 | AT3G59650.1 | mitochondrial ribosomal protein | 162 | M |
| | CD830700 | | | AT3G59650.2 | L51/S25/CI-B8 family protein | | |
| Contig1_17597_final_0_0_0_3_746\|sense\|_209_243 | gi\|150878730\|gb\|ES909188.1\| | 20.4 | 1.00E−04 | AT1G59540.1 | ZCF125; microtubule motor | 163 | |
| | ES909188 | | | AT1G59540.2 | | | |
| Contig1_1343_final_0_0_0_86_520\|sense\|_64_103 | gi\|150057077\|gb\|EE419840.1\| | 20.2 | 7.00E−04 | AT4G21110.1 | G10 family protein | 164 | |
| | EE419840 | | | | | | |
| Contig1_2213_final_0_0_0_96_1157\| | gi\|150927189\|gb\|ES957652.1\| | 19.6 | 5.00E−04 | AT4G37210.1 | tetratricopeptide repeat (TPR)- | 165 | |
| sense\|_798_832 | ES957652 | | | AT4G37210.2 | containing protein | | |
| Contig1_71772_remaining_0_0_0_2_454\| | gi\|125934956\|gb\|EL589211.1\| | 19.5 | 0.001 | AT4G11790.1 | Ran-binding protein 1 domain- | 166 | |
| sense\|_281_315 | EL589211 | | | | containing protein | | |
| Contig1_62949_remaining_0_0_0_3_107\| | gi\|150871714\|gb\|ES902175.1\| | 18.8 | 0.004 | AT2G35040.1 | AICARFT/IMPCHase bienzyme | 167 | |
| sense\|_305_339 | ES902175 | | | AT2G35040.2 | family protein | | |
| Contig1_42742_remaining_0_0_0_80_511\| | gi\|150162948\|gb\|EE555762.1\| | 18.4 | 5.00E−04 | AT1G08880.1 | G-H2AX/GAMMA- | 168 | |
| sense\|_113_168 | EE555762 | | | | H2AX/H2AXA/HTA5 | | |
| Contig4_9922_final_0_0_0_2_1498\|sense\|_780_817 | gi\|119430188\|gb\|DY020917.1\| | 17.5 | 3.00E−04 | AT2G21790.1 | R1/RNR1 (RIBONUCLEOTIDE | 169 | |
| | DY020917 | | | | REDUCTASE 1) | | |
| OL105_108R_O15_OL10216R_047.ab1_AAFC_0_0_0_3_551\| | gi\|32495522\|gb\|CD813582.1\| | 17.4 | 0.004 | AT2G17980.1 | ATSLY1; protein transporter | 170 | |
| sense\|_147_181 | CD813582 | | | | | | |
| Contig3_1253_final_0_0_0_2_529\|sense\|_11_45 | gi\|32511884\|gb\|CD829944.1\| | 16.9 | 0.004 | AT1G12000.1 | pyrophosphate-fructose-6-phosphate | 171 | |
| | CD829944 | | | | 1-phosphotransferase beta subunit, | | |
| | | | | | putative | | |
| 65JKBNM0_T3_013_G02_17MAR2005_004.ab1_PBI_0_0_0_105_329\| | gi\|21843795\|gb\|BQ704376.1\| | 16.8 | 6.00E−04 | AT2G40550.1 | E2F TARGET GENE 1 | 172 | |
| sense\|_28_62 | BQ704376 | | | | | | |
| Contig2_11526_final_0_0_0_81_653\| | gi\|122028534\|gb\|EH417399.1\| | 16.4 | 9.00E−04 | AT3G17940.1 | aldose 1-epimerase family protein | 173 | |
| sense\|_594_628 | EH417399 | | | | | | |
| Contig2_11169_final_0_0_0_61_831\| | gi\|75970744\|gb\|AM057198.1\| | 16.3 | 2.00E−04 | AT4G27490.1 | 3′ exoribonuclease family domain 1- | 174 | |
| sense\|_737_771 | AM057198 | | | | containing protein | | |
| DC2273R_AAFC_0_0_0_49_441\|sense\|_257_291 | gi\|151326308\|gb\|EV226299.1\| | 15.8 | 0.002 | AT2G01140.1 | fructose-bisphosphate aldolase, | 175 | |
| | EV226299 | | | | putative | | |
| Contig2_12321_final_0_0_0_93_686\| | gi\|65298993\|gb\|CN829207.1\| | 15.1 | 0.001 | AT5G11900.1 | eukaryotic translation initiation factor | 176 | T |
| sense\|_610_644 | CN829207 | | | | SUI1 family protein | | |
| Contig6_8389_final_0_0_0_214_621\| | gi\|150061558\|gb\|EE424297.1\| | 14.1 | 0.043 | AT1G10840.1 | TIF3H1 (eIF3 subunit H1) | 177 | T |
| sense\|_209_243 | EE424297 | | | AT1G10840.2 | | | |
| 39RDBRT_UP_069_E04_30NOV2005_024.ab1_PBI_0_0_0_321_497\| | gi\|150116248\|gb\|EE517220.1\| | 13.6 | 9.00E−04 | AT1G63470.1 | DNA-binding family protein | 178 | |
| sense\|_126_161 | EE517220 | | | | | | |
| Contig1_2842_final_0_0_0_87_653\|sense\|_324_359 | gi\|150898448\|gb\|ES928906.1\| | 13.2 | 0.009 | AT1G76010.1 | nucleic acid binding | 179 | |
| | ES928906 | | | | | | |
| Contig1_68996_remaining_0_0_0_3_422\| | gi\|150132975\|gb\|EE533945.1\| | 12.8 | 1.00E−04 | AT5G19400.1 | expressed protein | 180 | |
| sense\|_109_143 | EE533945 | | | AT5G19400.2 | | | |
| | | | | AT5G19400.3 | | | |
| 72ETGS24_UP_003_B03_18MAY2005_029.ab1_PBI_0_0_0_2_247\| | gi\|150080405\|gb\|EE443144.1\| | 12.6 | 7.00E−04 | AT3G43920.1 | DCL3 (DICER-LIKE 3) | 181 | |
| sense\|_81_115 | EE443144 | | | AT3G43920.2 | | | |
| | | | | AT3G43920.3 | | | |
| Contig5_72_final_0_0_0_63_380\|sense\|_761_799 | gi\|150152399\|gb\|EE556824.1\| | 12.2 | 0.003 | AT2G27710.1 | 60S acidic ribosomal protein P2 | 182 | T |
| | EE556824 | | | AT2G27710.2 | (RPP2B) | | |
| | | | | AT2G27710.3 | | | |
| | | | | AT2G27710.4 | | | |
| Contig1_2482_final_0_0_0_22_615\|sense\|_558_592 | gi\|32494572\|gb\|CD812632.1\| | 11.7 | 0.036 | AT5G62290.1 | nucleotide-sensitive chloride | 183 | |
| | CD812632 | | | AT5G62290.2 | conductance regulator (ICIn) family | | |
| | | | | | protein | | |
| Contig3_3191_final_0_0_0_2_634\|sense\|_468_502 | gi\|95828892\|gb\|DW999367.1\| | 11.4 | 0.001 | AT3G13670.1 | protein kinase family protein | 184 | |
| | DW999367 | | | | | | |
| 24RDBNH_UP_037_B04_20FEB2004_030.ab1_PBI_0_0_0_108_497\| | gi\|150105739\|gb\|EE506711.1\| | 11.2 | 0.007 | AT2G03680.1 | SPR1 (SPIRAL1) | 185 | |
| sense\|_401_435 | EE506711 | | | AT2G03680.2 | | | |
| 36RDBRG_UP_062_B10_29NOV2005_078.ab1_PBI_0_0_0_3_356\| | gi\|150120217\|gb\|EE521189.1\| | 11.2 | 0.013 | AT3G03580.1 | pentatricopeptide (PPR) repeat- | 186 | |
| sense\|_42_76 | EE521189 | | | | containing protein | | |
| Contig1_1450_final_0_0_0_121_576\| | gi\|56837824\|gb\|CX190400.1\| | 11.0 | 0.001 | AT1G14400.1 | UBC1 (UBIQUITIN CARRIER | 187 | |
| sense\|_539_573 | CX190400 | | | AT1G14400.2 | PROTEIN 1) | | |
| LD6126F_AAFC_0_0_0_1_255\|sense\|_191_225 | gi\|151188650\|gb\|EV102123.1\| | 10.6 | 2.00E−04 | AT5G54670.1 | kinesin-like protein A | 188 | |
| | EV102123 | | | | | | |
| Contig2_3405_final\|senAnsen\|_57_91 | gi\|151179078\|gb\|EV092585.1\| | 10.5 | 0.002 | AT2G27450.1 | NLP1 (NITRILASE-LIKE PROTEIN 1) | 189 | |
| | EV092585 | | | AT2G27450.2 | | | |
| Contig1_11805_final_0_0_0_3_392\|sense\|_340_377 | gi\|83833547\|gb\|CX281770.1\| | 10.1 | 0.003 | AT3G07050.1 | GTP-binding family protein | 190 | |
| | CX281770 | | | | | | |
| CL3599F_AAFC_0_0_0_46_291\|sense\|_133_167 | gi\|151293685\|gb\|EV200346.1\| | 10.0 | 0.002 | AT2G04660.1 | APC2 (anaphase-promoting | 191 | |
| | EV200346 | | | | complex/cyclosome 2) | | |
| Contig2_2378_final_0_0_0_20_379\|sense\|_190_225 | gi\|150887215\|gb\|ES917673.1\| | 10.0 | 0.003 | AT1G26880.1 | 60S ribosomal protein L34 (RPL34A) | 192 | M |
| | ES917673 | | | AT1G26880.2 | | | |
| Contig1_23244_remaining_0_0_0_3_1172\| | gi\|112354818\|gb\|AM390327.1\| | 9.5 | 7.00E−04 | AT2G01750.1 | ATMAP70-3 (microtubule-associated | 193 | |
| sense\|_512_546 | AM390327 | | | AT2G01750.2 | proteins 70-3) | | |
| gi_113704882_NCBI_0_0_0_9_479\| | gi\|113704882\|gb\|AM394050.1\| | 8.9 | 0.001 | AT5G46070.1 | GTP binding/GTPase | 194 | |
| sense\|_408_446 | AM394050 | | | | | | |
| 49RDOAT_UP_052_H12_27JAN2006_082.ab1_PBI_0_0_0_1_576\| | gi\|150917098\|gb\|ES947559.1\| | 8.7 | 0.002 | AT2G36200.1 | kinesin motor protein-related | 195 | |
| sense\|_423_457 | ES947559 | | | AT2G36200.2 | | | |
| BNSCS2CT_UP_031_F12_07JAN2005_086.ab1_PBI_0_0_0_3_122\| | gi\|126479535\|gb\|EE464699.1\| | 8.1 | 0.026 | AT2G45280.1 | ATRAD51C (Arabidopsis thaliana | 196 | |
| sense\|_63_102 | EE464699 | | | AT2G45280.2 | Ras Associated with Diabetes protein | | |
| | | | | | 51C) | | |
| Contig2_2455_final_0_0_0_1_951\|sense\|_145_179 | gi\|150055308\|gb\|EE418162.1\| | 8.1 | 0.03 | AT1G06720.1 | expressed protein | 197 | |
| | EE418162 | | | | | | |
| Contig3_6028_final_0_0_0_72_608\|sense\|_13_47 | gi\|65293342\|gb\|CN735527.1\| | 7.8 | 0.026 | AT2G38130.1 | ATMAK3 (Arabidopsis thaliana MAK3 | 198 | |
| | CN735527 | | | AT2G38130.2 | homologue); N-acetyltransferase | | |
| 46RDOAG_UP_018_D05_27SEP2004_041.ab1_PBI_0_0_0_2_349\| | gi\|150130383\|gb\|EE531355.1\| | 7.7 | 0.028 | AT2G38770.1 | EMB2765 | 199 | |
| sense\|_22_56 | EE531355 | | | | | | |
| Contig2_5461_final_0_0_0_2_118\|sense\|_451_488 | gi\|150140559\|gb\|EE541522.1\| | 7.5 | 0.011 | AT2G27040.1 | AGO4 (ARGONAUTE 4) | 200 | |
| | EE541522 | | | AT2G27040.2 | | | |
| 59ACAB6_UP_012_H10_23JUL2004_066.ab1_PBI_0_0_0_35_592\| | gi\|65295054\|gb\|CN737235.1\| | 7.5 | 0.005 | AT4G30480.1 | tetratricopeptide repeat (TPR)- | 201 | M |
| sense\|_392_429 | CN737235 | | | AT4G30480.2 | containing protein | | |
| | | | | AT4G30480.3 | | | |
| Contig31_1833_final_0_0_0_43_531\| | gi\|65293240\|gb\|CN735425.1\| | 7.2 | 0.002 | AT5G48580.1 | FKBP15-2 (FK506-binding protein 15 kD- | 202 | |
| sense\|_524_562 | CN735425 | | | | 2) | | |
| 31ETGS12_UP_014_B10_07FEB2005_078.ab1_PBI_0_0_0_98_346\| | gi\|150061888\|gb\|EE424627.1\| | 6.9 | 0.039 | AT5G11500.1 | expressed protein | 203 | |
| sense\|_75_111 | EE424627 | | | AT5G11500.2 | | | |
| Contig22_1329_final_0_0_0_41_679\| | gi\|54419450\|gb\|CV545628.1\| | 6.6 | 0.025 | AT1G02780.1 | EMB2386 (EMBRYO DEFECTIVE | 204 | |
| sense\|_10_45 | CV545628 | | | | 2386) | | |
| 47RDOAH_UP_043_A05_20AUG2004_047.ab1_PBI\| | gi\|95861799\|gb\|DY029554.1\| | 6.5 | 0.009 | AT2G19480.1 | NAP1;2/NFA2 (NUCLEOSOME | 205 | |
| senAnsen\|_25_59 | DY029554 | | | AT2G19480.2 | ASSEMBLY PROTEIN1;2) | | |
| | | | | AT2G19480.3 | | | |
| Contig1_7160_final_0_0_0_4_477\|sense\|_117_151 | gi\|151310405\|gb\|EV210442.1\| | 6.2 | 0.025 | AT3G23300.1 | dehydration-responsive protein- | 206 | |
| | EV210442 | | | | related | | |
| ML3653F_AAFC\|senAnsen\|_18_52 | gi\|65296856\|gb\|CN827070.1\| | 6.2 | 0.019 | AT2G21870.1 | Identical to Probable ATP synthase | 207 | M |
| | CN827070 | | | AT2G21870.2 | 24 kDa subunit, mitochondrial | | |
| | | | | | precursor | | |
| RL5583R_AAFC_0_0_0_249_686\| | gi\|151209129\|gb\|EV122170.1\| | 6.0 | 0.01 | AT5G11500.1 | expressed protein | 208 | |
| sense\|_274_309 | EV122170 | | | AT5G11500.2 | | | |
| Contig1_7648_final_0_0_0_49_726\|sense\|_589_623 | gi\|65294419\|gb\|CN736602.1\| | 5.9 | 0.003 | AT4G31930.1 | mitochondrial glycoprotein family | 209 | M |
| | CN736602 | | | | protein/MAM33 family protein | | |
| 9RDBNGA_UP_072_E09_03APR2005_071.ab1_PBI_0_0_0_2_577\| | gi\|150144783\|gb\|EE545746.1\| | 5.8 | 1.00E−03 | AT4G02390.1 | APP (ARABIDOPSIS POLY(ADP- | 210 | |
| sense\|_25_9 | EE545746 | | | | RIBOSE) POLYMERASE) | | |
| Contig3_10313_final_0_0_0_3_500\|sense\|_448_482 | gi\|65293469\|gb\|CN735652.1\| | 5.8 | 0.002 | AT3G24830.1 | 60S ribosomal protein L13A | 211 | T |
| | CN735652 | | | | (RPL13aA) | | |
| Contig26_886_final_0_0_0_69_281\|sense\|_5_39 | gi\|32494420\|gb\|CD812480.1\| | 5.7 | 0.013 | AT2G31490.1 | NDU8: plant specific subunit | 212 | M |
| | CD812480 | | | | | | |
| BNRoot1_UP_134_I10_10DEC2003_054.ab1_GHI1_0_0_0_1_198\| | gi\|151316804\|gb\|EV216841.1\| | 5.6 | 0.008 | AT1G69420.1 | zinc finger (DHHC type) family | 213 | |
| sense\|_311_345 | EV216841 | | | AT1G69420.2 | protein | | |
| Contig8_2355_final_0_0_0_94_486\|sense\|_9_43 | gi\|150041851\|gb\|EE404725.1\| | 5.4 | 0.008 | AT4G31720.1 | TAFII15 (SALT TOLERANCE | 214 | |
| | EE404725 | | | AT4G31720.2 | DURING GERMINATION 1) | | |
| 8RDBRH_UP_023_D08_17SEP2003_058.ab1_PBI_0_0_0_75_263\| | gi\|119425262\|gb\|DY013379.1\| | 5.4 | 0.023 | AT3G17210.1 | stable protein 1-related | 215 | |
| sense\|_233_269 | DY013379 | | | | | | |
| Contig2_4733_final_0_0_0_64_573\|sense\|_408_442 | gi\|32509330\|gb\|CD827390.1\| | 5.4 | 0.037 | AT5G52920.1 | PKP-BETA1/PKP1/PKP2 | 216 | |
| | CD827390 | | | | (PLASTIDIC PYRUVATE KINASE 1) | | |
| Contig4_632_final_0_0_0_2_280\|sense\|_162_196 | gi\|113704618\|gb\|AM394581.1\| | 5.3 | 0.002 | AT5G61130.1 | glycosyl hydrolase family protein 17 | 217 | |
| | AM394581 | | | | | | |
| VA1513F_AAFC\|senAnsen\|_16_55 | gi\|56834987\|gb\|CX187563.1\| | 5.2 | 0.033 | AT3G48000.1 | ALDH2B4 (ALDEHYDE | 218 | |
| | CX187563 | | | | DEHYDROGENASE 2) | | |
| gi_37620869_NCBI_0_0_0_2_373\| | gi\|37620869\|gb\|CA991574.1\| | 5.2 | 0.025 | AT1G18840.1 | IQD30; calmodulin binding | 219 | |
| sense\|_94_128 | CA991574 | | | AT1G18840.2 | | | |
| 11FGYSDB_UP_005_G06_17OCT2003_036.ab1_PBI_0_0_0_1_360\| | gi\|32501027\|gb\|CD819087.1\| | 5.1 | 0.002 | AT5G59880.1 | ADF3 (ACTIN DEPOLYMERIZING | 220 | |
| sense\|_336_371 | CD819087 | | | AT5G59880.2 | FACTOR 3) | | |
| Contig1_3137_final_0_0_0_70_741\|sense\|_48_82 | gi\|150052413\|gb\|EE415272.1\| | 5.1 | 0.019 | AT5G19680.1 | leucine-rich repeat family protein | 221 | |
| | EE415272 | | | | | | |
| Contig4_2857_final_0_0_0_1_387\|sense\|_459_496 | gi\|150089907\|gb\|EE490879.1\| | 5.1 | 0.034 | AT2G17630.1 | phosphoserine aminotransferase, | 222 | |
| | EE490879 | | | | putative | | |
| Contig3_7278_final_0_0_0_73_705\|sense\|_488_522 | gi\|95851397\|gb\|DY023048.1\| | 4.9 | 0.025 | AT2G05710.1 | aconitate hydratase, cytoplasmic, | 223 | |
| | DY023048 | | | | putative | | |
| Contig157_601_final_0_0_0_73_258\| | gi\|32494504\|gb\|CD812564.1\| | 4.8 | 0.022 | AT5G56670.1 | 40S ribosomal protein S30 (RPS30A) | 224 | T |
| sense\|_10_44 | CD812564 | | | | | | |
| EX20LIB7_UP_002_H10_01OCT2004_066.ab1_PBI\| | gi\|150097423\|gb\|EE498395.1\| | 4.7 | 0.013 | AT1G76010.1 | nucleic acid binding | 225 | |
| senAnsen\|_29_63 | EE498395 | | | | | | |
| gi_37621592_NCBI_0_0_0_3_260\| | gi\|37621592\|gb\|CA992297.1\| | 4.7 | 0.01 | AT3G57290.1 | EIF3E | 226 | |
| sense\|_381_415 | CA992297 | | | | | | |
| Contig2_2953_final_0_0_0_1_684\|sense\|_88_122 | gi\|150888173\|gb\|ES918631.1\| | 4.6 | 0.023 | AT2G37340.1 | RSZ33 (ARGININE/SERINE-RICH | 227 | |
| | ES918631 | | | AT2G37340.2 | ZINC KNUCKLE-CONTAINING | | |
| | | | | AT2G37340.3 | PROTEIN 33) | | |
| Contig535_4314_final_0_0_0_88_522\| | gi\|113704806\|gb\|AM395294.1\| | 4.6 | 0.012 | AT2G32060.1 | 40S ribosomal protein S12 (RPS12C) | 228 | T |
| sense\|_66_102 | AM395294 | | | AT2G32060.2 | | | |
| | | | | AT2G32060.3 | | | |
| Contig1_803_final_0_0_0_67_429\|sense\|_264_298 | gi\|32503997\|gb\|CD822057.1\| | 4.6 | 0.045 | AT2G36930.1 | zinc finger (C2H2 type) family protein | 229 | |
| | CD822057 | | | | | | |
| Contig1_1787_final_0_0_0_142_870\| | gi\|32496491\|gb\|CD814551.1\| | 4.4 | 0.044 | AT5G23420.1 | HMGB6 (High mobility group B 6) | 230 | |
| sense\|_550_584 | CD814551 | | | AT5G23420.2 | | | |
| 36RDBRG_UP_089_H08_20JAN2006_050.ab1_PBI_0_0_0_34_351\| | gi\|83818317\|gb\|CX266540.1\| | 4.4 | 0.035 | AT5G18800.1 | NADH-ubiquinone oxidoreductase 19 kDa | 231 | M |
| sense\|_464_499 | CX266540 | | | AT5G18800.2 | subunit (NDUFA8) family protein | | |
| Contig1_17648_final_0_0_0_1_723\|sense\|_513_547 | gi\|151248819\|gb\|EV159239.1\| | 4.3 | 0.008 | AT5G39900.1 | GTP binding/translation elongation | 232 | |
| | EV159239 | | | | factor | | |
| CD3489F_AAFC\|senAnsen\|_191_225 | gi\|150056134\|gb\|EE418988.1\| | 4.3 | 0.021 | AT2G19480.1 | NAP1;2/NFA2 (NUCLEOSOME | 233 | |
| | EE418988 | | | AT2G19480.2 | ASSEMBLY PROTEIN1;2) | | |
| | | | | AT2G19480.3 | | | |
| Contig1_83793_remaining_0_0_0_2_898\| | gi\|95837756\|gb\|DY012168.1\| | 4.3 | 0.021 | AT3G62360.1 | expressed protein | 234 | |
| sense\|_361_395 | DY012168 | | | | | | |
| Contig125_4314_final_0_0_0_227_760\| | gi\|150098125\|gb\|EE499097.1\| | 4.2 | 0.048 | AT2G34480.1 | 60S ribosomal protein L18A | 235 | T |
| sense\|_29_63 | EE499097 | | | | (RPL18aB) | | |
| 9RDBNGA_UP_166_C01_10MAR2006_011.ab1_PBI\| | gi\|150927026\|gb\|ES957489.1\| | 4.2 | 0.039 | AT3G60770.1 | 40S ribosomal protein S13 (RPS13A) | 236 | T |
| senAnsen\|_9_48 | ES957489 | | | | | | |
| BNAEN3GH_UP_236_D07_29NOV2006_057.ab1_PBI_0_0_0_4_201\| | gi\|150995919\|gb\|EV009734.1\| | 4.2 | 0.023 | AT1G74560.1 | NRP1 (NAP1-RELATED PROTEIN 1) | 237 | |
| sense\|_24_58 | EV009734 | | | AT1G74560.2 | | | |
| Contig4_11587_final_0_0_0_3_452\|sense\|_64_100 | gi\|65294412\|gb\|CN736595.1\| | 4.2 | 0.001 | AT1G04170.1 | EIF2 GAMMA subunit | 238 | T |
| | CN736595 | | | | | | |
| gi_113704726_NCBI_0_0_0_135_233\| | gi\|113704726\|gb\|AM395006.1\| | 4.2 | 0.008 | AT1G79030.1 | DNAJ heat shock N-terminal domain- | 239 | |
| sense\|_24_58 | AM395006 | | | | containing protein/S-locus protein, | | |
| | | | | | putative | | |
| Contig3_62_final_0_0_0_153_1616\|sense\|_929_963 | gi\|65297892\|gb\|CN828106.1\| | 4.1 | 0.003 | AT1G11680.1 | CYP51G1 (CYTOCHROME P450 51) | 240 | |
| | CN828106 | | | | | | |
| Contig1_3071_final_0_0_0_9_761\|sense\|_628_662 | gi\|125931919\|gb\|EL588692.1\| | 4.0 | 0.008 | AT5G24840.1 | methyltransferase | 241 | |
| | EL588692 | | | | | | |
| Contig1_84646_remaining_0_0_0_3_194\| | gi\|151214581\|gb\|EV127622.1\| | 3.9 | 0.008 | AT3G52590.1 | UBQ1 (EARLY-RESPONSIVE TO | 242 | |
| sense\|_35_69 | EV127622 | | | | DEHYDRATION 16, UBIQUITIN | | |
| | | | | | EXTENSION PROTEIN 1) | | |
| Contig4_709_final_0_0_0_2_388\|sense\|_227_261 | gi\|56842240\|gb\|CX194816.1\| | 3.9 | 0.049 | AT4G33650.1 | ADL2 (ARABIDOPSIS DYNAMIN- | 243 | |
| | CX194816 | | | AT4G33650.2 | LIKE 2) | | |
| Contig6_4508_final_0_0_0_80_271\|sense\|_4_38 | gi\|83819835\|gb\|CX268058.1\| | 3.8 | 0.049 | AT2G20490.1 | EDA27/NOP10 (embryo sac | 244 | |
| | CX268058 | | | AT2G20490.2 | development arrest 27) | | |
| ESS3313R_AAFC_0_0_0_39_356\| | gi\|151271042\|gb\|EV181403.1\| | 3.7 | 0.044 | AT4G24820.1 | 26S proteasome regulatory subunit, | 245 | |
| sense\|_577_611 | EV181403 | | | AT4G24820.2 | putative (RPN7) | | |
| | | | | AT4G24830.1 | | | |
| | | | | AT4G24830.2 | | | |
| 37RDBRH_UP_020_C12_31MAR2004_092.ab1_PBI_0_0_0_1_372\| | gi\|150122821\|gb\|EE523793.1\| | 3.7 | 0.012 | AT1G75660.1 | XRN3 (5′-3′ exoribonuclease 3) | 246 | |
| sense\|_338_372 | EE523793 | | | | | | |
| LD3466R_AAFC\|senAnsen\|_182_217 | gi\|151037175\|gb\|EV050951.1\| | 3.6 | 0.029 | AT4G31880.1 | expressed protein | 247 | |
| | EV050951 | | | AT4G31880.2 | | | |
| Contig2_10070_final_0_0_0_37_834\| | gi\|95838418\|gb\|DY012500.1\| | 3.5 | 0.043 | AT5G22650.1 | HD2B (HISTONE DEACETYLASE | 248 | |
| sense\|_365_399 | DY012500 | | | AT5G22650.2 | 2B) | | |
| Contig27_1883_final_0_0_0_56_262\| | gi\|83822180\|gb\|CX270403.1\| | 3.5 | 0.039 | AT3G59540.1 | 60S ribosomal protein L38 (RPL38A) | 249 | T |
| sense\|_272_306 | CX270403 | | | AT2G43460.1 | | | |
| Contig6_874_final_0_0_0_45_707\|sense\|_4_38 | gi\|65283735\|gb\|CN725933.1\| | 3.4 | 0.01 | AT1G66580.1 | 60S ribosomal protein L10 (RPL10C) | 250 | T |
| | CN725933 | | | | | | |
| Contig3_702_final_0_0_0_59_331\|sense\|_320_355 | gi\|32494127\|gb\|CD812187.1\| | 3.1 | 0.008 | AT2G20820.1 | expressed protein | 251 | |
| | CD812187 | | | AT2G20820.2 | | | |
| Contig1_6137_final_0_0_0_2_235\|sense\|_104_139 | gi\|150130317\|gb\|EE531289.1\| | 3.0 | 0.039 | AT1G69250.1 | nuclear transport factor 2 (NTF2) | 252 | |
| | EE531289 | | | | family protein | | |
| Contig3_673_final_0_0_0_61_714\|sense\|_19_53 | gi\|32508529\|gb\|CD826589.1\| | 3.0 | 0.044 | AT3G53970.1 | proteasome inhibitor-related | 253 | |
| | CD826589 | | | AT3G53970.2 | | | |
| Contig1_416_final_0_0_0_45_407\|sense\|_504_543 | gi\|32494036\|gb\|CD812096.1\| | 2.8 | 0.015 | AT5G57290.1 | 60S acidic ribosomal protein P3 | 254 | T |
| | CD812096 | | | AT5G57290.3 | (RPP3B) | | |
| BNZB_UP_034_D10_10MAY2004_074.ab1_PBI\| | gi\|150153496\|gb\|EE558749.1\| | 2.7 | 0.045 | AT4G39520.1 | GTP-binding protein, putative | 255 | |
| senAnsen\|_289_326 | EE558749 | | | | | | |
| DL2930R_AAFC_0_0_0_8_211\|sense\|_249_287 | gi\|151015701\|gb\|EV029515.1\| | 2.6 | 0.044 | AT3G48000.1 | ALDH2B4 (ALDEHYDE | 256 | |
| | EV029515 | | | | DEHYDROGENASE 2) | | |
| Contig4_613_final_0_0_0_47_499\|sense\|_448_483 | gi\|32512821\|gb\|CD830881.1\| | 2.6 | 0.039 | AT5G23290.1 | c-myc binding protein, | 257 | |
| | CD830881 | | | | putative/prefoldin, putative | | |
| Contig5_8154_final_0_0_0_122_457\| | gi\|125935004\|gb\|EL589238.1\| | 2.5 | 0.041 | AT1G77940.1 | 60S ribosomal protein L30 (RPL30B) | 258 | T |
| sense\|_64_98 | EL589238 | | | | | | |
| Contig3_6281_final_0_0_0_118_603\| | gi\|126505238\|gb\|EE482879.1\| | 2.5 | 0.02 | AT5G60790.1 | ATGCN1 (Arabidopsis thaliana | 259 | |
| sense\|_476_510 | EE482879 | | | | general control non-repressible 1) | | |
| Contig1_17404_final_0_0_0_90_296\| | gi\|32500912\|gb\|CD818972.1\| | 2.5 | 0.028 | AT1G15120.1 | ubiquinol-cytochrome C reductase | 260 | M |
| sense\|_61_95 | CD818972 | | | AT1G15120.2 | complex 7.8 kDa protein, putative | | |
| Contig1_4945_final_0_0_0_42_314\|sense\|_25_59 | gi\|126493362\|gb\|EE453044.1\| | 2.5 | 0.049 | AT5G25570.1 | expressed protein | 261 | |
| | EE453044 | | | AT5G25570.2 | | | |
| | | | | AT5G25570.3 | | | |
| Contig1_15045_final_0_0_0_79_426\| | gi\|95853955\|gb\|DY026350.1\| | 2.5 | 0.017 | AT3G52090.1 | ATRPB13.6 (Arabidopsis thaliana | 262 | |
| sense\|_23_57 | DY026350 | | | AT3G52090.2 | RNA polymerase II 13.6 kDa subunit) | | |
| 8RDBRH_UP_017_B02_16SEP2003_014.ab1_PBI_0_0_0_82_372\| | gi\|119424723\|gb\|DY010125.1\| | 2.4 | 0.031 | AT1G21190.1 | small nuclear ribonucleoprotein, | 263 | |
| sense\|_372_410 | DY010125 | | | | putative | | |
| BNARO5GH_T3_005_F09_29NOV2006_069.ab1_PBI_0_0_0_1_654\| | gi\|150074447\|gb\|EE437186.1\| | 2.4 | 0.035 | AT1G80350.1 | ERH3 (ECTOPIC ROOT HAIR 3) | 264 | |
| sense\|_147_181 | EE437186 | | | | | | |
| Contig9_3432_final_0_0_0_86_739\|sense\|_646_681 | gi\|56842813\|gb\|CX195389.1\| | 2.4 | 0.028 | AT5G52240.1 | MSBP1 (MEMBRANE STEROID | 265 | |
| | CX195389 | | | AT5G52240.2 | BINDING PROTEIN 1) | | |
| Contig3_3111_final_0_0_0_56_262\|sense\|_268_306 | gi\|95843033\|gb\|DY017359.1\| | 2.3 | 0.036 | AT2G43460.1 | 60S ribosomal protein L38 (RPL38A) | 266 | T |
| | DY017359 | | | | | | |
| Contig5_3111_final_0_0_0_56_262\|sense\|_92_127 | gi\|150073135\|gb\|EE435874.1\| | 2.2 | 0.022 | AT2G43460.1 | 60S ribosomal protein L38 (RPL38A) | 267 | T |
| | EE435874 | | | | | | |
| 55ACACPE_UP_018_B06_23DEC2004_046.ab1_PBI_0_0_0_2_373\| | gi\|150048273\|gb\|EE411134.1\| | 2.1 | 0.033 | AT1G75660.1 | XRN3 (5′-3′ exoribonuclease 3) | 268 | |
| sense\|_81_115 | EE411134 | | | | | | |
| Contig563_4314_final_0_0_0_55_672\| | gi\|20374824\|gb\|BG543844.1\| | 2.1 | 0.049 | AT3G49010.1 | ATBBC1 (breast basic conserved 1); | 269 | T |
| sense\|_242_276 | BG543844 | | | AT3G49010.2 | structural constituent of ribosome | | |
| | | | | AT3G49010.3 | | | |
| | | | | AT3G49010.4 | | | |
| | | | | AT3G49010.5 | | | |
| 58ACPE48_UP_012_F08_21SEP2004_054.ab1_PBI_0_0_0_1_435\| | gi\|150055143\|gb\|EE417997.1\| | 2.1 | 0.041 | AT5G58410.1 | binding/protein binding/zinc ion | 270 | |
| sense\|_45_79 | EE417997 | | | | binding | | |
| OL33_36R_C01_OL3074R_012.ab1_AAFC_0_0_0_74_352\| | gi\|122029022\|gb\|EH417887.1\| | 2.0 | 0.044 | AT1G54210.1 | ATG12a (AUTOPHAGY 12) | 271 | |
| sense\|_355_393 | EH417887 | | | AT1G54210.2 | | | |

The B. napus Probe Id, as present on the Combimatrix 90k microarray, is depicted in column 1.
Column 2 is the gene name database reference.
Column 3 depicts the fold change (FC) in expression vs. the control line.
Column 4 indicates the Q-values.
Column 5 is the likely Arabidopsis thaliana homologue (the AGI codes are shown).
Column 6 describes the gene based on homology with other proteins found in nucleotide databases.
Column 8 indicates proteins with mitochondrial (M) or translational (T) function.

TABLE 26
Chloroplast network: FC ≧ 2 (110vs115) and Pearson correlation coefficient >0.8.

| | | FC | | | | | |
|---|---|---|---|---|---|---|---|
| | | (110 | | | | | |
| | | vs. | | | | Seq ID | |
| Probe Id | Name | 115) | Q.value | AGI | annotation | No | function |
| Contig1_86029_remaining_0_0_0_423_857\| | gi\|122026038\|gb\|EH414903.1\| | 43.7 | 3.00E−04 | AT2G24270.1 | ALDH11A3 (Aldehyde | 272 | |
| sense\|_383_417 | EH414903 | | | AT2G24270.2 | dehydrogenase 11A3) | | |
| | | | | AT2G24270.3 | | | |
| | | | | AT2G24270.4 | | | |
| Contig1_46741_remaining_0_0_0_70_612\| | gi\|95839565\|gb\|DY013041.1\| | 27.1 | 0.008 | AT2G39290.1 | PGP1/PGPS1/PGS1 | 273 | |
| sense\|_273_307 | DY013041 | | | | (PHOSPHATIDYLGLYCEROL | | |
| | | | | | PHOSPHATE SYNTHASE 1) | | |
| 39RDBRT_UP_083_H09_24JAN2006_065.ab1_PBI\| | gi\|150906031\|gb\|ES936489.1\| | 23.0 | 2.00E−04 | AT3G53900.2 | uracil | 274 | C |
| senAnsen\|_40_74 | ES936489 | | | | phosphoribosyltransferase, | | |
| | | | | | putative | | |
| 63JKCOT5_T3_002_H03_04JAN2005_017.ab1_PBI_0_0_0_48_851\| | gi\|95843146\|gb\|DY017404.1\| | 18.9 | 0.001 | AT5G01090.1 | legume lectin family protein | 275 | |
| sense\|_657_691 | DY017404 | | | | | | |
| Contig2_1157_final_0_0_0_47_979\|sense\|_122_159 | gi\|151288312\|gb\|EV194973.1\| | 17.1 | 2.00E−04 | AT1G72640.1 | binding/catalytic | 276 | C |
| | EV194973 | | | AT1G72640.2 | | | |
| BNother_UP_037_O20_10DEC2003_031.ab1_GHI1_0_0_0_8_121\| | gi\|150112715\|gb\|EE513687.1\| | 15.6 | 0.002 | AT3G13750.1 | BGAL1 (BETA | 277 | |
| sense\|_9_43 | EE513687 | | | | GALACTOSIDASE 1) | | |
| gi_1048276_NCBI\|senAnsen\|_77_111 | gi\|1048276\|gb\|H74983.1\| | 14.7 | 4.00E−04 | AT1G11410.1 | S-locus protein kinase, | 278 | |
| | H74983 | | | | putative | | |
| BNYS2DCT_UP_033_D04_03MAR2005_026.ab1_PBI_0_0_0_18_212\| | gi\|126486472\|gb\|EE475971.1\| | 14.5 | 5.00E−04 | AT2G30570.1 | PSBW (PHOTOSYSTEM II | 279 | C |
| sense\|_245_279 | EE475971 | | | | REACTION CENTER W) | | |
| gi_72287793_NCBI_0_0_0_147_263\| | gi\|72287793\|gb\|AM061028.1\| | 14.2 | 0.003 | AT1G02280.1 | TOC33 (PLASTID PROTEIN | 280 | C |
| sense\|_93_127 | AM061028 | | | AT1G02280.2 | IMPORT 1) | | |
| Contig1_55592_remaining_0_0_0_37_579\| | gi\|119433019\|gb\|DY030447.1\| | 12.7 | 0.017 | AT5G50420.1 | expressed protein | 281 | |
| sense\|_497_531 | DY030447 | | | | | | |
| Contig1_84291_remaining\|senAnsen\|_124_161 | gi\|150160953\|gb\|EE551103.1\| | 11.9 | 0.002 | AT5G55480.1 | glycerophosphoryl diester | 282 | |
| | EE551103 | | | | phosphodiesterase family | | |
| | | | | | protein | | |
| Contig1_2300_final_0_0_0_300_515\| | gi\|122028216\|gb\|EH417081.1\| | 11.8 | 5.00E−04 | AT4G02920.1 | expressed protein | 283 | |
| sense\|_87_122 | EH417081 | | | AT4G02920.2 | | | |
| 9RDBNGA_UP_176_D11_11MAR2006_089.ab1_PBI_0_0_0_344_475\| | gi\|32502702\|gb\|CD820762.1\| | 11.8 | 0.005 | AT5G28750.1 | thylakoid assembly protein, | 284 | C |
| sense\|_368_404 | CD820762 | | | | putative | | |
| BNARO5GH_T3_014_H06_30NOV2006_034.ab1_PBI_0_0_0_101_892\| | gi\|150876088\|gb\|ES906550.1\| | 11.0 | 0.002 | AT2G39930.1 | ATISA1/ISA1 (ISOAMYLASE | 285 | |
| sense\|_825_859 | ES906550 | | | | 1) | | |
| Contig2_938_final_0_0_0_33_1028\|sense\|_593_627 | gi\|126475471\|gb\|EE458095.1\| | 10.6 | 1.00E−04 | AT2G43950.1 | OEP37; ion channel | 286 | C |
| | EE458095 | | | AT2G43950.2 | | | |
| | | | | AT2G43950.3 | | | |
| CD24F_AAFC\|senAnsen\|_118_157 | gi\|151283976\|gb\|EV190637.1\| | 10.3 | 0.012 | AT5G01410.1 | PDX1 (PYRIDOXINE | 287 | |
| | EV190637 | | | | BIOSYNTHESIS 1.3) | | |
| Contig1_74342_remaining_0_0_0_2_931\| | gi\|65297168\|gb\|CN827382.1\| | 10.2 | 0.001 | AT3G15570.1 | phototropic-responsive NPH3 | 288 | |
| sense\|_275_309 | CN827382 | | | | family protein | | |
| Contig4_9061_final_0_0_0_7_366\|sense\|_226_260 | gi\|56839524\|gb\|CX192100.1\| | 10.0 | 0.008 | AT5G08000.1 | E13L3 (GLUCAN ENDO-1,3- | 289 | |
| | CX192100 | | | | BETA-GLUCOSIDASE-LIKE | | |
| | | | | | PROTEIN 3) | | |
| Contig1_80141_remaining_0_0_0_3_572\| | gi\|151211305\|gb\|EV124350.1\| | 8.1 | 0.039 | AT5G16590.1 | leucine-rich repeat | 290 | |
| sense\|_167_201 | EV124350 | | | | transmembrane protein | | |
| | | | | | kinase, putative | | |
| BNZB_UP_077_A02_02JUN2004_016.ab1_PBI\| | gi\|150155330\|gb\|EE561957.1\| | 7.7 | 0.038 | AT1G50170.1 | ATSIRB; sirohydrochlorin | 291 | C |
| senAnsen\|_267_302 | EE561957 | | | | ferrochelatase | | |
| UC459R_AAFC_0_0_0_138_635\|sense\|_431_465 | gi\|151277941\|gb\|EV188300.1\| | 6.8 | 1.00E−03 | AT3G19810.1 | expressed protein | 292 | C |
| | EV188300 | | | | | | |
| 38RDBRM_UP_061_B11_07DEC2005_093.ab1_PBI_0_0_0_194_541\|sense\|_356_391 | gi\|150127164\|gb\|EE528136.1\| | 6.8 | 0.02 | AT2G35880.1 | expressed protein | 293 | |
| | EE528136 | | | | | | |
| Contig1_18049_final_0_0_0_14_424\| | gi\|126473095\|gb\|EE454234.1\| | 6.3 | 0.006 | AT2G33810.1 | SPL3 (SQUAMOSA | 294 | |
| sense\|_246_281 | EE454234 | | | | PROMOTER BINDING | | |
| | | | | | PROTEIN-LIKE 3) | | |
| Contig4_1978_final_0_0_0_61_1263\| | gi\|151294706\|gb\|EV201369.1\| | 6.2 | 0.004 | AT4G12730.1 | FLA2 | 295 | |
| sense\|_1195_1229 | EV201369 | | | | | | |
| gi_54419678_NCBI_0_0_0_2_409\| | gi\|54419678\|gb\|CV545743.1\| | 5.9 | 0.011 | AT3G20680.1 | expressed protein | 296 | C |
| sense\|_337_371 | CV545743 | | | | | | |
| JKBNHS1_UP_015_C04_02JUL2004_028.ab1_PBI_0_0_0_33_575\| | gi\|83833659\|gb\|CX281882.1\| | 5.8 | 0.031 | AT1G10522.1 | expressed protein | 297 | C |
| sense\|_242_276 | CX281882 | | | AT1G10522.2 | | | |
| gi_75974640_NCBI_0_0_0_11_637\| | gi\|75974640\|gb\|AM0606532.1\| | 5.8 | 0.05 | AT1G54040.1 | ESP (EPITHIOSPECIFIER | 298 | |
| sense\|_341_375 | AM060652 | | | AT1G54040.2 | PROTEIN) | | |
| Contig3_9211_final_0_0_0_90_632\|sense\|_552_557 | gi\|122030955\|gb\|EH419820.1\| | 5.7 | 0.011 | AT5G62790.1 | DXR (1-DEOXY-D- | 299 | C |
| | EH419820 | | | AT5G62790.2 | XYLULOSE 5-PHOSPHATE | | |
| | | | | | REDUCTOISOMERASE) | | |
| Contig2_519_final_0_0_0_123_431\|sense\|_416_455 | gi\|32504311\|gb\|CD822371.1\| | 5.5 | 0.023 | AT2G37240.1 | antioxidant/oxidoreductase | 300 | C |
| | CD822371 | | | | | | |
| 47RDOAH_UP_037_D11_17AUG2004_089.ab1_PBI_0_0_0_2_478\| | gi\|95860699\|gb\|DY029073.1\| | 5.1 | 0.002 | AT3G25860.1 | LTA2 (PLASTID E2 SUBUNIT | 301 | C |
| sense\|_432_466 | DY029073 | | | | OF PYRUVATE | | |
| | | | | | DECARBOXYLASE) | | |
| BNShoot_UP_130_K05_10DEC2003_078.ab1_GHI1_0_0_0_3_242\| | gi\|56838609\|gb\|CX191185.1\| | 5.0 | 0.001 | AT5G64040.1 | PSAN (photosystem\|reaction | 302 | C |
| sense\|_84_118 | CX191185 | | | AT5G64040.2 | center subunit PSI-N) | | |
| Contig1_9466_final_0_0_0_61_1452\| | gi\|112354580\|gb\|AM390360.1\| | 4.8 | 0.019 | AT5G42390.1 | metalloendopeptidase | 303 | C |
| sense\|_1030_1064 | AM390360 | | | | | | |
| BNARO6GH_T3_024_B08_07DEC2006_062.ab1_PBI_0_0_0_123_845\| | gi\|150880452\|gb\|ES910913.1\| | 4.8 | 0.045 | AT3G49940.1 | LBD38 (LOB DOMAIN- | 304 | |
| sense\|_529_563 | ES910913 | | | | CONTAINING PROTEIN 38) | | |
| Contig2_5769_final_0_0_0_2_226\|sense\|_118_152 | gi\|29690060\|gb\|CB686335.1\| | 4.6 | 0.01 | AT2G33380.1 | RD20 (RESPONSIVE TO | 305 | |
| | CB686335 | | | AT2G33380.2 | DESSICATION 20) | | |
| OL33_36R_J10_OL3229R_065.ab1_AAFC_0_0_0_3_638\| | gi\|122029360\|gb\|EH418225.1\| | 4.4 | 0.014 | AT3G15520.1 | peptidyl-prolyl cis-trans | 306 | C |
| sense\|_57_91 | EH418225 | | | | isomerase TLP38, chloroplast | | |
| Contig1_17419_final_0_0_0_638_874\| | gi\|151182351\|gb\|EV095859.1\| | 4.0 | 0.003 | AT5G57345.1 | expressed protein | 307 | |
| sense\|_336_370 | EV095859 | | | | | | |
| gi_56835891_NCBI_0_0_0_1_267\| | gi\|56835891\|gb\|CX188467.1\| | 3.6 | 0.047 | AT4G27440.1 | PORB | 308 | C |
| sense\|_236_273 | CX188467 | | | AT4G27440.2 | (PROTOCHLOROPHYLLIDE | | |
| | | | | | OXIDOREDUCTASE B) | | |
| 37RDBRH_UP_004_C03_26MAR2004_027.ab1_PBI_0_0_0_4_573\| | gi\|95828875\|gb\|DW999350.1\| | 3.6 | 0.003 | AT4G21850.2 | MSRB2 (METHIONINE | 309 | C |
| sense\|_132_166 | DW999350 | | | AT4G21860.1 | SULFOXIDE REDUCTASE B | | |
| | | | | AT4G21860.2 | 2) | | |
| | | | | AT4G21860.3 | | | |
| EX20LIB6_UP_103_F10_14JUL2004_070.ab1_PBI_0_0_0_3_110\| | gi\|150944423\|gb\|ES973868.1\| | 3.6 | 0.032 | AT5G38360.1 | esterase/lipase/thioesterase | 310 | |
| sense\|_81_115 | ES973868 | | | | family protein | | |
| BNAEN3GH_UP_109_E02_15SEP2006_008.ab1_PBI\| | gi\|150986716\|gb\|EV000534.1\| | 3.1 | 0.049 | AT5G35630.3 | GS2 (GLUTAMINE | 311 | C |
| senAnsen\|_169_208 | EV000534 | | | AT5G35630.2 | SYNTHETASE 2) | | |
| | | | | AT5G35630.1 | | | |
| 37RDBRH_UP_068_D09_05DEC2005_073.ab1_PBI\| | gi\|150126159\|gb\|EE527131.1\| | 3.1 | 0.031 | AT2G42590.1 | GRF9 (GENERAL | 312 | |
| senAnsen\|_98_132 | EE527131 | | | AT2G42590.2 | REGULATORY FACTOR 9) | | |
| | | | | AT2G42590.3 | | | |
| BNZB_UP_113_D08_19AUG2004_058.ab1_PBI\| | gi\|151291352\|gb\|EV198013.1\| | 3.1 | 0.011 | AT1G30380.1 | PSAK (PHOTOSYSTEM\| | 313 | C |
| senAnsen\|_25_59 | EV198013 | | | | SUBUNIT K) | | |
| Contig4_2526_final_0_0_0_43_477\|sense\|_338_372 | gi\|54417751\|gb\|CV544784.1\| | 3.0 | 0.049 | AT5G01650.1 | macrophage migration | 314 | |
| | CV544784 | | | AT5G01650.2 | inhibitory factor family protein | | |
| Contig8_4093_final_0_0_0_2_334\|sense\|_307_345 | gi\|151311021\|gb\|EV211058.1\| | 3.0 | 0.018 | AT4G36250.1 | ALDH3F1 (ALDEHYDE | 315 | |
| | EV211058 | | | | DEHYDROGENASE 3F1) | | |
| Contig3_798_final_0_0_0_2_532\|sense\|_510_546 | gi\|150925074\|gb\|ES955537.1\| | 2.8 | 0.039 | AT3G06730.1 | thioredoxin family protein | 316 | C |
| | ES955537 | | | | | | |
| Contig1_15755_final_0_0_0_30_551\| | gi\|54418529\|gb\|CV545169.1\| | 2.8 | 0.03 | AT5G64380.1 | fructose-1,6-bisphosphatase | 317 | |
| sense\|_157_191 | CV545169 | | | | family protein | | |
| Contig1_2768_final_0_0_0_425_640\| | gi\|150041563\|gb\|EE404438.1\| | 2.8 | 0.036 | AT5G50110.1 | methyltransferase-related | 318 | |
| sense\|_9_43 | EE404438 | | | | | | |
| Contig1_4928_remaining_0_0_0_3_602\| | gi\|65299729\|gb\|CN829943.1\| | 2.7 | 0.025 | AT1G12860.1 | basic helix-loop-helix (bHLH) | 319 | |
| sense\|_257_291 | CN829943 | | | | family protein | | |
| Contig1_87659_remaining_0_0_0_54_224\| | gi\|122041313\|gb\|EH430178.1\| | 2.6 | 0.039 | AT3G22231.1 | PCC1 (PATHOGEN AND | 320 | |
| sense\|_337_371 | EH430178 | | | | CIRCADIAN CONTROLLED | | |
| | | | | | 1) | | |
| BNShoot_UP_125_J24_10DEC2003_087.ab1_GHI1_0_0_0_26_301\| | gi\|65287110\|gb\|CN729306.1\| | 2.5 | 0.022 | AT1G20340.1 | DRT112 (DNA-damage- | 321 | C |
| sense\|_259_293 | CN729306 | | | | repair/toleration protein 112) | | |
| Contig1_2745_final_0_0_0_86_799\|sense\|_278_312 | gi\|151311097\|gb\|EV211134.1\| | 2.4 | 0.04 | AT4G00050.1 | UNE10 (unfertilized embryo | 322 | |
| | EV211134 | | | | sac 10) | | |
| Contig1_1281_final_0_0_0_3_551\|sense\|_62_97 | gi\|150058902\|gb\|EE421648.1\| | 2.3 | 0.039 | AT4G29750.1 | expressed protein | 323 | C |
| | EE421648 | | | | | | |
| gi_21844485_NCBI_0_0_0_2_454\| | gi\|21844485\|gb\|BQ705066.1\| | 2.3 | 0.039 | AT3G22150.1 | pentatricopeptide (PPR) | 324 | C |
| sense\|_17_51 | BQ705066 | | | | repeat-containing protein | | |
| BNARO4GH_T3_019_D05_24NOV2006_041.ab1_PBI_0_0_0_6_821\|sense\|_322_356 | gi\|150874000\|gb\|ES904458.1\| | 2.0 | 0.05 | AT1G04040.1 | acid phosphatase class B | 325 | |
| | ES904458 | | | | family protein | | |

The B. napus Probe Id, as present on the Combimatrix 90k microarray, is depicted in column 1.
Column 2 is the gene name database reference.
Column 3 depicts the fold change (FC) in expression vs. the control line.
Column 4 indicates the Q-values.
Column 5 is the likely Arabidopsis thaliana homologue (the AGI codes are shown).
Column 6 describes the gene based on homology with other proteins found in nucleotide databases.
Column 8 indicates proteins with mitochondrial (M) or translational (T) function.

TABLE 27
Mitochondrial network linked to genes involved in translation: FC ≧ 2 (112vs115) and Pearson correlation coefficient >0.8.

| | | FC | | | | | |
|---|---|---|---|---|---|---|---|
| | | (110 vs. | | | | Seq ID | |
| Probe Id | Name | 115) | Q. value | AGI | annotation | No | function |
| Contig24_1883_final_0_0_0_62_268\| | gi\|83822180\|gb\|CX270403.1\| | 72.3 | 1.00E−04 | AT3G59540.1 | 60S ribosomal protein L38 | 148 | T |
| sense\|_276_311 | CX270403 | | | AT2G43460.1 | (RPL38A) | | |
| Contig1_17115_final_0_0_0_2_430\|sense\|_81_115 | gi\|125943277\|gb\|EL592992.1\| | 46.4 | 2.00E−04 | AT5G14520.1 | pescadillo-related | 151 | |
| | EL592992 | | | | | | |
| 49RDOATR_UP_046_H11_240CT2004_081.ab1_PBI_0_0_0_15_254\| | gi\|150149702\|gb\|EE552105.1\| | 44.4 | 2.00E−04 | AT1G31730.1 | epsilon-adaptin, putative | 149 | |
| sense\|_222_256 | EE552105 | | | | | | |
| Contig2_8545_final_0_0_0_204_728\| | gi\|32517661\|gb\|CD835721.1\| | 42.4 | 1.00E−04 | AT4G34460.1 | AGB1 (GTP BINDING | 150 | |
| sense\|_615_649 | CD835721 | | | AT4G34460.2 | PROTEIN BETA 1) | | |
| | | | | AT4G34460.3 | | | |
| | | | | AT4G34460.4 | | | |
| Contig4_1758_final_0_0_0_13_1137\| | gi\|95840952\|gb\|DY016483.1\| | 40.5 | 2.00E−04 | AT3G15000.1 | hypothetical protein | 153 | |
| sense\|_617_652 | DY016483 | | | | | | |
| Contig1_8940_final_0_0_0_257_1270\| | gi\|150131955\|gb\|EE532927.1\| | 39.2 | 2.00E−04 | AT1G52730.1 | transducin family protein/WD- | 326 | |
| sense\|_879_913 | EE532927 | | | AT1G52730.2 | 40 repeat family protein | | |
| Contig1_1343_final_0_0_0_86_520\|sense\|_64_103 | gi\|150057077\|gb\|EE419840.1\| | 34.7 | 5.00E−04 | AT4G21110.1 | G10 family protein | 164 | |
| | EE419840 | | | | | | |
| Contig2_1039_final_0_0_0_136_780\| | gi\|126501180\|gb\|EE470903.1\| | 34.4 | 4.00E−04 | AT3G25040.1 | ER lumen protein retaining | 155 | |
| sense\|_788_822 | EE470903 | | | | receptor, putative/HDEL | | |
| | | | | | receptor, putative | | |
| gi_21843860_NCBI\|senAnsen\|_184_220 | gi\|21843860\|gb\|BQ704441.1\| | 30.4 | 3.00E−04 | AT4G10610.1 | RBP37 (RNA-BINDING | 159 | |
| | BQ704441 | | | AT4G10610.2 | PROTEIN 37); RNA binding | | |
| | | | | | (CID12) | | |
| Contig3_1253_final_0_0_0_2_529\|sense\|_11_45 | gi\|32511884\|gb\|CD829944.1\| | 29.7 | 0.003 | AT1G12000.1 | pyrophosphate-fructose-6- | 171 | |
| | CD829944 | | | | phosphate 1- | | |
| | | | | | phosphotransferase beta | | |
| | | | | | subunit, putative | | |
| Contig1_702_final_0_0_0_72_341\|sense\|_332_368 | gi\|83833667\|gb\|CX281890.1\| | 28.5 | 2.00E−04 | AT2G20820.1 | expressed protein | 256 | |
| | CX281890 | | | AT2G20820.2 | | | |
| Contig4_782_final_0_0_0_48_404\|sense\|_190_224 | gi\|119424582\|gb\|DY009984.1\| | 27.5 | 2.00E−04 | AT2G19740.1 | 60S ribosomal protein L31 | 157 | T |
| | DY009984 | | | | (RPL31A) | | |
| gi_32499613_NCBI_0_0_0_6_317\| | gi\|32499613\|gb\|CD817673.1\| | 26.2 | 3.00E−04 | AT1G63810.1 | nucleolar RNA-associated | 160 | |
| sense\|_168_202 | CD817673 | | | | family protein | | |
| Contig1_42742_remaining_0_0_0_80_511\| | gi\|150162948\|gb\|EE555762.1\| | 23.6 | 6.00E−04 | AT1G08880.1 | G-H2AX/GAMMA- | 168 | |
| sense\|_133_168 | EE555762 | | | | H2AX/H2AXA/HTA5 | | |
| Contig3_6771_final_0_0_0_34_390\|sense\|_8_46 | gi\|32512640\|gb\|CD830700.1\| | 22.2 | 9.00E−04 | AT3G59650.1 | mitochondrial ribosomal | 162 | M |
| | CD830700 | | | AT3G59650.2 | protein L51/S25/CI-B8 family | | |
| | | | | | protein | | |
| Contig1_2213_final_0_0_0_96_1157\| | gi\|150927189\|gb\|ES957652.1\| | 21.6 | 7.00E−04 | AT4G37210.1 | tetratricopeptide repeat (TPR)- | 165 | |
| sense\|_798_832 | ES957652 | | | AT4G37210.2 | containing protein | | |
| Contig4_9922_final_0_0_0_2_1498\|sense\|_780_817 | gi\|119430188\|gb\|DY020917.1\| | 21.6 | 4.00E−04 | AT2G21790.1 | R1/RNR1 | 169 | |
| | DY020917 | | | | (RIBONUCLEOTIDE | | |
| | | | | | REDUCTASE 1) | | |
| Contig1_71772_remaining_0_0_0_2_454\| | gi\|125934956\|gb\|EL589211.1\| | 21.3 | 0.002 | AT4G11790.1 | Ran-binding protein 1 domain- | 166 | |
| sense\|_281_315 | EL589211 | | | | containing protein | | |
| Contig1_17597_final_0_0_0_3_746\|sense\|_209_243 | gi\|150878730\|gb\|ES909188.1\| | 21.1 | 3.00E−04 | AT1G59540.1 | ZCF125; microtubule motor | 163 | |
| | ES909188 | | | AT1G59540.2 | | | |
| Contig13_5494_final_0_0_0_90_383\| | gi\|95828809\|gb\|DW999284.1\| | 20.2 | 0.009 | AT1G14980.1 | CPN10 (CHAPERONIN 10) | 161 | M |
| sense\|_50_84 | DW999284 | | | | | | |
| 72ETGS24_UP_003_B03_18MAY2005_029.ab1_PBI_0_0_0_2_247\| | gi\|150080405\|gblEE443144.1\| | 19.3 | 6.00E−04 | AT3G43920.1 | DCL3 (DICER-LIKE 3) | 181 | |
| sense\|_81_115 | EE443144 | | | AT3G43920.2 | | | |
| | | | | AT3G43920.3 | | | |
| Contig5_72_final_0_0_0_63_380\|sense\|_761_799 | gi\|150152399\|gb\|EE556824.1\| | 19.1 | 0.002 | AT2G27710.1 | 60S acidic ribosomal protein | 182 | T |
| | EE556824 | | | AT2G27710.2 | P2 (RPP2B) | | |
| | | | | AT2G27710.3 | | | |
| | | | | AT2G27710.4 | | | |
| 46RDOAG_UP_018_D05_27SEP2004_041.ab1_PBI_0_0_0_2_349\| | gi\|150130383\|gb\|EE531355.1\| | 17.4 | 0.01 | AT2G38770.1 | EMB2765 | 199 | |
| sense\|_22_56 | EE531355 | | | | | | |
| 39RDBRT_UP_069_E04_30NOV2005_024.ab1_PBI_0_0_0_321_497\| | gi\|150116248\|gb\|EE517220.1\| | 17.2 | 1.00E−03 | AT1G63470.1 | DNA-binding family protein | 178 | |
| sense\|_126_161 | EE517220 | | | | | | |
| Contig1_62949_remaining_0_0_0_3_107\| | gi\|150871714\|gb\|ES902175.1\| | 17.2 | 0.008 | AT2G35040.1 | AICARFT/IMPCHase | 167 | |
| sense\|_305_339 | ES902175 | | | | bienzyme family protein | | |
| Contig2_11169_final_0_0_0_61_831\| | gi\|75970744\|gb\|AM057198.1\| | 17.0 | 3.00E−04 | AT4G27490.1 | 3′ exoribonuclease family | 174 | |
| sense\|_737_771 | AM057198 | | | | domain 1-containing protein | | |
| Contig1_23244_remaining_0_0_0_3_1172\| | gi\|112354818\|gb\|AM390327.1\| | 16.4 | 5.00E−04 | AT2G01750.1 | ATMAP70-3 (microtubule- | 193 | |
| sense\|_512_546 | AM390327 | | | AT2G01750.2 | associated proteins 70-3) | | |
| DC2273R_AAFC_0_0_0_49_441\|sense\|_257_291 | gi\|151326308\|gb\|EV226299.1\| | 16.3 | 0.004 | AT2G01140.1 | fructose-bisphosphate | 175 | |
| | EV226299 | | | | aldolase, putative | | |
| OL105_108R_015_OL10216R_047.ab1_AAFC_0_0_0_3_551\| | gi\|32495522\|gb\|CD813582.1\| | 14.7 | 0.009 | AT2G17980.1 | ATSLY1; protein transporter | 170 | |
| sense\|_147_181 | CD813582 | | | | | | |
| CL3599F_AAFC_0_0_0_46_291\|sense\|_133_167 | gi\|151293685\|gb\|EV200346.1\| | 14.3 | 0.002 | AT2G04660.1 | APC2 (anaphase-promoting | 191 | |
| | EV200346 | | | | complex/cyclosome 2) | | |
| Contig1_2482_final_0_0_0_22_615\|sense\|_558_592 | gi\|32494572\|gb\|CD812632.1\| | 14.0 | 0.039 | AT5G62290.1 | nucleotide-sensitive chloride | 183 | |
| | CD812632 | | | AT5G62290.2 | conductance regulator (ICIn) | | |
| | | | | | family protein | | |
| Contig2_2378_final_0_0_0_20_379\|sense\|_190_225 | gi\|150887215\|gb\|ES917673.1\| | 13.3 | 0.002 | AT1G26880.1 | 60S ribosomal protein L34 | 192 | T |
| | ES917673 | | | AT1G26880.2 | (RPL34A) | | |
| Contig3_3191_final_0_0_0_2_634\|sense\|_468_502 | gi\|95828892\|gb\|DW999367.1\| | 13.2 | 0.002 | AT3G13670.1 | protein kinase family protein | 184 | |
| | DW999367 | | | | | | |
| BNARO5GH_T3_002_B11_29NOV2006_093.ab1_PBI\| | gi\|150877188\|gb\|ES907652.1\| | 12.8 | 0.03 | AT5G19680.1 | leucine-rich repeat family | 221 | |
| senAnsen\|_257_291 | ES907652 | | | | protein | | |
| Contig2_2455_final_0_0_0_1_951\|sense\|_145_179 | gi\|150055308\|gb\|EE418162.1\| | 12.7 | 0.019 | AT1G06720.1 | expressed protein | 197 | |
| | EE418162 | | | | | | |
| Contig1_11805_final_0_0_0_3_392\|sense\|_340_377 | gi\|83833547\|gb\|CX281770.1\| | 11.6 | 0.004 | AT3G07050.1 | GTP-binding family protein | 190 | |
| | CX281770 | | | | | | |
| 49RDOAT_UP_052_H12_27JAN2006_082.ab1_PBI_0_0_0_1_576\| | gi\|150917098\|gb\|ES947559.1\| | 11.6 | 0.002 | AT2G36200.1 | kinesin motor protein-related | 195 | |
| sense\|_423_457 | ES947559 | | | AT2G36200.2 | | | |
| Contig1_68996_remaining_0_0_0_3_422\| | gi\|150132975\|gb\|EE533945.1\| | 11.4 | 3.00E−04 | AT5G19400.1 | expressed protein | 180 | |
| sense\|_109_143 | EE533945 | | | | | | |
| Contig2_12321_final_0_0_0_93_686\| | gi\|65298993\|gb\|CN829207.1\| | 10.7 | 0.004 | AT5G11900.1 | eukaryotic translation initiation | 176 | T |
| sense\|_610_644 | CN829207 | | | | factor SUI1 family protein | | |
| Contig31_1833_final_0_0_0_43_531\| | gi\|65293240\|gb\|CN735425.1\| | 9.0 | 0.002 | AT5G48580.1 | FKBP15-2 (FK506-binding | 202 | |
| sense\|_524_562 | 5.1\|CN735425 | | | | protein 15 kD-2) | | |
| ES1560F_AAFC_0_0_0_2_202\|sense\|_64_98 | gi\|126367165\|gb\|DN965146.1\| | 8.9 | 0.035 | AT4G34200.1 | EDA9 (embryo sac | 327 | |
| | DN965146 | | | | development arrest 9) | | |
| BNSCS2CT_UP_031_F12_07JAN2005_086.ab1_PBI_0_0_0_3_122\| | gi\|126479535\|gb\|EE464699.1\| | 8.8 | 0.033 | AT2G45280.1 | ATRAD51C (Arabidopsis | 196 | |
| sense\|_63_102 | EE464699 | | | AT2G45280.2 | thaliana Ras Associated with | | |
| | | | | | Diabetes protein 51C) | | |
| 47RDOAH_UP_043_A05_20AUG2004_047.ab1_PBI\| | gi\|95861799\|gb\|DY029554.1\| | 8.7 | 0.007 | AT2G19480.1 | NAP1; 2/NFA2 | 205 | |
| senAnsen\|_25_59 | DY029554 | | | AT2G19480.2 | (NUCLEOSOME ASSEMBLY | | |
| | | | | AT2G19480.3 | PROTEIN1; 2) | | |
| gi_113704882_NCBI_0_0_0_9_479\| | gi\|113704882\|gb\|AM394050.1\| | 8.5 | 0.003 | AT5G46070.1 | GTP binding/GTPase | 194 | |
| sense\|_408_446 | AM394050 | | | | | | |
| Contig3_6028_final_0_0_0_72_608\|sense\|_13_47 | gi\|65293342\|gb\|CN735527.1\| | 8.4 | 0.033 | AT2G38130.1 | ATMAK3 (Arabidopsis thaliana | 198 | |
| | CN735527 | | | AT2G38130.2 | MAK3 homologue); N- | | |
| | | | | | acetyltransferase | | |
| 24RDBNH_UP_037_B04_20FEB2004_030.ab1_PBI_0_0_0_108_497\| | gi\|150105739\|gb\|EE506711.1\| | 8.4 | 0.019 | AT2G03680.1 | SPR1 (SPIRAL1) | 185 | |
| sense\|_401_435 | EE506711 | | | AT2G03680.2 | | | |
| Contig2_5461_final_0_0_0_2_118\|sense\|_451_488 | gi\|150140559\|gb\|EE541522.1\| | 7.8 | 0.016 | AT2G27040.1 | AGO4 (ARGONAUTE 4) | 200 | |
| | EE541522 | | | AT2G27040.2 | | | |
| LD6126F_AAFC_0_0_0_1_255\|sense\|_191_225 | gi\|151188650\|gb\|EV102123.1\| | 7.8 | 5.00E−04 | AT5G54670.1 | kinesin-like protein A | 188 | |
| | EV102123 | | | | | | |
| 59ACAB6_UP_012_H10_23JUL2004_066.ab1_PBI_0_0_0_35_592\| | gi\|65295054\|gb\|CN737235.1\| | 7.5 | 0.008 | AT4G30480.1 | tetratricopeptide repeat (TPR)- | 201 | M |
| sense\|_392_429 | CN737235 | | | AT4G30480.2 | containing protein | | |
| | | | | AT4G30480.3 | | | |
| Contig22_1329_final_0_0_0_41_679\| | gi\|54419450\|gb\|CV545628.1\| | 7.4 | 0.03 | AT1G02780.1 | EMB2386 (EMBRYO | 204 | |
| sense\|_10_45 | CV545628 | | | | DEFECTIVE 2386) | | |
| Contig1_2413_final_0_0_0_43_411\|sense\|_888_922 | gi\|151189343\|gb\|EV102816.1\| | 7.2 | 0.046 | AT4G34360.1 | protease-related | 328 | |
| | EV102816 | | | | | | |
| Contig1_3137_final_0_0_0_70_741\|sense\|_48_82 | gi\|150052413\|gb\|EE415272.1\| | 7.1 | 0.012 | AT5G19680.1 | leucine-rich repeat family | 221 | |
| | EE415272 | | | | protein | | |
| 9RDBNGA_UP_072_E09_03APR2005_071.ab1_PBI_0_0_0_2_577\| | gi\|150144783\|gb\|EE545746.1\| | 6.8 | 0.001 | AT4G02390.1 | APP (ARABIDOPSIS | 210 | |
| sense\|_25_59 | EE545746 | | | | POLY(ADP-RIBOSE) | | |
| | | | | | POLYMERASE) | | |
| ML3653F_AAFC\|senAnsen\|_18_52 | gi\|65296856\|gb\|CN827070.1\| | 6.5 | 0.026 | AT2G21870.1 | Identical to Probable ATP | 207 | M |
| | CN827070 | | | AT2G21870.2 | synthase 24 kDa subunit, | | |
| | | | | | mitochondrial precursor | | |
| Contig3_10313_final_0_0_0_3_500\|sense\|_448_482 | gi\|65293469\|gb\|CN735652.1\| | 6.5 | 0.002 | AT3G24830.1 | 60S ribosomal protein L13A | 211 | T |
| | CN735652 | | | | (RPL13aA) | | |
| BNAEN3GH_UP_236_D07_29NOV2006_057.ab1_PBI_0_0_0_4_201\| | gi\|150995919\|gb\|EV009734.1\| | 6.3 | 0.012 | AT1G74560.1 | NRP1 (NAP1-RELATED | 237 | |
| sense\|_24_58 | EV009734 | | | AT1G74560.2 | PROTEIN 1) | | |
| Contig4_709_final_0_0_0_2_388\|sense\|_227_261 | gi\|56842240\|gb\|CX194816.1\| | 6.2 | 0.022 | AT4G33650.1 | ADL2 (ARABIDOPSIS | 243 | |
| | CX194816 | | | AT4G33650.2 | DYNAMIN-LIKE 2) | | |
| Contig2_2953_final_0_0_0_1_684\|sense\|_88_122 | gi\|150888173\|gb\|ES918631.1\| | 6.1 | 0.016 | AT2G37340.1 | RSZ33 (ARGININE/SERINE- | 227 | |
| | ES918631 | | | AT2G37340.2 | RICH ZINC KNUCKLE- | | |
| | | | | AT2G37340.3 | CONTAINING PROTEIN 33) | | |
| Contig8_2355_final_0_0_0_94_486\|sense\|_9_43 | gi\|150041851\|gb\|EE404725.1\| | 6.0 | 0.01 | AT4G31720.1 | TAFII15 (SALT TOLERANCE | 214 | |
| | EE404725 | | | AT4G31720.2 | DURING GERMINATION 1) | | |
| Contig1_7648_final_0_0_0_49_726\|sense\|_589_623 | gi\|65294419\|gb\|CN736602.1\| | 5.9 | 0.004 | AT4G31930.1 | mitochondrial glycoprotein | 209 | M |
| | CN736602 | | | | family protein/MAM33 family | | |
| | | | | | protein | | |
| Contig1_17648_final_0_0_0_1_723\|sense\|_513_547 | gi\|151248819\|gb\|EV159239.1\| | 5.9 | 0.005 | AT5G39900.1 | GTP binding/translation | 323 | |
| | EV159239 | | | | elongation factor | | |
| Contig4_2857_final_0_0_0_1_387\|sense\|_459_496 | gi\|150089907\|gb\|EE490879.1\| | 5.8 | 0.036 | AT2G17630.1 | phosphoserine | 222 | |
| | EE490879 | | | | aminotransferase, putative | | |
| Contig2_4733_final_0_0_0_64_573\|sense\|_408_442 | gi\|32509330\|gb\|CD827390.1\| | 5.7 | 0.044 | AT5G52920.1 | PKP-BETA1/PKP1/PKP2 | 216 | |
| | CD827390 | | | | (PLASTIDIC PYRUVATE | | |
| | | | | | KINASE 1) | | |
| Contig1_803_final_0_0_0_67_429\|sense\|_264_298 | gi\|32503997\|gb\|CD822057.1\| | 5.4 | 0.042 | AT2G36930.1 | zinc finger (C2H2 type) family | 229 | |
| | CD822057 | | | | protein | | |
| Contig125_4314_final_0_0_0_227_760\| | gi\|150098125\|gb\|EE499097.1\| | 5.3 | 0.039 | AT2G34480.1 | 60S ribosomal protein L18A | 235 | T |
| sense\|_29_63 | EE499097 | | | | (RPL18aB) | | |
| Contig1_83793_remaining_0_0_0_2_898\| | gi\|95837756\|gb\|DY012168.1\| | 5.2 | 0.018 | AT3G62360.1 | expressed protein | 234 | |
| sense\|_361_395 | DY012168 | | | | | | |
| Contig1_1257_final_0_0_0_43_660\|sense\|_630_669 | gi\|119420299\|gb\|DY002740.1\| | 5.2 | 0.036 | AT5G48760.1 | 60S ribosomal protein L13A | 329 | T |
| | DY002740 | | | AT5G48760.2 | (RPL13aD) | | |
| LD3466R_AAFC\|senAnsen\|_182_217 | gi\|151037175\|gb\|EV050951.1\| | 5.1 | 0.015 | AT4G31880.1 | expressed protein | 247 | |
| | EV050951 | | | AT4G31880.2 | | | |
| 9RDBNGA_UP_166_C01_10MAR2006_011.ab1_PBI\| | gi\|150927026\|gb\|ES957489.1\| | 5.1 | 0.035 | AT3G60770.1 | 40S ribosomal protein S13 | 236 | T |
| senAnsen\|_9_48 | ES957489 | | | | (RPS13A) | | |
| Contig26_886_final_0_0_0_69_281\|sense\|_5_39 | gi\|32494420\|gb\|CD812480.1\| | 5.0 | 0.029 | AT2G31490.1 | NDU8: plant specific subunit | 212 | M |
| | CD812480 | | | | | | |
| 37RDBRH_UP_020_C12_31MAR2004_092.ab1_PB1_0_0_0_1_372\| | gi\|150122821\|gb\|EE523793.1\| | 5.0 | 0.007 | AT1G75660.1 | XRN3 (5′-3′ exoribonuclease | 246 | |
| sense\|_338_372 | EE523793 | | | | 3) | | |
| Contig535_4314_final_0_0_0_88_522\| | gi\|113704806\|gb\|AM395294.1\| | 4.9 | 0.016 | AT2G32060.1 | 40S ribosomal protein S12 | 228 | T |
| sense\|_66_102 | AM395294 | | | AT2G32060.2 | (RPS12C) | | |
| | | | | AT2G32060.3 | | | |
| Contig4_11587_final_0_0_0_3_452\|sense\|_64_100 | gi\|65294412\|gb\|CN736595.1\| | 4.7 | 0.001 | AT1G04170.1 | EIF2 GAMMA subunit | 238 | T |
| | CN736595 | | | | | | |
| Contig157_601_final_0_0_0_73_258\| | gi\|32494504\|gb\|CD812564.1\| | 4.7 | 0.033 | AT5G56670.1 | 40S ribosomal protein S30 | 224 | T |
| sense\|_10_44 | CD812564 | | | | (RPS30A) | | |
| gi_37621592_NCBI_0_0_0_3_260\| | gi\|37621592\|gb\|CA992297.1\| | 4.7 | 0.016 | AT3G57290.1 | EIF3E | 226 | T |
| sense\|_381_415 | CA992297 | | | | | | |
| Contig25_411_final_0_0_0_10_1182\| | gi\|150047757\|gb\|EE410618.1\| | 4.5 | 0.034 | AT2G23350.1 | polyadenylate-binding protein, | 330 | T |
| sense\|_1037_1071 | EE410618 | | | | putative/PABP, putative | | |
| Contig27_1883_final_0_0_0_56_262\| | gi\|83822180\|gb\|CX270403.1\| | 4.4 | 0.029 | AT3G59540.1 | 60S ribosomal protein L38 | 249 | T |
| sense\|_272_306 | CX270403 | | | AT2G43460.1 | (RPL38A) | | |
| CD3489F_AAFC\|senAnsen\|_191_225 | gi\|150056134\|gb\|EE418988.1\| | 4.2 | 0.032 | AT2G19480.1 | NAP1; 2/NFA2 | 233 | |
| 225 | EE418988 | | | AT2G19480.2 | (NUCLEOSOME ASSEMBLY | | |
| | | | | AT2G19480.3 | PROTEIN1; 2) | | |
| Contig1_2118_final_0_0_0_85_657\|sense\|_24_58 | gi\|125938690\|gb\|EL591018.1\| | 4.2 | 0.042 | AT3G02560.1 | 40S ribosomal protein S7 | 331 | T |
| | EL591018 | | | AT3G02560.2 | (RPS7B) | | |
| Contig1_84646 _remaining_0_0_0_3_194\| | gi\|151214581\|gb\|EV127622.1\| | 4.1 | 0.01 | AT3G52590.1 | UBQ1 (EARLY-RESPONSIVE | 242 | |
| sense\|_35_69 | EV127622 | | | | TO DEHYDRATION 16, | | |
| | | | | | UBIQUITIN EXTENSION | | |
| | | | | | PROTEIN 1) | | |
| gi_112352213_NCBI_0_0_0_3_569\| | gi\|112352213\|gb\|AM389238.1\| | 3.9 | 0.037 | AT3G01610.1 | CDC48C (EMBRYO | 332 | |
| sense\|_89_123 | AM389238 | | | | DEFECTIVE 1354); ATPase | | |
| Contig3_62_final_0_0_0_153_1616\|sense\|_929_963 | gi\|65297892\|gb\|CN828106.1\| | 3.9 | 0.006 | AT1G11680.1 | CYP51G1 (CYTOCHROME | 240 | |
| | CN828106 | | | | P450 51) | | |
| Contig1_15982_final_0_0_0_77_598\| | gi\|65295338\|gb\|CN737519.1\| | 3.8 | 0.039 | AT4G38680.1 | CSDP2/GRP2 (COLD SHOCK | 333 | |
| sense\|_57_92 | CN737519 | | | | DOMAIN PROTEIN 2, | | |
| | | | | | GLYCINE RICH PROTEIN 2); | | |
| | | | | | nucleic acid binding | | |
| gi_56837718_NCBI\|senAnsen\|_30_65 | gi\|56837718\|gb\|CX190294.1\| | 3.8 | 0.035 | AT5G28640.1 | AN3 (ANGUSITFOLIA3) | 334 | |
| | CX190294 | | | | | | |
| Contig1_416_final_0_0_0_45_407\|sense\|_504_543 | gi\|32494036\|gb\|CD812096.1\| | 3.7 | 0.008 | AT5G57290.3 | 60S acidic ribosomal protein | 254 | T |
| | CD812096 | | | AT5G57290.2 | P3 (RPP3B) | | |
| | | | | AT5G57290.1 | | | |
| Contig6_874_final_0_0_0_45_707\|sense\|_4_38 | gi\|65283735\|gb\|CN725933.1\| | 3.6 | 0.013 | AT1G66580.1 | 60S ribosomal protein L10 | 250 | T |
| | CN725933 | | | | (RPL10C) | | |
| Contig3_673_final_0_0_0_61_714\|sense\|_19_53 | gi\|32508529\|gb\|CD826589.1\| | 3.4 | 0.039 | AT3G53970.1 | proteasome inhibitor-related | 253 | |
| | CD826589 | | | AT3G53970.2 | | | |
| Contig3_702_final_0_0_0_59_331\|sense\|_320_355 | gi\|32494127\|gb\|CD812187.1\| | 3.4 | 0.01 | AT2G20820.1 | expressed protein | 251 | |
| | CD812187 | | | AT2G20820.2 | | | |
| Contig20_4314_final_0_0_0_48_476\| | gi\|32523927\|gb\|CD841987.1\| | 3.3 | 0.042 | AT4G29410.1 | 60S ribosomal protein L28 | 335 | T |
| sense\|_472_506 | CD841987 | | | AT4G29410.2 | (RPL28C) | | |
| Contig2_706_final_0_0_0_279_596\|sense\|_31_65 | gi\|72289524\|gb\|AM059100.1\| | 3.3 | 0.006 | AT3G14080.1 | small nuclear | 336 | |
| | AM059100 | | | AT3G14080.2 | ribonucleoprotein, putative/ | | |
| | | | | | snRNP, putative/Sm protein, | | |
| | | | | | putative | | |
| 8RDBRH_UP_017_B02_16SEP2003_014.ab1_PBI_0_0_0_82_372\| | gi\|119424723\|gb\|DY010125.1\| | 3.1 | 0.016 | AT1G21190.1 | small nuclear | 263 | |
| sense\|_372_410 | DY010125 | | | | ribonucleoprotein, putative | | |
| Contig1_11872_final_0_0_0_2_409\|sense\|_120_154 | gi\|150902170\|gb\|ES932633.1\| | 3.1 | 0.049 | AT5G56220.1 | nucleoside-triphosphatase/ | 337 | |
| | ES932633 | | | | nucleotide binding | | |
| Contig3_3111_final_0_0_0_56_262\|sense\|_268_306 | gi\|95843033\|gb\|DY017359.1\| | 3.1 | 0.016 | AT2G43460.1 | 60S ribosomal protein L38 | 266 | T |
| | DY017359 | | | | (RPL38A) | | |
| Contig1_3071_final_0_0_0_9_761\|sense\|_628_662 | gi\|125931919\|gb\|EL588692.1\| | 3.1 | 0.034 | AT5G24840.1 | methyltransferase | 241 | |
| | EL588692 | | | | | | |
| BNZB_UP_034_D10_10MAY2004_074.ab1_PBI\| | gi\|150153496\|gb\|EE558749.1\| | 3.0 | 0.039 | AT4G39520.1 | GTP-binding protein, putative | 255 | |
| senAnsen\|_289_326 | EE558749 | | | | | | |
| 49RDOAT_UP_009_F11_28SEP2004_085.ab1_PBI_0_0_0_3_302\| | gi\|150133463\|gb\|EE534426.1\| | 3.0 | 0.05 | AT3G62300.1 | agenet domain-containing | 338 | |
| sense\|_198_233 | EE534426 | | | AT3G62300.2 | protein | | |
| Contig5_10552_final_0_0_0_421_1149\| | gi\|95829402\|gb\|DY005324.1\| | 3.0 | 0.033 | AT5G05000.1 | ATTOC34/OEP34 | 339 | M |
| sense\|_614_649 | DY005324 | | | AT5G05000.2 | (Translocase of chloroplast 34) | | |
| | | | | AT5G05000.3 | | | |
| gi_113705911_NCBI_0_0_0_1_177\| | gi\|32504918\|gb\|CD822978.1\| | 3.0 | 0.036 | AT1G32750.1 | HAF01 (HISTONE | 340 | |
| sense\|_2_36 | CD822978 | | | | ACETYLTRANSFERASE OF | | |
| | | | | | THE TAFII250 FAMILY 1) | | |
| gi_54420692_NCBI_0_0_0_3_365\| | gi\|50885538\|gb\|CO749674.1\| | 2.9 | 0.049 | AT2G46020.1 | ATBRM/BRM/CHR2 | 341 | |
| sense\|_348_383 | CO749674 | | | AT2G46020.2 | (ARABIDOPSIS THALIANA | | |
| | | | | | BRAHMA) | | |
| Contig1_15045_final_0_0_0_79_426\| | gi\|95853955\|gb\|DY026350.1\| | 2.9 | 0.012 | AT3G52090.1 | ATRPB13.6 (Arabidopsis | 262 | |
| sense\|_23_57 | DY026350 | | | AT3G52090.2 | thaliana RNA polymerase II | | |
| | | | | | 13.6 kDa subunit) | | |
| Contig563_4314_final_0_0_0_55_672\| | gi\|20374824\|gb\|BG543844.1\| | 2.8 | 0.023 | AT3G49010.1 | ATBBC1 (breast basic | 269 | T |
| sense\|_242_276 | BG543844 | | | AT3G49010.2 | conserved 1); structural | | |
| | | | | AT3G49010.3 | constituent of ribosome | | |
| | | | | AT3G49010.4 | | | |
| | | | | AT3G49010.5 | | | |
| DL2930R_AAFC_0_0_0_8_211\|sense\|_249_287 | gi\|151015701\|gb\|EV029515.1\| | 2.7 | 0.047 | AT3G48000.1 | ALDH2B4 (ALDEHYDE | 218 | |
| | EV029515 | | | | DEHYDROGENASE 2) | | |
| Contig1_4945_final_0_0_0_42_314\|sense\|_25_59 | gi\|126493362\|gb\|EE453044.1\| | 2.7 | 0.047 | AT5G25570.1 | expressed protein | 261 | |
| | EE453044 | | | AT5G25570.2 | | | |
| | | | | AT5G25570.3 | | | |
| Contig2_9037_final_0_0_0_5_442\|sense\|_7_41 | gi\|125928006\|gb\|EL586853.1\| | 2.7 | 0.038 | AT4G02030.1 | expressed protein | 342 | |
| | EL586853 | | | AT4G02030.2 | | | |
| BNAEN3GH_UP_180_A07_25NOV2006_063.ab1_PBI\| | gi\|150995574\|gb\|EV009390.1\| | 2.3 | 0.041 | AT5G20920.1 | EIF2 BETA (EMBRYO | 343 | T |
| senAnsen\|_244_283 | EV009390 | | | AT5G20920.2 | DEFECTIVE 1401) | | |
| | | | | AT5G20920.3 | | | |
| Contig2_1040_final_0_0_0_108_494\| | gi\|32494330\|gb\|CD812390.1\| | 2.3 | 0.039 | AT5G56940.1 | ribosomal protein S16 family | 344 | T |
| sense\|_536_570 | CD812390 | | | | protein | | |
| Contig2_3250_final_0_0_0_104_364\| | gi\|32505964\|gb\|CD824024.1\| | 2.2 | 0.043 | AT5G61220.1 | complex 1 family protein/LVR | 345 | |
| sense\|_448_484 | CD824024 | | | | family protein | | |
| 58ACPE48_UP_012_F08_21SEP2004_054.ab1_PBI_0_0_0_1_435\| | gi\|150055143\|gb\|EE417997.1\| | 2.2 | 0.048 | AT5G58410.1 | binding/protein binding/zinc | 270 | |
| sense\|_45_79 | EE417997 | | | | ion binding | | |
| Contig5_3111_final_0_0_0_56_262\|sense\|_92_127 | gi\|150073135\|gb\|EE435874.1\| | 2.1 | 0.043 | AT2G43460.1 | 60S ribosomal protein L38 | 267 | T |
| | EE435874 | | | | (RPL38A) | | |

The B. napus Probe Id, as present on the Combimatrix 90k microarray, is depicted in column 1.
Column 2 is the gene name database reference. Column 3 depicts the fold change (FC) in expression vs. the control line 115.
Column 4 indicates the Q-values.
Column 5 is the likely Arabidopsis thaliana homologue (the AGI codes are shown).
Column 6 describes the gene based on homology with other proteins found in nucleotide databases.
Column 8 indicates proteins with mitochondrial (M) or translational (T) function.

TABLE 28
Chloroplast network: FC ≧ 2 (112vs115) and Pearson correlation coefficient >0.8.

| | | FC (110 | Q. | | | Seq ID | |
|---|---|---|---|---|---|---|---|
| Probe Id | Name | vs. 115) | value | AGI | annotation | No | function |
| Contig1_86029_remaining_0_0_0_423_857\| | gi\|122026038\|gb\|EH414903.1\| | 65.1 | 4.00E−04 | AT2G24270.1 | ALDH11A3 (Aldehyde | 272 | |
| sense\|_383_417 | EH414903 | | | AT2G24270.2 | dehydrogenase 11A3) | | |
| | | | | AT2G24270.3 | | | |
| | | | | AT2G24270.4 | | | |
| 39RDBRT_UP_083_H09_24JAN2006_065.ab1_PBI\| | gi\|150906031\|gb\|ES936489.1\| | 31.0 | 3.00E−04 | AT3G53900.2 | uracil | 274 | C |
| senAnsen\|_40_74 | ES936489 | | | | phosphoribosyltransferase, | | |
| | | | | | putative | | |
| Contig1_46741_remaining_0_0_0_70_612\| | gi\|95839565\|gb\|DY013041.1\| | 27.0 | 0.012 | AT2G39290.1 | PGP1/PGPS1/PGS1 | 273 | |
| sense\|_273_307 | DY013041 | | | | (PHOSPHATIDYLGLYCEROL | | |
| | | | | | PHOSPHATE SYNTHASE 1) | | |
| BNother_UP_037_O20_10DEC2003_031.ab1_GHI1_0_0_0_8_121\| | gi\|150112715\|gb\|EE513687.1\| | 22.5 | 0.002 | AT3G13750.1 | BGAL1 (BETA | 277 | |
| sense\|_9_43 | EE513687 | | | | GALACTOSIDASE 1) | | |
| 63JKCOT5_T3_002_H03_04JAN2005_017.ab1_PBI_0_0_0_48_851\| | gi\|95843146\|gb\|DY017404.1\| | 18.7 | 0.002 | AT5G01090.1 | legume lectin family protein | 275 | |
| sense\|_657_691 | DY017404 | | | | | | |
| BNYS2DCT_UP_033_D04_03MAR2005_026.ab1_PBI_0_0_0_18_212\| | gi\|126486472\|gb\|EE475971.1\| | 15.9 | 7.00E−04 | AT2G30570.1 | PSBW (PHOTOSYSTEM II | 279 | C |
| sense\|_245_279 | EE475971 | | | | REACTION CENTER W) | | |
| Contig2_1157_final_0_0_0_47_979\|sense\|_122_159 | gi\|151288312\|gb\|EV194973.1\| | 15.6 | 3.00E−04 | AT1G72640.1 | binding/catalytic | 276 | C |
| | EV194973 | | | AT1G72640.2 | | | |
| gi_1048276_NCBI\|senAnsen\|_77_111 | gi\|1048276\|gb\|H74983.1\| | 13.0 | 9.00E−04 | AT1G11410.1 | S-locus protein kinase, | 278 | |
| | H74983 | | | | putative | | |
| gi_72287793_NCBI_0_0_0_147_263\| | gi\|72287793\|gb\|AM061028.1\| | 13.0 | 0.005 | AT1G02280.1 | TOC33 (PLASTID PROTEIN | 280 | C |
| sense\|_93_127 | AM061028 | | | AT1G02280.2 | IMPORT 1) | | |
| Contig1_2300_final_0_0_0_300_515\| | gi\|122028216\|gb\|EH417081.1\| | 12.9 | 7.00E−04 | AT4G02920.1 | expressed protein | 283 | |
| sense\|_87_122 | EH417081 | | | AT4G02920.2 | | | |
| 9RDBNGA_UP_176_D11_11MAR2006_089.ab1_PBI_0_0_0_344_475\| | gi\|32502702\|gb\|CD820762.1\| | 12.3 | 0.007 | AT5G28750.1 | thylakoid assembly protein, | 284 | C |
| sense\|_368_404 | CD820762 | | | | putative | | |
| Contig4_9061_final_0_0_0_7_366\|sense\|_226_260 | gi\|56839524\|gb\|CX192100.1\| | 11.8 | 0.009 | AT5G08000.1 | E13L3 (GLUCAN ENDO-1,3- | 289 | |
| | CX192100 | | | | BETA-GLUCOSIDASE-LIKE | | |
| | | | | | PROTEIN 3) | | |
| BNZB_UP_077_A02_02JUN2004_016.ab1_PBI\| | gi\|150155330\|gb\|EE561957.1\| | 11.5 | 0.027 | AT1G50170.1 | ATSIRB; sirohydrochlorin | 291 | |
| senAnsen\|_267_302 | EE561957 | | | | ferrochelatase | | |
| Contig2_938_final_0_0_0_33_1028\|sense\|_593_627 | gi\|126475471\|gb\|EE458095.1\| | 11.1 | 2.00E−04 | AT2G43950.1 | OEP37; ion channel | 286 | C |
| | EE458095 | | | AT2G43950.2 | | | |
| | | | | AT2G43950.3 | | | |
| Contig1_74342_remaining_0_0_0_2_931\| | gi\|65297168\|gb\|CN827382.1\| | 10.0 | 0.002 | AT3G15570.1 | phototropic-responsive NPH3 | 288 | |
| sense\|_275_309 | CN827382 | | | | family protein | | |
| UC459R_AAFC_0_0_0_138_635\|sense\|_431_465 | gi\|151277941\|gb\|EV188300.1\| | 9.6 | 8.00E−04 | AT3G19810.1 | expressed protein | 292 | C |
| | EV188300 | | | | | | |
| BNARO5GH_T3_014_H06_30NOV2006_034.ab1_PBI_0_0_0_101_892\| | gi\|150876088\|gb\|ES906550.1\| | 9.3 | 0.004 | AT2G39930.1 | ATISA1/ISA1 (ISOAMYLASE | 285 | |
| sense\|_825_859 | ES906550 | | | | 1) | | |
| gi_75974640_NCBI_0_0_0_11_637\| | gi\|75974640\|gb\|AM060652.1\| | 8.6 | 0.033 | AT1G54040.1 | ESP (EPITHIOSPECIFIER | 298 | |
| sense\|_341_375 | AM060652 | | | AT1G54040.2 | PROTEIN) | | |
| Contig1_958_final_0_0_0_96_698\|sense\|_961_996 | gi\|150881473\|gb\|ES911934.1\| | 7.5 | 0.044 | AT5G62350.1 | invertase/pectin | 346 | |
| | ES911934 | | | | methylesterase inhibitor family | | |
| | | | | | protein/DC 1.2 homolog | | |
| | | | | | (FL5-2I22) | | |
| 73ETGS36_UP_019_B03_21MAY2005_029.ab1_PBI_0_0_0_3_431\| | gi\|150083518\|gb\|EE446257.1\| | 7.4 | 0.036 | AT1G18485.1 | pentatricopeptide (PPR) | 347 | |
| sense\|_297_331 | EE446257 | | | | repeat-containing protein | | |
| Contig4_1978_final_0_0_0_61_1263\| | gi\|151294706\|gb\|EV201369.1\| | 7.0 | 0.004 | AT4G12730.1 | FLA2 | 295 | |
| sense\|_1195_1229 | EV201369 | | | | | | |
| CD24F_AAFC\|senAnsen\|_118_157 | gi\|151283976\|gb\|EV190637.1\| | 6.9 | 0.041 | AT5G01410.1 | PDX1 (PYRIDOXINE | 287 | |
| | EV190637 | | | | BIOSYNTHESIS 1.3) | | |
| Contig2_519_final_0_0_0_123_431\|sense\|_416_455 | gi\|32504311\|gb\|CD822371.1\| | 6.9 | 0.02 | AT2G37240.1 | antioxidant/oxidoreductase | 300 | C |
| | CD822371 | | | | | | |
| Contig1_18049_final_0_0_0_14_424\| | gi\|126473095\|gb\|EE454234.1\| | 6.8 | 0.008 | AT2G33810.1 | SPL3 (SQUAMOSA | 294 | |
| sense\|_246_281 | EE454234 | | | | PROMOTER BINDING | | |
| | | | | | PROTEIN-LIKE 3) | | |
| JKBNHS1_UP_015_C04_02JUL2004_028.ab1_PBI_0_0_0_33_575\| | gi\|83833659\|gb\|CX281882.1\| | 6.2 | 0.038 | AT1G10522.1 | expressed protein | 297 | C |
| sense\|_242_276 | CX281882 | | | AT1G10522.2 | | | |
| BNShoot_UP_130_K05_10DEC2003_078.ab1_GHI1_0_0_0_3_242\| | gi\|56838609\|gb\|CX191185.1\| | 6.1 | 1.00E−03 | AT5G64040.1 | PSAN (photosystem I reaction | 302 | C |
| sense\|_84_118 | CX191185 | | | AT5G64040.2 | center subunit PSI-N) | | |
| Contig1_70154_remaining_0_0_0_1_861\| | gi\|56840647\|gb\|CX193223.1\| | 5.9 | 0.037 | AT3G28040.1 | leucine-rich repeat | 348 | |
| sense\|_689_723 | CX193223 | | | | transmembrane protein | | |
| | | | | | kinase, putative | | |
| 47RDOAH_UP_037_D11_17AUG2004_089.ab1_PBI_0_0_0_2_478\| | gi\|95860699\|gb\|DY029073.1\| | 5.8 | 0.002 | AT3G25860.1 | LTA2 (PLASTID E2 SUBUNIT | 301 | C |
| sense\|_432_466 | DY029073 | | | | OF PYRUVATE | | |
| | | | | | DECARBOXYLASE) | | |
| Contig1_9466_final_0_0_0_61_1452\| | gi\|112354580\|gb\|AM390360.1\| | 5.6 | 0.019 | AT5G42390.1 | metalloendopeptidase | 303 | C |
| sense\|_1030_1064 | AM390360 | | | | | | |
| Contig3_9211_final_0_0_0_90_632\|sense\|_522_557 | gi\|122030955\|gb\|EH419820.1\| | 5.4 | 0.02 | AT5G62790.1 | DXR (1-DEOXY-D- | 299 | C |
| | EH419820 | | | AT5G62790.2 | XYLULOSE 5-PHOSPHATE | | |
| | | | | | REDUCTOISOMERASE) | | |
| Contig1_17419_final_0_0_0_638_874\| | gi\|151182351\|gb\|EV095859.1\| | 5.3 | 0.002 | AT5G57345.1 | expressed protein | 307 | |
| sense\|_336_370 | EV095859 | | | | | | |
| gi_56835891_NCBI_0_0_0_1_267\| | gi\|56835891\|gb\|CX188467.1\| | 5.2 | 0.026 | AT4G27440.1 | PORB | 308 | C |
| sense\|_236_273 | CX188467 | | | AT4G27440.2 | (PROTOCHLOROPHYLLIDE | | |
| | | | | | OXIDOREDUCTASE B) | | |
| OL33_36R_J10_OL3229R_065.ab1_AAFC_0 _0_0_3_638\| | gi\|122029360\|gb\|EH418225.1\| | 4.6 | 0.019 | AT3G15520.1 | peptidyl-prolyl cis-trans | 306 | C |
| sense\|_57_91 | EH418225 | | | | isomerase TLP38, chloroplast | | |
| Contig1_2768_final_0_0_0_425_640\| | gi\|150041563\|gb\|EE404438.1\| | 4.6 | 0.009 | AT5G50110.1 | methyltransferase-related | 318 | |
| sense\|_9_43 | EE404438 | | | | | | |
| 37RDBRH_UP_004_C03_26MAR2004_027.ab1_PBI_0_0_0_4_573\| | gi\|95828875\|gb\|DW999350.1\| | 4.1 | 0.003 | AT4G21850.2 | MSRB2 (METHIONINE | 309 | C |
| sense\|_132_166 | DW999350 | | | AT4G21860.1 | SULFOXIDE REDUCTASE B | | |
| | | | | AT4G21860.2 | 2) | | |
| | | | | AT4G21860.3 | | | |
| BNAEN3GH_UP_109_E02_15SEP2006_008.ab1_PBI\| | gi\|150986716\|gb\|EV000534.1\| | 3.9 | 0.034 | AT5G35630.3 | GS2 (GLUTAMINE | 311 | C |
| senAnsen\|_169_208 | EV000534 | | | AT5G35630.2 | SYNTHETASE 2) | | |
| | | | | AT5G35630.1 | | | |
| Contig8_4093_final_0_0_0_2_334\|sense\|_307_345 | gi\|151311021\|gb\|EV211058.1\| | 3.8 | 0.011 | AT4G36250.1 | ALDH3F1 (ALDEHYDE | 315 | |
| | EV211058 | | | | DEHYDROGENASE 3F1) | | |
| Contig2_5769_final_0_0_0_2_226\|sense\|_118_152 | gi\|29690060\|gb\|CB686335.1\| | 3.7 | 0.032 | AT2G33380.1 | RD20 (RESPONSIVE TO | 305 | |
| | CB686335 | | | AT2G33380.2 | DESSICATION 20) | | |
| BNZB_UP_113_D08_19AUG2004_058.ab1_PBI\| | gi\|151291352\|gb\|EV198013.1\| | 3.4 | 0.012 | AT1G30380.1 | PSAK (PHOTOSYSTEM I | 313 | C |
| senAnsen\|_25_59 | EV198013 | | | | SUBUNIT K) | | |
| 37RDBRH_UP_068_D09_05DEC2005_073.ab1_PBI\| | gi\|150126159\|gb\|EE527131.1\| | 3.3 | 0.035 | AT2G42590.1 | GRF9 (GENERAL | 312 | |
| senAnsen\|_98_132 | EE527131 | | | AT2G42590.2 | REGULATORY FACTOR 9) | | |
| | | | | AT2G42590.3 | | | |
| Contig1_15742_final_0_0_0_1_525\|sense\|_128_163 | gi\|119430585\|gb\|DY023779.1\| | 3.3 | 0.038 | AT1G17840.1 | ABCG11/COF1/DSO/WBC11 | 349 | |
| | DY023779 | | | | (DESPERADO); ATPase, | | |
| | | | | | coupled to transmembrane | | |
| | | | | | movement of substances | | |
| Contig1_4928_remaining_0_0_0_3_6_02\| | gi\|65299729\|gb\|CN829943.1\| | 3.1 | 0.021 | AT1G12860.1 | basic helix-loop-helix (bHLH) | 319 | |
| sense\|_257_291 | CN829943 | | | | family protein | | |
| BNShoot_UP_125_J24_10DEC2003_087.ab1_GHI1_0_0_0_26_301\| | gi\|65287110\|gb\|CN729306.1\| | 2.8 | 0.02 | AT1G20340.1 | DRT112 (DNA-damage- | 321 | C |
| sense\|_259_293 | CN729306 | | | | repair/toleration protein 112) | | |
| BL145F_AAFC\|senAnsen\|_8_45 | gi\|65284741\|gb\|CN726939.1\| | 2.8 | 0.043 | AT1G78370.1 | ATGSTU20 (Arabidopsis | 350 | |
| | CN726939 | | | | thaliana Glutathione S- | | |
| | | | | | transferase (class tau) 20); | | |
| | | | | | glutathione transferase | | |
| Contig2_6482_final_0_0_0_67_708\|sense\|_522_556 | gi\|150058996\|gb\|EE421742.1\| | 2.8 | 0.042 | AT3G09250.1 | DNA binding/nuclease | 351 | |
| | EE421742 | | | AT3G09250.2 | | | |
| Contig1_2745_final_0_0_0_86_799\|sense\|_278_312 | gi\|151311097\|gb\|EV211134.1\| | 2.6 | 0.039 | AT4G00050.1 | UNE10 (unfertilized embryosac | 322 | |
| | EV211134 | | | | 10) | | |
| Contig1_10484_final_0_0_0_433_1281\| | gi\|150878759\|gb\|ES909217.1\| | 2.6 | 0.036 | AT2G47160.1 | BOR1 (REQUIRES HIGH | 352 | |
| sense\|_1254_1288 | ES909217 | | | AT2G47160.2 | BORON 1) | | |
| gi_21844485_NCBI_0_0_0_2_454\| | gi\|21844485\|gb\|BQ705066.1\| | 2.4 | 0.041 | AT3G22150.1 | pentatricopeptide (PPR) | 324 | C |
| sense\|_17_51 | BQ705066 | | | | repeat-containing protein | | |
| EX20LIB3_UP_008_H12_04FEB2004_082.ab1_PBI_0_0_0_1_309\| | gi\|32503461\|gb\|CD821521.1\| | 2.4 | 0.049 | AT3G21055.1 | PSBTN (photosystem II | 353 | C |
| sense\|_96_130 | CD821521 | | | | subunit T) | | |

The B. napus Probe Id, as present on the Combimatrix 90k microarray, is depicted in column 1.
Column 2 is the gene name database reference.
Column 3 depicts the fold change (FC) in expression vs. the control line 115.
Column 4 indicates the Q-values. Column 5 is the likely Arabidopsis thaliana homologue (the AGI codes are shown).
Column 6 describes the gene based on homology with other proteins found in nucleotide databases.
Column 8 indicates proteins with mitochondrial (M) or translational (T) function.

1. A method for the production of a plant with a high energy use efficiency comprising the steps of:
i) providing a population of plants of the same plant species,
ii) obtaining a nucleic acid sample from said plants,
iii) determining a gene expression profile by quantifying the mRNA presence of:
a. at least two genes comprising a nucleotide sequence having 70%-100% nucleic acid identity to any one of the nucleotide sequences of Seq ID No 1-61; and/or
b. at least two genes comprising a nucleotide sequence having 70%-100% nucleic acid identity to any one of the nucleotide sequences of Seq ID No 62-134; and/or
c. at least two genes comprising a nucleotide sequence having 70-100% nucleic acid identity to any one of the nucleotide sequences of Seq ID No 147-353;
iv) identifying at least one plant from said population having an at least increased 1.5 fold mRNA presence of said at least two genes comprising a nucleotide sequence having 70%-100% nucleic acid identity to any one of said nucleotide sequences of Seq ID No 62-134 with respect to the average mRNA presence of said genes in said population and/or having an at least decreased 0.66 fold mRNA presence of said at least two genes comprising a nucleotide sequence having 70%-100% nucleic acid identity to any one of said nucleotide sequences of Seq ID No 1-61 with respect to the average mRNA presence of said genes in said population and/or having an at least 2.0 fold mRNA presence of said at least two genes comprising a nucleotide sequence having 70%-100% nucleic acid identity to any one of said nucleotide sequences of Seq ID No 147-353 with respect to the average mRNA presence of said genes in said population.

2. The method of claim 1 wherein said population of plants are genetically identical.

3. The method of claim 1 wherein said population of plants are doubled haploid plants.

4. The method of claim 1 wherein said population of plants are produced by vegetative reproduction.

5. The method of claim 1 wherein said population of plants are inbred plants.

6. The method according to claim 1 wherein said produced plant is used to create further propagating material.

7. The method of claim 6 wherein said produced plant and said other plant are inbred plants.

8. The method according to claim 1 wherein said plant having a high energy use efficiency is a Brassica oilseed rape, tomato, rice, wheat, cotton, corn or soybean plant.

9. The method according to claim 1 wherein said quantification of the mRNA expression level is determined by microarray analysis.

10. The method according to claim 1 wherein said quantification of the mRNA expression level is determined by RT-PCR.

11. A method for producing a population of plants or seeds with a high energy use efficiency comprising selecting a population of plants according to claim 1.

12. A method for increasing harvest yield comprising the steps of producing a population of plants or seeds according to claim 11, growing said plants or seeds in a field and producing a harvest from said plants or seeds.

13. A method for producing a hybrid plant or hybrid seed with high energy use efficiency comprising selecting a population of plants with high energy use efficiency according to claim 11 for at least one parent inbred plant, interplanting plants of said population with another inbred plant, isolating hybrid seed resulting from said interplanting, and optionally, grow hybrid plants from said seed.

14. The method according to claim 13, wherein a population of plants with high energy use efficiency is selected for both parent inbred plants.

15. The method according to claim 13, wherein said one parent plant is a male sterile plant and maintaining said male sterile plant requires the use of a maintainer line further characterized in that a population of plants with high energy use efficiency according to claim 11 is also selected for the maintainer line.

16. A kit comprising the necessary tools for carrying out the method of claim 1.

17. A method for obtaining a biological or chemical compound which is capable of generating a plant with high energy use efficiency comprising the steps of:
i) providing a population of plants of the same plant species,
ii) treating a subset of said population of plants with a biological or chemical compound,
iii) obtaining a nucleic acid sample from said plants,
iv) determining a gene expression profile by quantifying the mRNA presence of:
a. at least two genes comprising a nucleotide sequence having 70%-100% nucleic acid identity to any one of the nucleotide sequences of Seq ID No 1-61; and/or
b. at least two genes comprising a nucleotide sequence having 70%-100% nucleic acid identity to any one of the nucleotide sequences of Seq ID No 62-134; and/or
c. at least two genes comprising a nucleotide sequence having 70-100% nucleic acid identity to any one of the nucleotide sequences of Seq ID No 147-353.
iv) selecting a compound which results in an at least increased 1.5 fold mRNA presence of said at least two genes comprising a nucleotide sequence having 70%-100% nucleic acid identity to any one of said nucleotide sequences of Seq ID No 62-134 in a plant from said population with respect to the untreated plants in said population and/or which result in an at least decreased 0.66 fold mRNA presence of said at least two genes comprising a nucleotide sequence having 70%-100% nucleic acid identity to any one of said nucleotide sequences of Seq ID No 1-61 in said plant from said population with respect to untreated plants of said population and/or which result in an at least 2.0 fold mRNA presence of said at least two genes comprising a nucleotide sequence having 70%-100% nucleic acid identity to any one of said nucleotide sequences of Seq ID No 147-353 in said plant from said population with respect to untreated plants of said population.

18. A gene expression profile indicative for high energy use efficiency comprising the expression level of at least two genes comprising a nucleotide sequence having 70%-100% nucleic acid identity to any one of the nucleotide sequences of Seq ID No 1-61 and/or at least two genes comprising a nucleotide sequence having 70%-100% nucleic acid identity to any one of the nucleotide sequences of Seq ID No 62-134 and/or at least two genes comprising a nucleotide sequence having 70-100% nucleic acid identity to any one of the nucleotide sequences of Seq ID No 147-353.

19. Use of the gene expression profile of claim 18 in the method of claim 1.

20. Use of the gene expression profile of claim 18 in the method of claim 17.
